(19) United States
(12) Patent Application Publication
(10) Pub. No.: US 2023/0108685 A1
(43) Pub. Date: Apr. 6, 2023

(54) Biomarkers For Endometriosis

(71) Applicant: Queen Mary University of London, London (GB)

(72) Inventors: Finbar Cotter, London (GB); Christine Deguara, London (GB); Colin Davis, London (GB)

(21) Appl. No.: 17/720,503

(22) Filed: Apr. 14, 2022

Related U.S. Application Data
(63) Continuation of application No. 16/874,831, filed on May 15, 2020, now abandoned.
Application No. 16/874,831 is a continuation of application No. 16/132,844, filed on Sep. 17, 2018, now abandoned.
Application No. 16/132,844 is a continuation of application No. 15/121,861, filed on Aug. 26, 2016, now abandoned.

(30) Foreign Application Priority Data
Feb. 27, 2014 (GB) 1403489.6

(51) Int. Cl. C12Q 1/6883; C12Q 1/6886; G01N 33/564; G01N 33/68

(52) U.S. Cl. CPC C12Q 1/6883; C12Q 1/6886; G01N 33/564; G01N 33/6893; C12Q 2600/158; C12Q 2600/178; G01N 2800/364

(57) ABSTRACT

The presence of certain auto antibodies and miRNAs indicates that a subject has endometriosis. The auto-antibodies recognise antigens listed in Table 1. The miRNAs are also listed in Table 1.

### TECHNICAL FIELD

This Invention relates to biomarkers useful for the diagnosis and/or prognosis of endometriosis. The biomarkers ae also useful for monitoring treatment of the disease and can also be used as targets for therapeutic intervention.

### BACKGROUND

Endometriosis Is a common gynaecological condition in which uterine or uterine-Ike endometrial and/or stromal cells are found exterior to the uterus in various sites and tissues of the body. These sites are numerous and can vary from pelvic infiltrations (superficial or deeply infiltrating) to ovarian cysts (also known as endometriomas), vaginal, vulval, bowel and bladder infiltrations, skin and scar lesions and lesions in the lymphatic system, brain and lungs (though these ae rarer). It can potentially be found in any site of the body and presents with non-specific signs and symptoms. Symptoms of endometriosis vary widely and can range from pain, e.g. pelvic pain, dysmenorrhea, dyspareunia, dyschesia, dysuria, dyschesia, fatigue and infertility causing numerous physical and psychosocial co-morbidities and severely limiting the qualify of patient's life [1]. Endometriosis is a chronic and possibly hormonally responsive condition which recurs, potentially leaving scars and fibrosis in the tissue it affects.

Endometriosis is found almost exclusively in women, generally in their reproductive years. The average age at diagnosis is 25-29 years, but there is an estimated delay in diagnosis of disease quoted at around 6.7 years in one multicentre trial [2]. US studies report an average delay in diagnosis of 11.7 years and UK studies estimate 8 years of diagnostic delay [3]. The disease in itself affects around 5%-15% of women in their reproductive age varying in prevalence between populations. It is estimated to affect over 70 million women worldwide 14). Diagnostic incidence rates have been quoted in 28.4% (95% Cl: 20-32.7%) of African to 54.5% (95% Cl: 44.2-64.7%) in South American countries [5]. Endometriosis is prevalent in 0.5-5% of fertile women and 25-40% in infertile women making it a leading cause of infertility [6,7]. In one Norwegian study the lifetime prevalence was documented at 2.2% 181.

There are no defined causative factors but risk factors associated with disease vary, and suggestions include age (rare before and after menarche and menopause respectively), social class and race [9](Increased incidence in higher social classes). In utero exposure to multiple pregnancies and diethylstilbestrol [10], infertility [11], low parity [11], oral contraceptive use and age of commencement or stopping of oral contraceptive pill [12], family history [13,14], smoking [15], diet [7], exercise [16], body mass index [17], dioxin [18], a history of immune disorders [19], association with non-Hodgkin's lymphoma [20], association with pigmentary traits [21], links with melanoma [22] and association with ovarian (serous, mucinous, endometroid, clear cell, other subtypes) and endometrial cancer have at been published. Most studies describing links to diet, exercise and familial inheritance show conflicting evidence and are by no means definitive.

Theories of causation vary with literature supporting or refuting each respectively. Theories include those of: in situ development, mullerianosis [23], accentuation by genital tract anomalies [24], genetic predisposition [25], coelomic metaplasia [26], induction [27], transplantation [28], retrograde menstruation [29], endometrial stem cells [30], physiological phenomenon [31], alterations in the endometrium [32], exogenous endometrial hormonal production [33], angiogenesis [34], the evasion of endometrial tissue om the immune system [35], cellular protection from apoptosis [36,37], the potential of endometrium to implant [38] and invade [39], and differences in peritoneal fluid [40].

The diagnosis of endometriosis is a combination of physician vigilance and knowledge, patient history, clinical symptoms/signs at examination and use of radiology (Including trans-vaginal and trans-abdominal ultrasound to assess for endometriomas or macroscopic lesions). MRI and CT scanning are used for deep lesions and the most utilised serum marker is the non-specific CA125 which has poor correlation with presence or severity of disease. Biomarkers reported in literature range from the use of cytokines, non-cytokines, serum and endometrial biomarkers, the presence of nerve fibres within tissues [41], gene aberrations and mRNAs. However, none of them is currently used and validated in the clinical setting.

There is the postulated use of proteomic analysis of endometrium and patient blood using 2-DIGE and SELDI-TOF MS technology [42, 43, 44, 45]. No biomarkers have so far been identified from this. Peripheral blood studies using SELDI TOF MS report proteins altered in endometriosis [46, 47, 48, 49, 50], but none of them provides sufficient sensitivity and specificity for use in clinical settings.

The gold standard for diagnosis and treatment of endometrial peritoneal lesions is invasive surgical laparoscopy enabling the visualisation of lesions. Endometriosis is removed by diathermy or/and peritoneal and/or cyst stripping. This has substantial patient morbidly and possibly mortality.

An early non-invasive test would enable easier diagnosis and treatment, prevent chronic effects of disease that include pain, scarring, psychological trauma and infertility; reduce surgical patient morbidity and mortality and enable the monitoring of the response of disease to therapeutics and management.

There is a need for new or improved in vitro tests with good specificity and sensitivity to enable non-invasive diagnosis of endometriosis. It is an object of the invention to provide further and improved biomarkers for the diagnosis of endometriosis e.g. using in vitro detection techniques.

### DISCLOSURE OF THE INVENTION

The invention is based on the identification of correlations between endometriosis and the increased or decreased levels of certain proteins and small non-coding miRNAs.

The inventors have identified miRNAs for which the expression profiles can be used to indicate that a subject has endometriosis. These miRNAs are present at significantly different levels in subjects with endometriosis and without endometriosis. Detection of the presence or absence of these miRNAs. and/or of changes in their levels over time can thus be used to indicate that a subject has endometriosis, or has the potential to develop endometriosis in the future. These miRNAs function as biomarkers of endometriosis.

The Inventors have also identified antigens for which the level of auto-antibodies can be used to indicate that a subject has endometriosis. Auto-antibodies against these antigens are present at significantly different levels in subjects with endometriosis and without endometriosis. Detection of the presence or absence of these auto-antibodies, and/or of changes in their levels over time, can thus be used to indicate that a subject has endometriosis. The auto-antibodies and their antigens also function as biomarkers of endometriosis.

Detection of these biomarkers in a subject sample can be used to improve the diagnosis, prognosis and monitoring of endometriosis. Advantageously, the invention can be used to distinguish between endometriosis and other forms of intra-abdominal inflammation.

The inventors have identified 343 such biomarkers (Table 1) and the invention uses at least one of these to assist in the diagnosis of endometriosis by measuring level(s) of the biomarker(s). The biomarker can be a protein or a mRNA. A protein biomarker can be (i) auto-antibody which binds to an antigen in Table 1 and/or (ii) an antigen in Table 1, but is preferably the former.

Thus the invention provides a method for analysing a subject sample, comprising a step of determining the level of a Table 1 biomarker in the sample, wherein the level of the biomarker provides a diagnostic indicator of whether the subject has endometriosis.

Analysis of a single Table 1 biomarker can be performed, and detection of the auto-antibody/antigen or mRNA can provide a useful diagnostic indicator for endometriosis even without considering any of the other Table 1 biomarkers.

The sensitivity and specificity of diagnosis can be improved, however, by combining data for multiple biomarkers. It is thus preferred to analyse more than one Table 1 biomarker. Analysis of two or more different biomarkers (a “pane”) can enhance the sensitivity and/or specificity of diagnosis compared to analysis of a single biomarker. Each different biomarker in a panel is shown in a different row in Table 1, e.g. measuring both auto-antibody which binds to an antigen listed in Table 1 and the antigen itself is measurement of a single biomarker rather than of a panel.

The inventors found that the combination of the information from protein and mRNA biomarkers provides an enhancement in diagnostic utility, as measured by sensitivity, specificity and/or area under the Receiver Operating Characteristic (ROC) curve. Thus, the invention preferably uses at least one protein biomarker from Table 1 and at least one mRNA biomarker from Table 1 to assist in the diagnosis of endometriosis.

Thus the invention provides a method for analysing a subject sample, comprising a step of determining the levels of x different biomarkers of Table 1, wherein the levels of the biomarkers provide a diagnostic indicator of whether the subject has endometriosis. The value of x is 2 or more e.g. 2, 3, 4, 5, 6, 7, 8, 9, 10, 11, 12, 13, 14, 15 or more (e.g. up to 343). These panels may include (I) any specific one of the 343 biomarkers in Table 1 in combination with (i) any of the other 342 biomarkers in Table 1. Preferably the panel includes at least one protein biomarker (e.g. auto-antibodies or an antigen) from Table 1 and at least one miRNA biomarker from Table 1. Preferably the panel includes protein biomarkers only (e.g. auto-antibodies or antigens) from Table 1. Preferably the panel includes mRNA biomarkers only from Table 1.

Suitable panels are described below and panels of particular interest include those listed in Tables 11 to 15. Preferred panels have from 2 to 6 biomarkers, as using >6 of them adds little to sensitivity and specificity.

The Table 1 biomarkers can be used in combination with one or more of: (a) known biomarkers for endometriosis, which may be auto-antibodies, antigens or miRNAs; and/or (b) other information about the subject from whom a sample was taken e.g. age, genotype (genetic variations can affect auto-antibody profiles [51] and considerable progress on the elucidation of the genetics of endometriosis has been made [52]), weight, other clinically-relevant data or phenotypic information; and/or (c) other diagnostic tests or clinical indicators for endometriosis. Such combinations can enhance the sensitivity and/or specificity of diagnosis. Known endometriosis biomarkers of particular interest include, but are not limited to, auto-antibodies against CA125, CA19-9 and/or any of the antigens listed in Table 5.

Thus the invention provides a method for analysing a subject sample, comprising a step of determining:
(a) the level(s) of y Table 1 biomarker(s), wherein the levels of the biomarkers provide a diagnostic indicator of whether the subject has endometriosis; and also one or more of:
(b) If a sample from the subject contains a known biomarker selected from the group consisting of auto-antibodies against antigens such as CA125, CA19-9 and/or any of the antigens listed in Table 5 (and optionally, any other known biomarkers e.g. see above); wherein detection of the known biomarker provides a second diagnostic indicator of whether the subject has endometriosis;
(c) the subject's age and/or gender,
and combining the different diagnostic indicators (and optionally age and/or gender) to provide an aggregate diagnostic indicator of whether the subject has endometriosis.

The samples used in (a) and (b) may be the same or different.

The value of y is 1 or more e.g. 1, 2, 3, 4, 5, 6, 7, 8, 9, 10, 11, 12, 13, 14, 15 (e.g. up to 343). When y>1 the invention uses a panel of different Table 1 biomarkers. When y>1, preferably the panel includes at least one protein biomarker (e.g. auto-antibodies or an antigen) from Table 1 and at least one mRNA biomarker from Table 1.

The invention also provides, in a method for diagnosing if a subject has endometriosis, an improvement consisting of determining in a sample from the subject the level(s) of y biomarker(s) of Table 1, wherein the level(s) of the biomarker(s) provide a diagnostic indicator of whether the subject has endometriosis. The biomarker(s) of Table 1 can be used in combination with known endometriosis biomarkers, as discussed above.

The invention also provides a method for diagnosing a subject as having endometriosis, comprising steps at: (i) determining the levels of y biomarkers of Table 1 in a sample from the subject; and (ii) comparing the determination from step (i) to data obtained from samples from subjects without endometriosis and/or from subjects with endometriosis, wherein the comparison provides a diagnostic indicator of whether the subject has endometriosis. The comparison in step (ii) can use a classifier algorithm as discussed in more detail below. The biomarkers measured in step (i) can be used in combination with known endometriosis biomarkers, as discussed above.

The invention also provides a method for monitoring development of endometriosis in a subject, comprising steps of: (i) determining the levels of z₁ biomarker(s) of Table 1 in a first sample from the subject taken at a first time; and (ii) determining the levels of z₂ biomarker(s) of Table 1 in a second sample from the subject taken at a second time, wherein: (a) the second time is later than the first time; (b) one or more of the z biomarker(s) were present in the first sample; and (c) a change in the level(s) of the biomarker(s) in the second sample compared with the first sample indicates that endometriosis is in remission or is progressing. Thus the method monitors the biomarker(s) over time, with changing levels indicating whether the disease is getting better or worse.

The disease development can be either an improvement or a worsening of the disease, and this method may be used in various ways e.g. to monitor the natural progress of a disease, or to monitor the efficacy of a therapy being administered to the subject. Thus a subject may receive a therapeutic agent or may receive surgery for removing endometriosis before the first time, at the first time, or between the first time and the second time. Increased levels of antibodies against a particular auto-antigen may be due to “epitope spreading”, in which additional antibodies or antibody classes are raised to antigens against which an antibody response has already been mounted [53].

The value of z₂ is 1 or more e.g. 1, 2, 3, 4, 5, 6, 7, 8, 9, 10, 11, 12, 13, 14, 15 (e.g. up to 343). The value of z₂ is 1 or more e.g. 1, 2, 3, 4, 5, 6, 7, 8, 9, 10, 11, 12, 13, 14, 15 (e.g. up to 343). The values of z₁ and z₂ may be the same or different. If they am different, it is usual that z₁>z₂ as the later analysis (z₂) can focus on biomarkers which were already detected in the earlier analysis; in other embodiments, however, z₂ can be larger than z₁, e.g. If previous data have indicated that an expanded panel should be used; in other embodiments z₂=z₁, e.g. so that, for convenience, the same panel can be used for both analyses. When z₁>1 or z₂>1, the biomarkers am different biomarkers. The z₁ and/or z₂ biomarker(s) can be used in combination with known endometriosis biomarkers, as discussed above.

The invention also provides a method for monitoring development of endometriosis in a subject, comprising steps of (i) determining the level of at least w₁ Table 1 biomarkers in a fir sample taken at a first time from the subject; and (I) determining the level of at least w₂ Table 1 biomarkers in a second sample taken at a second time from the subject, wherein: (a) the second time is later than the first time; (b) at least one biomarker is common to both the w₁ and w₂ biomarkers; (c) the level of at least one biomarker common to both the w₁ and w₂ biomarkers is different in the first and second samples, thereby indicating that the endometriosis is progressing or regressing. Thus the method monitors the range of biomarkers over time, with a broadening in the number of detected biomarkers indicating that the disease is getting worse. As mentioned above, this method may be used to monitor disease development in various ways.

The value of w₁ is 1 or more e.g. 1, 2, 3, 4, 5, 6, 7, 8, 9, 10, 11, 12, 13, 14, 15 (e.g. up to 343). The value of w₂ is 2 or more e.g. 1, 2, 3, 4, 5, 6, 7, 8, 9, 10, 11, 12, 13, 14, 15 (e.g. up to 343). The values of w₁ and w₂ may be the same or different. If they ae different, it is usual that w₂≥w₁, as the later analysis should focus on a biomarker panel that is at least as wide as the number already detected in the earlier analysts. There will usually be an overlap between the w₁ and w₂ biomarkers (including situations where they are the same, such that the same biomarkers are measured at two time points) but N is also possible for w₁ and w₂ to have no biomarkers in common. The w₁ and/or w₂ biomarker(s) can be used in combination with known endometriosis biomarkers, as discussed above.

Where the methods involve a first time and a second time, these times may differ by at least 1 day, 1 week, 1 month or 1 year. Samples may be taken regularly. The methods may involve measuring biomarkers in more than 2 samples taken at more than 2 time points i.e. there may be a 3rd sample, a 4th sample, a 5th sample, etc.

The Invention also provides a diagnostic device for use in diagnosis of endometriosis, wherein the device permits determination of the level(s) of y Table 1 biomarkers. The value of y is defined above. The device may also permit determination of whether a sample contains one or more of the known endometriosis biomarkers mentioned above.

The invention also provides a kit comprising (i) a diagnostic device of the invention and (ii) instructions for using the device to detect y of the Table 1 biomarkers. The value of y is defined above. The kit is useful in the diagnoses of endometriosis.

The invention also provides a kit comprising reagents for measuring the levels of x different Table 1 biomarkers. The kit may also include reagents for determining whether a sample contains one or more of the known endometriosis biomarkers mentioned above. The value of x is defined above. The kit is useful in the diagnosis of endometriosis.

The Invention also provides a kit comprising components for preparing a diagnostic device of the invention. For instance, the kit may comprise individual detection reagents for x different biomarkers, such that an array of those x biomarkers can be prepared.

The invention also provides a product comprising (i) one or more detection reagents which permit measurement of x different Table 1 biomarkers, and (ii) a sample from a subject.

The Invention also provides a software product comprising (i) code that accesses data attributed to a sample, the data comprising measurement of y Table 1 biomarkers, and (ii) code that executes an algorithm for assessing the data to represent a level of y of the biomarkers in the sample. The software product may also comprise (iii) code that executes an algorithm for assessing the result of step (ii) to provide a diagnostic indicator of whether the subject has endometriosis. As discussed below, suitable algorithms for use in part (iii) include support vector machine algorithms, artificial neural networks, tree-based methods, genetic programming, etc. The algorithm can preferably classify the data of part (ii) to distinguish between subjects with endometriosis and subjects without based on measured biomarker levels in samples taken from such subjects. The invention also provides methods for training such algorithms. The y biomarker(s) can be used in combination with known endometriosis biomarkers, as discussed above.

The invention also provides a computer which is loaded with and/or is running a software product of the invention.

The invention also extends to methods for communicating the results of a method of the invention. This method may involve communicating assay results and/or diagnostic results. Such communication may be to, for example, technicians, physicians or patients. In some embodiments, detection methods of the invention will be performed in one country and the results will be communicated to a recipient in a different country.

The Invention also provides an isolated antibody (preferably a human antibody) which recognises one of the antigens listed in Table 1. The invention also provides an isolated nucleic acid encoding the heavy and/or light chain of the antibody. The invention also provides a vector comprising this nucleic acid, and a host cell comprising this vector. The invention also provides a method for expressing the antibody comprising culturing the host ell under conditions which permit production of the antibody. The invention also provides derivatives of the human antibody e.g. F(ab′)₂ and F(ab) fragments, Fv fragments, single-chain antibodies such as single chain Fv molecules (scFv), minibodies, dAbs, etc.

The invention also provides the use of a Table 1 biomarker as a biomarker for endometriosis.

The invention also provides the use of x different Table 1 biomarkers as biomarkers for endometriosis. The value of x is defined above. These may include (i) any specific one of the 343 biomarkers in Table 1 in combination with (ii) any of the other 342 biomarkers in Table 1. Preferably the invention uses at least one protein biomarker (e.g. auto-antibodies or an antigen) from Table 1 and at least one mRNA biomarker from Table 1. Preferably the panel includes protein biomarkers only (e.g. auto-antibodies or antigens) from Table 1. Preferably the panel includes mRNA biomarkers only from Table 1.

The invention also provides the use as combined biomarkers for endometriosis of (a) at least y Table 1 biomarker(s) and (b) biomarkers including auto-antibodies against CA125, CA19-9 and/or any of the antigens from Table 5 (and optionally, any other known biomarkers e.g. see above). The value of y is defined above. When y>1 the invention uses a panel of biomarkers of the invention. When y>1, preferably the panel includes at least one protein biomarker (e.g. auto-antibodies or an antigen) from Table 1 and at least one miRNA biomarker from Table 1. Such combinations include those discussed above. Panels of particular interest include those listed in Tables 10 to 15.

Biomarkers of the Invention

Antigens

The inventors identified auto-antibodies against 121 different human antigens (as listed in Table 1) and these can be used as endometriosis biomarkers. Further details of the 121 antigens are given in Table 2. Within the 121 antigens, the human antigens mentioned in Tables 6 and 7 are particularly useful for distinguishing between samples from subjects with endometriosis and from subjects without endometriosis. Further auto-antibody biomarkers can be used in addition to these 121 (e.g. any of the biomarkers listed in Table 5).

The sequence listing provides an example of a natural coding sequence for these antigens. These specific coding sequences are not limiting on the invention, however, and auto-antibody biomarkers may recognise variants of polypeptides encoded by these natural sequences (e.g. allelic variants, polymorphic forms, mutants, splice variants, or gene fusions), provided that the variant has an epitope recognised by the auto-antibody. Details on allelic variants of or mutations in human genes are available from various sources, such as the ALFRED database [54] or, in relation to disease associations, the OMIM [55] and HGMD [56] databases. Details of splice variants of human genes are available from various sources, such as ASD [57].

miRNAs

The inventors identified 222 individual human miRNAs (as listed in Table 1), and these can be used as endometriosis biomarkers. Further details of these mRNAs are given in Table 3 and Table 4. Within the 222 miRNAs, the miRNAs mentioned in Tables 8, 9 and 16-21 am particularly useful for distinguishing between samples from subjects with endometriosis and from subjects without endometriosis.

Preferably, the invention uses any of the mRNAs mentioned in Tables 9 and 16-18.

Preferably, the invention uses any of the miRNAs listed in the group consisting of: ebv-miR-BART2-5p, hsa-let-7f, hsa-let-7g, hsa-miR-1260, hsa-miR142-3p, hsa-miR-197, hsa-miR-215, hsa-miR-223, hsa-miR-30b, hsa-miR-320c, hsa-miR34a, hsa-miR-497, hsa-miR-630, hsa-miR-663 and hsa-miR-720.

The specific sequences in Tables 3 and 4 are not limiting on the invention. The invention includes detecting and measuring the levels of polymorphic variants of these mRNAs. A database outlining in more detail the miRNAs listed herein is available: MiRBase [58, 59, 60, 61] or, in relation to target prediction, the DIANA-microT [62, 63], microRNA.org 1641, miRDB [65, 66], TargetScan [87] and PicTar [88] databases.

As mentioned above, detection of a single Table 1 biomarker can provide useful diagnostic information, but each biomarker might not individually provide information which is useful i.e. auto-antibodies against a Table 1 antigen may be present in some, but not all, subjects with endometriosis. An inability of a single biomarker to provide universal diagnostic results for all subjects does not mean that this biomarker has no diagnostic utility; rather, any such inability means that the test results (as in all diagnostic tests) have to be properly understood and interpreted.

To address the possibilty that a single biomarker might not provide universal diagnostic results, and to increase the overall confidence that an assay is giving sensitive and specific results across a disease population, it is advantageous to analyse a plurality of the Table 1 biomarkers (i.e. a panel). For instance, a negative signal for a particular Table 1 antigen is not necessarily indicative of the absence of endometrioses, confidence that a subject does not have endometriosis increases as the number of negative results increases. For example, if al 343 biomarkers are tested and are negative then the result provides a higher degree of confidence than if only 1 biomarker is tested and is negative. Thus biomarker panels are most useful for enhancing the distinction seen between diseased and non-diseased samples.

As mentioned above, though, preferred panels have from 2 to 6 biomarkers as the burden of measuring a higher number of markers is usually not rewarded by better sensitivity or specificity.

The inventors found that the combination of the information from protein and miRNA biomarkers improves diagnostic power, as measured by sensitivity, specificity and/or area under the Receiver Operating Characteristic (ROC) curve. Thus, the invention preferably uses at least one protein biomarker from Table 1 and at least one miRNA biomarker from Table 1 to assist in the diagnosis of endometriosis.

Preferred panels are given below, and are listed in Tables 11-15.

Some biomarkers of the invention have different relative differential expression profiles in endometriosis samples compared to a control. Pairs of these biomarkers, where one is up-regulated and the other is down-regulated relative to the same control sample, may provide a useful way of diagnosing or predicting endometriosis. For example, the inventors found that ebv-miR-BART2-5p is up-regulated in endometriosis samples vs. non-endometriosis samples (i.e. a negative control) and hsa-miR-564 is down-regulated in endometriosis samples vs. non-endometriosis samples (i.e. a negative control), so this pair would be useful. This divergent behaviour can enhance diagnosis or prediction of endometriosis when the pair of the biomarkers is assessed in the same sample.

The Subject

The invention is used, ether alone or in combination with other measurements or data concerning the subject, for diagnosing disease in a subject.

The subject may be pre-symptomatic for endometriosis or may already be displaying clinical symptoms, e.g. pelvic pain, dysmenorrhea, dyspareunia, dyschesia, fatigue and infertility. For pre-symptomatic subjects the invention is useful for predicting that symptoms may develop in the future if no preventative action is taken. For subjects already displaying clinical symptoms, the invention may be used to confirm or resolve another diagnosis. The subject may already have begun treatment for endometriosis.

In some embodiments the subject may already be known to be predisposed to development of endometriosis e.g. due to family or genetic links. In other embodiments, the subject may have no such predisposition, and may develop the disease as a result of environmental factors e.g. as a result of exposure to particular chemicals (such as toxins or pharmaceuticals), as a result of diet, as a result of infection, etc.

The subject will typically be a human being. In some embodiments, however, the invention is useful in non-human mammals, e.g. mouse, rat, rabbit, guinea pig, cat, dog, horse, pig, cow, or non-human primate (monkeys or apes, such as macaques or chimpanzees). In non-human embodiments, any detection antigens used with the invention will typically be based on the relevant non-human ortholog of the human auto-antigens disclosed herein. In some embodiments animals can be used experimentally to monitor the impact of a therapeutic on a particular biomarker.

The Sample

The Invention analyses samples from subjects. Many types of sample can include auto-antibodies and/or antigens or miRNAs suitable for detection by the invention. The sample can be a tissue sample, e.g. from regions of uterine or vaginal tissues. Alternatively, the sample can be or a body fluid sample, e.g. cervical discharge or peritoneal fluid.

In some embodiments, a method of the invention involves a step of obtaining the sample from the subject. In other embodiments, however, the sample is obtained separately from and prior to performing a method of the invention.

Detection of the biomarkers of the invention may be performed directly on a sample taken from a subject, or the sample may be treated between being taken from a subject and being analysed. For example, a blood sample may be treated to remove cells, leaving antibody-containing plasma for analysis, or to remove cells and various clotting factors, leaving antibody-containing serum for analysis. A bodily fluid sample, e.g. a blood sample, can be treated to extract circulating endometrial cells for analysis. Various types of sample may be subjected to treatments such as dilution, aliquoting, sub-sampling, heating, freezing, irradiation, eta between being taken from the body and being analysed. Addition of processing reagents is also typical for various sample types e.g. addition of anticoagulants to blood samples.

Biomarker Detection

Auto-Antibody Detection

The invention involves determining in a sample the level of auto-antibodies that bind to the antigens listed in Table 1. Immunochemical techniques for detecting antibodies against specific antigens are well known in the art, as are techniques for detecting specific antigens themselves. Detection of an antibody will typically involve contacting a sample with a detection antigen, wherein a binding reaction between the sample and the detection antigen indicates the presence of the antibody of interest. Detection of an antigen will typically involve contacting a sample with a detection antibody, wherein a binding reaction between the sample and the detection antibody indicates the presence of the antigen of interest. Detection of an antigen can also be determined by non-immunological methods, depending on the nature of the antigen e.g. If the antigen is an enzyme then its enzymatic activity can be assayed, or If the antigen is a receptor then its binding activity can be assayed, etc. For example, the CLK1 kinase can be assayed using methods known in the art.

A detection antigen can be a natural antigen recognised by the auto-antibody (e.g. a mature human protein disclosed in Table 1), or R may be an antigen comprising an epitope which is recognized by the auto-antibody. Where a detection antigen is a polypeptide, its amino acid sequence can vary from the natural sequences disclosed above, provided that it has the ability to specifically bind to an auto-antibody of the invention (i.e. the binding is not non-specific and so the detection antigen will not arbitrarily bind to antibodies in a sample). It may even have little in common with the natural sequence (e.g. a mimotope, an aptamer, etc.). Typically, though, a detection antigen will comprise an amino acid sequence (i) having at least 90% (e.g. ≥91%, ≥92%, ≥93%, ≥94%, ≥95%, ≥96%, ≥97%, ≥98%, ≥99%) sequence identity to the relevant SEQ ID NO disclosed herein (i.e. any one of SEQ ID NOs: 1-121) across the length of the detection antigen, and/or (ii) comprising at least one epitope from the relevant SEQ ID NO disclosed herein (i.e. any one of SEQ ID NOs: 1-121). Thus the detection antigen may be any of the variants discussed above.

Epitopes are the parts of an antigen that are recognised by and bind to the antigen binding sites of antibodies and are also known as “antigenic determinants”. An epitope-containing fragment may contain a linear epitope from within a SEQ ID NO and so may comprise a fragment of at least n consecutive amino acids of the SEQ ID NO, wherein n may be 7 or more (e.g. 8, 10, 12, 14, 16, 18, 20, 25, 30, 35, 40, 50, 80, 70, 80, 90, 100, 150, 200, 250 or more). B-cell epitopes can be identified empirically (e.g. using PEPSCAN [89,70] or similar methods), or they can be predicted e.g. using the Jameson-Wolf antigenic index [71], ADEPT [72], hydrophilicity [73], antigenic index [74], MAPITOPE [75], SEPPA [76], matt-based approaches [77], the amino acid pair antigenicity scale [78], or any other suitable method e.g. see ref. 79. Predicted epitopes can readily be tested for actual immunochemical reactivity with samples.

Detection antigens can be purified from human sources, but k is more typical to use recombinant antigens (e.g. particularly where the detection antigen uses sequences which are not present in the natural antigen e.g. for attachment). Various systems are available for recombinant expression, and the choice of system may depend on the auto-antibody to be detected. For example, prokaryotic expression (e.g. using E. coli) are useful for detecting many auto-antibodies, but if an auto-antibody recognises a glycoprotein then eukaryotic expression may be required. Similarly, if an auto-antibody recognises a specific discontinuous epitope then a recombinant expression system which provides correct protein folding may be required.

The detection antigen may be a fission polypeptide with a first region and a second region, wherein the first region can react with an auto-antibody in a sample and the second region can react with a substrate to immobilise the fusion polypeptide thereon.

A detection antibody for a biomarker antigen can be a monoclonal antibody or a polyclonal antibody. Typically it will be a monoclonal antibody. The detection antibody should have the ability to specifically bind to a Table 1 antigen (i.e. the binding is not non-specific and so the detection antibody will not arbitrarily bind to other antigens in a sample).

Various assay formats can be used for detecting biomarkers in samples. For example, the invention may use one or more of western blot, immunoprecipitation, silver staining, mass spectrometry (e.g. MALDI-MS), conductivity-based methods, dot blot, slot blot colorimetric methods, fluorescence-based detection methods, or any form of immunoassay, etc. The binding of antibodies to antigens can be detected by any means, including enzyme-biked assays such as ELISA, radioimmunoassays (RIA), immunoradiometric assays (IRMA), immunoenzymatic assays (IEMA), DELFIA™ assays, surface plasmon resonance or other evanescent light techniques (e.g. using planar waveguide technology), label-free electrochemical sensors, etc. Sandwich assays are typical for immunological methods.

In embodiments where multiple biomarkers are to be detected an array-based assay format is preferable, in which a sample that potentially contains the biomarkers is simultaneously contacted with multiple detection reagents (antibodies and/or antigens) in a single reaction compartment. Antigen and antibody arrays are well known in the art e.g. see references 80-88, including arrays for detecting auto-antibodies. Such arrays may be prepared by various techniques, such as those disclosed in references 87-91, which are particularly useful for preparing microarrays of correctly-folded polypeptides to facilitate binding interactions with auto-antibodies. It has been estimated that most B-cell epitopes are discontinuous and such epitopes are known to be important in diseases with an autoimmune component. For example. In autoimmune thyroid diseases, auto-antibodies arise to discontinuous epitopes on the immunodominant region on the surface of thyroid peroxidase and in Goodpasture disease auto-antibodies arise to two major conformational epitopes. Protein arrays which have been developed to present correctly-folded polypeptides displaying native structures and discontinuous epitopes are therefore particularly well suited to studies of diseases where auto-antibody responses occur [84].

Methods and apparatuses for detecting binding reactions on antigen arrays are now standard in the art. Preferred detection methods are flu detection methods. To detect auto-antibodies which have bound to immobilised antigens, a sandwich assay is typical, in which the primary antibody is an auto-antibody from the sample and the secondary antibody is a labelled anti-sample antibody (e.g. an anti-human antibody).

Where a biomarker is an auto-antibody the invention will generally detect IgG antibodies, but detection of auto-antibodies with other subtypes is also possible e.g. by using $ detection reagent which recognises the appropriate lass of auto-antibody (IgA, IgM, IgE or IgD rather than IgG). The assay format may be able to distinguish between different antibody subtypes and/or isotypes. Different subtypes [92] and isotypes [93] can influence auto-antibody repertoires. For instance, a sandwich assay can distinguish between different subtypes by using differentially-labeled secondary antibodies e.g. different libels for anti-IgG and anti-IgM.

As mentioned above, the invention provides a diagnostic device which permits determination of whether a sample contains Table 1 biomarkers. Such devices will typically comprise one or more antigen(s) and/or antibodies immobilised on a solid substrate (e.g. on glass, plastic, nylon. etc.). Immobilisation may be by covalent or non-covalent bonding (e.g. non-covalent bonding of a fusion polypeptide, as discussed above, to an immobilised functional group such as an avidin [89] or a bleomycin-family antibiotic [91]). Antigen arrays are a preferred foment, with detection antigens being individually addressable. The immobilised antigens will be able to react with auto-antibodies which recognise a Table 1 antigen.

In some embodiments, the solid substrate may comprise a strip, a slide, a bead, a well of a microtitre plate, a conductive surface suitable for performing mass spectrometry analysis [94], a semiconductive surface [95,96]. a surface plasmon resonance support, a planar waveguide technology support, a microfluidic devices, or any other device or technology suitable for detection of antibody-antigen binding.

Where the invention provides or uses an antigen array for detecting a panel of auto-antibodies as disclosed herein, in some embodiments the array may include only antigens for detecting these auto-antibodies. In other embodiments, however, the array may include polypeptides in addition to those useful for detecting the auto-antibodies. For example, an array may include one or more control polypeptides. Suitable positive control polypeptides include an anti-human immunoglobulin antibody, such as an anti-IgM antibody, an anti-IgG antibody, an anti-IgA antibody, an anti-IgE antibody or combinations thereof. Other suitable positive control polypeptides which can bind to sample antibodies include protein A or protein G, typically in recombinant form. Suitable negative control polypeptides include, but are not limited to, β-galactosidase, serum albumins (e.g. bovine serum albumin (BSA) or human serum albumin (HSA)), protein tags, bacterial proteins, yeast proteins, citrullinated polypeptides, etc. Negative control features on an array can also be polypeptide-free e.g. buffer alone, DNA, etc. An arrays control features are used during performance of a method of the invention to check that the method has performed as expected e.g. to ensure that expected proteins are present (e.g. a positive signal from serum proteins in a serum sample) and that unexpected substances are not present (e.g. a positive signal from an array spot of buffer alone would be unexpected).

In an antigen array of the invention, at least 10% (e.g. ≥20%, ≥30%, ≥40%, ≥50%, ≥60%, ≥70%, ≥80%, ≥90%, ≥95%, or more) of the total number of different proteins present on the array may be for detecting auto-antibodies as disclosed herein.

An antigen array of the invention may include one or more replicates of a detection antigen and/or control feature e.g. duplicates, triplicates or quadruplicates. Replicates provide redundancy, provide intra-array controls, and facilitate inter-array comparisons.

An antigen array of the invention may include detection antigens for more than just the 121 different auto-antibodies described here, but preferably it can detect antibodies against fewer than 10000 antigens (e.g. <5000, <4000, <3000, <2000, <1000, <500, <250, <100, etc.).

An array is advantageous because it allows simultaneous detection of multiple biomarkers in a sample. Such simultaneous detection is not mandatory, however, and a panel of biomarkers can also be evaluated in series. Thus, for instance, a sample could be split into sub-samples and the sub-samples could be assayed. In series. In this embodiment it may not be necessary to complete analysis of the whole panel e.g. the diagnostic indicators obtained on a subset of the panel may indicate that a patient has endometriosis without requiring analysis of any further members of the panel. Such incomplete analysis of the panel is encompassed by the invention because of the intention or potential of the method to analyse the complete panel.

As mentioned above, some embodiments of the invention can include a contribution from known tests for endometriosis, such as CA125, CA19-9 and/or any of the antigens listed in Table 5. Any known tests can be used e.g. laparoscopy, ultrasound, magnetic resonance imaging (MRI), etc.

miRNA Detection

Table 1 lists 222 human miRNAs. and methods of the invention can involve detecting and determining the level of these mRNA biomarker(s) in a sample. Further details of these miRNAs are provided in Tables 3 and 4. Tables 3 and 4 also include nucleotide sequences for these miRNAs, but polymorphisms of mRNA are known in the art and so the invention can also involve detecting and determining the level of a polymorphic miRNA variant of these listed mRNA sequences.

Techniques for detecting specific miRNAs are well known in the art, e.g. mic oar ay analysis and NanoString's nCounter technology, polymerase chain reaction (PCR)-based methods (e.g. reverse transcription PCR, RT-PCR), in-situ hybridisation (ISH)-based methods (e.g. fluorescent ISH, FISH), northern blotting, sequencing (e.g. nest-generation sequencing), fluorescence-based detection methods, etc. Any of the detection techniques mentioned above can be used with the invention. Where prognosis is the primary interest, a quantitative detection technique is preferred, e.g. real-time quantitative PCR (qPCR), TagMan® or SYBR® Green.

Detection of a miRNA typically involves contacting (‘hybridising’) a sample with a complementary detection probe (e.g. a synthetic oligonucleotide strand), wherein a specific (rather than non-specific) finding reaction between the sample and the complementary probe indicates the presence of the miRNA of interest, in some instances, the miRNA in the sample is amplified prior to detection, e.g. by reverse transcription of the miRNA to produce a complementary DNA (cDNA) strand, and the derived cDNA can be used as a template in the subsequent PCR reaction.

Thus, the invention provides nucleic acids, which can be used, for example, as hybridization probes for specific detection of miRNA in biological samples or as single-stranded primers to amplify the miRNA.

The term ‘nucleic acid’ includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic adds (PNAs) or phosphorothioates) or modified bases. Nucleic add according to the invention can take various forms (e.g. single-stranded, primers, probes, labelled eta). Primers and probes are generally single-stranded.

The nucleic acid can be identical or complementary to the mature miRNA sequences listed in Tables 3 and 4 (i.e. any one of SEQ ID NOs:122-343).

The nucleic acid can comprise a nucleotide sequence that has ≥50%, ≥60%, ≥70%, ≥75%, ≥80%, ≥85%, ≥90%, ≥95%, ≥96%, ≥97%, ≥98%, ≥99% or more identity to any one of SEQ ID NOs: 122-343. Identity between sequences is preferably determined by the Smith-Waterman homology search algorithm as described above.

The nucleic acid can comprise a nucleotide sequence that has ≥50%, ≥80%, ≥70%, ≥75%, ≥80%, ≥85%, ≥90%, ≥95%, ≥96%, ≥97%, λ≥98%, ≥99% or more complementarily to any one of SEQ ID NOs: 122-343.

The term “complementarily” when used in relation to nucleic acids refers to Watson-Crick base pairing. Thus the complement of C is G, the complement of G is C, the complement of A is T (or U), and the complement of T (or U) is A. It is abo possible to use bases such as I (the purine inosine) e.g. to complement pyrimidines (C or T).

Where a nucleic acid is DNA, it will be appreciated that “U” in a RNA sequence will be replaced by T in the DNA. Similarly, where a nucleic acid is RNA. It will be appreciated that “T” in a DNA sequence will be replaced by IP in the RNA.

The nucleic acid may be 12 or more. e.g. 12, 13, 14, 15, 16, 17 or 18, etc. (e.g. up to 50) nucleotides in length. The nucleic acid may be 1540 nucleotides in length, 10-25 nucleotides in length, 15-25 nucleotides in length, or 20.25 nucleotides in length.

The nucleic acid may include sequences that do not hybridise to the miRNA biomarkers, and/or amplified products thereof. For example, the nucleic acid may contain additional sequences at the 5′ end or at the 3′ end. The additional sequences can be a linker, e.g. for cloning or PCR purposes.

Nucleic acid of the invention may be attached to a solid support (e.g. a bead, plate, filter, film, side, microarray support, resin, etc). Nucleic acid of the invention may be labelled e.g. with a radioactive or fluorescent label, or a biotin label. This is particularly useful where the nucleic acid is to be used in detection techniques e.g. where the nucleic acid is a primer or as a probe. Methods for preparing fluorescent labeled probes, e.g. for fluorescent in-situ hybridisation FISH analysis, are known in the art, and FISH probes can be obtained commercially, e.g. from Exiqon.

The invention may use in-situ hybridisation (ISH)-based methods. e.g. fluorescent in-situ hybridisation (FISH). Hybridization reactions can be performed under conditions of different “stringency” following by washing. Preferably, the nucleic acid of the invention hybridize under high stringency conditions, such that the nucleic acid specifically hybridizes to a miRNA in an amount that is delectably stronger than non-specific hybridization. Relatively high stringency conditions include, for example, low salt and/or high temperature conditions, such as provided by about 0.02-0.1 M NaCl or the equivalent at temperatures of about 50-70° C. A stringent wash removes nonspecific probe binding and overloaded probes. Relatively stringent wash conditions include, for example, low salt and/or presence of detergent, e.g. 0.02% SDS in 1× Saline-Sodium Citrate (SSC) at about 50° C.

In embodiments where multiple biomarkers are to be detected, an array-based assay or PCR format is preferable, in which a sample that potentially contains the biomarkers is simultaneously contacted with multiple oligonucleotide, complementary detection probes or PCR primers/probes (‘multiplexed’) in a single reaction compartment, whereby a reaction compartment is defined as, but not linked to, a microlitre well, microfluidic chamber or detection pore. In other embodiments these multiple biomarkers could either be contacted with its complementary detection probe in separate, individual reaction compartments and/or, experiments could be separated over time and using different platform technologies in either multiplexed single reaction compartments or separate, individual reaction compartments. Microarray and PCR usage for the detection of miRNAs is well known in the art e.g. see reference 115. Microarrays may be prepared by various techniques, such as those disclosed in references 118, 117, 118. Methods based on nucleic acid amplification are also well known in the art.

Methods and apparatus for detecting binding reactions on DNA microarrays are now standard in the art. Preferred detection methods are fluorescence-based detection methods. To detect biomarkers which have bound to immobilised oligonucleotide strands on a glass substrate is typical e.g. in which the target mRNA is fluorescently labelled and then is hybridised to a complementary oligonucleotide strand (probe).

An array is advantageous because it allows simultaneous detection of multiple biomarkers in a sample. Such simultaneous detection is not mandatory, however, and a panel of biomarkers can also be evaluated in series. Thus, for instance, a sample could be spilt into sub-samples and the sub-samples could be assayed in series. In this embodiment it may not be necessary to complete analysis of the whole panel e.g. the diagnostic indicators obtained on a subset of the panel may indicate that a patient has endometriosis without requiring analysis of any further members of the panel. Such incomplete analysis of the panel is encompassed by the invention because of the intention or potential of the method to analyse the complete panel.

Data Interpretation

The invention involves a step of determining the level of Table 1 biomarker(s). In some embodiments of the invention this determination for a particular marker can be a simple yes/no determination, whereas other embodiments may require a quantitative or semi-quantitative determination, still other embodiments may involve a relative determination (e.g. a ratio relative to another marker, or a measurement relative to the same marker in a control sample), and other embodiments may involve a threshold determination (e.g. a yes/no determination whether a level is above or below a threshold). Usually biomarkers will be measured to provide quantitative or semi-quantitative results (whether as relative concentration, absolute concentration, fold charges, titre, relative fluorescence etc.) as this gives more data for use with classifier algorithms.

Usually the raw data obtained from an assay for determining the presence, absence, or level (absolute or relative) require some sod of manipulation prior to their use. For instance, the nature of most detection techniques means that some signal will sometimes be seen even if no biomarker is actually present and so this noise may be removed before the results are interpreted. Similarly, there may be a background level of the biomarker in the general population which needs to be compensated for. Data may need scaling or standardising to facilitate inter-experiments comparisons. These and similar issues, and techniques for dealing with them, are well known in the immunodiagnostic area.

Various techniques are available to compensate for background signal in a particular experiment. For example, replicate measurements will usually be performed (e.g. using multiple features of the same detection probe on a single array) to determine antra-assay variation, and average values from the replicates can be compared (e.g. the median value of binding to quadruplicate array features).

Furthermore, standard markers can be used to determine inter assay variation and to pen nit calibration and/or normalisation e.g. an array can include one or more standards for indicating whether measured signals should be proportionally increased or decreased. For example, an assay might include a step of analysing the level of one or more control markers) in a sample e.g. levels of an antigen or antibody unrelated to endometriosis. Signal may be adjusted according to distribution in a single experiment. For instance, signals in a single array experiment may be expressed as a percentage of interquartile differences e.g. as [observed signal−25th percentile]/[75th percentile−25th percentile]. This percentage may then be normalised e.g. using a standard quartile normalization matrix, such as disclosed in reference 97, le which all percentage values on a single array are ranked end replaced by the average of percentages for antigens with the same rank on ail arrays. Overall, this process gives data distributions with identical median and quartile values. Data transformations of this type are standard in the art for permitting valid inter-array comparisons despite variation between different experiments.

The level of an auto-antibody relative to a single baseline level may be defined as a fold difference. Normally k is desirable to use techniques that can indicate a change of at least 1.5-fold e.g. ≥1.75 fold, ≥2:2-fold, ≥2.5-fold, ≥5-fold, etc.

A control sample can also be used for data normalisation. For example, levels of an antibody or a mRNA unrelated to endometriosis can be measured in both the sample and a negative control, and signal from other antibodies can be adjusted accordingly. A negative control sample is from a subject that does not have any clinical presentation of endometriosis.

In some embodiments, rather than (or in addition to) comparing a biomarker against a ‘normal’ baseline, they will be compared to levels seen in a sample from a subject known to have endometriosis and known to have a particular biomarker (i.e. comparison to a positive control). For some biomarkers this comparison may be easier than using a lower negative control level. A preference for comparison against a negative or positive control may depend on the dynamic range between negative and positive signals.

Levels of the biomarkers in a control may be determined in parallel to determining levels in the sample. Rather than making a parallel determination, however, it can be more convenient to use an absolute control level based on empirical data. For example, the level of a particular biomarker may be measured in samples taken from a range of subjects without endometriosis. Those levels can be used to build a baseline across the range of subjects. This may involve normalization relative to a reference antibody. A population of negative control subjects can be used to provide a collection of baseline levels for subjects of different genders, ages, ethnic lies, habits (e.g. smokers, non-smokers), eta, so that, if there is variation across the population, a control can be matched to a particular subject as closely as possible. Thus, by analysing non-diseased samples from a sufficiently large number of subjects it is possible to establish an empirical baseline for any particular auto-antibody or miRNA, which can serve as the control level for comparison according to the invention. The control level is not necessarily a single value, but could be a range, against which a test value can be compared. For instance, if a particular auto-antibody titre is variable across non-diseased subjects, but is always in the range of 20-100 (arbitrary) units, a titre of 400 units in a sample would indicate a disease state.

As well as compensating for variation which is inherent between different experiments, it can also be important to compensate for background levels of a biomarker which are present in the general population. Again, suitable techniques am well known. For example, levels of a particular biomarker in a sample will usually be measured quantitatively or semi-quantitatively to permit comparison to the background level of that biomarker. Various controls can be used to provide a suitable baseline for comparison, and choosing suitable controls is routine in the diagnostic field. Further details of suitable controls are given below.

The measured level(s) of biomarker(s), after any compensation/normalisation/etc., can be transformed into a diagnostic result in various ways. This transformation may involve an algorithm which provides a diagnostic result as a function of the measured level(s). Where a panel is used then each individual biomarker may make a different contribution to the overall diagnostic result and so two biomarkers may be weighted differently.

The creation of algorithms for converting measured levels or raw data into scores or results is well known in the ad. For example, linear or non-linear classifier algorithms can be used. These algorithms can be trained using data from any particular technique for measuring the marker(s). Suitable training data will have been obtained by measuring the biomarkers in “case” and “control” samples i.e. samples from subjects known to suffer from endometriosis and from subjects known not to suffer from endometriosis. Most usefully the control samples will also include samples from subjects with a related disease which is to be distinguished from the disease of interest e.g. it is useful to train the algorithm with data from rheumatoid arthritis subjects and/or with data from subjects with connective tissue diseases other than endometriosis. The classifier algorithm is modified until it can distinguish between the case and control samples e.g. by adding or removing markers from the analysis, by changes in weighting, etc. Thus a method of the invention may include a step of analysing biomarker levels in a subject's sample by using a classifier algorithm which distinguishes between endometriosis subjects and non-endometriosis subjects based on measured biomarker levels in samples taken from such subjects.

Various suitable classifier algorithms are available e.g. linear discriminant analysis, naïve Bayes classifiers, perceptions, support vector machines (SW)[98] and genetic programming (GP) [99]. GP is particularly useful as it generally selects relatively small numbers of biomarkers and overcomes the problem of trapping in a local maximum which is inherent in many other classification methods. SWIM based approaches have previously been applied to endometriosis datasets [100]. The inventors have previously confirmed that both SW and GP approaches can be trained on the same biomarker panels to distinguish the auto-antibody/antigen biomarker profiles of case and control cohorts with similar sensitivity and specificity i.e. auto-antibody biomarkers are not dependent on a single method of analysis. Moreover, these approaches can potentially distinguish endometriosis subjects from subjects with (i) other forms of autoimmune disease and fie rheumatoid arthritis. The biomarkers in Table 1 can be used to train such algorithms to reliably make such distinctions. The classification performance (sensitivity and specificity, ROC analysis) of any putative biomarkers can be rigorously assessed using nested cross validation and permutation analyses prior to further validation. Biological support for putative biomarkers can be sought using tools and databases including Genespring (version 11.5.1), Biopax pathway for GSEA analysis and Pathway Studio (version 9.1).

It will be appreciated that, although there may be some biomarkers in Table 1 which always give a negative absolute signal when contacted with negative control samples (and thus any positive signal is immediately indicative of endometriosis), it is more common that a biomarker will give at least a low absolute signal (and thus that a disease-indicating positive signal requires detection of auto-antibody levels above that background levee. Thus references herein detecting a biomarker may not be references to absolute detection but rather (as is standard in the art) to a level above the levels seen in an appropriate negative control. Such controls may be assayed in parallel to a test sample but t can be more convenient to use an absolute control level based on empirical data, or to analyse data using an algorithm which can (e.g. by previous training) use biomarker levels to distinguish samples from disease patients vs. non-disease patients.

The level of a particular biomarker in a sample from an endometriosis-diseased subject may be above or below the level seen in a negative control sample. Antibodies that react with self-antigens occur naturally in healthy individuals and it is believed that these are necessary for survival of T- and B-cells in the peripheral immune system [101]. In a control population of healthy individuals there may thus be significant levels of circulating auto-antibodies against some of the antigens disclosed in Table 1 and these may occur at a significant frequency in the population. The level and frequency of these biomarkers may be altered in a disease cohort, compared with the control cohort. An analysis of the level and frequency of these biomarkers in the case and control populations may identify differences which provide diagnostic information. The level of auto-antibodies directed against a specific antigen may increase or decrease in an endometriosis sample, compared with a healthy sample.

When detecting combinations of protein and nucleic acid biomarkers, each class of biomarker is assayed and treated (e.g. data normalisation) as appropriate for that class, and then both the protein and the nucleic acid biomarkers are analysed together using an algorithm which classifies the sample.

In general, therefore, a method of the invention will involve determining whether a sample contains a biomarker level which is associated with endometriosis. Thus a method of the invention can include a step of comparing biomarker levels in a subject's sample to levels in (i) a sample from a patient with endometriosis and/or (ii) a sample from a patient without endometriosis. The comparison provides a diagnostic indicator of whether the subject has endometriosis. An aberrant level of one or more biomarker(s), as compered to known or standard expression levels of those biomarker(s) in a sample from a patient without endometriosis, indicates that the subject has endometriosis.

The level of a biomarker should be significantly different from that seen in a negative control. Advanced statistical tools (e.g. principal component analysis, unsupervised hierarchical clustering and linear modeling) can be used to determine whether two levels am the same or different. For example, an in vitro diagnosis will rarely be based on comparing a single determination. Rather, an appropriate number of determinations will be made with an appropriate level of accuracy to give a desired statistical certainty with an acceptable sensitivity and/or specificity. Antigen and/or antibody levels can be measured quantitatively to permit proper comparison, and enough determinations will be made to ensure that any difference in levels can be assigned a statistical significance to a level of p≤0.05 or better. The number of determinations will vary according to various criteria (e.g. the degree of variation in the baseline, the degree of up-regulation in disease states, the degree of noise, etc.) but, again, this fats within the normal design capabilities of a person of ordinary skill in this field. For example, interquartile differences of normalised data can be assessed, and the threshold for a positive signal (i.e. indicating the presence of a particular auto-antibody) can be defined as requiring that antibodies in a sample read with a diagnostic antigen at least 2.5-fold more strongly that the interquartile difference above the 75th percentile.

Other criteria are familiar to those skilled in the art and, depending on the assays being used, they may be more appropriate than quintile normalisation. Other methods to normalise data include data transformation strategies known in the an e.g. scaling, log normalisation, median normalisation, etc. For example, raw protein array data can be normalized by consolidating the replicates, transforming the data and applying median normalization which has been demonstrated to be appropriate for this type of analysis. Gene expression data can be subjected to background correction via 2D spatial correction and dye bias normalization via MvA lowess [102,103,104]. Normalized gene expression and proteomic data can be analysed for any potential signatures relating to differences between patient cohorts referring to levels of statistical significance (generally p<0.05), multiple testing correction and told changes within the expression data that could be indicative of biological effect (generally 2 fold in mRNA compared with a reference value).

The underlying aim of these data interpretation techniques is to distinguish between the presence of a Table 1 biomarker and of an arbitrary control biomarker, and also to distinguish between the response of sample from a endometriosis subject from a control subject. Methods of the invention may have sensitivity of at least 70% (e.g. >70%, >75%, >80%, >85%, >90%, >95%, >96%, >97%, >98%, >99%). Methods of the invention may have specificity of at least 70% (e.g. >70%, >75%, >60%, >85%, >90%, >95%, >96%, >97%, >98%, >99%). Advantageously, methods of the invention may have both specificity and sensitivity of at least 70% (e.g. >70%, >75%, >80%, >85%, >90%, >95%, >96%, >97%, >98%, >99%). As shown in the examples, the invention can consistently provide specificities above approximately 70% and sensitivities greater than approximately 70%.

Data obtained from methods of the invention, and/or diagnostic information based on those data, may be stored in a computer medium (e.g. in RAM, in non-volatile computer memory, on CD-ROM) and/or may be transmitted between computers e.g. over the kismet.

If a method of the invention indicates that a subject has endometriosis, further steps may then follow. For instance, the subject may undergo confirmatory diagnostic procedures, such as those involving physical inspection of the subject, and/or may be treated with therapeutic agent(s) suitable for treating endometriosis.

Preferably, the biomarkers of the invention can determine if a subject has peritoneum endometriosis. Thus, the invention also provides a method for detecting peritoneum endometriosis. The method can use any of the biomarkers listed in any of Tables 16, 18 and 20-21, preferably in Table 16 or 18.

Alternatively, the biomarkers of the invention can determine if a subject has ovarian endometriosis, such as endometriomas. Thus, the invention also provides a method for detecting ovarian endometriosis. The method can use any of the biomarkers listed in any of Tables 17-21, preferably in Table 17 or 18.

Monitoring the Efficacy of Therapy

As mentioned above, some methods of the invention involve testing samples from the same subject at two or more different points in time. In general, where the above text refers to the presence or absence of antibody(s), the invention also includes an increasing or decreasing level of the antibody(s) over time, or to a spread of antibodies in which additional antibodies or antibody classes are raised against a single auto-antigen. Methods which determine changes in antibody(s) over time can be used, for instance, to monitor the efficacy of a therapy being administered to the subject (e.g. in theranostics). The therapy may be administered before the first sample is taken, at the same time as the first sample is taken, or after the first sample is taken.

The invention can be used to monitor a subject who is receiving hormonal therapies. Hormonal treatments including the combine oral contraceptive pills, progesterone only pills and intrauterine progesterone (Levonorgestrel IUS), Progestins, Gestrinone, Danazol (despite its masculinisation side effects), GnRH analogues.

In related embodiments of the invention, the results of monitoring a therapy are used for future therapy prediction. For example, if treatment with a particular therapy is effective in reducing or eliminating disease symptoms in a subject, and is also shown to decrease levels of a particular auto-antibody in that subject, detection of that auto-antibody in another subject may indicate that this other subject will respond to the same therapy. Conversely, if a particular therapy was not effective in reducing or eliminating disease symptoms in a subject who had a particular auto-antibody or auto-antibody profile, detection of that auto-antibody or profile in another subject may indicate that this other subject will also fall to respond to the same therapy.

In other embodiments, the presence of auto-antibodies against a particular auto-antigen can be used as the basis of proposing or initiating a particular therapy. For instance, if is known that levels of a particular auto-antibody can be reduced by administering a particular therapy then that auto-antibody's detection may suggest that the therapy should begin. Thus the invention is useful in a theranostic setting. Normally at least one sample will be taken from a subject before a therapy begins.

Immunotherapy

Where the development of auto-antibodies to a newly-exposed auto-antigen is causative for a disease, early priming of the immune response can prepare the body to remove antigen-exposing cells when they arise, thereby removing the cause of disease before auto-antibodies develop dangerously. The auto-antigens listed in Table 2 are thus therapeutic targets for treating endometriosis. For example, one antigen known to be recognised by auto-antibodies is p53, and this protein is considered to be both a vaccine target and a therapeutic target for the modulation of cancer [105-107].

Thus the invention provides a method for raising an antibody response in a subject, comprising eliciting to the subject an immunogen which slicks antibodies which recognise an auto-antigen listed in Table 2. The method is suitable for immunoprophylaxis of endometriosis.

The invention also provides an immunogen for use in medicine, wherein the immunogen can elicit antibodies which recognise an auto-antigen listed in Table 2. Similarly, the invention also provides the use of an immunogen in the manufacture of a medicament for immunoprophylaxis of endometriosis, wherein the immunogen can elicit antibodies which recognise an auto-antigen listed in Table 2.

As discussed above for detection antigens, the immunogen may be the auto-antigen itself or may comprise an amino acid sequence having identity and/or comprising an epitope from the auto-antigen. Thus the immunogen may comprise an amino acid sequence (i) having at least 90% (e.g. ≥91%, ≥92%, ≥93%, ≥94%, ≥95%, ≥90%, ≥97%, ≥98%, ≥99%) sequence identity to the relevant SEQ ID NO disclosed herein (i.e. any of SEQ D NOs: 1-121), and/or (ii) comprising at least one epitope from the relevant SEQ ID NO disclosed herein (La. any of SEQ ID NOs: 1-121). Other immunogens may also be used, provided that they can sack antibodies which recognise the auto-antigen of interest.

As an alternative to immunising a subject with a polypeptide immunogen, it is possible to administer a nucleic acid (e.g. DNA or RNA) immunogen encoding the polypeptide, for in situ expression in the subject, thereby leading to the development of an antibody response.

The immunogen may be delivered in conjunction (e.g. in admixture) with an immunological adjuvant. Such adjuvants include, but are not limited to, insoluble aluminium salts, water-In-oil emulsions, oil-in-water emulsions such as MF59 and AS03, saponins. ISCOMs, 3-O-deacylated MPL (3dMPL), immunostimulatory oligonucleotides (e.g. including one or more CpG motifs), bacterial ADP-ribosylating toxins and detoxified derivatives thereof, cytokines, chitosan, biodegradable microparticles, liposomes, imidazoquinolones, phosphazenes (e.g. endometriosisPP), aminoalkylglucosaminide phosphates, gamma inulins, etc. Combinations of such adjuvants can also be used. The adjuvant(s) may be selected to elicit an immune response involving CD4 or CD8 T cells. The adjuvant(s) may be selected to bias an immune response towards a TH1 phenotype or a TH2 phenotype.

The immunogen may be delivered by any suitable route. For example, it may be delivered by parenteral injection (e.g. subcutaneously, intraperitoneally, intravenously, intramuscularly), or mucosally, such as by oral (e.g. tablet, spray), topical, transdermal, transcutaneous, intranasal, ocular, aural, pulmonary or other mucosal administration.

The immunogen may be administered in a liquid or sold form. For example, the immunogen may be formulated for topical administration (e.g. as an ointment, cream or powder), for oral administration (e.g. as a tablet or capsule, as a spray, or as a syrup), for pulmonary administration (e.g. as an inhaler, using a fine powder or a spray), as a suppository or pessary, as drops, or as an injectable solution or suspension.

RNA-Based Therapy

The miRNAs listed in Table 3 and 4 can be useful for RNA-based therapy, e.g., antisense therapy. There is literature precedent outlining the use of antisense therapy to manage cancer [108]. A synthetic nucleic acid complementary to a miRNA listed in Table 3 or Table 4 could be used to stimulate cell death of cancerous cells (either associated with endometriosis and/or aggressive endometriosis). Additionally, in vivo antisense therapy could be used to introduce a nucleic acid complementary to a miRNA listed in Table 3 or Table 4 to specifically bind to, and abrogate, overexpression of specific miRNA(s) associated with endometriosis and/or aggressive endometriosis.

Thus the invention provides a nucleic acid which hybridises to miRNA(s) listed in Table 3 or Table 4 (i.e. any of SEQ ID NOs: 122-343), and which is conjugated to a cytotoxic agent. The miRNA is preferably a human miRNA. My suitable cytotoxic agent can be used. These conjugates miRNAs can be used in methods of therapy.

Thus the invention provides a complementary nucleic which recognises a miRNA listed in Table 3 or Table 4 for the purposes of RNA-based therapies, e.g. antisense therapy.

Imaging

The biomarkers listed in Table 1 can be useful for imaging.

A labelled antibody against an auto antigen kited in Table 2 can be injected in vivo and the distribution of the antigen can then be detected. This method may identify the source of the auto-antigen (e.g. an area in the body where there is a high concentration of the antigen), potentially offering early identification of a pathological condition. Imaging techniques can also be used to monitor the progress or remission of disease, or the impact of a therapy.

The invention also provides a labelled antibody which recognises an auto-antigen listed in Table 2. The antibody may be a human antibody, as discussed above. Any suitable label can be used e.g. quantum dots, spin labels, fluorescent labels, etc.

A labelled, synthetic nucleic complementary to a miRNA(s) listed in Table 3 or Table 4 could be used for the identification, in ex via (e.g. tissue samples taken from biopsies), and in vivo (e.g. magnetic resonance imaging (MRI), positron emission tomography (PET) computed tomography (CT) scans of patients) samples of miRNAs associated ted with endometriosis and/or aggressive endometriosis. This may potentially offer a method for the early identification of endometriosis and/or aggressive endometriosis. Imaging techniques can also be used to monitor the progress or remission of disease, or the impact of a therapy.

The miRNA listed in Table 3 or Table 4 can be useful for analysing tissue samples by staining e.g. using standard FISH. A fluorescently labelled nucleic acid, complementary in sequence to the miRNAs outlined in Table 3 or Table 4 can be contacted with a tissue sample to visualise the location of the miRNA. A single sample could be stained against multiple miRNAs, and these different miRNAs may be differentially labeled to enable them to be distinguished. As an alternative, a plurality of different samples can each be stained with a single, labeled miRNA.

Thus the invention provides a labeled nucleic acid which can hybridise to miRNA(s) listed in Table 3 or Table 4. The mRNA is preferably a human miRNA. Any salable label can be used e.g. quantum dots, spin labels, fluorescent labels, dyes, etc. These labelled nucleic acids can be used in methods of in vivo and/or in vitro imaging.

Alternative Biomarkers

The invention refers to auto-antibody and antigen biomarkers, with assays of auto-antibodies against an antigen being used in preference to assays of the antigen itself. In addition to these biomarkers, however, the invention can be used with other biological manifestations of the Table 2 antigens. For example, the level of mRNA transcripts encoding a Table 2 antigen can be measured, particularly in tissues where that gene is not normally transcribed (such as in the potential disease tissue). Similarly, the chromosomal copy number of a gene encoding a Table 2 antigen can be measured e.g. to check fora gene duplication event. The level of a regulator of a Table 2 antigen can be measured e.g. to look at a microRNA regulator of a gene encoding the antigen. Furthermore, things which are regulated by or respond to a Table 2 antigen can be assessed e.g. if an antigen is a regulator of a metabolic pathway then disturbances in that pathway can be measured.

The invention also refers to mRNA biomarkers. In addition to these biomarkers, however, the invention can be used with other biological manifestations of the Table 3 and Table 4 miRNAs. For exempla, the expression level of mRNA transcripts which are a target of a Table 3 or a Table 4 mRNA can be measured, particularly in tissues where changes in transcription level can easily be determined (such as in the potential disease tissue). Similarly, the copy number variation of a chromosomal location of a Table 3 or a Table 4 mRNA can be measured e.g. to check for a chromosomal deletion or duplication events.

The level of a regulator of transcription for a Table 3 or a Table 4 mRNA can be measured e.g. the methylation status of the mRNA chromosomal region.

A single pre-miRNA precursor may lead to one or more mature miRNA sequences, such as sequences excised from the 5′ and 3′ arms of the hairpin. The invention can be used to look for other mature mRNA sequences from the same pre-mRNA precursor. For example, other mature miRNA sequences from the same precursor may be appropriate biomarkers as well.

Further possibilities will be apparent to the skilled reader.

Preferred Panels

Preferred embodiments of the invention are based on at least two different biomarkers i.e. a panel. Panels of particular interest consist of or comprise combinations of one or more biomarkers listed in Table 1, optionally in combination with at least 1 further biomarker(s) e.g. from Table 5 etc. Preferred panels have from 2 to 6 biomarkers in total. Panels of particular interest consist of or comprise the combinations of bio markers listed in any of Tables 10 to 15. The panels useful for the invention (e.g. the panels listed in Tables 10 to 15) can be expanded by adding further (i.e. one or more) biomarker(s) to create a larger panel. The further biomarkers can usefully be selected from known biomarkers (as discussed above e.g. see Table 5). In general the addition does not decrease the sensitivity or specificity of the panel shown in the Tables. Such panels include, but are not limited to:
A panel comprising a biomarker selected from Table 1.
A panel comprising a biomarker selected from Table 2, Table 3 or Table 4.
A panel comprising or consisting of 2 different biomarkers, namely: (I) a biomarker selected from Table 1 and (ii) a further biomarker selected from Table 2.
A panel comprising or consisting of 2 different biomarkers, namely: (I) a biomarker selected from Table 1 and (ii) a further biomarker selected from Table 3.
A panel comprising or consisting of 2 different biomarkers, namely: (i) a biomarker selected from Table 1 and (ii) a further biomarker selected from Table 4.
A panel comprising or consisting of 3 different biomarkers, namely: (i) a group of 2 biomarkers selected from Table 11 and (ii) a further biomarker selected from Table 1.
A panel comprising or consisting of 3 different biomarkers, namely: (i) a group of 2 biomarkers selected from Table 11 and (ii) a further biomarker selected from Table 2.
A panel comprising or consisting of 3 different biomarkers, namely: (i) a group of 2 biomarkers selected from Table 11 and (ii) a further biomarker selected from Table 3
A panel comprising or consisting of 3 different biomarkers, namely: (i) a group of 2 biomarkers selected from Table 11 and (ii) a further biomarker selected from Table 4
A panel comprising or consisting of 4 different biomarkers, namely: (i) a group of 3 biomarkers selected from Table 12 and (ii) a further biomarker selected from Table 1
A panel comprising or consisting of 4 different biomarkers, namely: m a group of 3 biomarkers selected from Table 12 and (ii) a further biomarker selected from Table 2
A panel comprising or consisting of 4 different biomarkers, namely: (i) a group of 3 biomarkers selected from Table 12 and (ii) a further biomarker selected from Table 3
A panel comprising or consisting of 4 different biomarkers, namely: (i) a group of 3 biomarkers selected from Table 12 and (ii) a further biomarker selected from Table 4
A panel comprising or consisting of 5 different biomarkers, namely: (i) a group of 4 biomarkers selected from Table 13 and (ii) a further biomarker selected from Table 1
A panel comprising or consisting of 5 different biomarkers, namely: (i) a group of 4 biomarkers selected from Table 13 and (ii) a further biomarker selected from Table 2
A panel comprising or consisting of 5 different biomarkers, namely: (i) a group of 4 biomarkers selected from Table 13 and (ii) a further biomarker selected from Table 3
A panel compressing or consisting of 5 different biomarkers, namely: (i) a group of 4 biomarkers selected from Table 13 and (ii) a further biomarker selected from Table 4
A panel comprising or consisting of 6 different biomarkers, namely: (i) a group of 5 biomarkers selected from Table 14 and (ii) a further biomarker selected from Table 1
A panel comprising or consisting of 6 different biomarkers, namely: (i) a group of 5 biomarkers selected from Table 14 and (ii) a further biomarker selected from Table 2.
A panel comprising or consisting of 6 different biomarkers, namely: (i) a group of 5 biomarkers selected from Table 14 and (ii) a further biomarker selected from Table 3
A panel comprising or consisting of 6 different biomarkers, namely: (i) a group of 5 biomarkers selected from Table 14 and (ii) a further biomarker selected from Table 4
A panel comprising or consisting of 7 different biomarkers, namely: (i) a group of 6 biomarkers selected from Table 15 and (ii) a further biomarker selected from Table 1
A panel comprising or consisting of 7 different biomarkers, namely: (i) a group of 6 biomarkers selected from Table 15 and (ii) a further biomarker selected from Table 2
A panel comprising or consisting of 7 different biomarkers, namely: (i) a group of 6 biomarkers selected from Table 15 and (ii) a further biomarker selected from Table 3
A panel comprising or consisting of 7 different biomarkers, namely: (i) a group of 6 biomarkers selected from Table 15 and (ii) a further biomarker selected from Table 4
A panel comprising or consisting of a group of 12 different biomarkers selected from Table 10.

This panel is particularly useful for diagnosis.

Preferred panels have between 2 and 6 biomarkers in total.

A preferred panel comprises hsa-miR-150 and hsa-miR-574-5p. Another preferred panel comprises hsa-miR-342-3p and hsa-miR-574-5p. Mother preferred panel comprises hsa-miR-150 and hsa-mill-342-3p. Another preferred panel comprises hsa-miR-150, hsa-miR-122 and hsa-miR-574-5p. Another preferred panel comprises TPM1, hsa-miR-150 and hsa-miR-574-5p.

Different biomarkers can have different relative differential expression profiles in an endometriosis sample compared to a control sample. Pairs of these biomarkers (i.e. where one is up-regulated and the other is down-regulated relative to the same control) may provide a useful way of diagnosing endometriosis. For example, the inventors found that ebv-miR-BART2-5p and tea-miR-564 are up- and down-regulated, respectively, in endometriosis samples vs. control samples (healthy, non-endometriosis samples), so this pair would be useful. This divergent behaviour can enhance diagnosis of endometriosis when a pair of the biomarkers is assessed in the same sample.

Thus, a method of the invention can include a step of determining the expression levels of a first and a second biomarker of the invention in a subject's sample, wherein the first bb marker is up-regulated in an endometriosis sample compared to a non-endometriosis sample and the second biomarker is down-regulated in an endometriosis sample compared to the same non-endometriosis sample.

A method of the invention can include: (i) determining the expression level of a first biomarker of the invention in a subject's sample, (ii) determining the expression level of a second biomarker of the invention in the subject's sample, wherein the first biomarker is up-regulated in an endometriosis sample compared to a non-endometriosis sample and the second biomarker is down-regulated in an endometriosis sample compared to the same non-endometriosis sample, and (ii) comparing the determinations of (i) and (ii) with a non-endometriosis sample, an endometriosis sample and/or an absolute value, wherein the comparison provides a diagnostic indicator of whether the subject has endometriosis. Aberrant levels of the first and the second biomarkers, as compared to the known or standard expression levels of them in the non-endometriosis sample or endometriosis sample, and/or the absolute value, indicate that the subject has endometriosis.

General

The term “comprising” encompasses “Including” as well as “consisting” e.g. a composition “comprising” X may consist exclusively of X or may include something additional e.g. X+Y.

References to an antibody's ability to “bind” an antigen mean that the antibody and antigen interact strongly enough to withstand standard washing procedures in the assay to question. Thus non-specific binding will be minimised or eliminated.

An assay's “sensitivity” is the proportion of true positives which are correctly identified i.e the proportion of endometriosis subjects with auto-antibodies against the relevant antigen who test positive.

An assay's “specificity” is the proportion of true negatives which are correctly identified by i.e. the proportion of subjects without endometriosis who test negative for antibodies against the relevant antigen. Unless specifically stated, a method comprising a step of mixing two or more components does not require any specific order of mixing. Thus components can be mooed in any order. Where there are three components then two components can be combined with each other, and then the combination may be combined with the third component, etc.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref. 109. A preferred alignment is determined by the Smith-Waterman homology search algorithm using an atone gap search with a gap open penalty of 12 and a gap extension penalty of 2. BLOSUM matrix of 82. The Smith-Waterman homology search algorithm is disclosed in ref. 110.

### BRIEF DESCRIPTION OF DRAWINGS

FIG. 1 shows a volcano plot displaying the p-value of a microarray t-test on the y-axis versus the fold change in antibody levels between case and controls on the x-axis. The most interesting features can be found in the top left and top right area of the volcano plot. A dotted line is plotted in the graph to differentiate between potential markers and insignificant events. These cut-offs can be varied but a typical minimum selection criteria of a p-value less than 0.05 and a fold change of greater than log 2 of 0.585 (1.5-fold) was used to identify candidate biomarkers. Global median normalised data and not raw data is used to derive the fold-change values. Large differences in raw RFUs translate to small changes in this value following normalisation. Several of the best-performing markers (CCNB1IP1, TPM1 and SepI9) in this analysis are indicated.

FIG. 2 shows box plots for normalised data for. (A) RAN. (8) STUB1, (C) TPM1 and (D) HSPD1.

FIG. 3 shows a volcano plot displaying the p-value of a microarray t-test on the y-axis versus the fold change in miRNA levels between case and controls on the x-axis. Several of the best-performing markers (hsa-miR-150, hsa-miR-122, hsa-miR-342-3p, hsa-miR-483-3p, hsa-miR-1290, hsa-miR-3194-5p, hsa-miR-3883-5p, hsa-miR-3937, hsa-miR-1224-5p and hsa-miR-3648) in this analysis are highlighted.

FIG. 4 shows the hierarchical clustering of the significant miRNAs according to the type of tissue (i.e. case vs. control), where black signifies high expression, white signifies intermediate expression, and grey signifies low expression.

FIG. 5 is a scatter plot showing the correlation between the data from the microarray and TaqMan® mRNA qPCR assays.

FIG. 6 shows receiver operating characteristic (ROC) curves for individual mRNAs from the microarray data: (A) hsa-miR-150; sensitivity=0.8, specificity=0.65, AUC=0.8; (B) hsa-miR-574-5p; sensitivity=0.73, specificity=0.71, AUC=0.8; (C) hsa-miR-342-3p; sensitivity=0.66, specificity=0.76, AUC=0.78.

FIG. 7 shows receiver operating characteristic (ROC) curves for panels from the microarray data: (A) hsa-miR-150 and hsa-miR-574-5p; sensitivity=0.88, specificity=78, AUC=0.91; (B) hsa-miR-342-3p and hsa-miR-574-5p; sensitivity=0.78, specificity=0.78, AUC=0.86; (C) hsa-miR-150 and hsa-miR-342-3p; sensitivity=0.86, specificity=0.78, AUC=0.81; (D) hsa-miR-150, hsa-miR-122 and hsa-miR-574-5p; sensitivity=0.83, specificity=0.76, AUC=0.9.

FIG. 8 shows a receiver operating characteristic (ROC) curve for a combination of TPM1, hsa-miR-150 and hsa-miR-574-5p; sensitivity=20.9, specificity=0.75, AUC=0.92.

FIG. 9 shows a Venn diagram depicting the mRNAs which are statistically significantly differentially expressed (P<0.05) in normal endometrium (NE), normal peritoneum (NP), peritoneum endometriosis (ES) and ovarian endometriomas (OvES). The number of miRNAs that were statistically significantly expressed in more than one group is indicated in overlapping areas corresponding to appropriate groups. Areas A-F are examples of such overlapping areas.

FIG. 10 shows quantitative PCR results of ebv-miR-BART2-5p miRNA expression in endometriosis, normal endometrium from subjects having endometriosis and normal endometrium from subjects not having endometriosis.

### MODES FOR CARRYING OUT THE INVENTION

Study 1

1. Detection of Auto-Antibodies in Serum of Subjects Suffering from Endometriosis

a. Array Preparation

The examples refer to use of a “functional protein” array technology which has the ability to display native, discontinuous epitopes [111,112]. Proteins are full-length, expressed with a folding tag in insect cells and screened for correct folding before being arrayed in a specific, oriented manner designed to conserve native epitopes. Each array contains approximately 1550 human proteins representing ˜1500 distinct genes chosen from multiple functional and disease pathways printed in quadruplicate together with control proteins. In addition to the proteins on each array, four control proteins for the BCCP-myc tag (BCCP, BCCP-myc, β-galactosidase-BCCP-myc and β-galactosidase-BCCP) were arrayed, along with additional controls including Cy3-labeled biotin-BSA, dilution series of biotinylated-IgG and biotinylated-IgM and buffer-only spots.

Incubation of the arrays with serum samples slows detection of binding of serum immunoglobulins to specific proteins on the arrays, enabling the identification of both autoantibodies and their cognate antigens [112].

b. Biomarker Confirmation

Serum samples were obtained from two groups of subjects:
1. serum samples from subjects diagnosed with endometriosis (n=36).
2. serum samples from age-matched healthy donors (n=35).

For autoantibody profiling, serum samples were incubated with arrays separately. All arrays were incubated for 2 hours at room temperature (RT, 20° C.), followed by washing three times in fresh Triton-BSA buffer at RT for 20 minutes. The washed slides were incubated in a labelled anti-human IgG antibody at RT for 2 hours. Slides were washed three times in Triton-BSA buffer for 5 minutes at RT, rinsed, and centrifuged for 2 minutes at 240 g.

The probed and dried arrays were scanned using an Agilent High-Resolution microarray scanner at 10 μm resolution. The resulting 20-bit tiff images were feature extracted using Agilent's Feature Extraction software version 10.5 or 10.7.3.1. The microarray scans produced images for each array that were used to determine the intensity of fluorescence bound to each protein spot which were used to normalize and score array data.

Raw median signal intensity (also referred to as the relative fluorescent unit, RFU) of each protein feature (also referred to as a spot or antigen) on the array was subtracted from the local median background intensity. Alternative analyses use other measures of spot intensity such as the mean fluorescence, total fluorescence, as known in the art. The results of QC analyses showed that the platform performed well within expected parameters with relatively low technical variation.

The raw array data was normalized by consolidating the replicates (median consolidation), followed by normal transformation and then global median normalisation. Outliers were identified and removed. There is no method of normalisation which is universally appropriate and factors such as study design and sample properties must be considered. For the current study median normalisation was used. Other normalisation methods include, amongst others, SAM, quantile normalisation [113], multiplication of net fluorescent intensities by a normalisation factor consisting of the product of the 1st quartile of alt intensities of a sample and the mean of the 1st quartiles of ail samples and the “VSN” method [114]. Such normalisation methods are known in the art of microarray analysis.

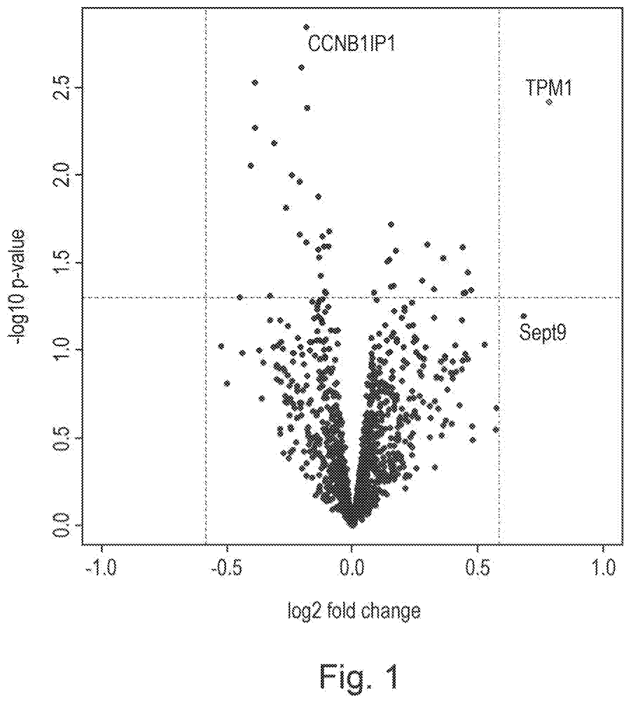

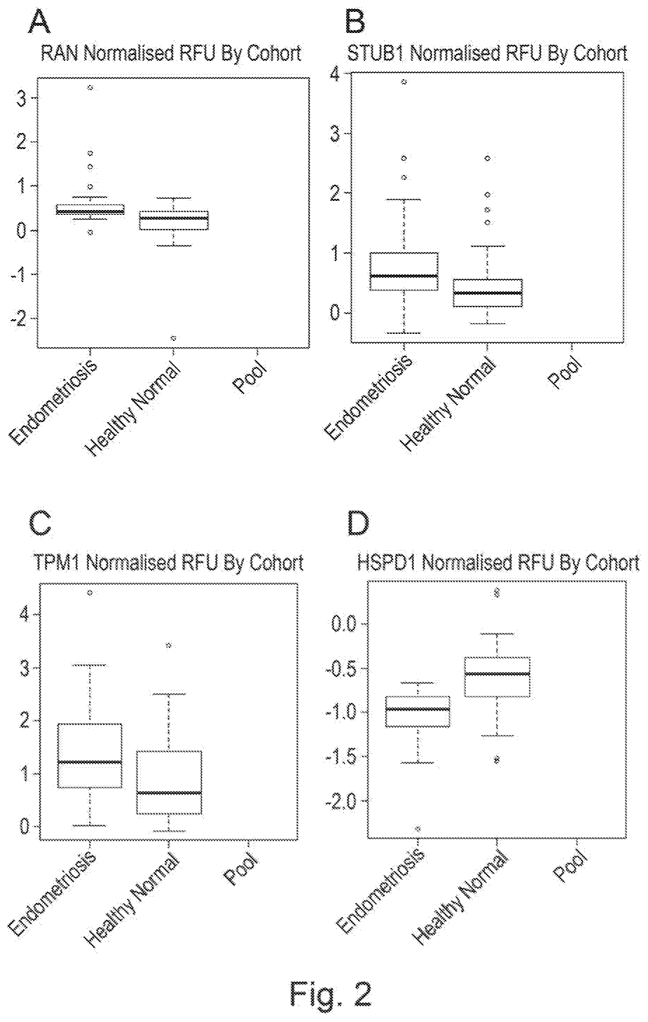

This normalised data was then used for the identification of individual candidate biomarkers and for the development of combinations of biomarkers (“panels”). Tools such as volcano plots (FIG. 1), scatter plots (FIG. 2) and boxplots were used to identify candidate biomarkers (Tables 6 and 8) with combinations of strong p-values and robust fold-changes when comparing case and control cohorts. Several proteins previously associated with endometriosis were identified (Table 5) including CDC42. EGFR, KIT, PPARG and WT1, thus validating this approach.

2 Detection of miRNA in Serum of Subjects Suffering from Endometriosis Using Microarrays

a. Array Preparation

For microarray fabrication and usage. Agilent Technologies' (‘Agilent’) miRNA microarray was used. The content of the microarray is continuously aligned with releases from the miRBase database [115, 116, 117, 118], representing all known miRNAs from human beings, as well as all know human viral mRNAs. These arrays are printed using Agilent's ink-jet in situ synthesis microarray fabrication machines.

b. Biomarker Confirmation

A set of 71 serum samples, sourced from patients with endometriosis (“case”; n=36) and normal (“control”; n=35) patients were processed to extract total RNA (including miRNA) using standard column filtration methodologies. The extracted serum samples were analysed using the Agilent miRNA microarray (G4870A-031181), according to their standard protocol, (manual part number G4170-90011, version 2.4). However, deviations from the standard protocol included labeling of the samples using 2.25 μl Cyanine 3-pCp, and hybridising the microarray slides for 44 hours.

The probed and dried arrays were then scanned using a microarray scanner capable of using an excitation wavelength suitable for the detection of the labelled miRNAs and to determine magnitude of miRNA binding to the complementary detection probe. The microarray scans produced images for each array that were used to determine the intensity of fluorescence bound to each oligonucleotide spot which were used to normalise and score array data.

The raw microarray scan image contains raw signal intensity (also referred to as the relative fluorescent unit, RFU) for each oligonucleotide spot (also referred to as a feature) on the array. These images were then feature extracted using Agilent's proprietary feature extraction software. Alternative analyses use other measures of spot intensity such as the mean fluorescence, total fluorescence, as known in the art.

The resulting average intensities of all oligonucleotide features on each array were then normalised to reduce the influence of technical bias (e.g. laser power variation, surface variation, input miRNA concentration, etc.) by a percentile normalisation procedure. Other methods for data normalisation suitable for the data include, amongst others, quantile normalisation [97]. Such normalisation methods are known in the art of microarray analysis.

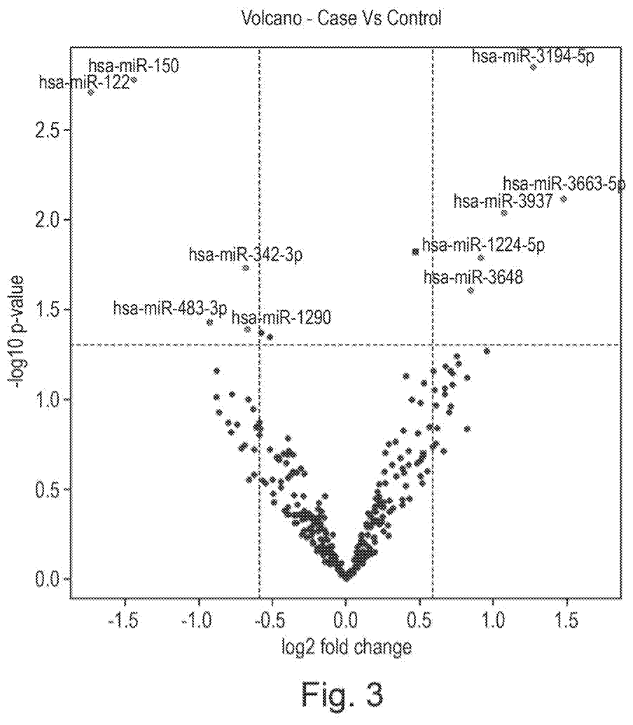

A linear model was fitted to evaluate statistical differences in miRNA expression between cases and controls. Volcano plot analysis of the miRNA microarray data is shown in FIG. 3. On the volcano plot (FIG. 3), the x-axis shows the log 2 fold change between case and control and the y-axis shows statistical significance (negative log in base 10 of p). Dots above the horizontal dashed line are a selection of significant hits.

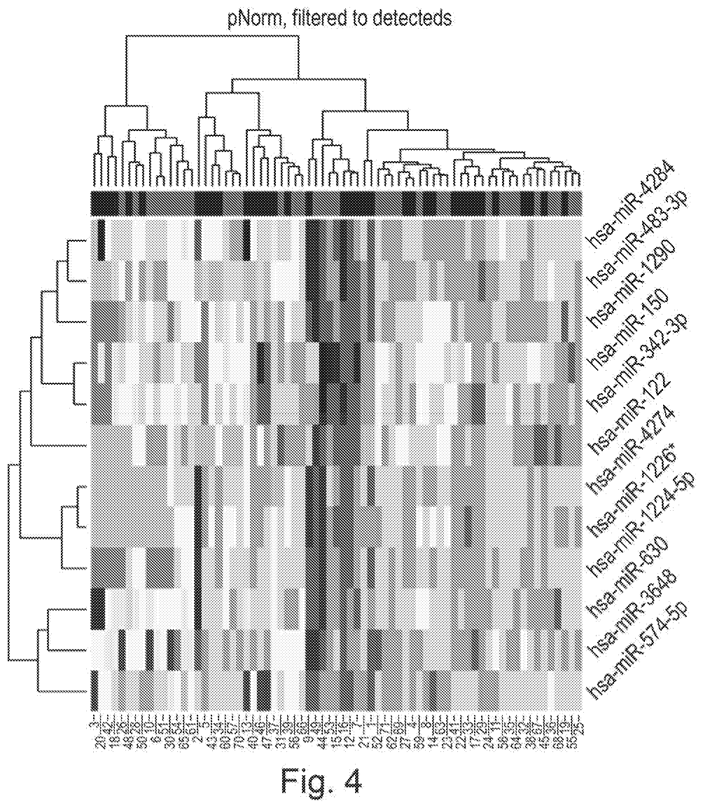

The hierarchical clustering of the significant mRNAs according to the type of tissue (i.e. case vs. control) is shown in FIG. 4, where black signifies high expression, white signifies intermediate expression, and grey signifies low expression.

3 Validation of miRNA in Serum of Subjects Suffering from Endometriosis Using qPCR

For quantitative PCR (“qPCR”) usage, Life Technologies' (“LifeTech”) TaqMan® miRNA assays were used. These assays am continuously aligned with releases from the MiRBase database, representing all known miRNAs from human beings, as well as all know human viral mRNAs. These TaqMan® mRNA assays employ a novel target-specific stern-loop reverse transcription primer to address the challenge of the short length of mature miRNA. The primer extends the 3′ end of the target to produce a template that can be used in standard TagMan® assay-based real-time PCR. Also, the stem-loop structure in the tail of the primer confers a key advantage to these assays: specific detection of the mature, biologically active mRNA.

Using the significant markers identified in the miRNA microarray experiments, a sub-selection of miRNAs were analysed using the LifeTech TaqMan® miRNA qPCR assays, according to their standard protocol, (manual pact number 4465407, revision date 30 Mar. 2012 (Rev. B)).

The TagMan® miRNA assays were scanned using a ViiA™ 7 Real-Time PCR System using an excitation wavelength suitable for the detection of the labelled mRNAs (for example, but not limited to, 6-FAM™ Dye). The qPCR scans produced traces for each TaqMan® mRNA assay which can be used, if applicable, to determine the amount of specific mRNA within a given sample, relative to a passive reference dye (for example, but not limited to, ROX).

The raw qPCR traces contain raw signal intensity (also referred to as ΔRn) to which an assay threshold (horizontal line) is applied after the raw traces have been baseline norms lied, which is necessary to remove aberrant signal. This threshold line intersects the qPCR trace at the point on the qPCR trace where the trace is logarithmic. From this, the qPCR cycle (Ct) can be determined. These qPCR traces are analysed using LifeTech's proprietary analysis software. Alternative analyses and analysis techniques are known in the art.

The median Ct and mean quantity was taken across the three sample replicates for each TaqMan® mRNA assay. Testing for statistically significant associations between the two groups (case vs control) was carried out by applying linear models to the normalised sample data to identify general miRNA changes between case samples and control samples. Statistical differences were calculated using a t-test.

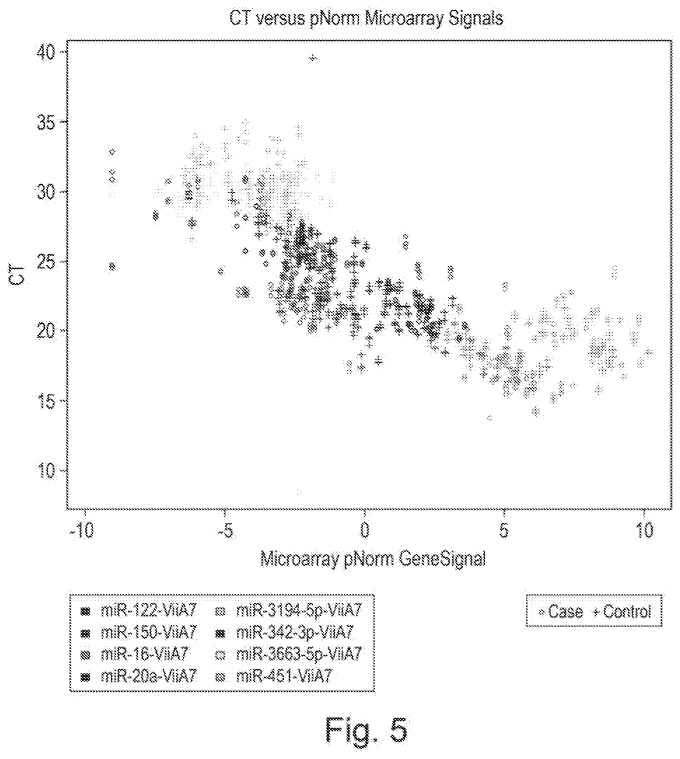

The data from the microarray and TaqMan® miRNA qPCR assays were analysed for Pearson correlation to assess the cross-platform robustness of the observed results (FIG. 5). FIG. 5 demonstrates that there is good correlation between a sub-set of the miRNA probes used for qPCR compared to those identified on the microarrays.

4. Multivariate Analysis: Combination of miRNA and Autoantibody Biomarkers in Serum of Subjects Suffering from Endometriosis

Panels of putative biomarkers were developed consisting of either autoantibodies alone, miRNAs alone or combinations containing both autoantibodies and miRNA species. Multivariate analysis was also performed incorporating data for galectin-3 and CA125 as variables however their inclusion did not improve on the performance of the miRNA and autoantibodies identified here. It is not possible to predict a priori which classifier will perform best with a given dataset, therefore data analysis was performed with 5 different feature ranking methods (1-5) plus forward and backward feature selection:
1. Entropy
2. Bhattacharyya
3. T-test
4. Wilcoxon
5. ROC
8. Forward selection
7. Backward selection

Other classification methods as known in the art could be used. Classifiers were then assessed for performance by referring to the combined sensitivity and specificity (S+S score) and area under the curve (AUC). Data were repeatedly split and analysis cycles repeated until a stable set of classifiers (“panels”) was identified. Nested cross validation was applied to the classification procedures in order to avoid overfilling of the study data. The performance of the classification was compared to a randomized set of case-control status samples (permutation assay) which should give no predictive performance and provides an indication of the background in the analysis. A figure close to 1.0 is expected for the null assay (equivalent to a sensitivity+specificity (S+S) score of 0.5+0.5, respectively) whereas an S+S score of 2.0 would indicate 100% sensitivity and 100% specificity. The difference between the values for the permutation analysis and the classifier performance indicates the relative strength of the classifier. The antigens and miRNAs identified from this study are provided in Tables 2 and 3.

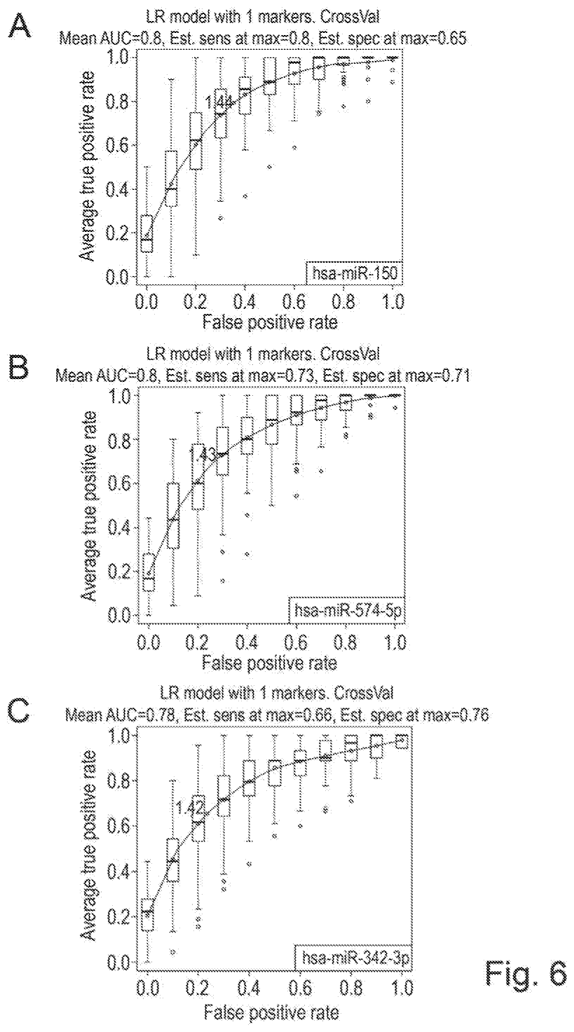

Table 8 shows the protein biomarkers that provided good performance, as judged by p value, fold-change, sensitivity, specificity, AUC. The best performing protein biomarkers are shown in Table 7. Table 8 shows the miRNA biomarkers that provided good performance, as judged by p value, fold-change, sensitivity, specificity, AUC. The best performing mRNA biomarkers are shown in Table 9. The ROC curves for some oldie best performing miRNA biomarkers (hsa miR-150, hsa-miR-574-5p and hsa-miR-342-3p) are shown in FIG. 6. The best performing protein and miRNA biomarkers are shown in Table 10. These represent biomarkers of particular interest as they correspond to the subset of biomarkers with the greatest predictive properties.

The analysis methods described above were used to build, test and identify combinations of biomarkers with greater sensitivity, specificity or AUC than the individual biomarkers disclosed in Table 1.

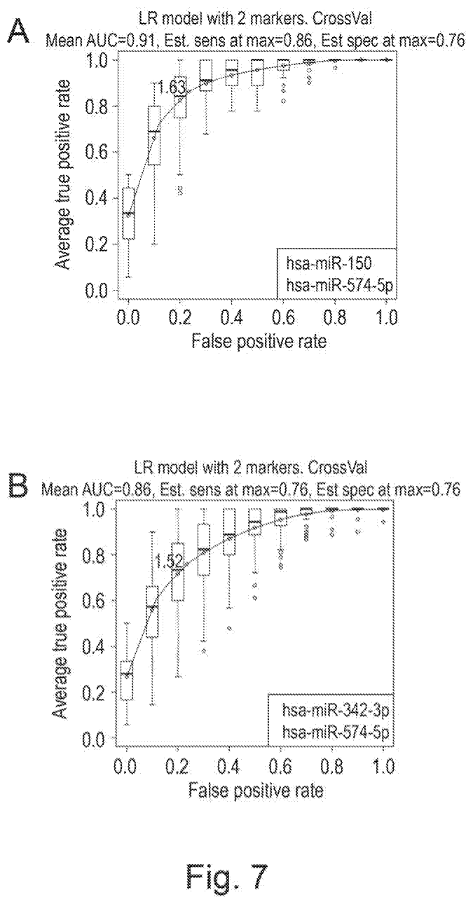

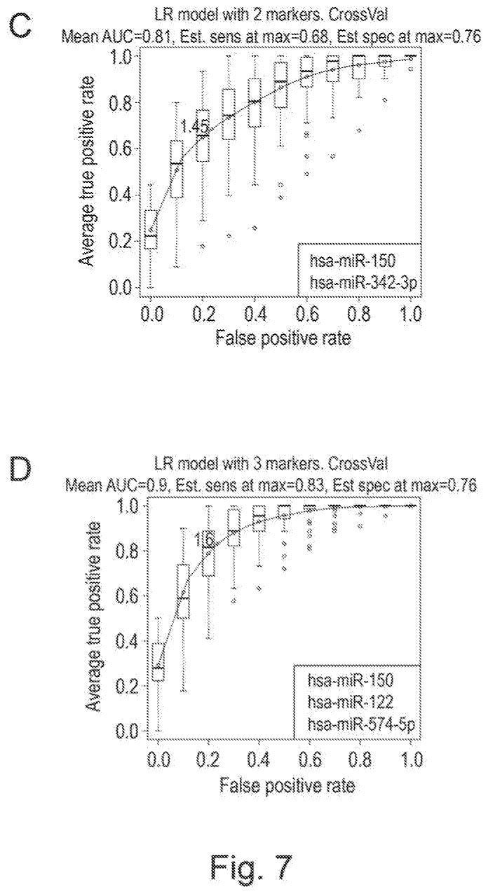

For each analysis, multiple combinations of putative biomarkers were derived and the performance of the derived panels was then ranked (rabies 11-15). Tables 11-15 show 2-mer, 3-mer, 4-mer, 5-mer and 8-mar panels that provide good performance. The ROC curves for some of the best performing combinations are shown in FIG. 7 (A=2-mer panel of hsa-miR-150 and hsa-miR-574-5p; B=2-mer panel of hsa-miR-342-3p and hsa-miR-574-5p; C=2-mer panel of hsa-miR-150 and hsa-miR-342-3p; D=3-mer panel of hsa-miR-150, hsa-miR-122 and hsa-miR-574-5p).

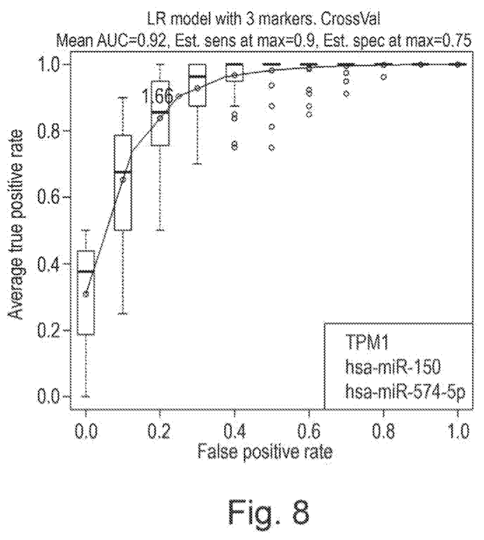

The biomarkers with the greatest diagnostic power, as judged by p value, fold-change, sensitivity, specificity, AUC and/or frequency of appearance in the panels derived were identified and combined into a single list of antigens and miRNAs. FIG. 8 show the ROC curve for a 3-mer panel of TPM1, hsa-miR 150 and hsa-miR-574-5p, which is one of the best performing combinations. Thus, panels containing a mixture of protein and miRNA biomarkers also provide good diagnostic performance.

Study 2

1. Detection of miRNA in Tissue Samples of Subjects Suffering from Endometriosis Using NanoString Technology

The fresh tissue samples used in this study include: peritoneal endometriosis (ES), ovarian endometriomas (OvES), normal endometrium (NE) and normal peritoneum (NP).

miRNA Extraction

Total RNA from specimens was extracted using the mirVana™ PARIS™ kit (Ambion) as per their supplied protocol. RNA quantification and integrity was assessed using the Eukaryote total RNA nano assay by Aligent Technologies™. Following validation, 100 ng of total RNA was used in the nCounter® miRNA Expression Assay (NanoString Technologies) enabling an ultrasensitive miRNA detection in total RNA across all biological levels of expression without the use of reverse transcription or amplification. 735 human and human-associated viral miRNAs derived from MiRBase were scanned. Unique multiplexed annealing of specific oligonucleoside tags were ligated onto their target miRNA followed by an enzymatic purification to remove all unligated tags. Excess unbound probes and RNA were washed using a two-step magnetic bead-based purification system on the nCounter Prep Station. Remaining miRNA was attached to a cartridge surface and polarised. The Cartridge was scanned and data collection was performed on the nCounter digital analyser. Digital images were processed and miRNA counts were tabulated.

Statistical Analysis

The R project (R version 2.12.1) (http://www.R-project.org) was used for statistical and clustering analysis. Quantified gene expression signal levels derived from the nCounter® miRNA Egression Assay (NanoString Technologies) were logarithmically transformed (base 2) and quantile-normalized prior to further exploration. In order to determine miRNA expression detectability threshold the log 2 signal values were ranked in increasing order and binned into 0.5 expression level categories. Subsequently, the differences in successive expression level frequencies were calculated and the expression value following the most significant signal increase was considered as miRNA expression detectability threshold. miRNA expressions at any level (above log 2 expression value of 3.25) were counted in each group and subsequently chi-square test was performed to assess if the numbers are significantly different from expected average. The global expression of miRNAs between all groups was not noted to be significantly different (chi square p-value 0.62664147).

Analysis of variance (ANOVA) was performed per each gene across all samples assigned to appropriate groups in order to identify differentially expressed mRNAs. The Benjamin & Hochberg multiple hypothesis testing correction was applied to control the false discovery rate (FDR). Genes that remained statistically significant (corrected p-value <0.05) were selected for unsupervised hierarchical clustering performed based on distances calculated by means of Spearman correlation coefficient and ward agglomeration method.

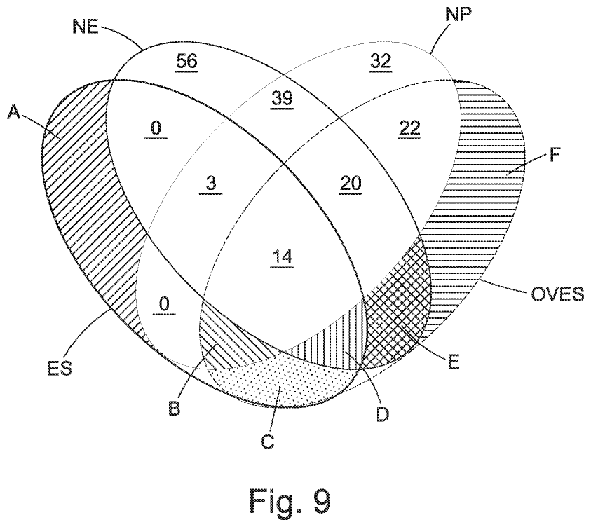

Subsequently, pairwise t-tests with corrections for multiple testing were applied as a post hoc analysis. T-test was performed between a particular sample group and all other sample groups. For example, ES samples were compered against all other groups (OvES, NP and NE), OvES samples were compared against all other groups (ES, NP and NE) etc. Computed t-statistics and signal intensity fold changes were further used to determine significantly up- and down-regulated miRNAs in each sample group and to generate Venn diagrams depicting the overlap between miRNAs differentially expressed in investigated groups (FIG. 9).

Area A in FIG. 9 shows that the levels of 2 miRNAs (ebv-miR-BART2-5p and hsa-miR-564, also listed in Table 16) are significantly different in ES samples, compared to that in OvES, NE and NP samples. Thus, these miRNAs are particularly useful for detecting peritoneum endometriosis. These miRNAs are particularly useful in practice because peritoneum endometriosis is relatively more difficult to detect using conventional techniques, e.g. ultrasound or MRI, compared to endometriomas.

Area C in FIG. 9 shows that the level of 1 miRNA (listed in Table 15) is significantly different in ES and OvES samples compared to that in NE and NP samples. Thus, these miRNAs are particularly useful for detecting endometriosis, irrespective of where the endometriosis is.

Area F in FIG. 9 shows that the levels of the miRNAs listed in Table 17 are significantly different in OvES samples compared to that in ES, NE and NP samples. Thus, these miRNAs are particularly useful for detecting endometriosis in the ovaries, e.g. endometriomas.

Area B in FIG. 9 shows that the levels of the miRNAs listed in Table 20 are significantly different in ES, OvES and NP samples compared to that in NE samples.

Area D in FIG. 9 shows that the levels of 2 miRNAs (listed in Table 21) are significantly different in ES, OvES and NE samples compared to that in NP samples.

Area E in FIG. 9 shows that the levels of the mRNAs fisted in Table 19 are significantly different in OvES and NE samples compared to that in ES and NP samples.

2 Validation of miRNA in Tissue Samples of Subjects Suffering From Endometriosis Using qPCR

PCR was performed to validate the microarray data. TaqMan® MicroRNA Assays (Applied Biosystems) were used.

cDNA was synthesized from total RNA using a commercially available specific EBV-miR-bart2-5p assay. RNU-44 and hsa-miR-26b were used as the mature miRNA endogenous controls. The miRNA 26b is a commonly expressed vertebrate mRNA and was used as a control primer for the reactions. Real time PCR amplification was performed on triplicates of each sample using the TaqMan2X Universal PCR Master Mix (Applied Biosystems).

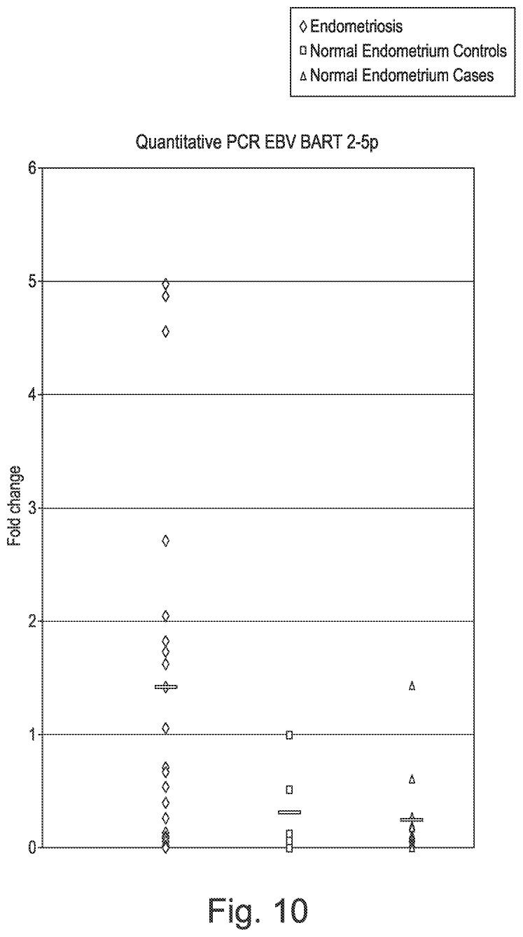

Significant differential expression of the ebv-miR-BART2-5p miRNA was observed between normal endometrium from controls, normal endometrium from subjects having endometriosis and endometriosis (FIG. 10). The level of expression of ebv-miR-BART2-5p in normal endometrium from non-endometriosis subjects is significantly different from that in endometriosis (p=0.0067). There was no statistical significance between normal endometrial samples from subjects having endometriosis and normal endometrial samples from non-endometriosis subjects (p=0.02).

It will be understood that the invention has been described by way of example only and modifications may be made whist remaining within the scope and spirit of the invention.

TABLE 1
Biomarkers useful with the invention

| No.⁽ⁱ⁾ | Symbol⁽ⁱⁱ⁾ | Name⁽ⁱⁱⁱ⁾ | Type |
|---|---|---|---|
| 1. | ACTB | Homo sapiens actin beta | Auto-antigen |
| 2. | ADD1 | Homo sapiens adducin 1 (alpha) | Auto-antigen |
| 3. | ADSL | Homo sapiens adenylosuccinate lyase | Auto-antigen |
| 4. | AK2 | Homo sapiens adenylate kinase 2 transcript variant AK2A | Auto-antigen |
| 5. | KLK3 | Homo sapiens kallikrein 3 (prostate specific antigen) | Auto-antigen |
| | | transcript variant 1 | |
| 8. | ATF1 | Homo sapiens activating transcription factor 1 | Auto-antigen |
| 7. | CKM | Homo sapiens creatine kinase muscle | Auto-antigen |
| 8. | CLK2 | Homo sapiens CDC-like kinase 2 transcript variant phclk2 | Auto-antigen |
| 9. | DDB2 | Homo sapiens damage-specific DNA binding protein 2 | Auto-antigen |
| | | 48 kDa | |
| 10. | DTYMK | Homo sapiens deoxythymidylate kinase (thymidylate | Autoantigen |
| | | kinase) | |
| 11. | DUSP4 | Homo sapiens dual specificity phosphatase 4 transcript | Auto-antigen |
| | | variant 1 | |
| 12. | E2F6 | Homo sapiens E2F transcription factors | Auto-antigen |
| 13. | EXT2 | Homo sapiens exostoses (multiple) 2 | Auto-antigen |
| 14. | FGFR4_ext | Homo sapiens fibroblast growth factor receptor 4 transcript | Auto-antigen |
| | | variant 3 | |
| 15. | FIGF | Homo sapiens c-fos induced growth factor (vascular | Auto-antigen |
| | | endothelial growth factor D) | |
| 16. | FKBP3 | Homo sapiens FK506 binding protein 3 25 kDa | Auto-antigen |
| 17. | GALK1 | Homo sapiens galactokinase 1 | Auto-antigen |
| 18. | GK2 | Homo sapiens glycerol kinase 2 | Auto-antigen |
| 19. | GRB7 | Homo sapiens growth factor receptor-bound protein 7 | Auto-antigen |
| 20. | GTF2B | Homo sapiens general transcription factor IIB | Auto-antigen |
| 21. | GTF2H1 | Homo sapiens general transcription factor IIH polypeptide | Auto-antigen |
| | | 1 62 kDa | |
| 22. | GTF2H2 | Homo sapiens general transcription factor IIH polypeptide | Auto-antigen |
| | | 2 44 kDa | |
| 23. | HSPD1 | Homo sapiens heat shock 60 kDa protein 1 (chaperonin) | Auto-antigen |
| 24. | IDI1 | Homo sapiens Isopentenyl-diphosphate delta isomerase | Auto-antigen |
| 25. | IFI16 | Homo sapiens interferon, gamma-inducible protein 16, | Auto-antigen |
| 26. | LDHA | Homo sapiens lactate dehydrogenase A | Auto-antigen |
| 27. | LYL1 | Homo sapiens lymphoblastic leukemia derived sequence 1 | Auto-antigen |
| 28. | MARK3 | Homo sapiens MAP/microtubule affinity-regulating kinase | Auto-antigen |
| | | 3 | |
| 29. | MPP3 | Homo sapiens membrane protein palmitoylated 3 (MAGUK | Auto-antigen |
| | | p55 subfamily member 3) | |
| 30. | TRIM37 | Homo sapiens tripartite motif-containing 37, | Auto-antigen |
| 31. | NCF2 | Homo sapiens neutrophil cytosolic factor 2 (65 kDa, chronic | Auto-antigen |
| | | granulomatous disease, autosomal 2), | |
| 32. | RPL10A | Homo sapiens ribosomal protein L10a | Auto-antigen |
| 33. | NFYA | Homo sapiens nuclear transcription factor Y alpha | Auto-antigen |
| 34. | NRAS | Homo sapiens neuroblastoma RAS viral (v-ras) oncogene | Auto-antigen |
| | | homolog | |
| 35. | NTRK3_ext | Homo sapiens neurotrophic tyrosine kinase receptor type 3 | Auto-antigen |
| | | transcript variant 3 | |
| 36. | OAS2 | Homo sapiens 2′-5′-oligoadenylate synthetase 2 69/71 kDa | Auto-antigen |
| 37. | endometriosis | Homo sapiens pyruvate carboxylase | Auto-antigen |
| 38. | CDK17 | Homo sapiens endometriosisTAIRE protein kinase 2 | Auto-antigen |
| 39. | PDE4A | Homo sapiens Homo sapiens phosphodiesterase 4A, | Auto-antigen |
| | | cAMP-specific (phosphodiesterase E2 dunce homolog, | |
| | | Dro | |
| 40. | PHF1 | Homo sapiens PHD finger protein 1 transcript variant 2 | Auto-antigen |
| 41. | PKM2 | Homo sapiens pyruvate kinase muscle transcript variant 1 | Auto-antigen |
| 42. | MAP2K5 | Homo sapiens mitogen-activated protein kinase kinase 5, | Auto-antigen |
| | | transcript variant A | |
| 43. | PRPS2 | Homo sapiens phospharibosyl pyrophosphate synthetase | Auto-antigen |
| | | 2 | |
| 44. | RAN | Homo sapiens RAN member RAS oncogene family | Auto-antigen |
| 45. | RB1 | Homo sapiens retinoblastoma 1 (including osteosarcoma) | Auto-antigen |
| 46. | RBMS1 | Homo sapiens Homo sapiens RNA binding motif single | Auto-antigen |
| | | stranded interacting protein 1 transcript variant | |
| 47. | RET_a | Home sapiens ret proto-oncogene (multiple endocrine | Auto-antigen |
| | | neoplasia and medullary thyroid carci | |
| 48. | RORC | Homo sapiens RAR-related orphan receptor C | Auto-antigen |
| 49. | RPL18 | Homo sapiens ribosomal protein L18 | Auto-antigen |
| 50. | RPL18A | Homo sapiens ribosomal protein L18a | Auto-antigen |
| 51. | RPL28 | Homo sapiens ribosomal protein L28 | Auto-antigen |
| 52. | RPL31 | Homo sapiens ribosomal protein L31 | Auto-antigen |
| 53. | RPL32 | Homo sapiens ribosomal protein L32 | Auto-antigen |
| 54. | S100A6 | Home sapiens S100 calcium binding protein A6 (calcyclin) | Auto-antigen |
| 55. | SCP2 | Home sapiens sterol carrier protein 2 transcript variant 2 | Auto-antigen |
| 56. | SIAH1 | Homo sapiens seven in absentia homolog 1 (Drosophila) | Auto-antigen |
| | | transcript variant 2 | |
| 57. | SMARCD1 | Homo sapiens SWI/SNF related matrix associated actin | Auto-antigen |
| | | dependent regulator of chromatin subfamily d member 1 | |
| 58. | SMARCE1 | Home sapiens SWI/SNF related matrix associated actin | Auto-antigen |
| | | dependent regulator of chromatin | |
| 59. | SOD2 | Hamo sapiens superoxide dismutase 2 mitochondrial | Auto-antigen |
| 60. | SOX2 | Homo sapiens SRY (sex determining region Y)-box 2 | Auto-antigen |
| 61. | SRPK1 | Homo sapiens SFRS protein kinase 1 | Auto-antigen |
| 62. | TPM1 | Homo sapiens tropomyosin 1 (alpha) | Auto-antigen |
| 63. | NR2C1 | Homo sapiens nuclear receptor subfamily 2 group C | Auto-antigen |
| | | member 1 | |
| 64. | TRIPS | Homo sapiens thyroid hormone receptor interactor 6, | Auto-antigen |
| 65. | UBA1 | Homo sapiens Homo sapiens ubiquitin-activating enzyme | Auto-antigen |
| | | E1 (A1S9T and BN75 temperature sensitivity compl | |
| 66. | VCL | Homo sapiens vinculin | Auto-antigen |
| 67. | ZNF41 | Homo sapiens zinc finger protein 41 transcript variant 2 | Auto-antigen |
| 66. | PAX8 | Homo sapiens paired box gene 8 transcript variant PAX8A | Auto-antigen |
| 69. | NRIP1 | Homo sapiens nuclear receptor interacting protein 1 | Auto-antigen |
| 70. | PIP4K2B | Homo sapiens phosphatidylinositol-4-phosphate 5-kinase | Auto-antigen |
| | | type II beta transcript variant 2 | |
| 71. | UXT | Homo sapiens ubiquitously-expressed transcript | Auto-antigen |
| 72. | API5 | Homo sapiens apoptosis inhibitor 5 | Auto-antigen |
| 73. | MKNK1 | Homo sapiens MAP kinase-interacting serine/threonine | Auto-antigen |
| | | kinase 1 | |
| 74. | SUCLA2 | Homo sapiens succinate-CoA ligase ADP-forming beta | Auto-antigen |
| | | subunit | |
| 75. | LDB1 | Homo sapiens LIM domain binding 1 | Auto-antigen |
| 76. | NAE1 | Homo sapiens amyloid beta precursor protein binding | Auto-antigen |
| | | protein 1 transcript variant 1 | |
| 77. | PAPSS2 | Homo sapiens 3′-phosphoadenosine 5′-phosphosulfate | Auto-antigen |
| | | synthase 2 | |
| 78. | USP10 | Homo sapiens ubiquitin specific protease 10 | Auto-antigen |
| 79. | PRPF4 | Homo sapiens Homo sapiens PRP4 pre- | Auto-antigen |
| 80. | AIM2 | Homo sapiens absent in melanoma 2 | Auto-antigen |
| 81. | TBPL1 | Homo sapiens TBP-like 1 | Auto-antigen |
| 82. | TRAF4 | Homo sapiens TNF receptor-associated factor 4 transcript | Auto-antigen |
| | | variant 1 | |
| 83. | SOCS5 | Homo sapiens suppressor of cytokine signaling 5 | Auto-antigen |
| 84. | ZSCAN12 | Homo sapiens zinc finger protein 305 | Auto-antigen |
| 85. | HDAC4 | Homo sapiens cDNA | Auto-antigen |
| 86. | KIAA0101 | Homo sapiens KIAA0101 gene product | Auto-antigen |
| 87. | IP6K1 | Homo sapiens Inositol hexaphosphate kinase 1 | Auto-antigen |
| 88. | RNF40 | Homo sapiens ring finger protein 40 transcript variant 1 | Auto-antigen |
| 89. | GPHN | Homo sapiens gephyrin | Auto-antigen |
| 90. | HRSP12 | Homo sapiens translational inhibitor protein p14.5 | Auto-antigen |
| 91. | STUB1 | Homo sapiens STIP1 homology and U-Box containing | Auto-antigen |
| | | protein 1 | |
| 92. | TRAIP | Homo sapiens TRAF interacting protein | Auto-antigen |
| 93. | PLK4 | Homo sapiens serine/threonine kinase 18 | Auto-antigen |
| 94. | CCNI | Homo sapiens cyclin i | Auto-antigen |
| 95. | 1L24 | Homo sapiens interleukin 24 transcript variant 1 | Auto-antigen |
| 96. | CDK20 | Homo sapiens cell cycle related kinase | Auto-antigen |
| 97. | PABendometriosis 1 | Homo sapiens poly(A) binding protein cytoplasmic 1 | Auto-antigen |
| 98. | MED4 | Homo sapiens vitamin D receptor interacting protein | Auto-antigen |
| 99. | NMET | Homo sapiens non-metastatic cells 7 protein expressed in | Auto-antigen |
| | | (nucleoside-diphosphate kinase) transcript variant 1 | |
| 100. | PHF11 | Homo sapiens PHD finger protein 11 | Auto-antigen |
| 101. | IRAK4 | Homo sapiens interleukin-1 receptor-associated kinase 4 | Auto-antigen |
| | | mRNA (cDNA clone MGC: 13330 ) | |
| 102. | TXNDC3 | Homo sapiens thioredoxin domain containing 3 | Auto-antigen |
| | | (spermatozoa) | |
| 103. | TAOK3 | Homo sapiens STE20-like kinase | Auto-antigen |
| 104. | STYXL1 | Homo sapiens dual specificity phosphatase 24 (putative) | Autoantigen |
| 105. | ASB1 | Homo sapiens ankyrin repeat and SOCS box-containing 1 | Autoantigen |
| 106. | MST4 | Homo sapiens Mst3 and SOK1-related kinase (MASK) | Autoantigen |
| 107. | PELO | Homo sapiens pelota homolog (Drosophila) | Autoantigen |
| 108. | ETNK2 | Homo sapiens ethanolamine kinase 2 | Autoantigen |
| 109. | RFK | Homo sapiens riboflavin kinase | Autoantigen |
| 110. | C9orf86 | Homo sapiens chromosome 9 open reading frame 86 | Auto-antigen |
| 111. | DDX55 | Homo sapiens DEAD (Asp-Glu-Ala-Asp) box polypeptide | Autoantigen |
| | | 55 | |
| 112. | CCNB1IP1 | Homo sapiens CDNA | Autoantigen |
| 113. | KLHL12 | Homo sapiens kelch-like protein C3IP1 | Autoantigen |
| 114. | SAV1 | Homo sapiens Salvador homolog 1 (Drosophila) | Autoantigen |
| 115. | CAMKV | Homo sapiens hypothetical protein MGC8407 | Autoantigen |
| 116. | NSBP1 | Homo sapiens nucleosomal binding protein 1, | Autoantigen |
| 117. | ALPK1 | Homo sapiens alpha-kinase 1 mRNA (cDNA clone | Autoantigen |
| | | MGC:71554) | |
| 118. | ELMOD3 | Homo sapiens RNA binding motif end ELMO domain 1 | Autoantigen |
| 119. | AIFM2 | Homo sapiens apoptosis-inducing factor (AIF)-like | Autoantigen |
| | | mitochondrion-associated inducer of death | |
| 120. | RHOT2 | Homo sapiens ras homolog gene family member T2 | Autoantigen |
| 121. | PYGO2 | Homo sapiens pygopus 2 | Auto-antigen |
| 122. | ebv-mR-BART12 | ebv-mIR-BART12 | miRNA |
| 123. | ebv-mR-BART14 | ebv-mIR-BART14 | miRNA |
| 124. | ebv-miR-BART16 | ebv-mIR-BART16 | miRNA |
| 125. | ebv-miR-BART20-5p | ebv-mIR-BART20-5p | miRNA |
| 126. | ebv-miR-BART2-5p | ebv-mIR-BART2-5p | miRNA |
| 127. | MIRLET7B | hsa-let-7b* | miRNA |
| 128. | MIRLET7F1 | haa-let-7f | miRNA |
| 129. | MIRLET7F2 | hsa-let-7f-1* | miRNA |
| 130. | MIRLET7G | hsa-let-7g | miRNA |
| 131. | MIR103A1 | hsa-miR-103a | miRNA |
| 132. | MIR10b | hsa-miR-10b | miRNA |
| 133. | MIR1183 | hsa-miR-1183 | miRNA |
| 134. | MIR1202 | hsa-miR-1202 | miRNA |
| 135. | MIR1207 | hsa-miR-1207-5p | miRNA |
| 136. | MIR122 | hsa-miR-122 | miRNA |
| 137. | MIR1224 | hsa-miR-1224-5p | miRNA |
| 138. | MIR1225 | hsa-miR-1225-3p | miRNA |
| 139. | MIR1225 | hsa-miR-1225-5p | miRNA |
| 140. | MIR1228 | hsa-miR-1226* | miRNA |
| 141. | MIR1228 | hsa-miR-1228* | miRNA |
| 142. | MIR1234 | hsa-miR-1234 | miRNA |
| 143. | MIR1237 | hsa-miR-1237 | miRNA |
| 144. | MIR1238 | hsa-miR-1238 | miRNA |
| 145. | MIR125a | hsa-miR-125a-5p | miRNA |
| 146. | MIR1280A | hsa-miR-1260 | miRNA |
| 147. | MIR1280b | hsa-miR-1250b | miRNA |
| 148. | MIR1280 | hsa-miR-1280 | miRNA |
| 149. | MIR1281 | hsa-miR-1281 | miRNA |
| 150. | MIR1290 | hsa-miR-1290 | miRNA |
| 151. | MIR1291 | hsa-miR-1291 | miRNA |
| 152. | MIR129-1/MIR129-2 | hsa-miR-129-3p | miRNA |
| 153. | MIR1301 | hsa-miR-1301 | miRNA |
| 154. | MIR135A1/ | hsa-miR-135a | miRNA |
| | MIR135A2 | | |
| 155. | MIR135A1/ | hsa-miR-135a* | miRNA |
| | MIR135A2 | | |
| 156. | MIR141 | hsa-miR-141 | miRNA |
| 157. | MIR142 | hsa-miR-142-3p | miRNA |
| 158. | MIR149 | hsa-miR-149 | miRNA |
| 159. | MIR150 | hsa-miR-150 | miRNA |
| 160. | MIR150 | hsa-miR-150* | miRNA |
| 161. | MIR1539 | hsa-miR-1539 | miRNA |
| 162. | MIR181A2 | hsa-miR-181a | miRNA |
| 163. | MIR1825 | hsa-miR-1825 | miRNA |
| 164. | MIR186 | hsa-miR-185 | miRNA |
| 165. | MIR18a | hsa-miR-18a | miRNA |
| 166. | MIR18b | hsa-miR-18b | miRNA |
| 167. | MIR191 | haa-miR-191* | miRNA |
| 168. | MIR197 | hsa-miR-197 | miRNA |
| 169. | MIR198 | hsa-miR-198 | miRNA |
| 170. | MIR205 | hsa-miR-205 | miRNA |
| 171. | MIR2116 | hsa-miR-2116* | miRNA |
| 172. | MIR215 | hsa-miR-215 | miRNA |
| 173. | MIR22 | hsa-miR-22 | miRNA |
| 174. | MIR223 | hsa-miR-223 | miRNA |
| 175. | MIR2276 | hsa-miR-2276 | miRNA |
| 176. | MIR23c | hsa-miR-23c | miRNA |
| 177. | MIR26A1/MIR26A2 | hsa-miR-26a | miRNA |
| 178. | MIR28 | hsa-miR-28-3p | miRNA |
| 179. | MIR28 | hsa-miR-28-5p | miRNA |
| 180. | MIR2981/MIR2982 | hsa-miR-29b | miRNA |
| 181. | MIR30a | hsa-miR-30a | miRNA |
| 182. | MIR30b | hsa-miR-30b | miRNA |
| 183. | MIR3131 | hsa-miR-3131 | miRNA |
| 184. | MIR3138 | hsa-miR-3138 | miRNA |
| 185. | MIR3149 | hsa-miR-3149 | miRNA |
| 186. | MIR3156-1/ | hsa-miR-3156-6p | miRNA |
| | MIR3156-2/ | | |
| | MIR3158-3 | | |
| 187. | MIR3180-1/ | hsa-miR-3180-5p | miRNA |
| | MIR3180-2/ | | |
| | MIR3180-3/ | | |
| | MIR3180-4/ | | |
| | MIR3180-5 | | |
| 188. | MIR3194 | hsa-miR-3194-5p | miRNA |
| 189. | MIR3195 | hsa-miR-3195 | miRNA |
| 190. | MIR3196 | hsa-miR-3196 | miRNA |
| 191. | MIR32 | hsa-miR-32 | miRNA |
| 192. | MIR320C1/ | hsa-miR-320c | miRNA |
| | MIR320C2 | | |
| 193. | MIR324 | hsa-miR-324-3p | miRNA |
| 194. | MIR328 | hsa-miR-328 | miRNA |
| 195. | MIR331 | hsa-miR-331-5p | miRNA |
| 196. | MIR33b | hsa-miR-33b* | miRNA |
| 197. | MIR342 | hsa-miR-342-3p | miRNA |
| 198. | MIR34a | hsa-miR-34a | miRNA |
| 199. | MIR3610 | hsa-miR-3610 | miRNA |
| 200. | MIR3648 | hsa-miR-3648 | miRNA |
| 201. | MIR3652 | haa-miR-3652 | miRNA |
| 202. | MIR3663 | hsa-miR-3663-5p | miRNA |
| 203. | MIR3667 | hsa-miR-3667-5p | miRNA |
| 204. | MIR382 | hsa-miR-382 | miRNA |
| 205. | MIR3911 | hsa-miR-3911 | miRNA |
| 206. | MIR3937 | hsa-miR-3937 | miRNA |
| 207. | MIR4257 | hsa-miR-4257 | miRNA |
| 208. | MIR425 | hsa-miR-425* | miRNA |
| 209. | MIR4270 | hsa-miR-4270 | miRNA |
| 210. | MIR4274 | hsa-miR-4274 | miRNA |
| 211. | MIR4281 | hsa-miR-4281 | miRNA |
| 212. | MIR4284 | hsa-miR-4284 | miRNA |
| 213. | MIR4288 | hsa-miR-4286 | miRNA |
| 214. | MIR429 | hsa-miR-429 | miRNA |
| 215. | MIR4290 | hsa-miR-4290 | miRNA |
| 216. | MIR4313 | hsa-miR-4313 | miRNA |
| 217. | MIR4323 | hsa-miR-4323 | miRNA |
| 218. | MIR466 | hsa-miR-466 | miRNA |
| 219. | MIR483 | hsa-miR-483-3p | miRNA |
| 220. | MIR484 | hsa-miR-484 | miRNA |
| 221. | MIR485 | hsa-miR-485-3p | miRNA |
| 222. | MIR486 | hsa-miR-488-5p | miRNA |
| 223. | MIR488 | hsa-miR-488 | miRNA |
| 224. | MIR497 | hsa-miR-497 | miRNA |
| 225. | MIR511-1 | hsa-miR-511 | miRNA |
| 226. | MIR511-2 | hsa-miR-512 | miRNA |
| 227. | MIR520C | hsa-miR-520c-3p | miRNA |
| 228. | MIR550A1/ | hsa-miR-550a | miRNA |
| | MIR550A2/ | | |
| | MIR550A3 | | |
| 229. | MIR556 | hsa-miR-556-3p | miRNA |
| 230. | MIR557 | hsa-miR-557 | miRNA |
| 231. | MIR561 | hsa-miR-561 | miRNA |
| 232. | MIR572 | hsa-miR-572 | miRNA |
| 233. | MIR574 | hsa-miR-574-3p | miRNA |
| 234. | MIR574 | hsa-miR-574-5p | miRNA |
| 235. | MIR595 | hsa-miR-595 | miRNA |
| 236. | MIR630 | hsa-miR-630 | miRNA |
| 237. | MIR642b | hsa-miR-642b | miRNA |
| 238. | MIR646 | hsa-miR-646 | miRNA |
| 239. | MIR659 | hsa-miR-659 | miRNA |
| 240. | MIR660 | haa-miR-660 | miRNA |
| 241. | MIR663A | hsa-miR-663 | miRNA |
| 242. | MIR664 | hsa-miR-664 | miRNA |
| 243. | MIR720 | hsa-miR-720 | miRNA |
| 244. | MIR762 | hsa-miR-762 | miRNA |
| 245. | MIR766 | hsa-miR-766 | miRNA |
| 246. | MIR877 | hsa-miR-877* | miRNA |
| 247. | MIR888 | hsa-miR-888 | miRNA |
| 248. | MIR933 | hsa-miR-933 | miRNA |
| 249. | MIR940 | hsa-miR-940 | miRNA |
| 250. | MIR95 | hsa-miR-95 | miRNA |
| 251. | hsv1-miR-H1* | hsv1-miR-H1*/hsv1-miR-H1-3p | miRNA |
| 252. | hsv1-miR-H2-3p | hsv1-miR-H2-3p | miRNA |
| 253. | hsv1-miR-H6-3p | hsv1-miR-H6-3p | miRNA |
| 254. | hsv1-miR-H7* | hsv1-miR-H7* | miRNA |
| 255. | hsv1-miR-H8 | hsv1-miR-HB | miRNA |
| 256. | hsv2-miR-H22 | hsv2-miR-H22 | miRNA |
| 257. | hsv2-miR-H | hsv2-miR-H25 | miRNA |
| 258. | hsv2-mIR-H6 | hsv2-miR-H6 | miRNA |
| 259. | hsv2-miR-H6* | hsv2-miR-H6* | miRNA |
| 260. | kshv-miR-K12-10a | kshv-miR-K12-10a | miRNA |
| 261. | kshv-miR-K12-12* | kshv-miR-K12-12* | miRNA |
| 262. | kshv-miR-K12-8* | kshv-miR-K12-8* | miRNA |
| 263. | MIR564 | hsa-miR-564 | miRNA |
| 264. | MIRLET7D | hsa-let-7d-5p | miRNA |
| 265. | MIRLET7D | hsa-let-7d-3p | miRNA |
| 266. | MIR29A | hsa-miR-29a | miRNA |
| 267. | MIR219 | hsa-miR-219-1-3p | miRNA |
| 268. | MIR296 | hsa-miR-296-3p | miRNA |
| 269. | MIR337 | hsa-miR-337-5p | miRNA |
| 270. | MIR369 | hsa-miR-369-3p | miRNA |
| 271. | MIR4S0 | hsa-miR-450b-5p | miRNA |
| 272. | MIR483 | hsa-miR-483-5p | miRNA |
| 273. | MIR507 | hsa-miR-507 | miRNA |
| 274. | MiR517C | hsa-miR-517c | miRNA |
| 275. | MIR519A | hsa-miR-519a | miRNA |
| 276. | MIR5190 | hsa-miR-519d | miRNA |
| 277. | MIR520G | hsa-miR-520g | miRNA |
| 278. | MIR576 | hsa-miR-576-5p | miRNA |
| 279. | MIR577 | hsa-miR-577 | miRNA |
| 280. | MIR604 | hsa-miR-604 | miRNA |
| 281. | MIR610 | haa-miR-610 | miRNA |
| 282. | MIR619 | hsa-miR-619 | miRNA |
| 283. | MIR1256 | hsa-miR-1256 | miRNA |
| 284. | MIR1278 | hsa-miR-1278 | miRNA |
| 285. | MIR1297 | hsa-miR-1297 | miRNA |
| 286. | MIRUS33 | hemv-miR-US33-Sp | miRNA |
| 287. | MIRH1 | hsv1-miR-H1-5p | miRNA |
| 288. | MIR877 | hsa-miR-877 | miRNA |
| 289. | LET7E | hsa-let-7e-5p | miRNA |
| 290. | LET7E | hsa-let-7e-3p | miRNA |
| 291. | MIR9 | hsa-miR-9 | miRNA |
| 292. | MIR92B | hsa-miR-92b | miRNA |
| 293. | MIR96 | hsa-miR-96-5p | miRNA |
| 294. | MIR96 | hsa-miR-96-3p | miRNA |
| 295. | MIR106A | hsa-miR-106a-5p | miRNA |
| 296. | MIR106A | hsa-miR-106a-3p | miRNA |
| 297. | MIR17 | hsa-miR-17-5p | miRNA |
| 298. | MIR17 | hsa-miR-17-3p | miRNA |
| 299. | MIR106B | hsa-miR-106b-5p | miRNA |
| 300. | MIR106B | hsa-miR-106b-3p | miRNA |
| 301. | MIR127 | hsa-miR-127-3p | miRNA |
| 302. | MIR128 | hsa-miR-128 | miRNA |
| 303. | MIR154 | hsa-miR-154 | miRNA |
| 304. | MIR155 | hsa-miR-155 | miRNA |
| 305. | MIR183 | hsa-miR-183-5p | miRNA |
| 305. | MIR183 | hsa-miR-183-3p | miRNA |
| 307. | MIR194 | hsa-miR-194 | miRNA |
| 308. | MIR196B | hsa-miR-196b-5p | miRNA |
| 309. | MIR196B | hsa-miR-196b-3p | miRNA |
| 310. | MIR203 | hsa-miR-203 | miRNA |
| 311. | MIR204 | hsa-miR-204 | miRNA |
| 312. | MIR221 | hsa-miR-221-5p | miRNA |
| 313. | MIR221 | hsa-miR-221-3p | miRNA |
| 314. | MIR378 | hsa-miR-378a | miRNA |
| 315. | MIR376C | hsa-miR-378c | miRNA |
| 316. | MIR377 | hsa-miR-377 | miRNA |
| 317. | MIR379 | hsa-miR-379 | miRNA |
| 318. | MIR423 | hsa-miR-423-3p | miRNA |
| 319. | MIR424 | hsa-miR-424-5p | miRNA |
| 320. | MIR424 | hsa-miR-424-3p | miRNA |
| 321. | MIR425 | hsa-miR-425-5p | miRNA |
| 322. | MIR425 | hsa-miR-425-3p | miRNA |
| 323. | MIR454 | hsa-miR-454-5p | miRNA |
| 324. | MIR454 | hsa-miR-454-3p | miRNA |
| 325. | MIR486 | hsa-miR-486-3p | miRNA |
| 326. | MIR509 | hsa-miR-509-3p | miRNA |
| 327. | MIR514 | hsa-miR-514 | miRNA |
| 328. | MIR542 | hsa-miR-542-3p | miRNA |
| 329. | MIR545 | hsa-miR-545-5p | miRNA |
| 330. | MIR545 | hsa-miR-545-3p | miRNA |
| 331. | MIR626 | hsa-miR-626 | miRNA |
| 332. | MIR758 | hsa-miR-758-5p | miRNA |
| 333. | MIR758 | hsa-miR-758-3p | miRNA |
| 334. | MIR876 | hsa-miR-876-3p | miRNA |
| 335. | MIR1185 | hsa-miR-1185 | miRNA |
| 336. | MIR1266 | hsa-miR-1266-5p | miRNA |
| 337. | MIR1266 | hsa-miR-1266-3p | miRNA |
| 338. | MIR199A | hsa-miR-199a-3p | miRNA |
| 339. | MIR199B | hsa-miR-199b-3p | miRNA |
| 349. | MIR625 | hsa-miR-625-5p | miRNA |
| 341. | MIRS25 | hsa-miR-825-3p | miRNA |
| 342. | MIR631 | hsa-miR-631 | miRNA |
| 343. | MIR635 | hsa-miR-635 | miRNA |

Columns

(i) This number is the SEQ ID NO as shown in the sequence listing. For an auto-antigen biomarker, the SEQ ID NO in the sequence listing provides the coding sequence for the auto-antigen biomarker. For a miRNA biomarker, the SEQ ID NO in the sequence listing provides the sequence of the mature, expressed miRNA biomarker, as shown in Tables 3 and 4.

(ii) The “Symbol” column gives the gene symbol which has been approved by the HGNC. The HONG aims to give unique and meaningful names to every miRNA end human gene. An additional dash-number suffix indicates pre-miRNAs that lead to identical mature miRNAs but that are located at different places in the genome.

(iii) The names for auto-antigens are taken from the Official Full Name provided by NCBI. An auto-antigen may have been referred to by one or more pseudonyms in the prior art. The invention relates to these auto-antigens regardless of their nomenclature. The names of the miRNA are taken from the specialist database, miRBase, according to version 16 (released, August 2010).

TABLE 2

| No. ⁽ⁱ⁾ | Symbol ⁽ⁱⁱ⁾ | ID ⁽ⁱⁱⁱ⁾ | Name^{(vi)} | HGNC ^{(v)} | GI ^{(vi)} |
|---|---|---|---|---|---|
| 1. | ACTS | 80 | Homo sapiens actin beta | 132 | 12854910 |
| 2. | ADD1 | 118 | Homo sapiens adducin 1 (alpha) | 243 | 33889910 |
| 3. | ADSL | 158 | Homo sapiens adenylosuccinate lyase | 291 | 12652984 |
| 4. | AK2 | 204 | Homo sapiens adenylate kinase 2 transcript | 362 | 39645192 |
| | | | variant AK2A | | |
| 5. | KLK3 | 354 | Home sapiens kallikrein 3 (prostate specific | 6384 | 34193547 |
| | | | antigen) transcript variant 1 | | |
| 6. | ATF1 | 466 | Homo sapiens activating transcription factor 1 | 783 | 20810444 |
| 7. | CKM | 1158 | Homo sapiens creatine kinase muscle | 1994 | 13938818 |
| 8. | CLK2 | 1196 | Homo sapiens CDC-like kinase 2 transcript | 2059 | 33873844 |
| | | | variant phcik2 | | |
| 9. | DDB2 | 1643 | Homo sapiens damage-specific DNA binding | 2718 | 34783036 |
| | | | protein 2 48 kDa | | |
| 10. | DTYMK | 1841 | Homo sapiens deoxythymidylate kinase | 3061 | 38114725 |
| | | | (thymidylate kinase) | | |
| 11. | DUSP4 | 1846 | Homo sapiens dual specificity phosphatase 4 | 3070 | 33369553 |
| | | | transcript variant 1 | | |
| 12. | E2F6 | 1876 | Homo sapiens E2F transcription factors | 3120 | 14249933 |
| 13. | EXT2 | 2132 | Home sapiens exostoses (multiple) 2 | 3513 | 14603195 |
| 14. | FGFR4_ | 2264 | Homo sapiens fibroblast growth factor | 3691 | 33873872 |
| | ext | | receptor 4 transcript variant 3 | | |
| 15. | FIGF | 2277 | Homo sapiens c-fos induced growth factor | 3708 | 20379761 |
| | | | (vascular endothelial growth factor D) | | |
| 16. | FKBP3 | 2287 | Homo sapiens FK506 binding protein 3 25 kDa | 3719 | 16740853 |
| 17. | GALK1 | 2584 | Home sapiens galactokinase 1 | 4118 | 12654058 |
| 18. | GK2 | 2712 | Homo sapiens glycerol kinase 2 | 4291 | 20987489 |
| 19. | GRB7 | 2886 | Homo sapiens growth factor receptor-bound | 4567 | 33870450 |
| | | | protein 7 | | |
| 20. | GTF28 | 2959 | Homo sapiens general transcription factor IIB | 4648 | 18088836 |
| 21. | GTF2H1 | 2965 | Homo sapiens general transcription factor IIH | 4655 | 33991027 |
| | | | polypeptide 1 62 kDa | | |
| 22 | GTF2H2 | 2966 | Homo sapiens general transcription factor IIH | 4656 | 40674449 |
| | | | polypeptide 2 44 kDa | | |
| 23. | HSPD1 | 3329 | Homo sapiens heat shock 60 kDa protein 1 | 5281 | 38197215 |
| | | | (chaperonin) | | |
| 24. | IDI1 | 3422 | Homo sapiens isopentenyl-diphosphate delta | 5387 | 34782883 |
| | | | isomerase | | |
| 25. | IFI16 | 3428 | Homo sapiens interferon, gamma-inducible | 5395 | 16877621 |
| | | | protein 16, | | |
| 26. | LDHA | 3939 | Homo sapiens lactate dehydrogenase A | 6535 | 45501321 |
| 27. | LYL1 | 4066 | Homo sapiens lymphoblastic leukemia derived | 6734 | 33988028 |
| | | | sequence 1 | | |
| 28. | MARK3 | 4140 | Homo sapiens MAP/microtubule affinity- | 6897 | 19353235 |
| | | | regulating kinase 3 | | |
| 29. | MPP3 | 4356 | Homo sapiens membrane protein | 7221 | 34785138 |
| | | | palmitoylated 3 (MAGUK p55 subfamily | | |
| | | | member 3) | | |
| 30. | TRIM37 | 4591 | Homo sapiens tripartite motif-containing 37, | 7523 | 23271191 |
| 31. | NCF2 | 4688 | Homo sapiens neutrophil cytosolic factor 2 | 7661 | 12804468 |
| | | | (65 kDa, chronic granulomatous disease, | | |
| | | | autosomal 2). | | |
| 32. | RPL10A | 4736 | Homo sapiens ribosomal protein L10a | 10299 | 13905015 |
| 33. | NFYA | 4800 | Homo sapiens nuclear transcription factor Y | 7804 | 24660183 |
| | | | alpha | | |
| 34. | NRAS | 4693 | Homo sapiens neuroblastoma RAS viral (v- | 7989 | 13528839 |
| | | | ras) oncogene homolog | | |
| 35. | NTRK3_ | 4916 | Homo sapiens neurotrophic tyrosine kinase | 8033 | 15489167 |
| | ext | | receptor type 3 transcript variant 3 | | |
| 36. | OAS2 | 4939 | Homo sapiens 2′-5′-oligoadenylate synthetase | 8067 | 29351562 |
| | | | 269/71 kDa | | |
| 37. | endometri- | 5091 | Hamo sapiens pyruvate carboxylase | 8636 | 33871110 |
| | osis | | | | |
| 38. | CDK17 | 5128 | Homo sapiens endometriosisTAIRE protein | 8750 | 21542570 |
| | | | kinase 2 | | |
| 39. | PDE4A | 5141 | Homo sapiens Homo sapiens | 8780 | 18043808 |
| | | | phosphodiesterase 4A, CAMP-specific | | |
| | | | (phosphodiesterase E2 dunce homolog, Dro | | |
| 40. | PHF1 | 5252 | Homo sapiens PHD finger protein 1 transcript | 8919 | 33874006 |
| | | | variant 2 | | |
| 41. | PKM2 | 5315 | Homo sapiens pyruvate kinase muscle | 9021 | 14043290 |
| | | | transcript variant 1 | | |
| 42. | MAP2K5 | 5607 | Homo sapiens mitogen-activated protein | 6845 | 33871775 |
| | | | kinase kinase 5, transcript variant A | | |
| 43. | PRPS2 | 5634 | Home sapiens phosphoribosyl pyrophosphate | 9465 | 26251732 |
| | | | synthetase 2 | | |
| 44, | RAN | 5901 | Homo sapiens RAN member RAS oncogene | 9846 | 33871120 |
| | | | family | | |
| 45. | RB1 | 5925 | Homo sapiens retinoblastoma 1 (including | 9884 | 24660139 |
| | | | osteosarcoma) | | |
| 46. | RBMS1 | 5937 | Homo sapiens Homo sapiens RNA binding | 9907 | 33869903 |
| | | | motif single stranded Interacting protein 1 | | |
| | | | transcript variant | | |
| 47. | RET_a | 5979 | Homo sapiens ret proto-oncogene (multiple | 9967 | 13279040 |
| | | | endocrine neoplasia and medullary thyroid | | |
| | | | carci | | |
| 48. | RORC | 6097 | Homo sapiens RAR-related orphan receptor C | 10280 | 21594879 |
| 49. | RPL18 | 6141 | Homo sapiens ribosomal protein L18 | 10310 | 38197133 |
| 50. | RPL18A | 6142 | Homo sapiens ribosomal protein L18a | 10311 | 38196939 |
| 51. | RPL28 | 6158 | Homo sapiens ribosomal protein L28 | 10330 | 15079502 |
| 52. | RPL31 | 6160 | Homo sapiens ribosomal protein L31 | 10334 | 40226052 |
| 53. | RPL32 | 6161 | Homo sapiens ribosomal protein L32 | 10338 | 15079341 |
| 54. | S100A6 | 6277 | Homo sapiens S100 calcium binding protein | 10496 | 33876209 |
| | | | A6 (calcyclin) | | |
| 55. | SCP2 | 6342 | Homo sapiens sterol carrier protein 2 | 10806 | 45501107 |
| | | | transcript variant 2 | | |
| 56. | SIAH1 | 6477 | Homo sapiens seven in absentia homolog 1 | 10857 | 27503513 |
| | | | (Drosophila) transcript variant 2 | | |
| 57. | SMARCD1 | 6602 | Homo sapiens SWI/SNF related matrix | 11106 | 33874464 |
| | | | associated actin dependent regulator of | | |
| | | | chromatin subfamily d member 1 | | |
| 58. | SMARCE1 | 6605 | Homo sapiens SWI/SNF related matrix | 11109 | 13937940 |
| | | | associated actin dependent regulator of | | |
| | | | chromatin | | |
| 59. | SOD2 | 6648 | Homo sapiens superoxide dismutase 2 | 11180 | 15214594 |
| | | | mitochondrial | | |
| 60. | SOX2 | 6657 | Homo sapiens SRY (sex determining region | 11195 | 33869633 |
| | | | Y)-box2 | | |
| 61. | SRPK1 | 6732 | Homo sapiens SFRS protein kinase 1 | 11305 | 23458344 |
| 62. | TPM1 | 7168 | Homo sapiens tropomyosin 1 (alpha) | 12010 | 33873609 |
| 63. | NR2C1 | 7181 | Homo sapiens nuclear receptor subfamily 2 | 7971 | 25304018 |
| | | | group C member 1 | | |
| 64. | TRIP6 | 7205 | Homo sapiens thyroid hormone receptor | 12311 | 13436460 |
| | | | interactor 6, | | |
| 65. | UBA1 | 7317 | Homo sapiens Homo sapiens ubiquitin- | 12489 | 33989140 |
| | | | activating enzyme E1 (A1S9T and BN75 | | |
| | | | temperature sensitivity compl | | |
| 66. | VCL | 7414 | Homo sapiens vinculin | 12865 | 24657578 |
| 67. | ZNF41 | 7592 | Homo sapiens zinc finger protein 41 transcript | 13107 | 21955337 |
| | | | variant 2 | | |
| 68. | PAX8 | 7849 | Homo sapiens paired box gene 8 transcript | 8622 | 33987990 |
| | | | variant PAXBA | | |
| 69. | NRIP1 | 8204 | Homo sapiens nuclear receptor interacting | 8001 | 25955638 |
| | | | protein 1 | | |
| 70. | PIP4K2B | 8396 | Homo sapiens phosphatidylinositol | 8998 | 20071985 |
| | | | phosphate 5-kinase type II beta transcript | | |
| | | | variant 2 | | |
| 71. | UXT | 8409 | Homo sapiens ubiquitously-expressed | 12641 | 14424496 |
| | | | transcript | | |
| 72. | API5 | 8539 | Homo sapiens apoptosis inhibitor 5 | 594 | 17389324 |
| 73. | MKNK1 | 8569 | Homo sapiens MAP kinase-interacting | 7110 | 33877125 |
| | | | serine/threonine kinase 1 | | |
| 74. | SUCLA2 | 8803 | Homo sapiens succinate-CoA ligase ADP- | 11448 | 34783884 |
| | | | forming beta subunit | | |
| 75. | LDB1 | 8861 | Homo sapiens LIM domain binding 1 | 6532 | 38197187 |
| 76. | NAE1 | 8883 | Homo sapiens amyloid beta precursor protein | 621 | 38197227 |
| | | | binding protein 1 transcript variant 1 | | |
| 77. | PAPSS2 | 9060 | Homo sapiens 3′-phosphoadenosine 5′- | 8504 | 33859502 |
| | | | phosphosulfate synthase 2 | | |
| 78. | USP10 | 9100 | Homo sapiens ubiquitin specific protease 10 | 12B08 | 12653004 |
| 79. | PRPF4 | 9128 | Hamo sapiens Homo sapiens PRP4 pre- | 17349 | 33876345 |
| 80. | AIM2 | 9447 | Hamo sapiens absent in melanoma 2 | 357 | 15012076 |
| 81. | TBPL1 | 9519 | Homo sapiens TBP-like 1 | 11589 | 33958482 |
| 82. | TRAF4 | 9618 | Homo sapiens TNF receptor-associated factor | 12034 | 12804686 |
| | | | 4 transcript variant 1 | | |
| 83. | SOCS5 | 9655 | Homo sapiens suppressor of cytokine | 15852 | 23273933 |
| | | | signaling 5 | | |
| 84. | ZSCAN12 | 9753 | Homo sapiens zinc finger protein 305 | 13172 | 27371192 |
| 85. | HDAC4 | 9759 | Homo sapiens CDNA | 14003 | 25058272 |
| 86. | KIAA0101 | 9768 | Homo sapiens KIAA0101 gene product | 28961 | 33873244 |
| 87. | IP6K1 | 9807 | Homo sapiens inositol hexaphosphate kinase | 18360 | 15277916 |
| | | | 1 | | |
| 88. | RNF40 | 9810 | Homo sapiens ring finger protein 40 transcript | 16867 | 13543993 |
| | | | variant 1 | | |
| 89. | GPHN | 10243 | Homo sapiens gephyrin | 15465 | 34783414 |
| 90. | HRSP12 | 10247 | Homo sapiens translational inhibitor protein | 16897 | 18397462 |
| | | | p14.5 | | |
| 91. | STUB1 | 10273 | Homo sapiens STIP1 homology and U-Box | 11427 | 14043118 |
| | | | containing protein 1 | | |
| 92. | TRAIP | 10293 | Homo sapiens TRAF interacting protein | 30764 | 17939475 |
| 93. | PLK4 | 10733 | Homo sapiens serine/threonine kinase 18 | 11397 | 23243308 |
| 94. | CCNL | 10983 | Homo sapiens cyclin I | 1595 | 38197480 |
| 95. | IL24 | 11009 | Homo sapiens interleukin 24 transcript variant | 11346 | 15307184 |
| | | | 1 | | |
| 96. | CDK20 | 23552 | Homo sapiens cell cycle related kinase | 21420 | 33988018 |
| 97. | PABendo- | 26966 | Homo sapiens poly(A) binding protein | 8554 | 33872187 |
| | metriosis | | cytoplasmic 1 | | |
| | 1 | | | | |
| 98. | MED4 | 29079 | Homo sapiens vitamin D receptor interacting | 17903 | 13528773 |
| | | | protein | | |
| 99. | NME7 | 29922 | Homo sapiens non-metastatic cells 7 protein | 20461 | 13937770 |
| | | | expressed in (nucleoside-diphosphate kinase) | | |
| | | | transcript variant 1 | | |
| 100. | PHF11 | 51131 | Homo sapiens PHD finger protein 11 | 17024 | 33880652 |
| 101. | IRAK4 | 51135 | Homo sapiens interleukin-1 receptor- | 17967 | 15426431 |
| | | | associated kinase 4 mRNA (cDNA clone | | |
| | | | MGC: 13330) | | |
| 102. | TXNDC3 | 51314 | Homo sapiens thioredoxin domain containing | 16473 | 22477641 |
| | | | 3 (spermatozoa) | | |
| 103. | TAOK3 | 51347 | Homo sapiens STE20-like kinase | 18133 | 33877128 |
| 104. | STYXL1 | 51657 | Homo sapiens dual specificity phosphatase 24 | 18165 | 33869206 |
| | | | (putative) | | |
| 105. | ASB1 | 51665 | Homo sapiens ankyrin repeat and SOCS box- | 16011 | 33878672 |
| | | | containing 1 | | |
| 100. | MST4 | 51765 | Homo sapiens Mst3 and SOK1-related kinase | na | 109633024 |
| | | | (MASK) | | |
| 107. | PELO | 53918 | Homo sapiens pelota homolog (Drosophila) | 8829 | 33870521 |
| 108. | ETNR2 | 55224 | Homo sapiens ethanolamine kinase 2 | 25575 | 33873304 |
| 109. | RFK | 55312 | Homo sapiens riboflavin kinase | 30324 | 13937919 |
| 110. | C9or86 | 55684 | Homo sapiens chromosome 9 open reading | 24703 | 18089283 |
| | | | frame 86 | | |
| 111. | ODX55 | 57696 | Homo sapiens DEAD (Asp-Glu-Ala-Asp) box | 20085 | 34190881 |
| | | | polypeptide 55 | | |
| 112. | CCNB1IP1 | 57820 | Homo sapiens cDNA | 19437 | 12654750 |
| 113. | KLHL12 | 59349 | Homo sapiens keich-like protein C3IP1 | 19360 | 13112018 |
| 114. | SAV1 | 60485 | Homo sapiens Salvador homolog 1 | 17795 | 18088227 |
| | | | (Drosophila) | | |
| 115. | CAMKV | 79012 | Homo sapiens hypothetical protein MGC8407 | 28788 | 33875513 |
| 116. | NSBP1 | 79366 | Homo sapiens nucleosomal binding protein 1, | 8013 | 13529139 |
| 117. | ALPK1 | 80216 | Homo sapiens alpha-kinase 1 mRNA (cDNA | 20917 | 38174241 |
| | | | clone MGO:71554) | | |
| 118. | ELMOD3 | 84173 | Homo sapiens RNA binding motif and ELMO | 26158 | 33877554 |
| | | | domain 1 | | |
| 119. | AIFM2 | 84883 | Homo sapiens apoptosis-inducing factor (AIF)- | 21411 | 13543983 |
| | | | like mitochondrion-associated inducer of death | | |
| 120. | RHOT2 | 89941 | Homo sapiens ras homolog gene family | 21189 | 15928948 |
| | | | member T2 | | |
| 121. | PYGO2 | 90780 | Home sapiens pygopus 2 | 30257 | 33991480 |

Columns

(i) This number is the SEQ ID NO: for the coding sequence for the auto-antigen biomarker, as shown in the sequence listing.

(ii) The “ID” column shows the Entrez GeneID number for the antigen marker. An Entrez GeneID value is unique across all taxa.

(iii) The “Symbol” column gives the gene symbol which has been approved by the HGNC. The HGNC aims to give unique and meaningful names to every miRNA and human gene.

(iv) This name is taken from the Official Full Name provided by NCBI. An auto-antigen may have been referred to by one or more pseudonyms in the prior art. The invention relates to these auto-antigens regardless of their nomenclature.

(v) The HUGO Gene Nomenclature Committee aims to give unique and meaningful names to every human gene. The HGNC number thus identifies a unique human gene. An additional dash-number suffix indicates pre-miRNAs that lead to identical mature miRNAs but that are located at different places in the genome.

(v) A “GI” number, or “GenInfo Identifier”, is a series of digits assigned consecutively to each sequence record processed by NCBI when sequences are added to its databases. The GI number bears no resemblance to the accession number of the sequence record. When a sequence is updated (e.g. for correction, or to add more annotation or information) it receives a new GI number. Thus the sequence associated with a given GI number is never changed.

TABLE 3

| No. | | | | |
|---|---|---|---|---|
| (i) | miRNA name⁽ⁱⁱ⁾ | Symbol⁽ⁱⁱⁱ⁾ | HGNC^{(iv)} | Sequence |
| 122. | ebv-miR-BART12 | ebv-miR- | | UCCUGUGGUGUUUGGUGUGGUU |
| | | BART12 | | |
| 123. | ebv-miR-BART14 | ebv-miR- | | UAAAUGCUGGAGUAGUAGGGAU |
| | | BART14 | | |
| 124. | ebv-miR-BART16 | ebv-miR- | | UUAGAUAGAGUGGGUGUGUGCUCU |
| | | BART16 | | |
| 125. | ebv-miR-BART20- | eby-miR- | | UAGCAGGCAUGUCUUCAUUCC |
| | 5p | BART20-6 | | |
| 126. | ebv-miR-BART2- | ebv-miR- | | UAUUUUCUGCAUUCGCCCUUGC |
| | 5p | BART2-5p | | |
| 127. | hsa-let-7b* | MIRLET7B | HGNC:31479 | UGAGGUAGUAGGUUGUGUGGUU |
| 128. | hsa-let-7f | MIRLET7F1 | HGNC:31483 | UGAGGUAGUAGAUUGUAUAGUU |
| 129. | hsa-let-7f-1* | MIRLET7F2 | HGNC:31484 | UGAGGUAGUAGAUUGUAUAGUU |
| 130. | hsa-let-7g | MIRLET7G | HGNC:31485 | UGAGGUAGUAGUUUGUACAGUU |
| 131. | hsa-miR-103a | MIR103A1 | HGNC:31490 | AGCAGCAUUGUACAGGGCUAUGA |
| 132. | hsa-miR-10b | MIR10b | HGNC:31498 | UACCCUGUAGAACCGAAUUUGUG |
| 133. | hsa-miR-1183 | MIR1183 | HGNC:35264 | CACUGUAGGUGAUGGUGAGAGUGGGCA |
| 134. | hsa-miR-1202 | MIR1202 | HGNC:35268 | GUGCCAGCUGCAGUGGGGGAG |
| 135. | hsa-miR-1207-5p | MIR1207 | HGNC:35273 | UGGCAGGGAGGCUGGGAGGGG |
| 136. | hsa-miR-122 | MIR122 | HGNC:31501 | UGGAGUGUGACAAUGGUGUUUG |
| 137. | hsa-miR-1224-5p | MIR1224 | HGNC:33923 | GUGAGGACUCGGGAGGUGG |
| 138. | hsa-miR-1225-3p | MIR1225 | HGNC:33931 | GUGGGUACGGCCCAGUGGGGGG |
| 130. | hsa-miR-1225-5p | MIR1225 | HGNC:33931 | GUGGGUACGGCCCAGUGGGGGG |
| 140. | hsa-miR-1226* | MIR1226 | HGNC:33922 | UCACCAGCCCUGUGUUCCCUAG |
| 141. | hsa-miR-1228* | MIR1228 | HGNC:33928 | UCACACCUGCCUCGCCCCCC |
| 142. | hsa-miR-1234 | MIR1234 | HGNC:33926 | UOGGCCUGACCACCCACCCCAC |
| 143. | hsa-miR-1237 | MIR1237 | HGNC:33927 | UCCUUCUGCUCCGUCCCCCAG |
| 144. | hsa-miR-1238 | MIR1238 | HGNC:33933 | CUUCCUCGUCUGUCUGCCCC |
| 145. | hsa-miR-125a-5p | MIR125a | HGNC:31505 | UCCCUGAGACCCUUUAACCUGUGA |
| 140. | hsa-miR-1260 | MIR1280A | HGNC:35325 | AUCCCACCUCUGCCACCA |
| 147. | hsa-miR-1260b | MIR1260b | HGNO:38258 | AUCCCACCACUGCCACCAU |
| 146. | hsa-miR-1280 | MIR1280 | HGNC:35368 | UCCCACCGCUGCCACCC |
| 140. | hsa-miR-1281 | MIR1281 | HGNC:35359 | UCGCCUCCUCCUCUCCC |
| 150. | hsa-miR-1290 | MIR1290 | HGNC:35283 | UGGAUUUUUGGAUCAGGGA |
| 151. | hsa-miR-1291 | MIR1291 | HGNC:35284 | UGGCCCUGACUGAAGACCAGCAGU |
| 152. | hsa-miR-129-3p | MIR129-1/ | HGNC:31512/ | CUUUUUGCGGUCUGGGCUUGC |
| | | MIR129-2 | 31513 | |
| 153. | hsa-miR-1301 | MIR1301 | HGNC:35253 | UUGCAGCUGCCUGGGAGUGACUUC |
| 154. | hsa-miR-135a | MIR135A1/ | HGNC:31520/ | UAUGGCUUUUUAUUCCUAUGUGA |
| | | MIR135A2 | 31521 | |
| 155. | hsa-miR-135a* | MIR135A1/ | HGNC:31520/ | UAUGGCUUUUUAUUCCUAUGUGA |
| | | MIR135A2 | 31521 | |
| 156. | hsa-miR-141 | MIR141 | HGNC:31528 | UAACACUGUCUGGUAAAGAUGG |
| 157. | hsa-miR-142-3p | MIR142 | HGNC:31529 | CAUAAAGUAGAAAGCACUACU |
| 158. | hsa-miR-149 | MIR149 | HGNC:31536 | UCUGGCUCCGUGUCUUCACUCCC |
| 159. | hsa-miR-150 | MIR150 | HGNO:31537 | UCUCCCAACCCUUGUACCAGUG |
| 160. | hsa-miR-150* | MIR150 | HGNC:31537 | UCUCCCAACCCUUGUACCAGUG |
| 161. | hsa-miR-1539 | MIR1539 | HGNC:35383 | UCCUGCGCGUCCCAGAUGCCC |
| 162. | hsa-miR-181a | MIR181A2 | HGNC:31549 | AACAUUCAACGCUGUCGGUGAGU |
| 163. | hsa-miR-1825 | MIR1825 | HGNC:35389 | UCCAGUGCCCUCCUCUCC |
| 164. | hsa-miR-186 | MIR186 | HGNC:31557 | CAAAGAAUUCUCCUUUUGGGCU |
| 165. | hsa-miR-18s | MIR18a | HGNC:31548 | UAAGGUGCAUCUAGUGCAGAUAG |
| 166. | hsa-miR-18b | MIR18 | HGNC:32025 | UAAGGUGCAUCUAGUGCAGUUAG |
| 167. | hsa-miR-191* | MIR191 | HGNC:31561 | CAACGGAAUCCCAAAAGCAGCUG |
| 168. | hsa-miR-197 | MIR197 | HGNC:31569 | UUCACCACCUUCUCCACCCAGC |
| 169. | hsa-miR-198 | MIR198 | HGNC:31570 | GGUCCAGAGGGGAGAUAGGUUC |
| 170. | hsa-miR-205 | MIR205 | HGNC:31583 | UCCUUCAUUCCACCGGAGUCUG |
| 171. | hsa-miR-2116* | MIR2116 | HGNC:37310 | GGUUCUUAGCAUAGGAGGUCU |
| 172. | hsa-miR-215 | MIR215 | HGNC:31592 | AUGACCUAUGAAUUGACAGAC |
| 173. | hsa-miR-22 | MIR22 | HGNC:31599 | AAGCUGCCAGUUGAAGAACUGU |
| 174. | hsa-miR-223 | MIR223 | HGNC:31603 | UGUCAGUUUGUCAAAUACCCCA |
| 175. | hsa-miR-2276 | MIR2276 | HGNC:37313 | UCUGCAAGUGUCAGAGGCGAGG |
| 176. | hsa-miR-230 | MIR23C | HGNC:38913 | AUCACAUUGCCAGUGAUUACCC |
| 177. | hsa-miR-26a | MIR26A1/ | HGNC:31610/ | UUCAAGUAAUCCAGGAUAGGCU |
| | | MIR2BA2 | 31811 | |
| 178. | hsa-miR-28-3p | MIR28 | HGNC:31615 | AAGGAGCUCACAGUCUAUUGAG |
| 179. | hsa-miR-28-5p | MIR28 | HGNC:31615 | AAGGAGCUCACAGUCUAUUGAG |
| 160. | hsa-miR-29b | MIR29B1/ | HGNC:31619/ | UAGGACCAUUUGAAAUGAGUGUU |
| | | MIR29B2 | 31620 | |
| 181. | hsa-miR-30 | MIR30a | HGNC:31624 | UGUAAACAUCCUCGACUGGAAG |
| 182. | hsa-miR-30b | MIR30b | HGNC:31625 | UGUAAACAUCCUACAGUCAGGU |
| 183. | hsa-miR-3131 | MIR3131 | HGNC:38347 | UCGAGGACUGGUGGAAGGGCCUU |
| 184. | hsa-miR-3138 | MIR3138 | HGNC:38341 | UGUGGACAGUGAGGUAGAGGGAGL |
| 185. | hsa-miR-3149 | MIR3149 | HGNC:38251 | UUUGUAUGGAUAUGUGUGUGUAU |
| 186. | hsa-miR-3156-5p | MIR3156-1/ | HGNC:38241/ | AAAGAUCUGGAAGUGGGAGACA |
| | | MIR3156- | 38213/ | |
| | | 2/ | 38229 | |
| | | MIR3156-3 | | |
| 187. | hsa-miR-3180-5p | MIR3180-1/ | HGNC:38382/ | UGGGGCGGAGCUUCCGGAG |
| | | MIR3180-2/ | 38343/ | |
| | | MIR3180-3/ | 38239/ | |
| | | MIR3180-4/ | 38920/ | |
| | | MIR3180-5 | 38989 | |
| 188. | hsa-miR-3194-5p | MIR3194 | HGNC:38346 | GGCCAGCCACCAGGAGGGCUG |
| 189. | hsa-miR-3195 | MIR3195 | HGNO:38250 | CGCGCCGGGCCCGGGUU |
| 190. | hsa-miR-3196 | MIR3196 | HGNC:38198 | CGGGGCGGCAGGGGCCUC |
| 191. | hsa-miR-32 | MIR32 | HGNC:31631 | UAUUGCACAUUACUAAGUUGCA |
| 192. | hsa-miR-320c | MIR320C1/ | HGNC:35248/ | AAAAGCUGGGUUGAGAGGGU |
| | | MIR320C2 | 35387 | |
| 193. | hsa-miR-324-3p | MIR324 | HGNC:31767 | CGCAUCCCCUAGGGCAUUGGUGU |
| 194. | hsa-miR-328 | MIR328 | HGNC:31770 | cugecccUcUCUGCCCUUCCGL |
| 195. | hsa-miR-331-5p | MIR331 | HGNC:31772 | CUAGGUAUGGUCCCAGGGAUCC |
| 196. | hsa-miR-33b* | MIR33b | HGNC:32791 | GUGCAUUGCUGUUGCAUUGC |
| 197. | hsa-miR-342-3p | MIR342 | HGNG:31778 | AGGGGUGCUAUCUGUGAUUGA |
| 198. | hsa-miR-34a | MIR34a | HGNC:31635 | UGGCAGUGUCUUAGCUGGUUGU |
| 199. | hsa-miR-3610 | MIR3610 | HGNC:38942 | GAAUCGGAAAGGAGGCGCCG |
| 200. | hsa-miR-3648 | MIR3648 | HGNC:38941 | AGCCGCGGGGAUCGCCGAGGG |
| 201. | hsa-miR-3652 | MIR3652 | HGNC:38894 | CGGCUGGAGGUGUGAGGA |
| 202. | hsa-miR-3663-5p | MIR3663 | HGNC:38958 | GCUGGUCUGCGUGGUGCUCGG |
| 203. | hsa-miR-3667-5p | MIR3667 | HGNC:38990 | AAAGACCCAUUGAGGAGAAGGU |
| 204. | hsa-miR-382 | MIR382 | HGNC:31875 | GAAGUUGUUCGUGGUGGAUUCG |
| 205. | hsa-miR-3011 | MIR3911 | HGNC:38962 | UGUGUGGAUCCUGGAGGAGGCA |
| 206. | hsa-miR-3937 | MIR3937 | HGNC:38970 | ACAGGCGGCUGUAGCAAUGGGGG |
| 207. | hsa-miR-4257 | MIR4257 | HGNC:38312 | QCAGAGGUGGGGACUGAG |
| 208. | hsa-miR-425* | MIR425 | HGNC:31882 | AAUGACACGAUCACUCCCGUUGA |
| 209. | hsa-miR-4270 | MIR4270 | HGNC:38377 | UCAGGGAGUGAGGGGAGGGC |
| 210. | hsa-miR-4274 | MIR4274 | HGNC:38194 | CAGCAGUCCCUCCCCCUG |
| 211. | hsa-miR-4281 | MIR4281 | HGNC:38357 | GGGUCCCGGGGAGGGGGG |
| 212. | hsa-miR-4284 | MIR4284 | HGNC:38322 | GGGCUCACAUCACCCCAU |
| 213. | hsa-miR-4286 | MIR4286 | HGNC:38186 | ACCCCACUCCUGGUACC |
| 214. | hsa-miR-429 | MIR429 | HGNC:13784 | UAAUACUGUCUGGUAAAACCGU |
| 215. | hsa-miR-4290 | MIR4290 | HGNC:38360 | UeCCCUCCUUUCUUOCCUC |
| 216. | hsa-miR-4313 | MIR4313 | HGNC:38310 | AGCCCCCUGGCCCCAAACCG |
| 217. | hsa-miR-4323 | MIR4323 | HGNC:38394 | CAGCCCCACAGCCUCAGA |
| 216. | hsa-miR-466 | MIR466 | HGNC:38359 | AUACACAUACACGCAACACACAU |
| 219. | hsa-miR-483-3p | MIR483 | HGNC:32340 | AAGACGGGAGGAAAGAAGGGAG |
| 220. | hsa-miR-484 | MIR484 | HGNC:32341 | UCAGGCUCAGUCCCCUCCCGAU |
| 221. | hsa-miR-485-3p | MIR485 | HGNC:32087 | AGAGGCUGGCCGUGAUGAAUUC |
| 222. | hsa-miR-486-5p | MIR486 | HGNC:32342 | UCCUGUACUGAGCUGCCCCGAG |
| 223. | hsa-miR-488 | MIR488 | HGNC:32073 | UUGAAAGGCUAUUUCUUGGUC |
| 224. | hsa-miR-497 | MIR497 | HGNC:32088 | CAGCAGCACACUGUGGUUUGU |
| 225. | hsa-miR-511 | MIR511-1 | HGNC:32077 | GUGUCUUUUGCUCUGCAGUCA |
| 226. | hsa-miR-512 | MIR511-2 | HGNC:32078 | GUGUCUUUUGCUCUGCAGUCA |
| 227. | hsa-miR-5200-3p | MIR520c | HGNC:32108 | CUCUAGAGGGAAGCACUUUCUG |
| 228. | hsa-miR-550a | MIR550A1/ | HGNC:32804/ | AGUGCCUGAGGGAGUAAGAGCCC |
| | | MIR550A2/ | 32805/ | |
| | | MIRS50A3 | 41878 | |
| 229. | hsa-miR-556-3p | MIR556 | HGNC:32812 | GAUGAGCUCAUUGUAAUAUGAG |
| 230. | hsa-miR-567 | MIR557 | HGNC:32813 | GUUUGCACGGGUGGGCCUUGUCU |
| 231. | hsa-miR-561 | MIRS61 | HGNC:32817 | CAAAGUUUAAGAUCCUUGAAGU |
| 232. | hsa-miR-572 | MIR572 | HGNC:32828 | GUCCGCUCGGCGGUGGCCCA |
| 233. | hsa-miR-574-3p | MIR574 | HGNC:32830 | UGAGUGUGUGUGUGUGAGUGUGU |
| 234. | hsa-miR-574-5p | MIR574 | HGNC:32830 | UGAGUGUGUGUGUGUGAGUGUGU |
| 235. | hsa-miR-595 | MIR595 | HGNC:32851 | GAAGUGUGCCGUGGUGUGUCU |
| 236. | hsa-miR-630 | MIR630 | HGNC:32886 | AGUAUUCUGUACCAGGGAAGGU |
| 237. | hsa-miR-642b | MIR642b | HGNC:38902 | AGACACAUUUGGAGAGGGACCC |
| 238. | hsa-miR-646 | MIR646 | HGNC:32902 | AAGCAGCUGCCUCUGAGGC |
| 239. | hsa-miR-659 | MIR659 | HGNC:32915 | CUUGGUUCAGGGAGGGUCCCCA |
| 240. | hsa-miR-660 | MIR680 | HGNC:32916 | UACCCAUUGCAUAUCGGAGUUG |
| 241. | hsa-miR-663 | MIRG63A | HGNC:32919 | AGGCGGGGCGCCGCGGGACCGC |
| 242. | hsa-miR-664 | MIR684 | HGNC:35370 | UAUUCAUUUAUCCCCAGCCUACA |
| 243. | hsa-miR-720 | MIR720 | HGNC:35375 | UCUCGCUGGGGCCUCCA |
| 244. | hsa-miR-762 | MIR762 | HGNC:37303 | GGGGCUGGGGCCGGGGCCGAGC |
| 245. | hsa-miR-766 | MIR766 | HGNC:33139 | ACUCCAGCCCCACAGCCUCAGC |
| 246. | hsa-miR-877* | MIR877 | HGNC:33660 | GUAGAGGAGAUGGCGCAGGG |
| 247. | hsa-miR-888 | MIR88B | HGNC:33648 | UACUCAAAAAGCUGUCAGUCA |
| 248. | hsa-miR-933 | MIR933 | HGNC:33676 | UGUGCGCAGGGAGACCUCUCCC |
| 249. | hsa-miR-940 | MIR940 | HGNC:33683 | AAGGCAGGGCCCCCGCUCCCC |
| 250. | hsa-miR-95 | MIR95 | HGNC:31647 | UUCAACGGGUAUUUAUUGAGCA |
| 251. | hsv1-miR-H1*/ | hsv1-miR- | | UACACCCCCCUGCCUUCCACCCU |
| | hsv1-miR-H1-3p | H1* | | |
| 252. | hsv-miR-H2-p | hsv1-miR- | | CCUGAGCCAGGGACGAGUGCGACU |
| | | H2-3p | | |
| 253. | hsv1-miR-H6-3p | hsv1-miR- | | CACUUCCCGUCCUUCCAUCCC |
| | | H6-3p | | |
| 254. | hsv1-miR-H7* | hsv1-miR- | | UUUGGAUCCCGACCCCUCUUC |
| | | H7* | | |
| 255. | hsv1-miR-H8 | hsv1-miR- | | UAUAUAGGGUCAGGGGGUUC |
| | | H8 | | |
| 256. | hsv2-miR-H22 | hsv2-miR- | | AGGGGUCUGGACGUGGGUGGGC |
| | | H22 | | |
| 257. | hsv2-miR-H25 | hsv2-miR- | | CUGCGCGGCGGAGACCGGGAC |
| | | H25 | | |
| 258. | hsv2-miR-H6 | hsv2-miR- | | AAUGGAAGGCGAGGGGAUGC |
| | | H6 | | |
| 259. | hsv2-miR-H6* | hsv2-miR- | | CCCAUCUUCUGCCCUUCCAUCCU |
| | | H6* | | |
| 260. | kshv-miR-K12-10a | kshv-miR- | | UAGUGUUGUCCCCCCGAGUGGC |
| | | K12-10a | | |
| 261. | kshv-miR-K12-12* | kshv-miR- | | UGGGGGAGGGUGCCCUGGUUGA |
| | | K12-12* | | |
| 262. | kshv-miR-K12-8* | kshv-miR- | | ACUCCCUCACUAACGCCCCGCU |
| | | K12-8* | | |

Columns for Tables 3 and 4

(i) The SEQ ID NO: for the sequence of the mature, expressed miRNA biomarker.

(ii) The “mRNA name” column gives the name of the human mRNA as provided by the specialist database, miRBase, according to version 15 (released. August 2010).

(iii) The “Symbol” column gives the gene symbol which has been approved by the HGNC. The HGNC aims to give unique and meaningful names to every miRNA and human gene. An additional dash-number suffix indicates pre-miRNAs that lead to identical mature miRNAs but that are located at different places in the genome.

(iv) The HGNC aims to give unique and meaningful names to every mRNA (and human gene). The HGNC number thus identifies a unique human gene. Inclusion on to HUGO is for human genes only.

TABLE 4

| No. | miRNA name⁽ⁱ⁾ | Symbol⁽ⁱⁱ⁾ | HGNC⁽ⁱⁱⁱ⁾ | Sequence |
|---|---|---|---|---|
| 126 | ebv-miR-BART2-5p | ebv-miR- | N/A | UAUUUUCUGCAUUCGCCCUUGC |
| | | BART2-5p | | |
| 263. | hsa-miR-564 | MIR564 | 32820 | AGGGACGGUGUCAGCAGGC |
| 264. | hsa-let-7d-5p | MIRLET7D | 31481 | AGAGGUAGUAGGUUGCAUAGUU |
| 265. | hsa-let-7d-3p | MIRLET7D | 31481 | CUAUACGACCUGCUGCCUUUCU |
| 266. | hsa-miR-29a | MIR29A | 31616 | UAGCACCAUCUGAAAUCGGUUA |
| 157 | hsa-miR-142-3p | MIR142 | 31529 | CAUAAAGUAGAAAGCACUACU |
| 168 | hsa-miR-197 | MIR197 | 31589 | UUCACCACCUUCUCCACCCAGC |
| 172 | hsa-miR-215 | MIR215 | 31592 | AUGACCUAUGAAUUGACAGAC |
| 267. | hsa-miR-219-1-3p | MIR-219-1 | 31597 | AGAGUUGAGUCUGGACGUCCCG |
| 268. | hsa-miR-296-3p | MIR296 | 31617 | GAGGGUUGGGUGGAGGCUCUCC |
| 192 | hsa-miR-320c | MIR320C | 35248 | AAAAGCUGGGUUGAGAGGGU |
| 269. | hsa-miR-337-5p | MIR337 | 31774 | GAACGGCUUCAUACAGGAGUU |
| 270. | hsa-miR-369-3p | MIR369 | 31783 | AAUAAUACAUGGUUGAUCUUU |
| 271. | hsa-miR-450b-5p | MIR450B | 33642 | UUUUGCAAUAUGUUCCUGAAUA |
| 272. | hsa-miR-483-5p | MIR483 | 32340 | AAGACGGGAGGAAAGAAGGGAG |
| 273. | hsa-miR-507 | MIR507 | 32144 | UUUUGCACCUUUUGGAGUGAA |
| 274. | hsa-miR-517c | MIR517C | 32124 | AUCGUGCAUCCUUUUAGAGUGU |
| 275. | hsa-miR-519a | MIR519A1/ | 32128/ | AAAGUGCAUCCUUUUAGAGUGU |
| | | MIR519A2 | 32132 | |
| 276. | hsa-miR-519d | MIR519D | 32112 | CAAAGUGCCUCCCUUUAGAGUG |
| 277. | hsa-miR-520g | MIR520G | 32116 | ACAAAGUGCUUCCCUUUAGAGUGU |
| 278. | hsa-miR-576-5p | MIR576 | 32832 | AUUCUAAUUUCUCCACGUCUUU |
| 279. | hsa-miR-577 | MIR577 | 32833 | UAGAUAAAAUAUUGGUACCUG |
| 280. | hsa-miR-604 | MIR504 | 32860 | AGGCUGCGGAAUUCAGGAC |
| 281. | hsa-miR-610 | MIR610 | 32866 | UGAGCUAAAUGUGUGCUGGGA |
| 282. | hsa-miR-619 | MIR619 | 32875 | GACCUGGACAUGUUUGUGCCCAGU |
| 283. | hsa-miR-1256 | MIR1256 | 35321 | AGGCAUUGACUUCUCACUAGCU |
| 284. | hsa-miR-1278 | MIR1278 | 35356 | UAGUACUGUGCAUAUCAUCUAU |
| 285. | hsa-miR-1297 | MIR1297 | 35289 | UUCAAGUAAUUCAGGUG |
| 286. | hcmv-miR-US33-5p | N/A | N/A | GAUUGUGCCCGGACCGUGGGCG |
| 287. | hsv1-miR-H1-5p | N/A | N/A | GAUGGAAGGACGGGAAGUGGA |
| 288. | hsa-miR-877 | MIR877 | 33660 | GUAGAGGAGAUGGCGCAGGG |
| 289. | hsa-let-7e-5p | MIRLET7E | 31482 | UGAGGUAGGAGGUUGUAUAGUU |
| 290. | hsa-let-7e-3p | MIRLET7E | 31482 | CUAUACGGCCUCCUAGCUUUCC |
| 128 | hsa-let-7f | MIRLET7F1 | 31483 | UGAGGUAGUAGAUUGUAUAGUU |
| 130 | hsa-let-7g | MIRLET7G | 31485 | UGAGGUAGUAGUUUGUACAGUU |
| 291. | hsa-miR-9 | MIRG-1/ | 31641/ | UCUUUGGUUAUCUAGCUGUAUGA |
| | | MIR9-2/ | 31642/ | |
| | | MIR9-3 | 31646 | |
| 182 | hsa-miR-30b | MIR30a | 31624 | UGUAAACAUCCUCGACUGGAAG |
| 198 | hsa-miR-34a | MIR34A | 31635 | UGGCAGUGUCUUAGCUGGUUGU |
| 292. | hsa-miR-92b | MIR92B | 32920 | UAUUGCACUCGUCCCGGCCUCC |
| 293. | hsa-miR-96-5p | MIR96 | 31648 | UUUGGCACUAGGACAUUUUUGCU |
| 294. | hsa-miR-96-3p | MIR96 | 31648 | AAUCAUGUGGAGUGCCAAUAUG |
| 295. | hsa-miR-106a-5p | MIR106A | 31494 | AAAAGUGCUUACAGUGCAGGUAG |
| 296. | hsa-miR-106a-3p | MIR106A | 31494 | CUGCAAUGUAAGGACUUCUUAC |
| 297. | hsa-miR-17-5p | MIR17 | 31547 | CAAAGUGCUUACAGUGCAGGUAG |
| 298. | hsa-miR-17-3p | MIR17 | 31547 | ACUGCAGUGAAGGCACUUGUAG |
| 299. | hsa-miR-106b-Sp | MIR106B | 31495 | UAAAGUGCUGACAGUGCAGAU |
| 300. | hsa-miR-106b-3p | MIR106B | 31495 | CCGCACUGUGGGUACUUGCUGC |
| 301. | hsa-miR-127-3p | MIR127 | 31509 | UCGGAUCCGUCUGAGCUUGGCU |
| 302. | hsa-miR-128 | MIR128-1/ | 31510/ | UCACAGUGAACCGGUCUCUUU |
| | | MIR128-2 | 31511 | |
| 303. | hsa-miR-154 | MIR154 | 31541 | UAGGUUAUCCGUGUUGCCUUCG |
| 304. | hsa-miR-155 | MIR155 | 31542 | UUAAUGCUAAUCGUGAUAGGGGU |
| 305. | hsa-miR-183-5p | MIR183 | 31554 | UAUGGCACUGGUAGAAUUCACU |
| 306. | hsa-miR-183-3p | MIR183 | 31554 | GUGAAUUACCGAAGGGCCAUAA |
| 307. | hsa-miR-194 | MIR194-1/ | 31564/ | UGUAACAGCAACUCCAUGUGGA |
| | | MIR194-2 | 31565 | |
| 308. | hsa-miR-196b-5p | MIR196B | 31790 | UAGGUAGUUUCCUGUUGUUGGG |
| 309. | hsa-miR-196b-3p | MIR196B | 31790 | UCGACAGCACGACACUGCCUUC |
| 310. | hsa-miR-203 | MIR203 | 31581 | GUGAAAUGUUUAGGACCACUAG |
| 311. | hsa-miR-204 | MIR204 | 31582 | UUCCCUUUGUCAUCCUAUGCCU |
| 312. | hsa-miR-221-5p | MIR221 | 31601 | ACCUGGCAUACAAUGUAGAUUU |
| 313. | hsa-miR-221-3p | MIR221 | 31601 | AGCUACAUUGUCUGCUGGGUUUC |
| 174 | hsa-miR-223 | MIR223 | 31603 | UGUCAGUUUGUCAAAUACCCCA |
| 314. | hsa-miR-376a | MIR376A1/ | 31869/ | AUCAUAGAGGAAAAUCCACGU |
| | | MIR376A2 | 32532 | |
| 315. | hsa-miR-376c | MIR376C | 31782 | AACAUAGAGGAAAUUCCACGU |
| 316. | hsa-miR-377 | MIR377 | 31870 | AUCACACAAAGGCAACUUUUGU |
| 317. | hsa-miR-379 | MIR379 | 31872 | UGGUAGACUAUGGAACGUAGG |
| 318. | hsa-miR-423-3p | MIR423 | 31880 | AGCUCGGUCUGAGGCCCCUCAGU |
| 319. | hsa-miR-424-5p | MIR424 | 31881 | CAGGAGCAAUUCAUGUUUUGAA |
| 320. | hsa-miR-424-3p | MIR424 | 31881 | CAAAACGUGAGGOGCUGCUAU |
| 321. | hsa-miR-425-5p | MIR425 | 31882 | AAUGACAGGAUGACUCCCGUUGA |
| 322. | hsa-miR-425-3p | MIR425 | 31882 | AUCGGGAAUGUCGUGUCCGCCC |
| 323. | hsa-miR-454-5p | MIR454 | 33137 | ACCCUAUCAAUAUUGUCUCUGG |
| 324. | hsa-miR-454-3p | MIR454 | 33137 | UAGUGCAAUAUUGCUUAUAGGGU |
| 325. | hsa-miR-486-3p | hsa-miR-486 | 32342 | CGGGGCAGCUCAGUACAGGAU |
| | | MIR486 | | |
| 326. | hsa-miR-509-3p | MIR509-3 | 33675 | UGAUUGGUACGUCUGUGGGUAG |
| 327. | hsa-miR-514 | MIR514-1/ | 32148/ | AUUGACACUUCUGUGAGUAGA |
| | | MIR514-2/ | 32149/ | |
| | | MIR514-3 | 32150 | |
| 328. | hsa-miR-542-3p | MIR542 | 32534 | UGUGACAGAUUGAUAACUGAAA |
| 329. | hsa-miR-545-5p | MIR545 | 32531 | UCAGUAAAUGUUUAUUAGAUGA |
| 330. | hsa-miR-545-3p | MIR545 | 32531 | UCAGCAAACAUUUAUUGUGUGC |
| 331. | hsa-miR-62S | MIR626 | 32882 | AGCUGUCUGAAAAUGUCUU |
| 236 | hsa-miR-830 | MIR830 | 32888 | AGUAUUCUGUACGAGGGAAGGU |
| 241 | hsa-miR-663 | MIR663A | 32919 | AGGCGGGGCGCCGCGGGACCGC |
| 243 | hsa-miR-720 | MIR720 | 35375 | UCUCGCUGGGGCCUCCA |
| 332. | hsa-miR-758-5p | MIR758 | 33133 | GAUGGUUGACCAGAGAGCACAC |
| 333. | hsa-miR-758-3p | MIR758 | 33133 | UUUGUGACCUGGUCCACUAACC |
| 334. | hsa-miR-876-3p | MIR876 | 33653 | UGGUGGUUUACAAAGUAAUUCA |
| 335. | hsa-miR-1185 | MIR1185-1/ | 35257/ | AGAGGAUACCCUUUGUAUGUU |
| | | MIR1185-2 | 35254 | |
| 146 | hsa-miR-1260 | MIR1260A | 35325 | AUCCCACCUCUGCCACCA |
| 336. | hsa-miR-1266-5p | MIR1266 | 35334 | CCUCAGGGCUGUAGAACAGGGCU |
| 337. | hsa-miR-1266-3p | MIR1266 | 35334 | CCCUGUUCUAUGCCCUGAGGGA |
| 338. | hsa-miR-199a-3p | MIR199A1/ | 31571/ | ACAGUAGUCUGCACAUUGGUUA |
| | | MIR199A2 | 31572 | |
| 339. | hsa-miR-199b-3p | MIR199B | 31573 | ACAGUAGUCUGCACAUUGGUUA |
| 224 | hsa-miR-497 | MIR497 | 32088 | CAGCAGCACACUGUGGUUUGU |
| 340. | hsa-miR-625-5p | MIR625 | 32881 | AGGGGGAAAGUUCUAUAGUCC |
| 341. | hsa-miR-825-3p | MIR625 | 32881 | GACUAUAGAACUUUCCCCCUCA |
| 342. | hsa-miR-831 | MIR631 | 32887 | AGACCUGGCCCAGACCUCAGC |
| 343. | hsa-miR-635 | MIR835 | 32891 | ACUUGGGCACUGAAACAAUGUCC |

TABLE 5

| No. | Symbol | ID | Name | HGNC | GI |
|---|---|---|---|---|---|
| (i) | (ii) | (iii) | (iv) | (v) | (vi) |
| 344. | CDC42 | 998 | Homo sapiens cell division cycle 42 (GTP | 1736 | 33990903 |
| | | | binding protein 25 kDa) transcript variant 1 | | |
| 345. | EGFR_ | 1956 | Homo sapiens epidermal growth factor receptor | 3236 | 83101669 |
| | int | | (erythroblastic leukemia viral (v-erb-b) | | |
| | | | oncogene homolog avian) | | |
| 346. | KIT_ex | 3815 | Homo sapiens v-kit Hardy-Zuckerman 4 feline | 6342 | 47938801 |
| | | | sarcoma viral oncogene homolog | | |
| 347. | PPARG | 5466 | Homo sapiens peroxisome proliferative | 9236 | 13905055 |
| | | | activated receptor gamma transcript variant 3 | | |
| 348. | WT1 | 7490 | Homo sapiens Wilms tumor 1 | 12706 | 34190661 |

Columns

(i)-(vi) are the same as those in Table 2.

TABLE 6

| Symbol (i) | ID (ii) | P.Value (iii) | Expression (iv) |
|---|---|---|---|
| CCNB1IP1 | 57820 | 8.16E−07 | Down |
| HSPD1 | 3329 | 1.96E−06 | Down |
| PRPF4 | 9128 | 2.96E−08 | Down |
| STYXL1 | 51657 | 2.22E−05 | Down |
| ETNK2 | 55224 | 2.23E−05 | Down |
| GALK1 | 2584 | 2.75E−05 | Down |
| RNF40 | 9810 | 2.95E−05 | Down |
| NTRK3_ext | 4916 | 4.98E−05 | Down |
| UBA1 | 7317 | 8.54E−05 | Up |
| GTF2H1 | 2965 | 9.23E−05 | Down |
| PDEAA | 5141 | 0.000115 | Down |
| SMARCE1 | 6605 | 0.000133 | Up |
| TBPL1 | 9519 | 0.000197 | Down |
| PIP4K2B | 8396 | 0.000252 | Down |
| LDB1 | 8861 | 0.000304 | Down |
| RHOT2 | 89941 | 0.00037 | Down |
| SMARCD1 | 6602 | 0.000384 | Down |
| SAV1 | 60485 | 0.000403 | Down |
| ZSCAN12 | 9753 | 0.000412 | Down |
| ASB1 | 51665 | 0.000414 | Down |
| KLHL12 | 59349 | 0.000428 | Up |
| UXT | 8409 | 0.000642 | Down |
| E2F6 | 1876 | 0.000747 | Up |
| RBMS1 | 5937 | 0.000829 | Up |
| MAP2K5 | 5607 | 0.000909 | Down |
| PAPSS2 | 9060 | 0.001015 | Down |
| RAN | 5901 | 0.001377 | Up |
| STUB1 | 10273 | 0.015531 | Up |
| TPM1 | 7168 | 0.022489 | Up |

Columns (Tables 6 & 7)

(i) The “ID” column shows the Entrez GeneID number for the antigen marker. An Entrez GeneID value is unique across all taxa.

(ii) The “Symbol” column gives the gene symbol which has been approved by the HGNC. The HGNC aims to give unique and meaningful names to every miRNA and human gene.

(iii) The represents the p-value of a microarray T-test derived from comparing case with control, as determined in study 1.

(iv) The biomarkers can be up-regulated (Le an increase in fold-change, when compared to control samples) or down-regulated (i.e. a decrease in fold-change, when compared to control samples), as determined in study 1.

TABLE 7

| Symbol (i) | ID (ii) | P.Value (iii) | Expression (iv) |
|---|---|---|---|
| TPM1 | 7168 | 0.002915091 | Up |
| RORC | 6097 | 0.006178486 | Down |
| HSPD1 | 3329 | 0.008954673 | Down |
| SRPK1 | 6732 | 0.015397291 | Down |
| PYGO2 | 90780 | 0.024303172 | Down |
| SOD2 | 6648 | 0.026978148 | Up |
| STUB1 | 10273 | 0.039435959 | Up |
| RAN | 5901 | 0.02988748 | Up |

TABLE 8

| | | Expression |
|---|---|---|
| miRNA Name (i) | P.Value (ii) | (iii) |
| hsa-miR-150 | 6.39411E−05 | Down |
| hsa-miR-122 | 0.025339825 | Down |
| hsa-miR-342-3p | 3.34153E−05 | Down |
| hsa-miR-3648 | 0.005587809 | Up |
| hsa-miR-574-5p | 2,92134E−05 | Down |
| hsa-miR-1224-5p | 0.005657118 | Up |
| hsa-miR-483-3p | 1.0516E−07 | Down |
| hsa-miR-1290 | 0.027502289 | Down |
| hsa-miR-630 | 7.28115E−07 | Down |
| hsa-miR-4284 | 6.22332E−06 | Down |
| hsa-miR-4274 | 0.005782413 | Down |
| hsa-miR-1539 | 0.016898976 | Down |
| hsa-miR-484 | 5.86031E−05 | Down |
| hsa-miR-150* | 0.0001524 | Up |
| hsa-miR-103a | 0.000151533 | Down |
| hsa-miR-595 | 3.69681E−08 | Down |
| hsa-let-7f | 0.011038922 | Down |
| hsa-miR-2116* | 0.009860572 | Down |
| hsa-let-7g | 0.018048758 | Down |
| hsa-miR-10b | 0.000371424 | Up |
| hsa-miR-557 | 0.001717151 | Up |
| hsa-miR-574-3p | 1.5527E−05 | Down |
| hsa-miR-29b | 0.021302443 | Down |
| hsa-miR-3875-3p | 0.02426579 | Down |
| hsa-miR-3196 | 0.000777606 | Up |
| hsa-miR-142-3p | 0.001847633 | Down |
| hsa-miR-762 | 0.003100257 | Up |
| hsv2-miR-H22 | 0.017619039 | Up |
| hsa-miR-1260 | 2.30838E−06 | Down |
| hsv1-miR-H17 | 0.047904609 | Up |
| hsa-miR-1281 | 7.78815E−06 | Down |
| hsa-miR-720 | 1.08053E−05 | Down |
| ebv-miR-BART12 | 5.95912E−05 | Down |
| hsa-miR-146a | 0.043521819 | Down |
| hsa-miR-1183 | 0.011113703 | Up |
| hsa-miR-1228* | 8.27861E−07 | Down |
| hsa-miR-3131 | 0.000773528 | Up |
| hsa-miR-1825 | 1.48649E−08 | Down |
| hsa-miR-30b | 0.006449318 | Down |
| kshv-miR-K12-12* | 2.87415E−0S | Down |
| hsv2-miR-H6 | 0.008254315 | Up |
| hsa-miR-26a | 2.87528E−05 | Down |
| hsa-miR-4281 | 8.77129E−05 | Up |
| hsa-miR-3911 | 0.012758018 | Up |
| ebv-miR-BART | 4.54055E−05 | Down |
| hsv1-miR-H1* | 3.24311E−06 | Down |
| hsa-miR-1202 | 0.000551047 | Up |
| hsa-miR-3610 | 0.006875895 | Up |
| hsv1-miR-H6-3p | 4.84168E−05 | Down |
| hsa-miR-3138 | 0.008993721 | Up |
| hsa-miR-1207-5p | 0.002819455 | Up |
| hsa-miR-3149 | 9.53187E−05 | Down |
| hsa-miR-4270 | 4.37146E−05 | Up |
| hsa-miR-3195 | 0.006823245 | Up |
| hsv1-miR-H8 | 0.002026819 | Down |
| hsa-miR-4257 | 0.002878782 | Down |
| hsa-miR-1225-5p | 3.S45S8E−05 | Up |
| hsa-miR-142-5p | 0.022979438 | Down |
| hsa-miR-320c | 0.004906198 | Up |
| hsa-miR-466 | 0.00012439 | Down |
| hsa-miR-3665 | 0.021943146 | Down |
| hsa-miR-3180-5p | 7.83908E−06 | Down |
| hsa-miR-572 | 3.35607E−05 | Down |
| hsa-miR-498 | 0.038998714 | Down |
| hsv1-miR-H7* | 1.53033E−06 | Down |
| hsa-miR-4290 | 8.97207E−05 | Down |
| hsa-miR-766 | 5.28053E−05 | Down |
| hsa-miR-550a | 2.463E−06 | Down |
| hsa-miR-642b | 0.000388557 | Up |
| hiv1-miR-H1 | 0.023890902 | Up |
| hsa-miR-4286 | 9.71844E−06 | Down |
| hsa-miR-1275 | 0.028532583 | Up |
| hsa-miR-328 | 5.89449E−06 | Down |
| hsa-miR-940 | 0.000858136 | Down |
| hsa-miR-425* | 1.95809E−06 | Down |
| hsa-miR-877* | 1.97343E−08 | Down |
| hsa-miR-1237 | 0.004737656 | Down |
| hsa-miR-197 | 0.006897737 | Down |
| hsa-miR-30a | 0.002379932 | Up |
| hsa-miR-23c | 8.16183E−05 | Down |
| hsa-miR-3652 | 8.4982E−05 | Down |
| hsa-miR-22 | 0.0051271 | Up |
| hsa-miR-181a | 0.001321192 | Down |
| hsa-miR-3663-3p | 0.022163221 | Up |
| hsv2-miR-H25 | 0.018411737 | Up |
| hsa-miR-485-3p | 3.09662E−05 | Down |
| hsa-miR-324-3p | 0.000347692 | Down |
| hsa-miR-486-5p | 0.008641648 | Up |
| hsa-miR-1238 | 0.001191715 | Down |
| hsa-miR-1280 | 0.000217071 | Down |
| hsa-miR-129* | 0.024770209 | Down |
| hsa-miR-1225-3p | 0.000183052 | Down |
| hsa-let-7f-1* | 0.0106806 | Down |
| hsa-miR-4313 | 0.000725719 | Down |
| hsa-miR-4323 | 0.000144211 | Down |
| hsa-miR-149 | 9.57722E−05 | Down |
| hsa-miR-199a-5p | 0.038394728 | Down |
| hsa-miR-19b | 0.034029748 | Up |
| hsa-miR-92a | 0.031607002 | Up |
| hsv2-miR-H6* | 6.38059E−05 | Down |
| hsa-miR-16-2* | 0.03334112 | Up |
| hsa-miR-933 | 1.81512E−07 | Down |
| hsa-miR-660 | 0.007009129 | Up |
| hsa-miR-494 | 0.049102428 | Down |
| hsa-miR-1260b | 0.000504765 | Down |
| hsa-miR-33b* | 2.24675E−06 | Down |
| hsa-miR-186 | 0.021144881 | Up |
| hsa-let-7b* | 0.000296882 | Down |
| hsa-miR-424 | 0.028889653 | Up |
| kshv-miR-K12-8* | 2.07786E−05 | Down |
| hsa-miR-191* | 0.002534839 | Down |
| hsa-miR-1234 | 0.0094781 | Down |

Columns (Tables 8 & 9)

(i) The “mRNA name” column gives the name of the human mRNA as provided by the specialist database, miRBase, according to version 16 (released, August 2010).

(ii) The “p-value” represents the p-value of a microarray T test derived from comparing case with control, as determined in study 1.

(iii) The biomarkers can be up-regulated (i.e. an Increase in fold-change, when compered to control samples) or down-regulated (i.e. a decrease in fold-change, when compared to control samples), as determined in study 1.

TABLE 9

| | | Expression |
|---|---|---|
| miRNA Name (i) | P.Value (ii) | (iii) |
| hsa-miR-150 | 0.000731818 | Down |
| hsa-miR-122 | 0.001736842 | Down |
| hsa-miR-1224-5p | 0.005657118 | Up |
| hsa-miR-342-3p | 0.007122454 | Down |
| hsa-miR-3648 | 0.038141456 | Up |
| hsa-miR-574-5p | 0.043514783 | Down |

TABLE 10

| | | S + S | Sensitivity | Specificity | Assay |
|---|---|---|---|---|---|
| Biomarker | AUC | (i) | (m) | (i) | (iv) |
| hsa-miR-122 | 0.66 | 1.25 | 0.4 | 0.85 | qPCR |
| | 0.65 | 1.19 | 0.55 | 0.65 | microarray |
| hsa-miR-150 | 0.8 | 1.44 | 0.8 | 0.65 | microarray |
| | 0.69 | 1.31 | 0.47 | 0.85 | qPCR |
| hsa-miR-342-3p | 0.78 | 1.42 | 0.66 | 0.76 | microarray |
| | 0.62 | 1.14 | 0.76 | 0.38 | qPCR |
| hsa-miR-1224-5p | 0.48 | 1.06 | 0.06 | 1 | microarray |
| hsa-miR-3194-5p | 0.62 | 1.14 | 0.68 | 0.46 | qPCR |
| hsa-miR-3648 | 0.63 | 1.21 | 0.85 | 0.35 | microarray |
| hsa-miR-3663-5p | 0.7 | 1.26 | 0.41 | 0.85 | qPCR |
| hsa-miR-574-5p | 0.8 | 1.43 | 0.73 | 0.71 | microarray |
| HSPD1 | 0.62 | 1.15 | 0.61 | 0.33 | autoAb |
| RAN | 0.54 | 1.1 | 0.87 | 0.22 | autoAb |
| STUB1 | 0.66 | 1.21 | 0.65 | 0.56 | autoAb |
| TPM1 | 0.67 | 1.25 | 0.69 | 0.56 | autoAb |

Columns (Tables 10 to 14)

(i) S+S is the sum of the sensitivity and specificity columns, as determined in study 1.

(ii) and (iii) These two columns show the sensitivity and specificity of a test based solely on the relevant biomarker (or, for Tables 11-15, panel) shown in the left-hand column in the same row when tippled to the samples used in study 1.

(iv) For miRNA analysis, data was generated using either a microarray (“microarray”) or qPCR (“qPCR”) platform, as described in study 1. Autoantibody (“autoAb”) biomarkers were identified using the protein array platform described in study 1. Where panels were developed incorporating both miRNA and autoantibody biomarkers as variables (see study 1), these are described as “combiAbMir.”

TABLE 11
(2-mers)

| | | | Sensitivity | Specificity | |
|---|---|---|---|---|---|
| Panel | AUG | S + S (i) | (ii) | (iii) | Assay (iv) |
| hsa-miR-150, hsa-miR-574-5p | 0.91 | 1.63 | 0.86 | 0.76 | microarray |
| hsa-miR-342-3p, hsa-miR-574-5p | 0.86 | 1.52 | 0.76 | 0.76 | micrarray |
| hsa-miR-122, hsa-miR-574-5p | 0.84 | 1.51 | 0.75 | 0.76 | microarray |
| hsa-miR-3648, hsa-miR-574-5p | 0.83 | 1.49 | 0.79 | 0.71 | microarray |
| hsa-miR-150, hsa-miR-342-3p | 0.81 | 1.45 | 0.68 | 0.76 | microarray |
| TPM1, hsa-miR-150 | 0.811875 | 1.4825 | 0.8575 | 0.625 | combiAbMir |
| hsa-miR-342-3p, hsa-miR-3648 | 0.81 | 1.45 | 0.69 | 0.76 | microarray |
| RAN, hsa-miR-574-5p | 0.810156 | 1.430625 | 0.805625 | 0.625 | combiAbMir |
| HSPD1, hsa-miR-342-3p | 0.809219 | 1.450625 | 0.575625 | 0.875 | combiAbMir |
| RAN, hsa-miR-342-3p | 0.807813 | 1.459375 | 0.709375 | 0.75 | combiAbMir |
| TPM1, hsa-miR-342-3p | 0.806406 | 1.445625 | 0.695625 | 0.75 | combiAbMir |
| hsa-miR-1224-5p, hsa-miR-342-3p | 0.80 | 1.45 | 0.68 | 0.76 | microarray |
| TPM1, hsa-miR-574-5p | 0.796406 | 1.435 | 0.81 | 0.625 | combiAbMir |
| HSPD1, hsa-miR-150 | 0.795625 | 1.42875 | 0.67875 | 0.75 | combiAbMir |
| RAN, hsa-miR-150 | 0.784063 | 1.385625 | 0.760625 | 0.625 | combiAbMir |
| hsa-miR-1224-5p, hsa-miR-574-5p | 0.78 | 1.42 | 0.66 | 0.76 | microarray |
| hsa-miR-150, hsa-miR-3648 | 0.78 | 1.39 | 0.74 | 0.65 | microarray |
| hsa-miR-150, hsa-miR-1224-5p | 0.78 | 1.39 | 0.68 | 0.71 | microarray |
| STUB1, hsa-miR-150 | 0.777344 | 1.404375 | 0.654375 | 0.75 | combiAbMir |
| HSPD1, hsa-miR-574-5p | 0.775 | 1.3875 | 0.7625 | 0.625 | combiAbMir |
| STUB1, hsa-miR-342-3p | 0.774531 | 1.41125 | 0.66125 | 0.75 | combiAbMir |
| hsa-miR-122, hsa-miR-342-3p | 0.77 | 1.40 | 0.63 | 0.76 | microarray |
| STUB1, hsa-miR-574-5p | 0.769688 | 1.38 | 0.63 | 0.75 | combiAbMir |
| hsa-miR-150, hsa-miR-122 | 0.77 | 1.38 | 0.61 | 0.76 | microarray |
| hsa-miR-122, hsa-miR-3663-5p | 0.76 | 1.37 | 0.60 | 0.77 | qPCR |
| hsa-miR-150, hsa-miR-3663-5p | 0.75 | 1.35 | 0.50 | 0.85 | qPCR |
| hsa-miR-342-3p, hsa-miR-3683-5p | 0.72 | 1.30 | 0.80 | 0.69 | qPCR |
| TPM1, hsa-miR-122 | 0.594688 | 1.28125 | 0.85825 | 0.825 | combiAbMir |
| TPM, HSPD1 | 0.59 | 1.28 | 0.84 | 0.44 | auto Ab |
| TPM1, hsa-miR-3648 | 0.884219 | 1.24 | 0.74 | 0.5 | combiAbMir |
| HSPD1, hsa-miR-3648 | 0.672656 | 1.254375 | 0.754375 | 0.5 | combiAbMir |
| hsa-miR-150, hsa-miR-3194-5p | 0.67 | 1.28 | 0.43 | 0.85 | qPCR |
| hsa-miR-122, hsa-miR-3648 | 0.67 | 1.23 | 0.82 | 0.41 | microarray |
| STUB1, hsa-miR-122 | 0.661094 | 1.265 | 0.64 | 0.625 | combiAbMir |
| RAN, hsa-miR-3648 | 0.661094 | 1.255625 | 0.880625 | 0.375 | combiAbMir |
| TPM1, STUB1 | 0.66 | 1.25 | 0.81 | 0.44 | autoAb |
| hsa-miR-3663-5p, hsa-miR-3194-5p | 0.86 | 1.18 | 0.64 | 0.54 | qPCR |
| HSPD1,STUB1 | 0.65 | 1.19 | 0.64 | 0.56 | autoAb |
| hsa-miR-342-3p, hsa-miR-3194-5p | 0.64 | 1.18 | 0.33 | 0.85 | qPCR |
| STUB1, hsa-miR-3648 | 0.639844 | 1.20125 | 0.82625 | 0.375 | combiAbMir |
| TPM1, hsa-miR-1224-5p | 0.839531 | 1.195625 | 0.570625 | 0.625 | combiAbMir |
| HSPD1, hsa-miR-122 | 0.539063 | 1.1925 | 0.4425 | 0.75 | combiAbMir |
| hsa-miR-122, hsa-miR-3194-5p | 0.84 | 1.26 | 0.41 | 0.85 | qPCR |
| HSPD1, RAN | 0.83 | 1.17 | 0.84 | 0.33 | autoAb |
| hsa-miR-150, hsa-miR-122 | 0.83 | 1.21 | 0.36 | 0.85 | qPCR |
| hsa-miR-150, hsa-miR-342-3p | 0.63 | 1.21 | 0.36 | 0.85 | qPCR |
| TPM1, RAN | 0.63 | 1.18 | 0.73 | 0.44 | autoAb |
| hsa-miR-122, hsa-miR-342-3p | 0.62 | 1.20 | 0.35 | 0.85 | qPCR |
| RAN, hsa-miR-122 | 0.617813 | 1.159375 | 0.534375 | 0.625 | combiAbMir |
| STUB1, hsa-miR-1224-5p | 0.612188 | 1.173125 | 0.423125 | 0.75 | combiAbMir |
| hsa-miR-122, hsa-miR-1224-5p | 0.59 | 1.11 | 0.70 | 0.41 | microarray |
| HSPD1, hsa-miR-1224-5p | 0.587031 | 1.120625 | 0.620625 | 0.5 | combiAbMir |
| RAN, STUB1 | 0.59 | 1.15 | 0.82 | 0.33 | autoAb |
| hsa-miR-1224-5p, hsa-miR-3648 | 0.58 | 1.11 | 0.81 | 0.20 | microarray |
| RAN, hsa-miR-1224-5p | 0.537344 | 1.084375 | 0.834375 | 0.25 | combiAbMir |

TABLE 12
(3-mers)

| | | | Sensitivity | Specificity | |
|---|---|---|---|---|---|
| Panel | AUC | S + S (i) | (ii) | (iii) | Assay (iv) |
| RAN, hsa-miR-150, hsa-miR-574-5p | 0.923594 | 1.6575 | 0.7825 | 0.875 | combiAbMir |
| TPM1, hsa-miR-150, hsa-miR-574-5p | 0.923594 | 1.655 | 0.905 | 0.75 | combiAbMir |
| hsa-miR-150, hsa-miR-342-3p, hsa- | 0.90 | 1.61 | 0.85 | 0.76 | microarray |
| miR-574-5p | | | | | |
| STUB1, hsa-miR-150, hsa-miR-574-5p | 0.900781 | 1.61625 | 0.86625 | 0.75 | combiAbMir |
| hsa-miR-150, hsa-miR-122, hsa-miR- | 0.90 | 1.60 | 0.83 | 0.76 | microarray |
| 574-5p | | | | | |
| HSPD1, hsa-miR-150, hsa-miR-574-5p | 0.897188 | 1.600625 | 0.850625 | 0.75 | combiAbMir |
| hsa-miR-122, hsa-miR-342-3p, hsa- | 0.90 | 1.60 | 0.78 | 0.82 | microarray |
| miR-574-5p | | | | | |
| TPM1, hsa-miR-342-3p hsa-miR-574- | 0.892969 | 1.57125 | 0.69625 | 0.875 | combiAbMir |
| 5p | | | | | |
| hsa-miR-150, hsa-miR-1224-5p, hsa- | 0.89 | 1.59 | 0.83 | 0.76 | microarray |
| miR-574-5p | | | | | |
| hsa-miR-150, hsa-miR-3648, hsa-miR- | 0.89 | 1.59 | 0.83 | 0.76 | microarray |
| 574-5p | | | | | |
| RAN, hsa-miR-342-3p, hsa-miR-574- | 0.878906 | 1.55375 | 0.67875 | 0.875 | combiAbMir |
| 5p | | | | | |
| hsa-miR-1224-5p, hsa-miR-342-3p, | 0.88 | 1.57 | 0.81 | 0.76 | microarray |
| hsa-miR-574-5p | | | | | |
| TPM1, hsa-miR-122, hsa-miR-574-5p | 0.872344 | 1.55 | 0.8 | 0.75 | combiAbMir |
| STUB1, hsa-miR-342-3p, hsa-miR- | 0.867031 | 1.535 | 0.785 | 0.75 | combiAbMir |
| 574-5p | | | | | |
| RAN, hsa-miR-122, hsa-miR-574-5p | 0.864219 | 1 52875 | 0.77875 | 0 75 | combiAbMir |
| hsa-miR-342-3p, hsa-miR-3648, hsa- | 0.86 | 1.54 | 0.77 | 0.76 | microarray |
| miR-574-5p | | | | | |
| HSPD1, hsa-miR-342-3p, hsa-miR- | 0.862656 | 1.51875 | 0.78875 | 0.75 | combiAbMir |
| 574-5p | | | | | |
| RAN, hsa-miR-3648, hsa-miR-574-5p | 0.860781 | 1.515625 | 0.765625 | 0.75 | combiAbMir |
| hsa-miR-122, hsa-miR-3648, hsa-miR- | 0.85 | 1.51 | 0.75 | 0.76 | microarray |
| 574-5p | | | | | |
| TPM1, hsa-miR-3648, hsa-mR-574-5p | 0.8425 | 1.518125 | 0.768125 | 0.75 | combiAbMir |
| TPM1, RAN, hsa-miR-574-5p | 0.825156 | 1.45875 | 0.70875 | 0.75 | combiAbMir |
| hsa-miR-122, hsa-miR-1224-5p, hsa- | 0.82 | 1.49 | 0.72 | 0.76 | micrarray |
| miR-574-5p | | | | | |
| HSPD1, hsa-miR-122, hsa-miR-574-5p | 0.821719 | 1.46125 | 0.71125 | 0.75 | combiAbMir |
| STUB1, hsa-miR-122, hsa-miR-574-5p | 0.819375 | 1.47125 | 0.72125 | 0.75 | combiAbMir |
| TPM1, RAN, hsa-miR-342-3p | 0.815781 | 1.4575 | 0.5825 | 0.875 | combiAbMir |
| HSPD1, RAN, hsa-miR-574-5p | 0.814063 | 1.455 | 0.83 | 0.625 | combiAbMir |
| RAN, hsa-miR-342-3p, hsa-miR-3648 | 0.811094 | 1.43875 | 0.88875 | 0.75 | combiAbMir |
| TPM1, STUB1, hsa-miR-574-5p | 0.810938 | 1.454375 | 0.829375 | 0.625 | combiAbMir |
| TPM1, RAN, hsa-miR-150 | 0.810625 | 1.475 | 0.85 | 0.825 | combiAbMir |
| TPM, HSPD1, hsa-miR-342-3p | 0.809688 | 1.473125 | 0.723125 | 0.75 | combiAbMir |
| RAN, hsa-miR-1224-5p hsa-miR-574- | 0.80875 | 1.45625 | 0.70825 | 0.75 | combiAbMir |
| 5p | | | | | |
| hsa-miR-1224-5p, hsa-miR-3648, hsa- | 0.81 | 1.44 | 0.74 | 0.71 | microarray |
| miR-574-5p | | | | | |
| HSPD1, hsa-miR-342-3p, hsa-miR- | 0.803438 | 1.44 | 0.69 | 0.75 | combiAbMir |
| 3648 | | | | | |
| RAN, hsa-miR-150, hsa-miR-342-3p | 0.802813 | 1.458875 | 0.581875 | 0.875 | combiAbMir |
| TPM1, hsa-miR-150, hsa-miR-342-3p | 0.802188 | 1.438875 | 0.686875 | 0.75 | combiAbMir |
| TPM1, HSPD1, hsa-miR-574-5p | 0.802031 | 1.435 | 0.81 | 0.625 | combiAbMir |
| TPM, hsa-miR-342-3p, hsa-miR-3648 | 0.801094 | 1.439375 | 0.689375 | 0.75 | combiAbMir |
| TPM1, HSPD1, hsa-miR-150 | 0.800625 | 1.45375 | 0.82875 | 0.625 | combiAbMir |
| STUB1, hsa-miR-3648, hsa-miR-574- | 0.800469 | 1.425625 | 0.675625 | 0.75 | combiAbMir |
| 5p | | | | | |
| hsa-miR-150, hsa-miR-1224-5p, hsa- | 0.80 | 1.42 | 0.65 | 0.76 | microarray |
| miR-342-3p | | | | | |
| HSPD1, RAN, hsa-miR-150 | 0.799844 | 1.44 | 0.60 | 0.75 | combiAbMir |
| HSPD1, hsa-miR-150, hsa-miR-342-3p | 0.799219 | 1.419375 | 0.689375 | 0.75 | combiAbMir |
| RAN, hsa-miR-122, hsa-miR-342-3p | 0.798281 | 1.43125 | 0.68125 | 0.75 | combiAbMir |
| TPM1, hsa-miR-1224-5p, hsa-miR- | 0.798125 | 1.451875 | 0.826875 | 0.625 | combiAbMir |
| 574-5p | | | | | |
| RAN, STUB1, hsa-miR-574-5p | 0.7975 | 1.415625 | 0.665625 | 0.75 | combiAbMir |
| TPM1, STUB1, hsa-miR-342-3p | 0.797188 | 1.435625 | 0.685625 | 0.75 | combiAbMir |
| TPM1, hsa-miR-122, hsa-miR-342-3p | 0.796719 | 1.423125 | 0.548125 | 0.875 | combiAbMir |
| TPM1, hsa-miR-150, hsa-miR-3648 | 0.794063 | 1.4625 | 0.8375 | 0.625 | combiAbMir |
| TPM1, hsa-miR-1224-5p, hsa-miR- | 0.793906 | 1.44375 | 0.55875 | 0.875 | combiAbMir |
| 342-3p | | | | | |
| HSPD1, RAN, hsa-miR-342-3p | 0.793281 | 1.446875 | 0.571875 | 0.875 | combiAbMir |
| RAN, hsa-miR-1224-5p, hsa-miR-342- | 0.793125 | 1.425625 | 0.550625 | 0.875 | combiAbMir |
| 3p | | | | | |
| HSPD1, STUB1, hsa-miR-342-3p | 0.7925 | 1.4325 | 0.6825 | 0.75 | combiAbMir |
| HSPD1, hsa-miR-150, hsa-miR-1224- | 0.790781 | 1.419375 | 0.669375 | 0.75 | combiAbMir |
| 5p | | | | | |
| STUB1, hsa-mR-342-3p, hsa-miR- | 0.786875 | 1.40875 | 0.65875 | 0.75 | combiAbMir |
| 3648 | | | | | |
| HSPD1, hsa-miR-3648, hsa-miR-574- | 0.781563 | 1.389375 | 0.889375 | 0.5 | combiAbMir |
| 5p | | | | | |
| STUB1, hsa-miR-1224-5p, hsa-miR- | 0.781406 | 1.404375 | 0.654375 | 0.75 | combiAbMir |
| 574-5p | | | | | |
| STUB1, hsa-miR-122, hsa-miR-342-3p | 0.78125 | 1.420625 | 0.545625 | 0.875 | combiAbMir |
| RAN, hsa-miR-150, hsa-miR-1224-5p | 0.78125 | 1.39 | 0.765 | 0.625 | combiAbMir |
| TPM1, hsa-miR-150, hsa-miR-122 | 0.778438 | 1.436875 | 0.811875 | 0.625 | combiAbMir |
| TPM1, STUB1, hsa-miR-150 | 0.7775 | 1.424375 | 0.799375 | 0.625 | combiAbMir |
| hsa-miR-150, hsa-miR-342-3p, hsa- | 0.78 | 1.40 | 0.63 | 0.76 | microarray |
| miR-3648 | | | | | |
| hsa-miR-150, hsa-miR-122, hsa-miR- | 0.78 | 1.40 | 0.64 | 0.76 | microarray |
| 342-3p | | | | | |
| STUB1, hsa-miR-1224-5p, hsa-miR- | 0.775938 | 1.395625 | 0.520625 | 0.875 | combiAbMir |
| 342-3p | | | | | |
| RAN, hsa-miR-150, hsa-miR-3648 | 0.775156 | 1.38125 | 0.75625 | 0.625 | combiAbMir |
| TPM1, hsa-miR-150, hsa-miR-1224-5p | 0.774063 | 1.4225 | 0.7975 | 0.625 | combiAbMir |
| hsa-miR-122, hsa-miR-1224-5p, hsa- | 0.77 | 1.38 | 0.61 | 0.76 | microarray |
| miR-342-3p | | | | | |
| STUB1, hsa-miR-150, hsa-miR-342-3p | 0.770938 | 1.398125 | 0.648125 | 0.75 | combiAbMir |
| HSPD1, hsa-miR-150, hsa-miR-3648 | 0.769531 | 1.38375 | 0.75875 | 0.625 | combiAbMir |
| HSPD1, STUB1, hsa-miR-574-5p | 0.766719 | 1.355 | 0.73 | 0.625 | combiAbMir |
| RAN, STUB1, hsa-miR-342-3p | 0.764844 | 1.38375 | 0.63375 | 0.75 | combiAbMir |
| HSPD1, hsa-miR-1224-5p, hsa-miR- | 0.764531 | 1.37375 | 0.49875 | 0.875 | combiAbMir |
| 342-3p | | | | | |
| HSPD1, hsa-miR-122, hsa-miR-342-3p | 0.763438 | 1.393125 | 0.518125 | 0.875 | combiAbMir |
| hsa-miR-1224-5p, hsa-miR-342-3p, | 0.76 | 1.37 | 0.72 | 0.65 | microarray |
| hsa-miR-3648 | | | | | |
| hsa-miR-122, hsa-miR-342-3p, hsa- | 0.76 | 1.36 | 0.71 | 0.65 | microarray |
| miR-3648 | | | | | |
| HSPD1, hsa-miR-150, hsa-miR-122 | 0.761094 | 1.36875 | 0.61875 | 0.75 | combiAbMir |
| HSPD1, STUB1, hsa-miR-150 | 0.760938 | 1.3825 | 0.7575 | 0.625 | combiAbMir |
| STUB1, hsa-miR-150, hsa-miR-122 | 0.760625 | 1.364375 | 0.739375 | 0.625 | combiAbMir |
| hsa-miR-150, hsa-miR-1224-5p, hsa- | 0.76 | 1.35 | 0.71 | 0.65 | micrarray |
| miR-3648 | | | | | |
| RAN, hsa-miR-150, hsa-miR-122 | 0.755825 | 1.35875 | 0.73375 | 0.625 | combiAbMir |
| HSPD1, hsa-miR-1224-5p, hsa-miR- | 0.754688 | 1.361875 | 0.736875 | 0.625 | combiAbMir |
| 574-5p | | | | | |
| RAN, STUB1, hsa-miR-150 | 0.754531 | 1.371875 | 0.746875 | 0.625 | combiAbMir |
| STUB1, hsa-miR-150, hsa-miR-1224- | 0.754219 | 1.370625 | 0.745625 | 0.625 | combiAbMir |
| 5p | | | | | |
| STUB1, hsa-miR-150, hsa-miR-3648 | 0.748906 | 1.351875 | 0.726875 | 0.625 | combiAbMir |
| hsa-miR-150, hsa-miR-122, hsa-miR- | 0.75 | 1.35 | 0.65 | 0.71 | microarray |
| 1224-5p | | | | | |
| hsa-miR-150, hsa-miR-122, hsa-miR- | 0.75 | 1.35 | 0.71 | 0.65 | microarray |
| 3648 | | | | | |
| hsa-miR-150, hsa-miR-3663-5p, hsa- | 0.74 | 1.34 | 0.65 | 0.69 | qPCR |
| miR-3194-5p | | | | | |
| TPM1, HSPD1, hsa-miR-122 | 0.730938 | 1.346875 | 0.721875 | 0.625 | combiAbMir |
| hsa-miR-122, hsa-miR-3663-5p, hsa- | 0.72 | 1.32 | 0.47 | 0.85 | QPCR |
| miR-3194-5p | | | | | |
| hsa-miR-150, hsa-miR-122, hsa-miR- | 0.72 | 1.31 | 0.48 | 0.85 | qPCR |
| 3663-5p | | | | | |
| hsa-miR-122, hsa-miR-342-3p, hsa- | 0.72 | 1.32 | 0.63 | 0.89 | QPCR |
| miR-3663-5p | | | | | |
| hsa-miR-150, hsa-miR-342-3p, hsa- | 0.72 | 1.31 | 0.61 | 0.69 | qPCR |
| miR-3663-5p | | | | | |
| hsa-miR-342-3p, hsa-miR-3663-5p, | 0.70 | 1.28 | 0.59 | 0.69 | qPCR |
| hsa-miR-3194-5p | | | | | |
| TPM1, HSPD1, hsa-miR-3848 | 0.7 | 1.280625 | 0.780625 | 0.5 | combiAbMir |
| TPM1, RAN, hsa-miR-3648 | 0.697656 | 1.258125 | 0.758125 | 0.5 | combiAbMir |
| TPM1, hsa-miR-122, hsa-miR-3648 | 0.69375 | 1.2575 | 0.7575 | 0.5 | combiAbMir |
| HSPD1, RAN, hsa-miR-3648 | 0.688125 | 1.288125 | 0.913125 | 0.375 | combiAbMir |
| HSPD1, STUB1, hsa-miR-3648 | 0.686406 | 1.24875 | 0.74875 | 0.5 | combiAbMir |
| TPM1, HSPD1, RAN | 0.88 | 1.28 | 0.83 | 0.44 | autoAb |
| TPM1, HSPD1,STUB1 | 0.68 | 1.29 | 0.84 | 0.44 | autoAb |
| TPM1, STUB1, hsa-miR-122 | 0.680781 | 1.289375 | 0.789375 | 0.5 | combiAbMir |
| RAN, hsa-miR-122, hsa-miR-3648 | 0.68 | 1.263125 | 0.888125 | 0.375 | combiAbMir |
| TPM1, hsa-miR-1224-5p, hsa-miR- | 0.68 | 1.235 | 0.735 | 0.5 | combiAbMir |
| 3648 | | | | | |
| STUB1, hsa-miR-122, hsa-miR-3648 | 0.679375 | 1.2575 | 0.7575 | 0.5 | combiAbMir |
| HSPD1, hsa-miR-122, hsa-miR-3648 | 0.674531 | 1.21875 | 0.71875 | 0.5 | combiAbMir |
| TPM1, hsa-miR-122, hsa-miR-1224-5p | 0.674375 | 1.268125 | 0.643125 | 0.625 | combiAbMir |
| TPM1, STUB1, hsa-miR-3648 | 0.684375 | 1.234375 | 0.734375 | 0.5 | combiAbMir |
| RAN, STUB1, hsa-miR-3648 | 0.663594 | 1.245625 | 0.870625 | 0.375 | combiAbMir |
| TPM1, HSPD1, hsa-miR-1224-5p | 0.661875 | 1.22125 | 0.72125 | 0.5 | combiAbMir |
| TPM1, RAN, hsa-miR-122 | 0.655625 | 1.24 | 0.74 | 0.5 | combiAbMir |
| HSPD1, RAN, hsa-miR-122 | 0.655625 | 1.18375 | 0.80875 | 0.375 | combiAbMir |
| hsa-miR-150, hsa-miR-342-3p, hsa- | 0.65 | 1.25 | 0.41 | 0.85 | qPCR |
| miR-3194-5p | | | | | |
| hsa-miR-150, hsa-miR-122, hsa-miR- | 0.65 | 1.21 | 0.36 | 0.85 | qPCR |
| 342-3p | | | | | |
| HSPD1, RAN, STUB1 | 0.65 | 1.21 | 0.78 | 0.44 | autoAb |
| HSPD1, STUB1, hsa-miR-122 | 0.648281 | 1.20875 | 0.58375 | 0.625 | combiAbMir |
| hsa-miR-122, hsa-miR-1224-5p, hsa- | 0.64 | 1.17 | 0.82 | 0.35 | microarray |
| miR-3648 | | | | | |
| hsa-miR-150, hsa-miR-122, hsa-miR- | 0.64 | 1.25 | 0.41 | 0.85 | qPCR |
| 3194-5p | | | | | |
| TPMY, STUB1, hsa-miR-1224-5p | 0.635313 | 1 204375 | 0.704375 | 0.5 | combiAbMir |
| HSPD1, STUB1, hsa-miR-1224-5p | 0.63375 | 1.199375 | 0.699375 | 0.5 | combiAbMir |
| RAN, hsa-miR-1224-5p, hsa-miR-3648 | 0.632656 | 1.191875 | 0.816875 | 0.375 | combiAbMir |
| RAN, STUB1, hsa-miR-122 | 0.629063 | 1.196875 | 0.571875 | 0.625 | combiAbMir |
| TPM1, RAN, STUB1 | 0.62 | 1.19 | 0.64 | 0.56 | autoAb |
| TPM1, RAN, hsa-miR-1224-5p | 0.619218 | 1.151875 | 0.651875 | 0.5 | combiAbMir |
| hsa-miR-122, hsa-miR-342-3p, hsa- | 0.62 | 1.22 | 0.37 | 0.85 | qPCR |
| miR-3194-5p | | | | | |
| STUB1, hsa-miR-122, hsa-miR-1224- | 0.614688 | 1.1825 | 0.5575 | 0.625 | combiAbMir |
| 5p | | | | | |
| HSPD1, hsa-miR-122, hsa-miR-1224- | 0.614063 | 1.158125 | 0.408125 | 0.75 | combiAbMir |
| 5p | | | | | |
| HSPD1, RAN, hsa-miR-1224-5p | 0.613281 | 1.1725 | 0.7975 | 0.375 | combiAbMir |
| STUB1, hsa-miR-1224-5p, hsa-miR- | 0.609210 | 1.155 | 0.78 | 0.375 | combiAbMir |
| 3648 | | | | | |
| HSPD1, hsa-miR-1224-5p, hsa-miR- | 0.604375 | 1.15 | 0.9 | 0.25 | combiAbMir |
| 3648 | | | | | |
| RAN, hsa-miR-122, hsa-miR-1224-5p | 0.599063 | 1.11625 | 0.61625 | 0.5 | combiAbMir |
| RAN, STUB1, hsa-miR-1224-5p | 0.574531 | 1.136875 | 0.781875 | 0.375 | combiAbMir |

TABLE 13
(4-mers)

| | | | Sensitivity | Specificity | |
|---|---|---|---|---|---|
| Panel | AUC | S + S (i) | (ii) | (iii) | Assay (iv) |
| TPM1, hsa-miR-150, hsa-miR-342-3p, | 0.828594 | 1.8525 | 0.9025 | 0.75 | combiAbMir |
| hsa-miR-574-5p | | | | | |
| TPM1, hsa-miR-150, hsa-miR-122, hsa- | 0.928125 | 1.949375 | 0.899375 | 0.75 | combiAbMir |
| hsa-miR-574-5p | | | | | |
| RAN, hsa-miR-150, hsa-mR-3648, hsa- | 0.925469 | 1.64625 | 0.77125 | 0.875 | combiAbMir |
| hsa-miR-574-5p | | | | | |
| TPM1, RAN, hsa-miR-150, hsa-miR-574- | 0.923125 | 1.646875 | 0.896875 | 0.75 | combiAbMir |
| 5p | | | | | |
| TPM1, STUB1, hsa-miR-150, hsa-miR- | 0.919844 | 1.635 | 0.76 | 0.875 | combiAbMir |
| 574-5p | | | | | |
| RAN, hsa-miR-150, hsa-miR-122, hsa- | 0.914375 | 1.635 | 0.76 | 0.675 | combiAbMir |
| miR-574-5p | | | | | |
| TPM1, hsa-miR-150, hsa-miR-3648, hsa- | 0.913594 | 1.6125 | 0.8625 | 0.75 | combiAbMir |
| miR-574-5p | | | | | |
| RAN, STUB1, hsa-miR-150, hsa-miR-574. | 0.913438 | 1.635625 | 0.760625 | 0.875 | combiAbMir |
| 5p | | | | | |
| TPM1, hsa-miR-150, hsa-miR-1224-5p, | 0.913438 | 1.631875 | 0.881875 | 0.75 | combiAbMir |
| hsa-miR-574-5p | | | | | |
| HSPD1, RAN, hsa-miR-150, hsa-miR-574- | 0.912031 | 1.62375 | 0.74875 | 0.875 | combiAbMir |
| 5p | | | | | |
| RAN, hsa-miR-150, hsa-miR-342-3p, hsa- | 0.908594 | 1.619375 | 0.869375 | 0.75 | combiAbMir |
| miR-574-5p | | | | | |
| RAN, hsa-miR-150, hsa-miR-1224-5p, | 0.906719 | 1.618125 | 0.868125 | 0.75 | combiAbMir |
| hsa-miR-574-5p | | | | | |
| TPM1, hsa-miR-122, hsa-miR-342-3p, | 0.906563 | 1.61125 | 0.66125 | 0.75 | combiAbMir |
| hsa-miR-574-5p | | | | | |
| TPM1, HSPD1, hsa-miR-150, hsa-miR- | 0.906406 | 1.6125 | 0.8625 | 0.75 | combiAbMir |
| 574-5p | | | | | |
| HSPD1, hsa-miR-150, hsa-miR-342-3p, | 0.903906 | 1.6075 | 0.8575 | 0.75 | combiAbMir |
| hsa-miR-574-5p | | | | | |
| STUB1, hsa-miR-150, hsa-miR-342-3p, | 0.902031 | 1.615625 | 0.665625 | 0.75 | combiAbMir |
| hsa-miR-574-5p | | | | | |
| TPM1, RAN, hsa-miR-342-3p, hsa-miR- | 0.901406 | 1.584375 | 0.834375 | 0.75 | combiAbMir |
| 574-5p | | | | | |
| hsa-miR-150, hsa-miR-122, hsa-miR- | 0.90 | 1.61 | 0.84 | 0.76 | microarray |
| 3648, hsa-miR-574-5p | | | | | |
| HSPD1, hsa-miR-150, hsa-miR-122, hsa- | 0.900625 | 1.593125 | 0.843125 | 0.75 | combiAbMir |
| miR-574-5p | | | | | |
| HSPD1, hsa-miR-150, hsa-miR-1224-5p, | 0.898125 | 1.605625 | 0.855625 | 0.75 | combiAbMir |
| hsa-miR-574-5p | | | | | |
| hsa-miR-150, hsa-miR-122, hsa-miR-342- | 0.90 | 1.59 | 0.83 | 0.76 | microarray |
| 3p, hsa-miR-574-5p | | | | | |
| TPM1, HSPD1, hsa-miR-342-3p, hsa-miR- | 0.895781 | 1.58625 | 0.83625 | 0.75 | combiAbMir |
| 574-5p | | | | | |
| HSPD1, STUB1, hsa-miR-150, hsa-miR- | 0.695313 | 1.6 | 0.85 | 0.75 | combiAbMir |
| 574-5p | | | | | |
| TPM1, STUB1, hsa-miR-342-3p, hsa-miR- | 0.893125 | 1.579375 | 0.829375 | 0.75 | combiAbMir |
| 574-5p | | | | | |
| STUB1, hsa-miR-150, hsa-miR-3648, hsa- | 0.891563 | 1.595 | 0.845 | 0.75 | combiAbMir |
| miR-574-5p | | | | | |
| HSP01, hsa-miR-122, hsa-miR-342-3p, | 0.890938 | 1.6075 | 0.7325 | 0.875 | combiAbMir |
| hsa-miR-574-5p | | | | | |
| STUB1, hsa-miR-150, hsa-miR-122, hsa- | 0.890625 | 1.58375 | 0.83375 | 0.75 | combiAbMir |
| miR-574-5p | | | | | |
| HSPD1, hsa-miR-150, hsa-miR-3648, | 0.889063 | 1.5925 | 0.8425 | 0.75 | combiAbMir |
| hsa-miR-574-5p | | | | | |
| STUB1, hsa-miR-122, hsa-miR-342-3p, | 0.888594 | 1.585625 | 0.835625 | 0.75 | combiAbMir |
| hsa-miR-574-5p | | | | | |
| RAN, hsa-miR-122, hsa-miR-342-3p, hsa- | 0.886719 | 1.6 | 0.725 | 0.875 | combiAbMir |
| miR-574-5p | | | | | |
| hsa-miR-150, hsa-miR-1224-5p, hsa-miR- | 0.89 | 1.57 | 0.81 | 0.76 | microarray |
| 3648, hsa-miR-574-5p | | | | | |
| RAN, hsa-mR-342-3p, hsa-miR -3648, | 0.884375 | 1.560625 | 0.810625 | 0.75 | combiAbMir |
| hsa-miR-574-5p | | | | | |
| STUB'S, hsa-miR-150, hsa-miR-1224-5p, | 0.882344 | 1.575625 | 0.825625 | 0.75 | combiAbMir |
| hsa-miR-574-5p | | | | | |
| hsa-miR-150, hsa-miR-342-3p, hsa-miR- | 0.88 | 1.57 | 0.80 | 0.76 | microarray |
| 3648, hsa-miR-574-5p | | | | | |
| RAN, STUB1, hsa-miR-342-3p, hsa-miR- | 0.88125 | 1.555825 | 0.680625 | 0.875 | combiAbMir |
| 574-5p | | | | | |
| hsa-miR-150, hsa-miR-122, hsa-miR- | 0.88 | 1.56 | 0.80 | 0.76 | microarray |
| 1224-5p, hsa-miR-574-5p | | | | | |
| TPM1, hsa-miR-342-3p, hsa-miR-3648, | 0.880313 | 1.555625 | 0.805625 | 0.75 | combiAbMir |
| hsa-miR-574-5p | | | | | |
| TPM1, hsa-miR-122, hsa-miR-3648, hsa- | 0.877813 | 1.5725 | 0.8225 | 0.75 | combiAbMir |
| miR-574-5p | | | | | |
| TPM1, hsa-miR-1224-5p, hsa-miR-342- | 0.877813 | 1.556875 | 0.806875 | 0.75 | combiAbMir |
| 3p, hsa-miR-574-5p | | | | | |
| hsa-miR-150, hsa-miR-1224-5p, hsa-miR- | 0.88 | 1.57 | 0.81 | 0.76 | microarray |
| 342-3p, hsa-miR-574-5p | | | | | |
| hsa-miR-122, hsa-miR-342-3p, hsa-miR- | 0.88 | 1.56 | 0.80 | 0.76 | microarray |
| 3648, hsa-miR-574-5p | | | | | |
| HSPD1, STUB1, hsa-miR-342-3p hsa- | 0.874688 | 1.54625 | 0.79625 | 0.75 | combiAbMir |
| miR-574-5p | | | | | |
| RAN, hsa-miR-1224-5p, hsa-miR-342-3p, | 0.872031 | 1.565 | 0.69 | 0.875 | combiAbMir |
| hsa-miR-574-5p | | | | | |
| HSPD1, hsa-miR-1224-5p, hsa-miR-342- | 0.870156 | 1.5525 | 0.8025 | 0.75 | combiAbMir |
| 3p, hsa-miR-574-5p | | | | | |
| HSPD1, RAN, hsa-miR-342-3p, hsa-miR- | 0.87 | 1.546875 | 0.671875 | 0.875 | combiAbMir |
| 574-5p | | | | | |
| hsa-miR-122, hsa-miR -1224-5p, hsa-miR- | 0.87 | 1.55 | 0.73 | 0.82 | microarray |
| 342-3p, hsa-miR-574-5p | | | | | |
| TPM1, RAN, hsa-miR-3648, hsa-miR-574- | 0.8825 | 1.5425 | 0.7925 | 0.75 | combiAbMir |
| 5p | | | | | |
| TPM1, hsa-miR-122, hsa-miR-1224-5p, | 0.861094 | 1.536875 | 0.786875 | 0.75 | combiAbMir |
| hsa-miR-574-5p | | | | | |
| STUB1, hsa-miR-1224-5p, hsa-miR-342- | 0.859844 | 1.530625 | 0.780625 | 0.75 | combiAbMir |
| 3p, hsa-miR-574-5p | | | | | |
| hsa-miR-1224-5p, hsa-miR-342-3p, hsa- | 0.86 | 1.52 | 0.76 | 0.76 | microarray |
| miR-3648, hsa-miR-574-5p | | | | | |
| STUB1, hsa-miR-342-3p, hsa-miR-3648, | 0.855156 | 1.5025 | 0.7525 | 0.75 | combiAbMir |
| hsa-miR-574-5p | | | | | |
| RAN, hsa-miR-122, hsa-miR-3648, hsa- | 0.854219 | 1.5 | 0.75 | 0.75 | combiAbMir |
| miR-574-5 | | | | | |
| TPM1, HSPD1, hsa-miR-122, hsa-miR- | 0.852969 | 1.5 | 0.75 | 0.75 | combiAbMir |
| 574-5p | | | | | |
| HSPD1, hsa-miR-342-3p, hsa-miR-3648, | 0.850625 | 1.50625 | 0.75625 | 0.75 | combiAbMir |
| hsa-miR-574-5p | | | | | |
| TPM1, RAN, hsa-miR-122, hsa-miR-574- | 0.848281 | 1.4975 | 0.7475 | 0.75 | combiAbMir |
| 5p | | | | | |
| TPM1, STUB1, hsa-miR-122, hsa-miR- | 0.847813 | 1.509375 | 0.759375 | 0.75 | combiAbMir |
| 574-5p | | | | | |
| HSPD1, hsa-miR-122, hsa-miR-3648, | 0.846719 | 1.496875 | 0.621875 | 0.875 | combiAbMir |
| hsa-miR-574-5p | | | | | |
| STUB1, hsa-miR-122, hsa-miR-3648, hsa- | 0.841406 | 1.49875 | 0.74875 | 0.75 | combiAbMir |
| miR-574-5p | | | | | |
| HSPD1, RAN, hsa-miR-122, hsa-miR-574- | 0.839531 | 1.470625 | 0.720625 | 0.75 | combiAbMir |
| 5p | | | | | |
| RAN, hsa-miR-122, hsa-miR-1224-5p, | 0.837069 | 1.50375 | 0.75375 | 0.75 | combiAbMir |
| hsa-miR-574-5p | | | | | |
| HSPD1, hsa-miR-122, hsa-miR-1224-5p, | 0.837188 | 1.4925 | 0.6175 | 0.875 | combiAbMir |
| hsa-miR-574-5p | | | | | |
| hsa-miR-122, hsa-miR-1224-5p, hsa-miR- | 0.83 | 1.50 | 0.73 | 0.76 | microarray |
| 3648, hsa-miR-574-5p | | | | | |
| TPM1, HSPD1, RAN, hsa-miR-342-3p | 0.832656 | 1.505 | 0.755 | 0.75 | combiAbMir |
| HSPD1, RAN, hsa-miR-3648, hsa-miR- | 0.832656 | 1.440625 | 0.690625 | 0.75 | combiAbMir |
| 574-5p | | | | | |
| TPM1, RAN, hsa-miR-150, hsa-miR-342- | 0.832188 | 1.486875 | 0.736875 | 0.75 | combiAbMir |
| 3p | | | | | |
| RAN, hsa-miR-1224-5p, hsa-miR-3648, | 0.830625 | 1.468125 | 0.718125 | 0.75 | combiAbMir |
| hsa-miR-574-5p | 0.828125 | 1.485 | 0.735 | 0 75 | combiAbMir |
| TPM1, RAN, hsa-miR-342-3p, hsa-miR- | | | | | |
| 3648 | | | | | |
| TPM1, STUB1, hsa-miR-3648, hsa-miR- | 0.827969 | 1.476875 | 0.726875 | 0.75 | combiAbMir |
| 574-5p | | | | | |
| RAN, STUB1, hsa-miR-122, hsa-miR-574- | 0.8275 | 1.470625 | 0.720625 | 0.75 | combiAbMir |
| 5p | | | | | |
| HSPD1, STUB1, hsa-miR-122, hsa-miR- | 0.826563 | 1.470625 | 0.720625 | 0.75 | combiAbMir |
| 574-5p | | | | | |
| RAN, STUB1, hsa-miR-3648, hsa-miR- | 0.823125 | 1.428125 | 0.678125 | 0.75 | combiAbMir |
| 574-5p | | | | | |
| TPMY, HSPD1, STUB1, hsa-miR-342-3p | 0.822656 | 1.483125 | 0.733125 | 0.75 | combiAbMir |
| TPM1, hsa-miR-1224-5p, hsa-miR-3648, | 0.822031 | 1.4675 | 0.7175 | 0.75 | combiAbMir |
| hsa-miR-574-5p | | | | | |
| TPM1, HSPD1, hsa-miR-122, hsa-miR- | 0.821563 | 1.485625 | 0.735625 | 0.75 | combiAbMir |
| 342-3p | | | | | |
| TPM1, HSPD1, RAN, hsa-miR-150 | 0.818906 | 1.47375 | 0.84875 | 0.625 | combiAbMir |
| TPM1, HSPD1, hsa-miR-342-3p, hsa-miR- | 0.817813 | 1.45375 | 0.70375 | 0.75 | combiAbMir |
| 3648 | | | | | |
| TPM1, HSPD1, hsa-miR-3648, hsa-miR- | 0.817031 | 1.44375 | 0.81875 | 0.625 | combiAbMir |
| 574-5p | | | | | |
| TPM1, HSPD1, hsa-miR-150, hsa-miR- | 0.815938 | 1.4775 | 0.7275 | 0.75 | combiAbMir |
| 342-3p | | | | | |
| TPM1, HSPD1, RAN, hsa-miR-574-5p | 0.815 | 1.430625 | 0.805625 | 0.625 | combiAbMir |
| TPM1, HSPD1, STUB1, hsa-miR-574-5p | 0.812656 | 1.433125 | 0.808125 | 0.625 | combiAbMir |
| HSPD1, RAN, hsa-miR-342-3p, hsa-miR- | 0.810781 | 1.43875 | 0.68875 | 0.75 | combiAbMir |
| 3648 | | | | | |
| TPM1, HSPD1, hsa-miR-150, hsa-miR- | 0.810625 | 1.455625 | 0.830625 | 0.625 | combiAbMir |
| 122 | | | | | |
| TPM1, RAN, STUB1, hsa-miR-150 | 0.810156 | 1.470625 | 0.845625 | 0.625 | combiAbMir |
| TPM1, RAN, hsa-miR-1224-5p, hsa-miR- | 0.806094 | 1.44875 | 0.69875 | 0.75 | combiAbMir |
| 342-3p | | | | | |
| TPM1, RAN, hsa-miR-150, hsa-miR-1224- | 0.805469 | 1.469375 | 0.844375 | 0.625 | combiAbMir |
| 5p | | | | | |
| RAN, hsa-miR-150, hsa-miR-342-3p, hsa- | 0.805 | 1.424375 | 0.674375 | 0.75 | combiAbMir |
| miR-3648 | | | | | |
| TPM1, STUB1, hsa-miR-122, hsa-miR- | 0.803438 | 1.449375 | 0.699375 | 0.75 | combiAbMir |
| 342-3p | | | | | |
| RAN, hsa-miR-150, hsa-miR-122, hsa- | 0.802188 | 1.434375 | 0.559375 | 0.875 | combiAbMir |
| miR-342-3p | | | | | |
| STUB1, hsa-miR-122, hsa-miR-1224-5p, | 0.801875 | 1.463125 | 0.713125 | 0.75 | combiAbMir |
| hsa-miR-574-5p | | | | | |
| HSPD1, RAN, hsa-miR-150, hsa-miR- | 0.800781 | 1.4575 | 0.7075 | 0.75 | combiAbMir |
| 1224-5p | | | | | |
| TPM1, hsa-miR-122, hsa-miR-342-3p, | 0.800781 | 1.434375 | 0.684375 | 0.75 | combiAbMir |
| hsa-miR-3648 | | | | | |
| TPM1, RAN, hsa-miR-1224-5p, hsa-miR- | 0.8 | 1.42625 | 0.80125 | 0.625 | combiAbMir |
| 574-5p | | | | | |
| TPM1, HSPD1, hsa-mR-1224-5p, hsa- | 0.799375 | 1.44375 | 0.69375 | 0.75 | combiAbMir |
| miR-342-3p | | | | | |
| TPM1, RAN, STUB1, hsa-miR-574-5p | 0.798281 | 1.415 | 0.79 | 0.625 | combiAbMir |
| TPM1, HSPD1, hsa-miR-150, hsa-miR- | 0.796875 | 1.45125 | 0.82625 | 0.625 | combiAbMir |
| 3648 | | | | | |
| HSPD1, RAN, hsa-miR-1224-5p, hsa-miR- | 0.795938 | 1.43125 | 0.80625 | 0.625 | combiAbMir |
| 574-5p | | | | | |
| HSPD1, RAN, hsa-miR-150, hsa-miR-342- | 0.795625 | 1.449375 | 0.574375 | 0.875 | combiAbMir |
| 3p | | | | | |
| TPM1, HSPD1, STUB1, hsa-miR-150 | 0.794844 | 1.44125 | 0.31625 | 0.625 | combiAbMir |
| HSPD1, hsa-miR-150, hsa-miR-342-3p, | 0.79375 | 1.419375 | 0.669375 | 0.75 | combiAbMir |
| hsa-miR-3848 | | | | | |
| HSPD1, STUB1, hsa-miR-3648, hsa-miR- | 0.792813 | 1.4025 | 0.7775 | 0.625 | combiAbMir |
| 574-5p | | | | | |
| HSPD1, hsa-miR-150, hsa-miR-122, hsa- | 0.792656 | 1.426875 | 0.551875 | 0.875 | combiAbMir |
| miR-342-3p | | | | | |
| HSPD1, hsa-miR-1224-5p, hsa-miR-3648, | 0.7925 | 1.416875 | 0.791875 | 0.625 | combiAbMir |
| hsa-miR-574-5p | | | | | |
| RAN, STUB1, hsa-miR-342-3p, hsa-miR- | 0.7925 | 1.41125 | 0.66125 | 0.75 | combiAbMir |
| 3648 | | | | | |
| TPM1, RAN, hsa-miR-150, hsa-miR-3648 | 0.792344 | 1.4325 | 0.8075 | 0.825 | combiAbMir |
| HSPD1, RAN, STUB1, hsa-miR-574-5p | 0.790938 | 1.400625 | 0.775625 | 0.625 | combiAbMir |
| TPM1, hsa-miR-150, hsa-miR-342-3p, | 0.790469 | 1.405625 | 0.780625 | 0.625 | combiAbMir |
| hsa-miR-3848 | | | | | |
| TPM1, HSPD1, hsa-miR-150, hsa-miR- | 0.790313 | 1.421875 | 0.796875 | 0.625 | combiAbMir |
| 1224-5p | | | | | |
| TPM1, HSPD1, hsa-miR-1224-5p, hsa- | 0.790313 | 1.415625 | 0.790625 | 0.625 | combiAbMir |
| miR-574-5p | | | | | |
| HSPD1, STUB1, hsa-miR-150, hsa-miR- | 0.790156 | 1.43125 | 0.68125 | 0.75 | combiAbMir |
| 342-3p | | | | | |
| HSPD1, RAN, STUB1, hsa-miR-342-3p | 0.788438 | 1.424375 | 0.549375 | 0.875 | combiAbMir |
| TPM1, STUB1, hsa-miR-150, hsa-miR- | 0.788281 | 1.41375 | 0.66375 | 0.75 | combiAbMir |
| 342-3p | | | | | |
| TPM1, hsa-miR-150, hsa-miR-1224-5p, | 0.7875 | 1.406875 | 0.781875 | 0.625 | combiAbMir |
| hsa-miR-342-3p | | | | | |
| HSPD1, STUB1, hsa-miR-160.hsa-miR- | 0.787031 | 1.435625 | 0.685625 | 0.75 | combiAbMir |
| 122 | | | | | |
| HSPD1, STUB1, hsa-miR-342-3p, hsa- | 0.785938 | 1.414375 | 0.664375 | 0.75 | combiAbMir |
| miR-3648 | | | | | |
| RAN, hsa-miR-150, hsa-miR-1224-5p, | 0.785313 | 1.42 | 0.545 | 0.875 | combiAbMir |
| hsa-miR-342-3p | | | | | |
| TPM1, STUB1, hsa-miR-342-3p, hsa-miR- | 0.785313 | 1.40375 | 0.65375 | 0.75 | combiAbMir |
| 3648 | | | | | |
| HSPD1, hsa-miR-1224-5p, hsa-miR-342- | 0.785156 | 1.410625 | 0.660625 | 0.75 | combiAbMir |
| 3p hsa-miR-3648 | | | | | |
| hsa-miR-150, hsa-miR-122, hsa-miR-342- | 0.78 | 1.39 | 0.75 | 0.65 | microarray |
| 3p, hsa-miR-3648 | | | | | |
| TPM1, STUB1, hsa-miR-1224-5p, hsa- | 0.783506 | 1.424375 | 0.674375 | 0.75 | combiAbMir |
| miR-342-3p | | | | | |
| STUB1, hsa-miR-1224-5p hsa-miR-3648, | 0.78375 | 1.404375 | 0.654375 | 0.75 | combiAbMir |
| hsa-miR-574-5p | | | | | |
| HSPD1, hsa-miR-150, hsa-miR-122, hsa- | 0.783281 | 1.419375 | 0.689375 | 0.75 | combiAbMir |
| miR-1224-5p | | | | | |
| hsa-miR-150, hsa-miR-1224-5p, hsa-miR- | 0.78 | 1.40 | 0.75 | 0.65 | microarray |
| 342-3p, hsa-miR-3648 | | | | | |
| TPM1, STUB1, hsa-miR-1224-5p, hsa- | 0.732188 | 1.43125 | 0.68125 | 0.75 | combiAbMir |
| miR-574-5p | | | | | |
| HSPD1, RAN, hsa-miR-1224-5p, hsa-miR- | 0.702188 | 1.4275 | 0.5525 | 0.875 | combiAbMir |
| 342-3p | | | | | |
| TPM1, hsa-miR-150, hsa-miR-122, hsa- | 0.782188 | 1.4075 | 0.6575 | 0.75 | combiAbMir |
| miR-342-3p | | | | | |
| HSPD1, RAN, hsa-miR-122, hsa-miR-342- | 0.782188 | 1.405625 | 0.530625 | 0.875 | combiAbMir |
| 3p | | | | | |
| TPM1, RAN, hsa-miR-122, hsa-miR-342- | 0.781875 | 1.418125 | 0.543125 | 0.875 | combiAbMir |
| 3p | | | | | |
| RAN, STUB1, hsa-miR-1224-5p, hsa-miR- | 0.781719 | 1.413125 | 0.663125 | 0.75 | combiAbMir |
| 574-5p | | | | | |
| HSPD1, RAN, hsa-miR-150, hsa-miR- | 0.781719 | 1.396875 | 0.771875 | 0.625 | combiAbMir |
| 3648 | | | | | |
| TPM1, hsa-miR-1224-5p, hsa-miR-342- | 0.780938 | 1.39625 | 0.77125 | 0.625 | combiAbMir |
| 3p, hsa-miR-3648 | | | | | |
| RAN, STUB1, hsa-miR-1224-5p, hsa-miR- | 0.780469 | 1.396875 | 0.648875 | 0.75 | combiAbMir |
| 342-3p | | | | | |
| TPM1, RAN, STUB1, hsa-miR-342-3p | 0.780313 | 1.408125 | 0.658125 | 0.75 | combiAbMir |
| RAN, hsa-miR-122, hsa-miR-342-3p, hsa- | 0.779063 | 1.37625 | 0.75125 | 0.625 | combiAbMir |
| miR-3648 | 0.778125 | 1.40125 | 0.65125 | 0.75 | combiAbMir |
| HSPD1, STUBI, hsa-miR-150 hsa-miR- | | | | | |
| 1224-5p | | | | | |
| TPM1, hsa-miR-122, hsa-miR-1224-5p, | 0.777969 | 1.409375 | 0.650375 | 0.75 | combiAbMir |
| hsa-miR-342-3p | | | | | |
| HSPD1, RAN, hsa-miR-150, hsa-miR-122 | 0.777188 | 1.395 | 0.77 | 0.625 | combiAbMir |
| HSPD1, hsa-miR-150, hsa-miR-1224-5p, | 0.778875 | 1.395625 | 0.645825 | 0.75 | combiAbMir |
| hsa-miR-342-3p | | | | | |
| TPM1, hsa-miR-150, hsa-miR-1224-5p, | 0.773281 | 1.415625 | 0.790625 | 0.625 | combiAbMir |
| hsa-miR-3648 | | | | | |
| RAN, hsa-miR-122, hsa-miR-1224-5p, | 0.772344 | 1.40625 | 0.53125 | 0.875 | combiAbMir |
| hsa-miR-342-3p | | | | | |
| HSPD1, RAN, STUB1, hsa-miR-150 | 0.772344 | 1.384375 | 0.634375 | 0.75 | combiAbMir |
| HSPD1, STUB1, hsa-miR-122, hsa-miR- | 0.772188 | 1.381875 | 0.506875 | 0.875 | combiAbMir |
| 342-3p | | | | | |
| TPM1, STUB1, hsa-miR-150, hsa-miR- | 0.770469 | 1.426875 | 0.801875 | 0.625 | combiAbMir |
| 122 | | | | | |
| HSPD1, hsa-miR-122, hsa-miR-342-3p, | 0.770156 | 1.370625 | 0.620625 | 0.75 | combiAbMir |
| hsa-miR-3848 | | | | | |
| RAN, hsa-miR-1224-5p, hsa-miR-342-3p, | 0.769688 | 1.38125 | 0.75625 | 0.625 | combiAbMir |
| hsa-miR-3648 | | | | | |
| RAN, hsa-mR-150, hsa-miR-1224-5p, | 0.769063 | 1.37375 | 0.74875 | 0.625 | combiAbMir |
| hsa-miR-3648 | | | | | |
| hsa-miR-150, hsa-miR-122, hsa-miR- | 0.77 | 1.38 | 0.62 | 0.76 | microarray |
| 1224-5p, hsa-miR-342-3p | | | | | |
| TPM1, hsa-miR-150, hsa-miR-122, hsa- | 0.76875 | 1.40625 | 0.78125 | 0.625 | combiAbMir |
| miR-1224-5p | | | | | |
| HSPD1, STUB1, hsa-miR-1224-5p, hsa- | 0.768125 | 1.398125 | 0 523125 | 0.875 | combiAbMir |
| miR-342-3p | | | | | |
| RAN, STUB1, hsa-miR-122, hsa-miR-342- | 0.767969 | 1.37875 | 0.62875 | 0.75 | combiAbMir |
| 3p | | | | | |
| HSPD1, hsa-miR-150, hsa-miR-122, hsa- | 0.767344 | 1.37625 | 0.75125 | 0.625 | combiAbMir |
| miR-3648 | | | | | |
| HSPD1, STUB1, hsa-miR-1224-5p, hsa- | 0.766094 | 1.378125 | 0.628125 | 0.75 | combiAbMir |
| miR-574-5p | | | | | |
| TPM1, RAN, hsa-miR-150, hsa-miR-122 | 0.765938 | 1.405 | 0.78 | 0.625 | combiAbMir |
| TPM1, STUB1, hsa-miR-150, hsa-miR- | 0.764219 | 1.415 | 0.79 | 0.625 | combiAbMir |
| 1224-5p | | | | | |
| HSPD1, hsa-miR-122, hsa-miR-1224-5p, | 0.763594 | 1.37375 | 0.49875 | 0.875 | combiAbMir |
| hsa-miR-342-3p | | | | | |
| RAN, STUB1, hsa-miR-150, hsa-miR- | 0.763281 | 1.385 | 0.76 | 0.625 | combiAbMir |
| 1224-5p | | | | | |
| HSPD1, hsa-miR-150, hsa-miR-1224-5p, | 0.758438 | 1.37 | 0.745 | 0.625 | combiAbMir |
| hsa-miR-3648 | | | | | |
| RAN, STUB1, hsa-miR-150, hsa-miR-342- | 0.757813 | 1.376875 | 0.501875 | 0.875 | combiAbMir |
| 3p | | | | | |
| STUB1, hsa-miR-122, hsa-miR-1224-5p, | 0.757344 | 1.351875 | 0.601875 | 0.75 | combiAbMir |
| hsa-miR-342-3p | | | | | |
| RAN, STUB1, hsa-miR-150, hsa-miR- | 0.755781 | 1.373125 | 0.748125 | 0.625 | combiAbMir |
| 3648 | | | | | |
| hsa-miR-122, hsa-miR-1224-5p, hsa-miR- | 0.76 | 1.35 | 0.66 | 0.71 | microarray |
| 342-3p, hsa-miR-3848 | | | | | |
| TPM1, STUB1, hsa-mR-150, hsa-miR- | 0.755469 | 1.3925 | 0.7675 | 0.625 | combiAbMir |
| 3648 | | | | | |
| RAN, hsa-miR-150, hsa-miR-122, hsa- | 0.753594 | 1.35125 | 0.72625 | 0.625 | combiAbMir |
| miR-1224-5 | | | | | |
| TPM1, hsa-miR-150, hsa-miR-122, hsa- | 0.753438 | 1.37625 | 0.75125 | 0.625 | combiAbMir |
| miR-3648 | | | | | |
| RAN, STUB1, hsa-miR-150, hsa-miR-122 | 0.7525 | 1.355 | 0.73 | 0.625 | combiAbMir |
| STUB1, hsa-miR-122, hsa-miR-342-3p, | 0.7525 | 1.34375 | 0.59375 | 0.75 | combiAbMir |
| hsa-miR-3648 | | | | | |
| STUB1, hsa-miR-150, hsa-miR-122, hsa- | 0.75125 | 1.35375 | 0.60375 | 0.75 | combiAbMir |
| miR-342-3p | | | | | |
| STUB1, hsa-miR-150, hsa-miR-1224-5p, | 0.749688 | 1.34625 | 0.47125 | 0.875 | combiAbMir |
| hsa-miR-342-3p | | | | | |
| HSPD1, STUB1, hsa-miR-150, hsa-miR- | 0.74875 | 1.3525 | 0.7275 | 0.625 | combiAbMir |
| 3648 | | | | | |
| STUB1, hsa-miR-150, hsa-miR-122, hsa- | 0.74875 | 1.34375 | 0.71875 | 0.625 | combiAbMir |
| miR-3648 | | | | | |
| STUB1, hsa-miR-1224-5p, hsa-miR-342- | 0.746406 | 1.344375 | 0.719375 | 0.625 | combiAbMir |
| 3p, hsa-miR-3648 | | | | | |
| STUB1, hsa-miR-150, hsa-miR-342-3p, | 0.742969 | 1.3325 | 0.5825 | 0.75 | combiAbMir |
| hsa-miR-3648 | | | | | |
| RAN, hsa-miR-150, hsa-miR-122, hsa- | 0.740938 | 1.323125 | 0.698125 | 0.625 | combiAbMir |
| miR-3648 | | | | | |
| hsa-miR-150, hsa-miR-122, hsa-miR- | 0.74 | 1.32 | 0.68 | 0.65 | microarray |
| 1224-Sp, hsa-miR-3648 | | | | | |
| STUB1, hsa-miR-150, hsa-miR-122, hsa- | 0.736719 | 1,325 | 0.575 | 0.75 | combiAbMir |
| miR-1224-5p | | | | | |
| STUB1, hsa-miR-150, hsa-miR-1224-5p, | 0.730313 | 1.31375 | 0.68875 | 0.625 | combiAbMir |
| hsa-miR-3648 | | | | | |
| hsa-miR-150, hsa-miR-122, hsa-miR- | 0.73 | 1.30 | 0.45 | 0.85 | qPCR |
| 3663-5p, hsa-miR-3194-5p | | | | | |
| hsa-miR-150, hsa-miR-122, hsa-miR-342- | 0.72 | 1.31' | 0.47 | 0 85 | qPCR |
| 3p, hsa-miR-3663-5p | | | | | |
| hsa-miR-150, hsa-miR-342-3p, hsa-miR- | 0.72 | 1.31 | 0.61 | 0.69 | QPCR |
| 3663-5p, hsa-miR-3194-5p | | | | | |
| TPM1, RAN, hsa-miR-122, hsa-miR-3648 | 0.720156 | 1.309375 | 0.809375 | 0.5 | combiAbMir |
| TPM1, HSPD1, RAN, hsa-miR-122 | 0.718438 | 1.318125 | 0.693125 | 0.625 | combiAbMir |
| TPM1, HSPD1, RAN, hsa-miR-3648 | 0.715469 | 1.28125 | 0.78125 | 0.5 | combiAbMir |
| TPM1, RAN, hsa-miR-1224-5p, hsa-miR- | 0.710156 | 1.280625 | 0.780625 | 0.5 | combiAbMir |
| 3648 | | | | | |
| TPM1, hsa-miR-122, hsa-miR-1224-5p, | 0.709063 | 1.27875 | 0.77875 | 0.5 | combiAbMir |
| hsa-miR-3648 | | | | | |
| hsa-miR-122, hsa-miR-342-3p, hsa-miR- | 0.71 | 1.29 | 0.60 | 0.69 | qPCR |
| 3663-5p, hsa-miR-3194-5p | | | | | |
| TPM1, HSPD1, STUB1, hsa-miR-3648 | 0.704531 | 1.27625 | 0.77625 | 0.5 | combiAbMir |
| TPM1, HSPD1, hsa-miR-122, hsa-miR- | 0.6875 | 1.283125 | 0.658125 | 0.625 | combiAbMir |
| 3648 | | | | | |
| TPM1, HSPD1, STUB1, hsa-miR-122 | 0.896719 | 1.298125 | 0.673125 | 0.825 | combiAbMir |
| HSPD1, RAN, hsa-miR-122, hsa-miR- | 0.692656 | 1 270625 | 0.770625 | 0.5 | combiAbMir |
| 3648 | | | | | |
| TPM1, HSPD1, hsa-miR-1224-5p, hsa- | 0.688281 | 1.25875 | 0.75875 | 0.5 | combiAbMir |
| miR-3648 | | | | | |
| TPM1, STUB1, hsa-miR-122, hsa-miR- | 0.887656 | 1.269375 | 0.769375 | 0.5 | combiAbMir |
| 3648 | | | | | |
| TPM1, HSPD1, RAN, STUB1 | 0.69 | 1.28 | 0.83 | 0.44 | autoAb |
| TPM1, HSPD1, hsa-miR-122, hsa-miR- | 0.684063 | 1.28625 | 0.66125 | 0.625 | combiAbMir |
| 1224-5p | | | | | |
| HSPD1, RAN, hsa-miR-1224-5p, hsa-miR- | 0.682656 | 1.234375 | 0.734375 | 0.5 | combiAbMir |
| 3648 | | | | | |
| TPM1, HSPD1, STUB1, hsa-miR-1224-5p | 0.682344 | 1.28625 | 0.78625 | 0.5 | combiAbMir |
| TPM1, RAN, STUB1, hsa-miR-3648 | 0.68 | 1.25 | 0.75 | 0.5 | combiAbMir |
| HSPD1, STUB1, hsa-miR-122, hsa-miR- | 0.678125 | 1.249375 | 0.749375 | 0.5 | combiAbMir |
| 3648 | | | | | |
| RAN, STUB1, hsa-miR-122, hsa-miR- | 0.669531 | 1.255 | 0.755 | 0.5 | combiAbMir |
| 3648 | | | | | |
| TPM1, RAN, STUB1, hsa-miR-122 | 0.689219 | 1.273125 | 0.773125 | 0.5 | combiAbMir |
| HSPD1, RAN, STUB1, hsa-miR-3848 | 0.869063 | 1.235 | 0.86 | 0.375 | combiAbMir |
| STUB1, hsa-miR-122, hsa-miR-1224-5p, | 0.66875 | 1.234375 | 0.734375 | 0.5 | combiAbMir |
| hsa-miR-3648 | | | | | |
| TPM1, HSPD1, RAN, hsa-miR-1224-5p | 0.666094 | 1.2425 | 0.7425 | 0.5 | combiAbMir |
| HSPD1, RAN, STUB1, hsa-miR-122 | 0.663281 | 1.2375 | 0.7375 | 0.5 | combiAbMir |
| TPM1, STUB1, hsa-miR-1224-5p, hsa- | 0.66125 | 1.2 | 0.7 | 0.5 | combiAbMir |
| miR-3648 | | | | | |
| TPM1, STUB1, hsa-miR-122, hsa-miR- | 0.8575 | 1.270625 | 0.770625 | 0.5 | combiAbMir |
| 1224-5p | | | | | |
| TPM1, RAN, hsa-miR-122, hsa-miR-1224- | 0.651406 | 1.221875 | 0.596875 | 0.625 | combiAbMir |
| 5p | | | | | |
| HSPD1, hsa-miR-122, hsa-miR-1224-5p | 0.849219 | 1.1925 | 0.8175 | 0.375 | combiAbMir |
| hsa-miR-3648 | | | | | |
| hsa-miR-150, hsa-miR-122, hsa-miR-342- | 0.65 | 1.25 | 0.41 | 0.85 | QPCR |
| 3p, hsa-miR-3194-5p | | | | | |
| HSPD1, STUB1, hsa-miR-122, hsa-miR- | 0.642031 | 1.19375 | 0.56875 | 0.625 | combiAbMir |
| 1224-5p | | | | | |
| HSPD1, STUB1, hsa-miR-1224-5p, hsa- | 0.63125 | 1.16625 | 0.79125 | 0.375 | combiAbMir |
| miR-3648 | | | | | |
| HSPD1, RAN, hsa-miR-122, hsa-miR- | 0.628438 | 1.1475 | 0.6475 | 0.5 | combiAbMir |
| 1224-5p | | | | | |
| RAN, STUB1, hsa-mR-1224-5p, hsa-miR- | 0.622656 | 1.210625 | 0.835625 | 0.375 | combiAbMir |
| 3648 | | | | | |
| HSPD1, RAN, STUB1, hsa-miR-1224-5p | 0.618906 | 1.156875 | 0.701875 | 0.375 | combiAbMir |
| RAN, hsa-miR-122, hsa-miR-1224-5p, | 0.617656 | 1.18 | 0.805 | 0.375 | combiAbMir |
| hsa-miR-3648 | | | | | |
| TPM1, RAN, STUB1, hsa-miR-1224-5p | 0.614531 | 1.1775 | 0.8025 | 0.375 | combiAbMir |
| RAN, STUB1, hsa-miR-122, hsa-miR- | 0.603125 | 1.141875 | 0.516875 | 0.625 | combiAbMir |
| 1224-5p | | | | | |

TABLE 14
(5-mers)

| | | S + | Sensitivity | Specificity | |
|---|---|---|---|---|---|
| Panel | AUC | S (i) | (ii) | (iii) | Assay (iv) |
| hsa-miR-150, hsa-miR-122, hsa-miR-1224-5p, | 0.90 | 1.59 | 0.77 | 0.82 | microarray |
| hsa-miR-342-3p, hsa-miR-574-5p | | | | | |
| hsa-miR-150, hsa-miR-122, hsa-miR-342-3p, | 0.89 | 1.57 | 0.80 | 0.76 | microarray |
| hsa-miR-3648, hsa-miR-574-5p | | | | | |
| hsa-miR-150, hsa-miR-122, hsa-miR-1224-5p, | 0.88 | 1.57 | 0.80 | 0.76 | microarray |
| hsa-miR-3648, hsa-miR-574-5p | | | | | |
| hsa-miR-122, hsa-miR-1224-5p, hsa-miR-342- | 0.88 | 1.57 | 0.81 | 0.76 | micrarray |
| 3p, hsa-miR-3648, hsa-miR-574-5p | | | | | |
| hsa-miR-150, hsa-miR-1224-5p, hsa-miR-342- | 0.88 | 1.56 | 0.80 | 0.76 | microarray |
| 3p, hsa-miR-3648, hsa-miR-574-5p | | | | | |
| hsa-miR-150, hsa-miR-122, hsa-miR-1224-5p, | 0.77 | 1.38 | 0.73 | 0.65 | microarray |
| hsa-miR-342-3p, hsa-miR-3648 | | | | | |
| hsa-miR-150, hsa-miR-122, hsa-miR-342-3p, | 0.71 | 1.28 | 0.44 | 0.85 | qPCR |
| hsa-miR-3663-5p, hsa-miR-3194-5p | | | | | |

TABLE 15
(6-mers)

| | | S + | Sensitivity | Specificity | Assay |
|---|---|---|---|---|---|
| Panel | AUC | S (i) | (ii) | (iii) | (iv) |
| hsa-miR-150, hsa-miR-122, hsa-miR-1224- | 0.89 | 1.58 | 0.81 | 0.76 | microarray |
| 5p, hsa-miR-342-3p, hsa-miR-3648, | | | | | |
| hsa-miR-574-5 | | | | | |

TABLE 16
The levels of expression of the following 2 miRNAS are
significantly different in ES samples compared to
that in OvES, NE and NP samples in Study 2 (area A
in FIG. 9), as determined in study 2:

| | Expression in ES samples |
|---|---|
| | compared to that in OvES, NE |
| miRNA | and NP samples |
| ebv-miR-BART2-5p | Up |
| hsa-miR-564 | Down |

TABLE 17
The levels of expression of the following miRNAS are
significantly different in OvES samples compared to
that in ES, NE and NP samples in Study 2 (area F in
FIG. 9), as determined in study 2:

| | Expression in OvES samples |
|---|---|
| | compared to that in ES, NE and |
| miRNA | NP samples |
| hsa-let-7d-5p/3p | Up |
| hsa-miR-29a | Down |
| hsa-miR-142-3p | Down |
| hsa-miR-197 | Up |
| hsa-miR-215 | Down |
| hsa-miR-219-1-3p | Up |
| hsa-miR-296-3p | Up |
| hsa-miR-320c | Down |
| hsa-miR-337-5p | Down |
| hsa-miR-369-3p | Down |
| hsa-miR-450b-5p | Down |
| hsa-miR-483-5p | Up |
| hsa-miR-507 | Down |
| hsa-miR-517c/hsa-mR-519a | Down |
| hsa-miR-519d | Up |
| hsa-miR-520g | Down |
| hsa-miR-576-5p | Up |
| hsa-miR-577 | Down |
| hsa-miR-604 | Up |
| hsa-miR-610 | Down |
| hsa-miR-619 | Down |
| hsa-miR-1256 | Down |
| hsa-miR-1278 | Down |
| hsa-miR-1297 | Down |
| hcmv-miR-US33-5p | Down |
| hsv1-miR-H1-5p | Up |

TABLE 18
The level of expression of the following miRNA in OvES
and ES samples is significantly different from that
in NE and NP samples in Study 2 (area C in FIG. 9),
as determined in study 2:

| | Expression in OvES and ES |
|---|---|
| | samples compared to that in NE |
| miRNA | and NP samples |
| hsa-miR-877 | Down |

TABLE 19
The levels of expression of the following miRNAs in OvES
and NE samples are significantly different from
that in ES and NP samples in Study 2 (area E in
FIG. 9), as determined in study 2:

| | Expression in OvES and NE |
|---|---|
| | samples compared to that in ES |
| miRNA | and NP samples |
| hsa-let-7e-5p/3p | Up |
| hsa-let-7f | Up |
| hsa-let-7g | Up |
| hsa-miR-9 | Up |
| hsa-miR-30b | Up |
| hsa-miR-34a | Up |
| hsa-miR-92b | Up |
| hsa-miR-96-5p/3p | Up |
| hsa-miR-106a-5p/3p | Up |
| hsa-miR-17-5p/3p | Up |
| hsa-miR-106b-5p/3p | Up |
| hsa-miR-127-3p | Up |
| hsa-miR-128 | Up |
| hsa-miR-154 | Up |
| hsa-miR-155 | Up |
| hsa-miR-183-5p/3p | Up |
| hsa-miR-194 | Up |
| hsa-miR-196b-5p/3p | Up |
| hsa-miR-203 | Up |
| hsa-miR-204 | Up |
| hsa-miR-221-5p/3p | Up |
| hsa-miR-223 | Up |
| hsa-miR-376a | Up |
| hsa-miR-376c | Up |
| nsa-miR-377 | Up |
| hsa-miR-379 | Up |
| hsa-miR-423-3p | Up |
| hsa-miR-424-5p/3p | Up |
| hsa-miR-425-5p/3p | Up |
| hsa-miR-454-5p/3p | Up |
| hsa-miR-486-3p | Up |
| hsa-miR-509-3p | Up |
| hsa-miR-514 | Up |
| hsa-miR-542-3p | Up |
| hsa-miR-545-5p/3p | Up |
| hsa-miR-626 | Up |
| hsa-miR-630 | Up |
| hsa-miR-663 | Up |
| hsa-miR-720 | Up |
| hsa-miR-758-5p/3p | Up |
| hsa-miR-876-3p | Up |
| hsa-miR-1185 | Up |
| hsa-miR-1260 | Up |
| hsa-miR-1266-5/3 | Up |

TABLE 20
The levels of expression of the following miRNAs are
significantly different in OvES, ES and NP samples
compared to that in NE samples in Study 2 (area B in
FIG. 9), as determined in study 2:

| | Expression in OvES, ES and NP |
|---|---|
| miRNA | compared to that in NE samples |
| hsa-miR-199a-3p | Up |
| hsa-miR-199b-3p | Up |
| hsa-miR-497 | Up |
| hsa-miR-625-5p/3p | Up |

TABLE 21
The levels of expression of the following miRNAs are
significantly different in OvES, ES and NE samples
compared to that in NP samples in Study 2 (area D in
FIG. 9), as determined in study 2:

| | Expression in OVES, ES and NE |
|---|---|
| | samples compared to that in NP |
| miRNA | samples |
| hsa-miR-631 | Up |
| hsa-miR-635 | Up |

[1] Strathy et al, Fertil Steril. 1982; 38(6):067-672.
[2] Nnoaham et al, Fertil Steril. 2011; 96(2):366-373 e368.
[3] Hadfield et al, Hum Reprod. 1996; 11(4):878-880.
[4] Endometrioses Research Center. Understanding endometriosis: past present and future. The National women's health information council: 2005.
[5] Nnoaham et al, Human Reproduction. 2010; 25(suppl 1):i9-i11.
[6] Augoulea et al, Gynecol Endocrinol. 2009; 25(2):75-81.
[7] Missmer S A & Cramer D W. Obstet Gynecol Clin North Am. 2003; 30(1):1-19, vii.
[8] Moen M H & Schei B. Acts Obstet Gynecol Scand. 1997; 76(6):559-562.
[9] Risk factors for pelvic endometriosis in women with pelvic pain or infertility. Gruppo Italiano per lo Studio dell′ endometriosi. Eur J Obstet Gynecol Reprod Biol. 1999; 83(2):195-199.
[10] Missmer et al, Fertil Steril. 2004; 82(6):1501-1506.
[11] Parazzini et al, J Epidemiol Community Health. 1995; 49(1):61-64.
[12] Missmer et al, Obstet Gynecol. 2004:104(5 Pt 1)-965-974.
[13] Moen M H & Magnus P. Acta Obstet Gynecol Scand. 1993; 72(7):560-564.
[14] Moen M H. Acta Obstet Gynecol Scand. 1994; 73(1):59-62.
[15] Vessey et a, BMJ. 1993; 308(8871):182-184.
[16] Vitonis et al, Epidemiology. 2010; 21(1):16-23.
[17] Sangi-Haghpeykar H & Poindexter A N, 3rd. Obstet Gynecol. 1995; 85(6):963-992.
[18] Koninckx et al. Hum Reprod. 1994; 9(6):1001-1002.
[19] Sinaii et al, Hum Reprod. 2002; 17(10):2715-2724.
[20] Mataliotakis et al, J BUON. 2009; 14(4):699-701.
[21] Kvaskoff M et al, Int J Epidemiol. 2009; 38(4):1143-1153.
[22] Hornstein et al, Hum Reprod. 1997; 12(1):143-145.
[23] Signorile et al, J Exp Clin Cancer Res. 200928:49.
[24] Sanfillippo et al, Am J Obstet Gynecol. 1986; 154(1):39-43.
[25] Simpson et al, Obstet Gynecol Clin North Am. 2003; 30(1):21-40, vi.
[26] Cullen T S. Adenomyoma of the Uterus. W B Saunders. Philadelphia; 1908.
[27] Levander G & Normann P. Acta Obstet Gynecol Scand. 1955; 34(4):366-398.
[28] Javert C T. Cancer. 1949:2(3):399-410.
[29] Sampson J A. Am J Pathol. 1927; 3(2):93-110 143.
[30] Gargett C E. Hum Reprod Update. 2007; 13(1):87-101.
[31] Donnez J et al, Obstet Gynecol Clin North Am. 2003; 30(1):83-93, viii.
[32] Garai et al, Front Biosci. 2006; 11:595-619.
[33] Noble et al, J Clin Endocrinol Metab. 1996; 81(1):174-179.
[34] Taylor R N & Yu J, Torres P B, at al. Reprod Sci. 2009; 18(2):140-148.
[35] Oosterlynck et al, Fertil Steril. 1991; 56(1):45-51.
[36] Vinatiler et al, Eur J Obstet Gynecol Reprod Biol. 2001; 96(1):21-34.
[37] Tabibzadeh et al, Hum Reprod. 1996; 11(3):633-640.
[38] Lessey et al, J Clin Endocrinol Metab. 1994; 79(2):643-649.
[39] Sharpe-Timms et al, Fertil Steril. 1998; 69(6):1128-1134.
[40] McLaren et al, Hum Reprod. 1997; 12(6):1307-1310.
[41] Al-Jefout et al. Hum Reprod. 2009; 24(12):3019-3024.
[42] Kyama et al, Fertil Steril. 2011; 95(4):1338-1343 e1331-1333.
[43] Kyama et al, Fertil Steril. 2006; 86(1):203-209.
[44] Fassbender et al, Reprod Biol Endocrinol. 2010; 8:123.
[45] Fassbender et al, Obstet Gynecol. 2012; 119(2 Pt 1):276-285.
[46] Jing J. et al, Fertil Steril. 2009; 92(4):1221-1227.
[47] Liu et al, Fertil Steril. 2007; 87(4):988-990.
[48] Seeber et al, Fertil Steril. 2010; 93(7):2137-2144.
[49] Wolfier et al, Fertil Steril. 2009; 91(6):2331-2337.
[50] Zhang et al, Chin Med J (Engl). 2009; 122(4):373-376.
[51] Pappworth et al. (2009) Mol Immunol 46:1042-9.
[52] Guerra et al (2012) Arthritis Res Ther. 29:14(3):21
[53] Vanderlugt & Miller (1996) CurrOpinImmunol. 8:831-6.
[54] Cheung et al (2000) Nucleic Acids Res. 28(1):361-3, http://alfred.med.yale.edu/alfred/
[55] McKusick (1998) Mendelian Inheritance in Man. A Catalog of Human Genes and Genetic Disorders. Baltimore: Johns Hopkins University Press. 1998 (12th edition). See also http://wwvw.ncbi.nlm.nih.gov/omim/.
[56] Stenson et al. (2009) Genome Med 1:13.
[57] Stamm et al. (2006) Nucleic Acids Res 34: D46-D55.
[58] Griffiths-Jones (2004) Nucleic Acid Res 32 (Database Issue):D109-11.
[59] Griffiths-Jones et al. (2006) Nucleic Acid Res 34 (Database Issue):D140-4.
[60] Griffiths-Jones et al. (2008) Nucleic Acid Res 36 (Database Issue):D154-8.
[61] Kozomara & Griffiths-Jones (2011) Nucleic Acid Res 39 (Database Issue):D152-7. http://www.mirbase.org/
[62] Maragkakis et al. (2009) Nucleic Acid Res 37 (Web Server Issue):W273-6.
[63] Maragkakis of al. (2011) Nucleic Acid Res 39 (Web Server Issue):W145-8. http://diana.cslab.ece.ntua.gr/DianaTools/index.php?r=microtv4/index
[64] Betel et al. (2008) Nucleic Acid Res 36 (Database Issue):D149-53. http://www.microma.org/microma/home.do
[65] Wang & EI Naqa (2008) Bioinformatics 24:325-32.
[66] Wang (2008) RNA 14:1012-7. http://mirdb.org/miRDB/index.html
[67] Grimson et al. (2007) Mol Cell 27:91-105. http://www.targetscan.org/vert_50/
[68] Krek et al. (2005) Nat Genet 37:495-500. http://pictar.mdor-berlin.de/cgi-bin/PicTar_vertebrate.cgi
[69] Geysen et al. (1984) PNAS USA 81:3998-4002.
[70] Carter (1994) Methods Mol Biol 36:207-23.
[71] Jameson, B A et al. 1988, CABIOS 4(1):181-186.
[72] Maksyutov & Zagrebelnaya (1993) Comput Appl Biosci 9(3):291-7.
[73] Hopp (1993) Peptide Research 6:183-190.
[74] Welling et al. (1985) FEBS Left. 188:215-218.
[75] Bublil et at (2007) Proteins 68(1):294-304.
[76] Sun t al. (2009) Nucleic Acids Res 37:W612-6.
[77] Raddrizzani & Hammer (2000) Brief Bioinform 1(2):179-89.
[78] Chen et at (2007) Amino Acids 33(3):423-8.
[79] Reimer (2009) Methods Mol Biol 524:335-44.
[80] Boutell et at (2004) Proteomics 4:1950-8.
[81] Tassinari et alt (2008) Curr Opin Mol Thor 10:107-15.
[82] Stoevesandt et al (2009) Expert Rev Proteomics 6:145-57.
[83] Tao et at (2007) Comb Chem High Throughput Screen 10:706-18.
[84] Gnjatic et al. (2009) J Immunol Methods 341:50-8.
[85] Hartmann et al (2009) Anal Bioanal Chem 393:1407-16.
[86] Fall & Niessner (2009) Methods Mot Biol 509:107-22.
[87] WO01/57198.
[88] WO02/27327.
[89] Blackburn & Hart (2005) Methods Mol Biol 310:197-216
[90] WO03/064656.
[91] WO2004/046730.
[92] Stahl et al. (2006) Immunol Lett 102:50-9.
[93] Quintana (2008) PNAS USA 105:18889-94.
[94] Koopmann & Blackburn (2003) Rapid Commun Mass Spectrom. 17:455-62.
[95] WO01/61040.
[96] Oleinikov et al (2003) J Proteome Res. 2:313-9.
[97] Bolstad et a. (2003) Bioinformatics 19:185-93.
[98] Meyer et at (2003) Neurocomputing 55:169-86.
[99] Koza (1992), Genetic Programming: On the Programming of Computers by Means of Natural Selection, MIT Press.
[100] Wang & Japkowicz (2008) Lecture Notes in Computer Science 4994/2008, 38-47.
[101] Elkon & Casali (2008) Nat Clin Pract Rheumatol 4(9):491-8.
[102] Dudoit, 8, et al. (2002) Statistica Sinica 12, 111-139
Yang, Y H, at al. Normalization for Two-color cDNA Microarray Data. Science and Statistics: A Festschrilt for Terry Speed, Monograph Series.
[104] Yang, Y H et al. in Normalization for cDNA microarray data: a robust composite method addressing single and multiple slide systematic variation.
[105] Chada at al. (2003) CurrOpin Drug DiscovDevel. 6(2):169-73.
[106] Chène (2003) Nature Reviews Cancer 3, 102-109.
[107] Wang & EI-Deiry (2008) CurrOpinOncol. 20(1):90-4.
[108] Gleave & Monia (2005) Nat Rev Cancer 5:468-79.
[109] Current Protocols in Molecular Biology (F. M. Ausubel et al., eds., 1987) Supplement 30
[110] Smith & Waterman (1981) Adv. Appl. Math. 2: 482-489.
[111] Boutell et al. (2004) Proteomics 4:1950-8.
[112] Gnjatic at al. (2009) J Immunol Methods 341:50-8.
[113] Bolstad et al. (2003) Bioinformatics 19:185-93.
[114] Huber et al. (2002) Bioinformatics 18 suppl. 1 S96-S104.
[115] Pradervand et al. (2010) Biotechniques 48:219-22.
[116] WO93122480.
[117] WO03/020415.
[118] WO2005/037425

1. A method for analysing a subject sample, comprising a step of determining the level of x different biomarkers in the sample, wherein the levels of the biomarkers provide a diagnostic indicator of whether the subject has endometriosis; wherein x is 1 or more and wherein the x different biomarkers are selected from the group consisting of hsa-miR-150 and the other biomarkers listed in Table 1.

2. The method of claim 1, wherein the x different biomarkers are selected from the group consisting of ebv-miR-BART2-5p, hsa-let-7f, hsa-let-7g, hsa-miR-1260, hsa-miR142-3p, hsa-miR-197, hsa-miR-215, hsa-miR-223, hsa-miR-30b, hsa-miR-320c, hsa-miR34a, hsa-miR-497, hsa-miR-630, hsa-miR-663 and hsa-miR-720.

3. The method of claim 1 or claim 2, wherein x is 2 or more.

4. The method of claim 3, wherein x is 5 or more.

5. The method of claim 4, wherein x is 10 or more.

6. The method of any preceding claim, wherein the method also includes a step of determining if a sample from the subject contains autoantibodies against CA125 and/or CA19-9.

7. The method of any preceding claim, wherein the method involves comparing levels of the biomarkers in the subject sample to levels in (i) a sample from a patient with endometriosis and/or (ii) a sample from a patient without endometriosis.

8. The method of any preceding claim, wherein the method involves analysing levels of the biomarkers in the sample with a classifier algorithm which uses the measured levels of to distinguish between patients with endometriosis and patients without endometriosis.

9. The method of any preceding claim, wherein the subject is (i) pre-symptomatic for endometriosis or (ii) already displaying clinical symptoms of endometriosis.

10. The method of any preceding claim, wherein the sample is a body fluid.

11. The method of claim 10, wherein the sample is cervical discharge or peritoneal fluid.

12. The method of any preceding claim, wherein the presence of antibodies is determined using an immunoassay.

13. The method of claim 10, wherein the immunoassay utilises an antigen comprising an amino acid sequence (i) having at least 90% sequence identity to an amino acid sequence disclosed in Table 1, and/or (ii) comprising at least one epitope from an amino acid sequence disclosed in Table 1.

14. The method of claim 12 or claim 13, wherein the immunoassay utilises a fusion polypeptide with a first region and a second region, wherein the first region can react with an auto-antibody in a sample and the second region can react with a substrate to immobilise the fusion polypeptide thereon.

15. The method of any preceding claim, wherein the subject is a human.

16. The method of any one of claims 3 to 15, wherein the 2 or more different biomarkers are:
a) a panel comprising or consisting of 2 different biomarkers, namely: (i) a biomarker selected from Table 1 and (ii) a further biomarker selected from Table 2.
b) a panel comprising or consisting of 2 different biomarkers, namely: (i) a biomarker selected from Table 1 and (ii) a further biomarker selected from Table 3.
c) a panel comprising or consisting of 2 different biomarkers, namely: (i) group of 2 biomarkers from Table 1 and (ii) a further biomarker selected from Table 4.
d) a panel comprising or consisting of 3 different biomarkers, namely: (i) a group of 2 biomarkers selected from Table 11 and (ii) a further biomarker selected from Table 1.
e) a panel comprising or consisting of 3 different biomarkers, namely: (i) a group of 2 biomarkers selected from Table 11 and (ii) a further biomarker selected from Table 2.
f) a panel comprising or consisting of 3 different biomarkers, namely: (i) a group of 2 biomarkers selected from Table 11 and (ii) a further biomarker selected from Table 3.
g) a panel comprising or consisting of 3 different biomarkers, namely: (i) a group of 2 biomarkers selected from Table 11 and (ii) a further biomarker selected from Table 4.
h) a panel comprising or consisting of 4 different biomarkers, namely: (i) a group of 3 biomarkers selected from Table 12 and (ii) a further biomarker selected from Table 1.
i) a panel comprising or consisting of 4 different biomarkers, namely: (i) a group of 3 biomarkers selected from Table 12 and (ii) a further biomarker selected from Table 2.
j) a panel comprising or consisting of 4 different biomarkers, namely: (i) a group of 3 biomarkers selected from Table 12 and (ii) a further biomarker selected from Table 3.
k) a panel comprising or consisting of 4 different biomarkers, namely: (i) a group of 3 biomarkers selected from Table 12 and (II) a further biomarker selected from Table 4.
l) a panel comprising or consisting of 5 different biomarkers, namely: (i) a group of 3 biomarkers selected from Table 13 and (ii) a further biomarker selected from Table 1.
m) a panel comprising or consisting of 5 different biomarkers, namely: (i) a group of 4 biomarkers selected from Table 13 and (ii) a further biomarker selected from Table 2.
n) a panel comprising or consisting of 5 different biomarkers, namely: (i) a group of 4 biomarkers selected from Table 13 and (ii) a further biomarker selected from Table 3.
o) a panel comprising or consisting of 5 different biomarkers, namely: (i) a group of 4 biomarkers selected from Table 13 and (ii) a further biomarker selected from Table 4.
p) a panel comprising or consisting of 6 different biomarkers, namely: (i) a group of 5 biomarkers selected from Table 14 and (ii) a further biomarker selected from Table 1.
q) a panel comprising or consisting of 6 different biomarkers, namely: (i) a group of 5 biomarkers selected from Table 14 and (ii) a further biomarker selected from Table 2.
r) a panel comprising or consisting of 6 different biomarkers, namely: (i) a group of 5 biomarkers selected from Table 14 and (ii) a further biomarker selected from Table 3.
s) a panel comprising or consisting of 6 different biomarkers, namely: (i) a group of 5 biomarkers selected from Table 14 and (ii) a further biomarker selected from Table 4.
t) a panel comprising or consisting of 7 different biomarkers, namely: (i) a group of 6 biomarkers selected from Table 15 and (ii) a further biomarker selected from Table 1.
u) a panel comprising or consisting of 7 different biomarkers, namely: (i) a group of 6 biomarkers selected from Table 15 and (ii) a further biomarker selected from Table 2.
v) a panel comprising or consisting of 7 different biomarkers, namely: (i) a group of 6 biomarkers selected from Table 15 and (ii) a further biomarker selected from Table 3.
w) a panel comprising or consisting of 7 different biomarkers, namely: (i) a group of 6 biomarkers selected from Table 15 and (ii) a further biomarker selected from Table 4.
x) a panel comprising or consisting of a group of 12 different biomarkers selected from Table 10.

17. A diagnostic device for use in diagnosis of endometriosis, wherein the device permits determination of the level(s) of 1 or more Table 1 biomarkers.

18. A kit comprising reagents for measuring the levels of at least 2 different Table 1 biomarkers.

19. The use of a Table 1 biomarker as a diagnostic biomarker for endometriosis.

20. A method for raising an antibody response in a subject, comprising eliciting to the subject an immunogen which elicits antibodies which recognise an auto-antigen listed in Table 1.


SEQUENCE LISTING (348 sequences)

SEQ ID NO: 1 - DNA, 1805 bp, Homo sapiens
cgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaaaaaaaaaaaaaaa

SEQ ID NO: 2 - DNA, 3764 bp, Homo sapiens
gctgcgggccaggggacgggggcggagccggagccggagccgacgggcggtggccgcactgggaccccggaatcccgcgcgctgcccacgattcgcttctgaggaacctagaaagattgtacaatgaatggtgattctcgtgctgcggtggtgacctcaccacccccgaccacagcccctcacaaggagaggtacttcgaccgagtagatgagaacaacccagagtacttgagggagaggaacatggcaccagaccttcgccaggacttcaacatgatggagcaaaagaagagggtgtccatgattctgcaaagccctgctttctgtgaagaattggaatcaatgatacaggagcaatttaagaaggggaagaaccccacaggcctattggcattacagcagattgcagattttatgaccacgaatgtaccaaatgtctacccagcagctccgcaaggagggatggctgccttaaacatgagtcttggtatggtgactcctgtgaacgatcttagaggatctgattctattgcgtatgacaaaggagagaagttattacggtgtaaattggcagcgttttatagactagcagatctctttgggtggtctcagcttatctacaatcatatcacaaccagagtgaactccgagcaggaacacttcctcattgtcccttttgggcttctttacagtgaagtgactgcatccagtttggttaagatcaatctacaaggagatatagtagatcgtggaagcactaatctgggagtgaatcaggccggcttcaccttacactctgcaatttatgctgcacgcccggacgtgaagtgcgtcgtgcacattcacaccccagcaggggctgcggtctctgcaatgaaatgtggcctcttgccaatctccccggaggcgctttcccttggagaagtggcttatcatgactaccatggcattctggttgatgaagaggaaaaagttttgattcagaaaaatctggggcctaaaagcaaggttcttattctccggaaccatgggctcgtgtcagttggagagagcgttgaggaggccttctattacatccataaccttgtggttgcctgtgagatccaggttcgaactctggccagtgcaggaggaccagacaacttagtcctgctgaatcctgagaagtacaaagccaagtcccgttccccagggtctccggtaggggaaggcactggatcgcctcccaagtggcagattggtgagcaggaatttgaagccctcatgcggatgctcgataatctgggctacagaactagcagcagcgggagaagacaagatggctgaactctggccggggcgacgaagcttccgaggaagggcagaatggaagcagtcccaagtcgaagactaagtggactaaagaggatggacatagaacttccacctctgctgtccctaacctgtttgttccattgaacactaacccaaaagaggtccaggagatgaggaacaagatccgagagcagaatttacaggacattaagacggctggccctcagtcccaggttttgtgtggtgtagtgatggacaggagcctcgtccagggagagctggtgacggcctccaaggccatcattgaaaaggagtaccagccccacgtcattgtgagcaccacgggccccaaccccttcaccacactcacagaccgtgagctggaggagtaccgcagggaggtggagaggaagcagaagggctctgaagagaatctggacgaggctagagaacagaaagaaaagagtcctccagaccagcctgcggtcccccacccgcctcccagcactcccatcaagctggaggaagaccttgtgccggagccgactactggagatgacagtgatgctgccacctttaagccaactctccccgatctgtcccctgatgaaccttcagaagcactcggcttcccaatgttagagaaggaggaggaagcccatagacccccaagccccactgaggcccctactgaggccagccccgagccagccccagacccagccccggtggctgaagaggctgccccctcagctgtcgaggagggggccgccgcggaccctggcagcgatgggtctccaggcaagtccccgtccaaaaagaagaagaagttccgtaccccgtcctttctgaagaagagcaagaagaagagtgactcctgaaagccctgcgctaacactgtcctgtccggagcgaccctggctctgccagcgtccccggccacgtctgtgctctgtccttgtgtaatggaatgcaaaaaagccaagccctccgcctagaggtcccctcacgtgaccagccccgtgtagccccgggctgacccagtgtgtgctcagcagccccaccccaccctgccccttgtcctctcagagcctcagcttctgggggagacatgctctccccacaggggggaggcactaagtcatggtcctggctggaaggtactgaaggcttctgcagctttggctgcacgtcaccctcctgagcctcacctttcctgccgtccctcctgttgtgaaatcaccacattctgtctctgcttggcttcccctccaccctaaagtctcaggtgacggactcagactcctggcttcatgtggcattctctctgctcagtgatctcacttaaatctatatacaaagccttggtcccgtgaaaacactcgtgtgcccaccagcggccttgaagaggcaggtctgggccagatgctgggcaggaaaccccagcggcagatgggcctgtgtgcacccaacgtgatgctatgcatgtctgaccgacgatccctcgaccagaatcagattcaggagctcagtttctttttcacttgggtctctggattcctgtcatagggaaggtatatcaggaggggaagaggcctttctagaattttctttgagcaggtttacaatttagcttacatttttcgactgtgaacgtgaataggctgctttttgctttcttctttccagaccccacagtagagcacttttcacttatttgggggaggcttcaggggactgttctcaccttaactcagccagaaagatgccctagttgtgatcaaaggtaactcgaggtggagggtagccctggggcccctcgacatcaccgtcattgatggagcctgaaccgtgtgctcctcggcagatgctgttgttgttacttccctccaagaggctggaaaagggctcagagctgctgagcaggaaccggagggtgacccatttcaggaggtgccggtaccagcctgactaggtacaggcaagcttgtgtgggcccaacaggcccttggtagagctggtgccagatgtgggctcagatcctgggcatgatgggccgagccacctcggatcccactgattggccagccgagcgagaaccaggctgctgcatggcactgaccgccgcttccagcttcctctgagccgcagggcctgctacgcgggcaagcgtgctgcctctcttctgtgtcgttttgttgccaaggcagaatgaaaagtccttaaccgtggactcttcctttatcccctcctttaccccacatatgcaatgacttttaattttcacttttgtagtttaatcctttgtattacaacatgaaatatagttgcatatatggacaccgacttgggaggacaggtcctgaatgtcctttctccagtgtaacatgttttactcacaaataaaattctttcagcaagttccttgtctaaaaaaaaaaaaaaaaaaa

SEQ ID NO: 3 - DNA, 1576 bp, Homo sapiens
gtccaccctggcggggtcgcagggttgggatggcggctggaggcgatcatggttcgcccgacagctaccgctcacctcttgcctcccgctatgccagcccggagatgtgcttcgtgtttagcgacaggtataaattccggacatggcggcagctgtggctgtggctggcggaggccgagcagacattgggtttgcctatcacagatgaacaaatccaggagatgaaatcaaacctggagaacatcgacttcaagatggcagctgaggaagagaaacgtttacgacatgatgtgatggctcacgtgcacacatttggccactgctgtccaaaagctgcaggcattattcaccttggtgctacttcttgctatgttggagacaatactgacttgattattcttagaaatgcacttgacctgcttttgccaaagcttgccagagtgatctctcggcttgccgactttgctaaggaacgagccagtctacccacattaggtttcacacatttccagcctgcacagctgaccacagttgggaaacgttgctgtctttggattcaggatctttgcatggatctccagaacttgaagcgtgtccgagatgacctgcgcttccggggagtaaagggtaccactggcactcaggccagtttcctgcagctctttgagggagatgaccataaggtagagcagcttgacaagatggtgacagaaaaggcaggatttaagagagctttcatcatcacagggcagacatatacacgaaaagtggatattgaagtactgtctgtgctggctagcttgggggcatcagtgcacaagatttgcaccgacatacgcctcctggcaaacctcaaggagatggaggaaccctttgaaaaacagcagattggctcaagtgcgatgccatataagcggaatcccatgcgttcagaacgttgctgcagtcttgcccgccacctgatgacccttgtcatggacccgctacagacagcatctgtccagtggtttgaacgcacactggatgatagtgccaaccgacggatctgtttggccgaggcatttcttaccgcagatactatattgaatacgctgcagaacatttctgaaggattggtcgtgtaccccaaagtaattgaacggcgcattcggcaagagctgcctttcatggccacagagaacatcatcatggccatggtcaaagctggaggtagccgccaggattgccatgagaaaatcagagtgctttctcagcaggcagcttctgtggttaagcaggaagggggtgacaatgacctcatagagcgtatccaggttgatgcctacttcagtcccattcactcccagttggatcatttactggatccttcttctttcactggtcgtgcctcccagcaggtgcagagattcttagaagaggaggtgtatcccctgttaaaaccatatgaaagcgtgatgaaggtgaaagcagaattatgtctgtagagttggaagagaattaaacgaaaatcattgttaaaaaaaaaaaaaaaaaaaaaaaaaaaaaaaaaaaaaaaaaaaaaaaaaaaaaaaaaaaa

SEQ ID NO: 4 - DNA, 885 bp, Homo sapiens
cttcggcggacatggctcccagcgtgccagcggcagaacccgagtatcctaaaggcatccgggccgtgctgctggggcctcccggggccggtaaagggacccaggcacccagattggctgaaaacttctgtgtctgccatttagctactggggacatgctgagggccatggtggcttctggctcagagctaggaaaaaagctgaaggcaactatggatgctgggaaactggtgagtgatgaaatggtagtggagctcattgagaagaatttggagacccccttgtgcaaaaatggttttcttctggatggcttccctcggactgtgaggcaggcagaaatgctcgatgacctcatggagaagaggaaagagaagcttgattctgtgattgaattcagcatcccagactctctgctgatccgaagaatcacaggaaggctgattcaccccaagagtggccgttcctaccacgaggagttcaaccctccaaaagagcccatgaaagatgacatcaccggggaacccttgatccgtcgatcagatgataatgaaaaggccttgaaaatccgcctgcaagcctaccacactcaaaccaccccactcatagagtactacaggaaacgggggatccactccgccatcgatgcatcccagacccccgatgtcgtgttcgcaagcatcctagcagccttctccaaagccacatgtaaagacttggttatgtttatctaatgttgggtccaagaaggaatttctttccatccctgtgaggcaatgggtgggaatgataggacaggcaaagagaagcttcctcaggctagcaaaaatatcatttgatgtattgattaaaaaagcacttgcttgatgtaaaaaaaaaaaaaaaaaa

SEQ ID NO: 5 - DNA, 1541 bp, Homo sapiens
cccacgcgtccgcccacgcgtccgcccacgcgtccgcggacgcgtgggggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtgaaaaaaaaaaaaaaaaaaaaaaaaaaaaaaaaaaaaaaaaaaaaaaaaaaaaaa

SEQ ID NO: 6 - DNA, 1313 bp, Homo sapiens
gaggcggcagccacagttgattatggaagattcccacaagagtaccacgtcagagacagcacctcaacctggttcagcagttcagggagctcacatttctcatattgctcaacaggtatcatctttatcagaaagtgaggagtcccaggactcatccgacagcataggctcctcacagaaagcccacgggatcctagcacggcgcccatcttacagaaaaattttgaaagacttatcttctgaagatacacggggcagaaaaggagacggagaaaattctggagtttctgctgctgtcacttctatgtctgttccaactcccatctatcagactagcagcggacagtatattgccattgccccaaatggagccttacagttggcaagtccaggcacagatggagtacagggacttcagacattaaccatgacaaattcaggcagtactcagcaaggtacaactattcttcagtatgcacagacctctgatggacagcagatacttgtgcccagcaatcaggtggtcgtacaaactgcatcaggagatatgcaaacatatcagatccgaactacaccttcagctacttctctgccacaaactgtggtgatgacatctcctgtgactctcacctctcagacaactaagacagatgacccccaattgaaaagagaaataaggttaatgaaaaacagagaagctgctcgagaatgtcgcagaaagaagaaagaatatgtgaaatgcctggaaaaccgagttgcagtcctggaaaatcaaaataaaactctaatagaagagttaaaaactttgaaggatctttattccaataaaagtgtttgattcctaagaaagaaaatatttttgtggacatgcataaaaattaaatggatttcctagtggagttttataaattaaaaggtcaaaactgaagctttttatttaggcttttccaaatcaaggataaatatcttacgcacgatatctagtgacagaggagaaagtggaaaatgacctcaaggaagctacgggcacaactggaagccttgtagaaattaaacatattcaaggagcaagaaatgaactttcagcagtctaaattttctaaataaccaatagttgccaatctaaagtggcagagaagatgaaatttgataaactgaattttttttaaaaatccatttaccctacaggtttgcatttgtttgctgaaatttacctttttttagttatatatatgtgtgtgtgtgtgtgtaatttctgccaataaattctaaattacaaagataaaaaaaaaaaaaaaaaaaaaaaaaaaaaa

SEQ ID NO: 7 - DNA, 1619 bp, Homo sapiens
gggtcagtgtcacctccaggatacagacagccccccttcagcccagcccagccaggtctcctacaccgccaccatgccattcggtaacacccacaacaagttcaagctgaattacaagcctgaggaggagtaccccgacctcagcaaacataacaaccacatggccaaggtactgacccttgaactctacaagaagctgcgggacaaggagactccatctggcttcactgtagacgatgtcatccagacaggagtggacaacccaggtcaccccttcatcatgaccgtgggctgcgtggctggtgatgaggagtcctacgaagttttcaaggaactctttgaccccatcatctcggatcgccacgggggctacaaacccactgacaagcacaagactgacctcaaccatgaaaacctcaagggtggagacgacctggaccccaactacgtgctcagcagccgcgtccgcactggccgcagcatcaagggctacacgttgcccccacactgctcccgtggcgagcgccgggcggtggagaagctctctgtggaagctctcaacagcctgacgggcgagttcaaagggaagtactaccctctgaagagcatgacggagaaggagcagcagcagctcatcgatgaccacttcctgttcgacaagcccgtgtccccgctgctgctggcctcaggcatggcccgcgactggcccgacgcccgtggcatctggcacaatgacaacaagagcctcctggtgtgggtgaacgaggaggatcacctccgggtcatctccatggagaaggggggcaacatgaaggaggttttccgccgcttctgcgtagggctgcagaagattgaggagatctttaagaaagctggccaccccttcatgtggaaccagcacctgggctacgtgctcacctgcccatccaacctgggcactgggctgcgtggaggcgtgcatgtgaagctggcgcacctgagcaagcaccccaagttcgaggagatcctcacccgcctgcgtctgcagaagaggggtacaggtggcgtggacacagctgccgtgggctcagtatttgacgtgtccaacgctgatcggctgggctcgtccgaagtagaacaggtgcagctggtggtggatggtgtgaagctcatggtggaaatggagaagaagttggagaaaggccagtccatcgacgacatgatccccgcccagaagtaggcgcctgcccacctgccaccgactgctggaacccagccagtgggagggcctggcccaccagagtcctgctccctcactcctcgccccgccccctgtcccagagtcccacctgggggctctctccacccttctcagagttccagtttcaaccagagttccaaccaatgggctccatcctctggattctggccaatgaaatatctccctggcagggtcctcttcttttcccagagctccaccccaaccaggagctctagttaatggagagctcccagcacactcggagcttgtgctttgtctccacgcaaagcgataaataaaagcattggtggccttaaaaaaaaaaaaaaaaaaaaaaaaaaaaaaaaacaaaaaaaaaaaaaaaaaaaaaaaaaaaaaa

SEQ ID NO: 8 - DNA, 1965 bp, Homo sapiens
ccgcgggggtgccgggactccggccgcagtgtcgccgccatcacggacttcctgtgggacaagcgcacgggcctcgccgccagaacgatgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaagcagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcgcacagcagccggagagccaagagtgtagaggacgacgctgagggccacctcatctaccacgtcggggactggctacaagagcgatatgaaatcgttagcaccttaggagaggggaccttcggccgagttgtacaatgtgttgaccatcgcaggggtggggctcgagttgccctgaagatcattaagaatgtggagaagtacaaggaagcagctcgacttgagatcaacgtgctagagaaaatcaatgagaaagaccctgacaacaagaacctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtgacgatcaggccctgggcccccctgcatcttttatagcagtgggtgtccagtccaggacactggtgcttttttatacaagagaacgagccagagttcactccttcctcctggctctctatatacctgtgaatatgtgaaatagtgtaaatatgaaagaacttgtacctatcacttcaacccctgccttgtacataatactattccatccacacagtttccaccctcacctgccccctcatacggagttggatgggggccgagtgaggtaaccaggtggcatctaccccatgttttataaggaattttgtacagtctttgtgaaataaaataacgtgcttcatttgacccccaaaaaaaaaaaaaaaaaaaaaaaaaaaaaaaa

SEQ ID NO: 9 - DNA, 1695 bp, Homo sapiens
taccccttcacacggaggacgcgatggctcccaagaaacgcccagaaacccagaagacctccgagattgtattacgccccaggaacaagaggagcaggagtcccctggagctggagcccgaggccaagaagctctgtgcgaagggctccggtcctagcagaagatgtgactcagactgcctctgggtggggctggctggcccacagatcctgccaccatgccgcagcatcgtcaggaccctccaccagcataagctgggcagagcttcctggccatctgtccagcaggggctccagcagtcctttttgcacactctggattcttaccggatattacaaaaggctgccccctttgacaggagggctacatccttggcgtggcacccaactcaccccagcaccgtggctgtgggttccaaagggggagatatcatgctctggaattttggcatcaaggacaaacccaccttcatcaaagggattggagctggagggagcatcactgggctgaagtttaaccctctcaataccaaccagttttacgcctcctcaatggagggaacaactaggctgcaagactttaaaggcaacattctacgagtttttgccagctcagacaccatcaacatctggttttgtagcctggatgtgtctgctagtagccgaatggtggtcacaggagacaacgtggggaacgtgatcctgctgaacatggacggcaaagagctttggaatctcagaatgcacaaaaagaaagtgacgcatgtggccctgaacccatgctgtgattggttcctggccacagcctccgtagatcaaacagtgaaaatttgggacctgcgccaggttagagggaaagccagcttcctctactcgctgccgcacaggcatcctgtcaacgcagcttgtttcagtcccgatggagcccggctcctgaccacggaccagaagagcgagatccgagtttactctgcttcccagtgggactgccccctgggcctgatcccgcaccctcaccgtcacttccagcacctcacacctatcaaggcagcctggcatcctcgctacaacctcattgttgtgggccgatacccagatcctaatttcaaaagttgtaccccttatgaattgaggacgatcgacgtgttcgatggaaactcagggaagatgatgtgtcagctctatgacccagaatcttctggcatcagttcgcttaatgaattcaatcccatgggggacacgctggcctctgcaatgggttaccacattctcatctggagccaggaggaagccaggacacggaagtgagagacactaaagaaggtgtgggccagacaaggccttggagcccacacatgggatcaagtcctgcaagcagaggtggcgatttgttaaagggccaaaagtatccaaggttagggttggagcaggggtgctgggacctggggcactgtgggactgggacacttttatgttaatgctctggacttgcctccagagactgctccagagttggtgacacagctgtcccaagggcccctctgtatctagcctggaaccaaggttatcttggaactaaatgacttttctcctctcagtgggtggtagcagagggatcaagcagttatttgatttgtgctcacttttgatatggccaataaaaccataccgactgaaaaaaaaaaaaaaaaaaaaa

SEQ ID NO: 10 - DNA, 1064 bp, Homo sapiens
cgccagcggcgggcggtggacagtcatggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatccgcactgccacagagaagccgctgggggagctatggaagtgacccaaggctgcccactggagacgcctctccctgcagtcccccgagaggtgggagactcgcggaaggccccgtccccagcggagtccagaccccacaacttcaggagctctttcccggcagcagagatctgcaggctgcctcttctgccccggagctggggtgcactggggacccccgtggtggggaccttggcagtgtggacatgagcagagcgatggagcagtctcctgccctctcccctgtcctgatggcactctgttgtattttcttactgaagttcagtgataactctgagcagtttcattgtgatcactgtaaatggtaatcagttggaattctcctaaatgtcttccagacactagtaaaaaaagacctgaaaattaaaaaaaaaaaaaaaaaa

SEQ ID NO: 11 - DNA, 2091 bp, Homo sapiens
gctgagcgccggaggagcgtaggcagggcagcgctggcgccagcggcgacaggagccgcgcgaccggcaaaaatacacgggaggccgtcgccgaaaagagtccgcggtcctctctcgtaaacacactctcctccaccggcgcctccccctccgctctgcgcgccgcccggctgggcgcccgaggccgctccgactgctatgtgaccgcgaggctgcgggaggaaggggacagggaagaagaggctctcccgcgggagcccttgaggaccaagtttgcggccacttctgcaggcgtcccttcttagctctcgcctgcccctttctgcagcctaggcggcccaggttctcttctcttcctcgcgcgcccagccgcctcggttcccggcgaccatggtgacgatggaggagctgcgggagatggactgcagtgtgctcaaaaggctgatgaaccgggacgagaatggcggcggcgcgggcggcagcggcagccacggcaccctggggctgccgagcggcggcaagtgcctgctgctggactgcagaccgttcctggcgcacagcgcgggctacatcctaggttcggtcaacgtgcgctgtaacaccatcgtgcggcggcgggctaagggctccgtgagcctggagcagatcctgcccgccgaggaggaggtacgcgcccgcttgcgctccggcctctactcggcggtcatcgtctacgacgagcgcagcccgcgcgccgagagcctccgcgaggacagcaccgtgtcgctggtggtgcaggcgctgcgccgcaacgccgagcgcaccgacatctgcctgctcaaaggcggctatgagaggttttcctccgagtacccagaattctgttctaaaaccaaggccctggcagccatcccacccccggttccccccagcgccacagagcccttggacctgggctgcagctcctgtgggaccccactacacgaccaggggggtcctgtggagatccttcccttcctctacctcggcagtgcctaccatgctgcccggagagacatgctggacgccctgggcatcacggctctgttgaatgtctcctcggactgcccaaaccactttgaaggacactatcagtacaagtgcatcccagtggaagataaccacaaggccgacatcagctcctggttcatggaagccatagagtacatcgatgccgtgaaggactgccgtgggcgcgtgctggtgcactgccaggcgggcatctcgcggtcggccaccatctgcctggcctacctgatgatgaagaaacgggtgaggctggaggaggccttcgagttcgttaagcagcgccgcagcatcatctcgcccaacttcagcttcatggggcagctgctgcagttcgagtcccaggtgctggccacgtcctgtgctgcggaggctgctagcccctcgggacccctgcgggagcggggcaagacccccgccacccccacctcgcagttcgtcttcagctttccggtctccgtgggcgtgcactcggcccccagcagcctgccctacctgcacagccccatcaccacctctcccagctgttagagccgccctgggggccccagaaccagagctggctcccagcaagggtaggacgggccgcatgcgggcagaaagttgggactgagcagctgggagcaggcgaccgagctccttccccatcatttctccttggccaacgacgaggccagccagaatggcaataaggactccgaatacataataaaagcaaacagaacactccaacttagagcaataacggctgccgcagcagccagggaagaccttggtttggtttatgtgtcagtttcacttttccgatagaaatttcttacctcatttttttaagcagtaaggcttgaagtgatgaaacccacagatcctagcaaatgtgcccaaccagctttactaaagggggaggaagggagggcaaagggatgagaagacaagtttcccaaaagtgcctggttctgtgtacttgtccctttgttgtcgttgttgtagttaaaggaatttcattttttaaaagaaaaaaaaaaaaaaaaaaaaaaaaaaaaaaaa

SEQ ID NO: 12 - DNA, 1826 bp, Homo sapiens
gcgggccgggcgacttgagatccgcgggtctccctcctctttttccgtctgcgtcgggagctcccgggcacgtgaggccgtgccgcgtttactggcgggcgggacggcctagccgggcggcgcctcggaggaagccgcggaccccttaggtgctgggcccttggaaatcggcgcgtggggggcggtgctcgagctgagcgcgagagggcgggagagctcgtggggtgcgaggggagcaggacgcccggccgggcagcatgagtcagcagcggccggcgaggaagttacccagtctcctcctggacccgacggaggagacggttcgccgtcggtgccgagaccccatcaacgtggagggcctgctgccatcaaaaataaggattaatttagaagataatgtacaatatgtgtccatgagaaaagctctaaaagtgaagagacctcgttttgatgtatcgctggtttatttaactcgaaaatttatggatcttgtcagatctgctcccgggggtattcttgacttaaacaaggttgcaacgaaactgggagtccgaaagcggagagtgtatgacatcaccaatgtcttagatggaatcgacctcgttgaaaagaaatccaagaaccatattagatggataggatctgatcttagcaattttggagcagttccccaacaaaagaagctacaggaggaactttctgacttatcagcaatggaagatgctttggatgagttaattaaggattgtgctcagcagctgtttgagttaacagatgacaaagaaaatgaaagactagcatatgtgacctatcaagacattcatagcattcaggccttccatgaacagatcgtcattgcagttaaagctccagcagaaaccagattggatgttccagctcccagagaagactctatcacagtgcacataaggagcaccaacggacctatcgatgtctatttgtgtgaagtggagcagggtcagaccagtaacaaaaggtctgaaggtgtcgggacctcttcatctgagagcactcatccagaaggccctgaggaagaagaaaatcctcagcaaagtgaagaattgcttgaagtaagcaactgatggcatttgagaatttatgtatcactgagttttttgggaatatcttcgtggagaattacgcatcaaatttgattctcagagcaataaattatccatgaagtgctctcgttctcagtagcggcatcatggccagtagtgtctttgaggagttcaccacttagattactgagtaattgtggtttccacatttgaaaacaactccttttataattattcactgctttttgtcagtgaaatagacatcttgcctcctgaagtagcttcatcacagagtgtcatgaagacagacagtcaggctgaaatggacagttctttgtggactctacccttcccttcaaggagtatgtcatatatcacaaaagaaattgccttacactggttcatgtttgcagttactgttgtacattgcatagatgtacacacgaatttaaatgtgatgtctttgtatatatctgtataatgttgagattacttacgaaatatgtctgagtgacacttttcacccttgtacagccaaaataatgtatatatggaaagtgacagacaaattctctaatctctttggtacctataacttattagaatcctctggatgagggttagaagagactttttccaaacttctacatgtagaagtatcataaatgtgctacacatttatgtttgtggatttaattaaagtattttaatatggttttcagtgctaaaaaaaaaaaaaaaaaa

SEQ ID NO: 13 - DNA, 3069 bp, Homo sapiens
gtcagacggcccggatcccggttaccggcccctcgctcgctgctcgccagcccagactcggccctggcagtggcggctggcgattcggaccgatccgacctgggcggaggtggcccgcgccccgcggcatgagccggtgaccaagctgggggccgagcgggaggcagccgtggccgaggagtgtgaggaagaggctgtctgtgtcattatgtgtgcgtcggtcaagtataatatccggggtcctgccctcatcccaagaatgaagaccaagcaccgaatctactatatcaccctcttctccattgtcctcctgggcctcattgccactggcatgtttcagttttggccccattctatcgagtcctcaaatgactggaatgtagagaagcgcagcatccgtgatgtgccggttgttaggctgccagccgacagtcccatcccagagcggggggatctcagttgcagaatgcacacgtgttttgatgtctatcgctgtggcttcaacccaaagaacaaaatcaaggtgtatatctatgctctgaaaaagtacgtggatgactttggcgtctctgtcagcaacaccatctcccgggagtataatgaactgctcatggccatctcagacagtgactactacactgatgacatcaaccgggcctgtctgtttgttccctccatcgatgtgcttaaccagaacacactgcgcatcaaggagacagcacaagcgatggcccagctctctaggtgggatcgaggtacgaatcacctgttgttcaacatgttgcctggaggtcccccagattataacacagccctggatgtccccagagacagggccctgttggctggtggcggcttttctacgtggacttaccggcaaggctacgatgtcagcattcctgtctatagtccactgtcagctgaggtggatcttccagagaaaggaccaggtccacggcaatacttcctcctgtcatctcaggtgggtctccatcctgagtacagagaggacctagaagccctccaggtcaaacatggagagtcagtgttagtactcgataaatgcaccaacctctcagagggtgtcctttctgtccgtaagcgctgccacaagcaccaggtcttcgattacccacaggtgctacaggaggctactttctgtgtggttcttcgtggagctcggctgggccaggcagtattgagcgatgtgttacaagctggctgtgtcccggttgtcattgcagactcctatattttgcctttctctgaagttcttgactggaagagagcatctgtggttgtaccagaagaaaagatgtcagatgtgtacagtattttgcagagcatcccccaaagacagattgaagaaatgcagagacaggcccggtggttctgggaagcgtacttccagtcaattaaagccattgccctggccaccctgcagattatcaatgaccggatctatccatatgctgccatctcctatgaagaatggaatgaccctcctgctgtgaagtggggcagcgtgagcaatccactcttcctcccgctgatcccaccacagtctcaagggttcaccgccatagtcctcacctacgaccgagtagagagcctcttccgggtcatcactgaagtgtccaaggtgcccagtctatccaaactacttgtcgtctggaataatcagaataaaaaccctccagaagattctctctggcccaaaatccgggttccattaaaagttgtgaggactgctgaaaacaagttaagtaaccgtttcttcccttatgatgaaatcgagacagaagctgttctggccattgatgatgatatcattatgctgacctctgacgagctgcaatttggttatgaggtctggcgggaatttcctgaccggttggtgggttacccgggtcgtctgcatctctgggaccatgagatgaataagtggaagtatgagtctgagtggacgaatgaagtgtccatggtgctcactggggcagctttttatcacaagtattttaattacctgtatacctacaaaatgcctggggatatcaagaactgggtagatgctcatatgaactgtgaagatattgccatgaacttcctggtggccaacgtcacgggaaaagcagttatcaaggtaaccccacgaaagaaattcaagtgtcctgagtgcacagccatagatgggctttcactagaccaaacacacatggtggagaggtcagagtgcatcaacaagtttgcttcagtcttcgggaccatgcctctcaaggtggtggaacaccgagctgaccctgtcctgtacaaagatgactttcctgagaagctgaagagcttccccaacattggcagcttatgaaacgtgtcattggtggaggtctgaatgtgaggctgggacagagggagagaacaaggcctcccagcactctgatgtcagagtagtaggttaagggtggaaggttgacctacttggatcttggcatgcacccacctaacccactttctcaagaacaagaacctagaatgaatatccaagcacctcgagctatgcaacctctgttcttgtatttcttatgatctcttatgggttcttctcgaaaatgccaagtggaagactttgtggcatgctccagatttaaatccagctgaggctccctttgttttcagttccatgtaacaatctggaaggaaacttcacggacaggaagactgctggagaagagaagcgtgttagcccatttgaggtctggggaatcatgtaaagggtacccagacctcacttttagttatttacatcaatgagttctttcagggaaccaaacccagaattcggtgcaaaagccaaacatcttggtgggatttgataaatgccttgggacctggagtgctgggcttgtgcacaggaagagcaccagccgctgagtcaggatcctgtcagttccatgagctattcctctttggtttggctttttgatatgattaaagttattttttattcctttttaaaaaaaaaaaaaaaaaaaaaaaaaaaaaaaaaaaaaaaaaaaaaaaaaaaaaaaaaaa

SEQ ID NO: 14 - DNA, 3175 bp, Homo sapiens
cgctcgcggccacgccgccgtcgcgggtacattcctcgctcccggccgaggagcgctcgggctgtctgcggaccctgccgcgtgcaggggtcgcggccggctggagctgggagtgaggcggcggaggagccaggtgaggaggagccaggtgagcaggaccctgtgctgggcgcggagtcacgcaggctcgaggaaggcagttggtgggaagtccagcttgggtccctgagagctgtgagaaggagatgcggctgctgctggccctgttgggggtcctgctgagtgtgcctgggcctccagtcttgtccctggaggcctctgaggaagtggagcttgagccctgcctggctcccagcctggagcagcaagagcaggagctgacagtagcccttgggcagcctgtgcgtctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacccctcgaataggcacagttacccccagcaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgctataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgacggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgctgccagaggaggaccccacatggaccgcagcagcgcccgaggccaggtatacggacatcatcctgtacgcgtcgggctccctggccttggctgtgctcctgctgctggccgggctgtatcgagggcaggcgctccacggccggcacccccgcccgcccgccactgtgcagaagctctcccgcttccctctggcccgacagttctccctggagtcaggctcttccggcaagtcaagctcatccctggtacgaggcgtgcgtctctcctccagcggccccgccttgctcgccggcctcgtgagtctagatctacctctcgacccactatgggagttcccccgggacaggctggtgcttgggaagcccctaggcgagggctgctttggccaggtagtacgtgcagaggcctttggcatggaccctgcccggcctgaccaagccagcactgtggccgtcaagatgctcaaagacaacgcctctgacaaggacctggccgacctggtctcggagatggaggtgatgaagctgatcggccgacacaagaacatcatcaacctgcttggtgtctgcacccaggaagggcccctgtacgtgatcgtggagtgcgccgccaagggaaacctgcgggagttcctgcgggcccggcgccccccaggccccgacctcagccccgacggtcctcggagcagtgaggggccgctctccttcccagtcctggtctcctgcgcctaccaggtggcccgaggcatgcagtatctggagtcccggaagtgtatccaccgggacctggctgcccgcaatgtgctggtgactgaggacaatgtgatgaagattgctgactttgggctggcccgcggcgtccaccacattgactactataagaaaaccagcaacggccgcctgcctgtgaagtggatggcgcccgaggccttgtttgaccgggtgtacacacaccagagtgacgtgtggtcttttgggatcctgctatgggagatcttcaccctcgggggctccccgtatcctggcatcccggtggaggagctgttctcgctgctgcgggagggacatcggatggaccgacccccacactgccccccagagctgtacgggctgatgcgtgagtgctggcacgcagcgccctcccagaggcctaccttcaagcagctggtggaggcgctggacaaggtcctgctggccgtctctgaggagtacctcgacctccgcctgaccttcggaccctattccccctctggtggggacgccagcagcacctgctcctccagcgattctgtcttcagccacgaccccctgccattgggatccagctccttccccttcgggtctggggtgcagacatgagcaaggctcaaggctgtgcaggcacataggctggtggccttgggccttggggctcagccacagcctgacacagtgctcgaccttgatagcatggggcccctggcccagagttgctgtgccgtgtccaagggccgtgcccttgcccttggagctgccgtgcctgtgtcctgatggcccaaatgtcagggttctgctcggcttcttggaccttggcgcttagtccccatcccgggtttggctgagcctggctggagagctgctatgctaaacctcctgcctcccaataccagcaggaggttctgggcctctgaaccccctttccccacacctccccctgctgctgctgccccagcgtcttgacgggagcattggcccctgagcccagagaagctggaagcctgccgaaaacaggagcaaatggcgttttataaattatttttttgaaataaaaaaaaaaaaaaaaaaaaaaaaaaaaaaaaaaaaaaaaaaaaaaaaaaaaaaaaaaaaaaaaaaaaaaaaaa

SEQ ID NO: 15 - DNA, 2106 bp, Homo sapiens
caaaaaagtttcaatccagagttgggttccagctttctgtagctgtaagcattggtggccacaccacctccttacaaagcaactagaacctgcggcatacgttggagagatttttttaattttctggacatgaagtaaatttagagtgctttctaatttcaggtagaagacatgtccaccttctgattatttttggagcatgagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaaaaaaaaaaaaaaaaaaaaaaaaaaaaaaaaaaaaaaaaaaaaaaaaaaaa

SEQ ID NO: 16 - DNA, 1420 bp, Homo sapiens
gtcagcctacatttttttaagtaaggagtcctgatgttcaactattaaatctttattataggttatagactggactttaaaggcatagctgccttaaatgtgggtaatggttgataaatgtcgctaccatcagaggtcgcttcacaaggtgtctaattcagaatcgaagatctctagcggtcttctactcagtcgactgcagagacgaccgcggtaggtttttcaacccggactcaacaaagaagccctcatagcacagccttgacgtcactcaaagacaggccccaatcccgactgctagagcagaggcccgcccctcgacctccgtgagtggccccgccccgccctagggggcggggcgatgccgaactgcggcgggctggctcagtaaagcggaggcagcgggggaagatggcggcggccgttccacagcgggcgtggaccgtggagcagctgcgcagtgagcagctgcccaagaaggacattatcaagtttctgcaggaacacggttcagattcgtttcttgcagaacataaattattaggaaacattaaaaatgtggccaagacagctaacaaggaccacttggttacagcctataaccatctttttgaaactaagcgttttaagggtactgaaagtataagtaaagtgtctgagcaagtaaaaaatgtgaagcttaatgaagataaacccaaagaaaccaagtctgaagagaccctggatgagggtccaccaaaatatactaaatctgttctgaaaaagggagataaaaccaactttcccaaaaagggagatgttgttcactgctggtatacaggaacactacaagatgggactgtttttgatactaatattcaaacaagtgcaaagaagaagaaaaatgccaagcctttaagttttaaggtcggagtaggcaaagttatcagaggatgggatgaagctctcttgactatgagtaaaggagaaaaggctcgactggagattgaaccagaatgggcttacggaaagaaaggacagcctgatgccaaaattccaccaaatgcaaaactcacttttgaagtggaattagtggatattgattgaaatagcagtgcttcagctctaaggatattagcaacaatgataaaacttggccttgaagaaatttacacaactagttagaacttgttactattgtaaaggaagagtcaactggaaaattcaaggagttaataaaatttgtttacttggtcccagcttttgagagataaatcccttatgaatccctggtctaaaatactttcctacagctgtgtaaaatactggtcaaggagaactttttccttttacctcatgttgtaaacttaagtggctcaataaaaattgatccactgtcaaaaaaaaaaaaaaaaaaaaaaaaaaaaaaaaaaaaaaaaaa

SEQ ID NO: 17 - DNA, 1385 bp, Homo sapiens
cggggctgtgccggagcagctgtgcagagctgcaggcgcgcgtcatggctgctttgagacagccccaggtcgcggagctgctggccgaggcccggcgagccttccgggaggagttcggggccgagcccgagctggccgtgtcagcgccgggccgcgtcaacctcatcggggaacacacggactacaaccagggcctggtgctgcctatggctctggagctcatgacggtgctagtgggcagcccccgcaaggatgggctggtgtctctcctcaccacctctgagggtgccgatgagccccagcggctgcagtttccactgcccacagcccagcgctcgctggagcctgggactcctcggtgggccaactatgtcaagggagtgattcagtactacccagctgcccccctccctggcttcagtgcagtggtggtcagctcagtgcccctggggggtggcctgtccagctcagcatccttggaagtggccacgtacaccttcctccagcagctctgtccagactcgggcacaatagctgcccgcgcccaggtgtgtcagcaggccgagcacagcttcgcagggatgccctgtggcatcatggaccagttcatctcacttatgggacagaaaggccacgcgctgctcattgactgcaggtccttggagaccagcctggtgccactctcggaccccaagctggccgtgctcatcaccaactctaatgtccgccactccctggcctccagcgagtaccctgtgcggcggcgccaatgtgaagaagtggcccgggcgctgggcaaggaaagcctccgggaggtacaactggaagagctagaggctgccagggacctggtgagcaaagagggcttccggcgggcccggcacgtggtgggggagattcggcgcacggcccaggcagcggccgccctgagacgtggcgactacagagcctttggccgcctcatggtggagagccaccgctcactcagagacgactatgaggtgagctgcccagagctggaccagctggtggaggctgcgcttgctgtgcctggggtttatggcagccgcatgacgggcggtggcttcggtggctgcacggtgacactgctggaggcctccgctgctccccacgccatgcggcacatccaggagcactacggcgggactgccaccttctacctctctcaagcagccgatggagccaaggtgctgtgcttgtgaggcacccccaggacagcacacggtgagggtgcggggcctgcaggccagtcccacggctctgtgcccggtgccatcttccatatccgggtgctcaataaacttgtgcctccaaaaaaaaaaaaaaaaaaaaaaaaaaaaaaaaaaaaaaaaaaaaaaaaaaaa

SEQ ID NO: 18 - DNA, 1893 bp, Homo sapiens
tactggtgtcatggcagccccgaagacagcagctgtggggccgttggtgggagcggtggtccagggcaccaactccactcgctttctggttttcaattcaaaaacagcggaactacttagtcatcacaaagtggaattaacacaagagttcccaaaagaaggatgggtggaacaagaccctaaagaaattcttcagtctgtctacgagtgtatagcgagaacgtgtgagaaacttgacgaactgaatattgatatatccaacataaaagctgttggtgtcagcaatcagagggaaaccactgtaatctgggacaagttaacaggagagcctctctacaatgctgtggtgtggcttgatctaagaacccagactactgttgaggatcttagtaaaaaaattccaggaaatagtaacttcgtcaagtctaagacaggccttccactcagcacttacttcagtgcagtaaaacttcgttggatgcttgacaatgtgagaaacgtccaaaaggctgttgaagaaggtagagctctttttggtaccattgattcatggcttatctggagtttgacaggaggagttaatggaggcgtgcattgtacagatgtaacaaatgcaagtaggacaatgctttttaatatccattctttggaatgggataaagagctctgtgacttttttgaaattccaatggaccttcttccaaatgtcttcagttcttctgagatctatggcctaattaaaactggagccctggaaggtgtgccaatatctgggtgtttgggggaccaatgtgctgcattagtaggacaaatgtgcttccaggagggacaagccaaaaacacctatggaacaggttgcttcttactgtgtaatacgggtcgtaaatgtgtgttttctgaacatggccttttgaccacagtagcttacaaactaggcagagagaagccagcatattatgcactggaaggttctgttgctatagcaggtgctgttattcgttggctaagagacaatcttggaattatagagacctcaggagacattgaaagacttgctaaagaagtaggaacttcttatggctgttactttgtcccagccttttcagggttatatgcaccttattgggagcccagtgcaagagggatactctgtggcctcactcagtttaccaataaatgtcatattgcttttgctgcattagaagctgtttgtttccaaacccgagagattttggaagccatgaaccgtgactgtggaattccacttcgtcatttgcaggtagatggaggaatgaccaacaacaaagttcttatgcagctacaagcagatattcttcatattccagtaataaaaccctttatgcctgaaacaactgcactaggagctgccatggcagcaggggctgcagagggagtaagcgtttggagccttgaaccccaggctttgtcagttctcaggatggaacgatttgaaccacagatccaggccacagaaagtgaaattcgttatgccacatggaagaaagccgtaatgaagtcaatgggttgggttaccagtcagtctcctgaaggtggtgatccttctatcttctctagtctgcctttgggattttttatagtgagtagcatggtaatgctaattggagcaagatatatctcgggtgtgccataataataccagctatggagtcccaagatgtaaacattttacataatgaaagcatatagcagttctgcctcttaatataatgacactattatagactctgattttgtttataaaccacttgctacatgacccatgaaaaccctagacttgtgacctgaaataaagaaaatacgttagaagagcaaaaaaaaaaaaaaaaaaaaaaaaaaaaaaaaaaaaaaaaa

SEQ ID NO: 19 - DNA, 2116 bp, Homo sapiens
cgtctccctccctgaagacgtggtcccagccgggtgtcctgacgctcggggttcaggacaagggcacacaactggttccgttaagcccctctctcgctcagacgccatggagctggatctgtctccacctcatcttagcagctctccggaagacctttgcccagcccctgggacccctcctgggactccccggccccctgatacccctctgcctgaggaggtaaagaggtcccagcctctcctcatcccaaccaccggcaggaaacttcgagaggaggagaggcgtgccacctccctcccctctatccccaaccccttccctgagctctgcagtcctccctcacagagcccaattctcgggggcccctccagtgcaagggggctgctcccccgcgatgccagccgcccccatgtagtaaaggtgtacagtgaggatggggcctgcaggtctgtggaggtggcagcaggtgccacagctcgccacgtgtgtgaaatgctggtgcagcgagctcacgccttgagcgacgagacctgggggctggtggagtgccacccccacctagcactggagcggggtttggaggaccacgagtccgtggtggaagtgcaggctgcctggcccgtgggcggagatagccgcttcgtcttccggaaaaacttcgccaagtacgaactgttcaagagctccccacactccctgttcccagaaaaaatggtctccagctgtctcgatgcacacactggtatatcccatgaagacctcatccagaacttcctgaatgctggcagctttcctgagatccagggctttctgcagctgcggggttcaggacggaagctttggaaacgctttttctgcttcttgcgccgatctggcctctattactccaccaagggcacctctaaggatccgaggcacctgcagtacgtggcagatgtgaacgagtccaacgtgtacgtggtgacgcagggccgcaagctctacgggatgcccactgacttcggtttctgtgtcaagcccaacaagcttcgaaatggccacaaggggcttcggatcttctgcagtgaagatgagcagagccgcacctgctggctggctgccttccgcctcttcaagtacggggtgcagctgtacaagaattaccagcaggcacagtctcgccatctgcatccatcttgtttgggctccccacccttgagaagtgcctcagataataccctggtggccatggacttctctggccatgctgggcgtgtcattgagaacccccgggaggctctgagtgtggccctggaggaggcccaggcctggaggaagaagacaaaccaccgcctcagcctgcccatgccagcctccggcacgagcctcagtgcagccatccaccgcacccaactctggttccacgggcgcatttcccgtgaggagagccagcggcttattggacagcagggcttggtagacggcctgttcctggtccgggagagtcagcggaacccccagggctttgtcctctctttgtgccacctgcagaaagtgaagcattatctcatcctgccgagcgaggaggagggccgcctgtacttcagcatggatgatggccagacccgcttcactgacctgctgcagctcgtggagttccaccagctgaaccgcggcatcctgccgtgcttgctgcgccattgctgcacgcgggtggccctctgaccaggccgtggactggctcatgcctcagcccgccttcaggctgcccgccgcccctccacccatccagtggactctggggcgcggccacaggggacgggatgaggagcgggagggttccgccactccagttttctcctctgcttctttgcctccctcagatagaaaacagcccccactccagtccactcctgacccctctcctcaagggaaggccttgggtggccccctctccttctcctagctctggaggtgctgctctagggcagggaattatgggagaagtgggggcagcccaggcggtttcacgccccacactttgtacagaccgagaggccagttgatctgctctgttttatactagtgacaataaagattattttttgatacaaaaaaaaaaaaaaaaaaaaaaaa

SEQ ID NO: 20 - DNA, 1322 bp, Homo sapiens
acattcgcttcctgctttcggtgtgtctgttgtgtcttgttgcgggcaccgcagtcgccgtgaagatggcgtctaccagccgtttggatgctcttccaagagtcacatgtccaaaccatccagatgcgattttagtggaggactacagagccggtgatatgatctgtcctgaatgtggcttggttgtaggtgaccgggttattgatgtgggatctgaatggcgaactttcagcaatgacaaagcaacaaaagatccatctcgagttggagattctcagaatcctcttctgagtgatggagatttgtctaccatgattggcaagggcacaggagctgcaagttttgacgaatttggcaattctaagtaccagaatcggagaacaatgagcagttctgatcgggcaatgatgaatgcattcaaagaaatcactaccatggcagacagaatcaatctacctcgaaatatagttgatcgaacaaataatttattcaagcaagtatatgaacagaagagcctgaagggaagagctaatgatgctatagcttctgcttgtctctatattgcctgtagacaagaaggggttcctaggacatttaaagaaatatgtgccgtatcacgaatttctaagaaagaaattggtcggtgttttaaacttattttgaaagcgctagaaaccagtgtggatttgattacaactggggacttcatgtccaggttctgttccaacctttgtcttcctaaacaagtacagatggcagctacacatatagcccgtaaagctgtggaattggacttggttcctgggaggagccccatctctgtggcagcggcagctatttacatggcctcacaggcatcagctgaaaagaggacccaaaaagaaattggagatattgctggtgttgctgatgttacaatcagacagtcctatagactgatctatcctcgagccccagatctgtttcctacagacttcaaatttgacaccccagtggacaaactaccacagctataaattgaggcagctaacgtcaaattcttgaatacaaaactttgcctgttgtacatagcctatacaaaatgctgggttgagcctttcatgaggaaaaacaaaagacatggtacgcattccagggctgaatactattgcttggcattctgtatgtatatactagtgaaacatatttaatgatttaaatttcttatcaaatttcttttgtagcaatctaggaaactgtattttggaagatatttgaaattatgtaattcttgaataaaacatttttcaaaacacaaaaaaaaaaaaaaaaaaaaaaaaaaa

SEQ ID NO: 21 - DNA, 2989 bp, Homo sapiens
gaccccctagtaacagaggcggtggctactgctgcggccactgggtttcggcctcttcccagcagcggctctaagaagcgcagcggaactcgaccggatccaacccagttagttacttcctgtctagagttgtagcttccacctgcaccttctagccaccatggcaacctcatctgaagaagttttgctgattgtaaagaaagtgcgtcaaaagaagcaggatggagctctgtacctcatggcagaaagaattgcttgggcacctgaaggcaaagatagatttacaatcagccatatgtatgcagatattaaatgccagaaaattagtccagaaggaaaagctaaaattcagcttcagctggtcctacatgcaggggacacaactaacttccatttttccaatgaaagcacagcagtgaaagagcgagatgcagtaaaagaccttcttcagcagctgctgcccaaattcaagaggaaagcaaataaagaactggaagagaagaacagaatgctgcaagaagatcctgttttgtttcagctttataaagaccttgttgtgagtcaagtgatcagtgctgaggaattctgggccaatcgtttaaatgtgaatgcaacagatagttcttccacatccaatcataagcaggatgttggcatttctgctgcatttctggctgatgtccggccccaaactgatggctgtaacggtctaagatataatttaacttctgatatcattgagtccatatttaggacctatccagcagtaaaaatgaaatatgcagaaaatgttccccacaacatgacagagaaggaattctggacacgttttttccagtcccattattttcacagggatcggctgaatacagggtcaaaggatctctttgcagaatgtgccaaaatagatgaaaaaggcctaaaaacaatggtttcattaggagtgaaaaacccactactagatttaacagctttggaagataaaccattagatgagggctatggcatttcctctgtgccatctgcttccaattctaaatccataaaagagaatagtaatgctgccatcatcaagagatttaaccatcacagtgccatggtcctggcagctggactcagaaaacaagaagcacaaaatgaacaaactagtgagcccagcaacatggatggaaattccggagatgcagactgctttcagccagcagtcaaaagggcgaaattacaagagtccattgaatatgaagacttggggaaaaataattctgtaaaaacgattgcactaaacctcaagaagtcagataggtattatcatggtccaactccaatccagtcactacagtatgcaacaagtcaggacattattaattcttttcaaagtattagacaagaaatggaagcttatacacccaagttaactcaggttctctcaagtagtgctgccagtagtaccatcacagcactgtcacctggaggggcacttatgcagggaggaacacagcaagccataaaccagatggtgccaaatgatattcaatctgaattgaaacacttatatgtagctgttggagaacttctacgacatttctggtcctgctttcctgttaatacgccattcctagaagaaaaggtagtgaaaatgaaaagtaatttggaacgattccaagttacgaagctctgtccattccaagaaaagattcggagacagtatttaagcacaaatttggtaagtcacatagaagagatgctccagacagcctacaacaagctccacacatggcagtcacggcgtctgatgaagaaaacgtgaggtggccatgatgcttacaggttttgtgagattgagagaactatgacctgcagcaactctggaaacctggcctgacagacaagcagatgacctcacaggagtgataagaaacatctgctccacgccaactcccagagctgatgctattgtacttgcacattggagactgaaaggaaagaagggactaaatgctggggaggtaaattaagacagaaccaaatgagctaagttgcaaatatatatatatacacacacacacatatatgtacatgtgtatgtacatatatattttaaaagactgtttactgcagttgctcaggaactgcttttgattcacattaagctgctttcagaaattaaaaaaacactttttaaagggtgcattgataaaatctgaggttttttggttgtcgtttttttctgtgtacatttttttcctaagtttatggcacagggtagaccttaagtattcctcctccatccttcattcttcaccctccattggatcctcaagttttaatgaattccaattataccttacatcagcaagttaaaaaaagtactttaaaataaagcaaagggagactgttgctcaaccatcaggaaacagttgtcagaagacatcattggttctgtgtttcctacggaaataagaaacgataaatattgcactgaatgtttgtggtttggagtccctgaataataaagagggaatatatttgcagaaagtcgcatagggttttttaatgcagaattttgtcagaagacaatggcgctgcatgtttttctttgagtgcaaatgtacattgctaagatttttttaagatggcatgtgctttgaaaagaagatattgcatttttaagagtttaaaaatcttatgagtgagaaatattaaaaaaatcttattttcacctctttagaagaaataaaagatgtttctcctatctccttttctctagtatttgactgttactgtccttggcgaatcgataatcattgcatagtgactgaaaagcctaagtgcaaaaaaaaaaaaaaaaagatgttcttgtttctgaacttcgtgccatattttgttcctgatgggatcaacttaatgtttaagactttagatgtcttgtattaaaaattacacaaaaaaagtaaaactttttatacttaaaaaaaaaaaaaaaaaaaaa

SEQ ID NO: 22 - DNA, 1843 bp, Homo sapiens
ggtccgcgtgtggaagtctgtgaggcgcagaggtggggcaggccgtctgactagctaggcggctgggagcgttttcgtggcggggaacggaggttgaattgccctgcctgggctcatagggaaggaggatgtgaaggagcttgtgaaggcagaggaaggctggagtgcagtggcacaaccttggctcgctccagtgtctacctcccaggttcaagtgattctcctgcctcagcctcccgagtagctgggattacagattattgaataataaaatacagttttgaaaaaaatggatgaagaacctgaaagaactaagcgatgggaaggaggctatgaaagaacatgggagattcttaaagaagatgaatctggatcacttaaagctacaatagaagacattctattcaaggcaaagagaaaaagagtatttgagcaccatggacaagttcgacttggaatgatgcgccacctttatgtggtagtagatggatcaagaacaatggaagaccaagatttaaagcctaatagactgacgtgtactttaaagttgttggaatactttgtagaggaatattttgatcaaaatcctattagtcagattggaataattgtaactaagagtaaaagagctgaaaaattgactgaactttcaggaaacccaagaaaacatataacgtctttgaaggaagctgtggatatgacctgccatggagagccatctctttataattccctaagcatggctatgcagactctaaaacacatgcctggacatacaagtcgagaagtactaatcatctttagcagccttacaacttgcgatccatctaatatttatgatttaatcaagaccctaaaggcagctaaaattagagtatctgttattggattgtctgcagaagttcgcgtttgcactgtacttgctcgtgaaactggtggcacgtaccatgttattttagatgaaagccattacaaagagttgctcacacatcatcttagtcctcctcctgctagctcaagttctgaatgctcacttattcgtatgggatttcctcagcacaccattgcttctttatctgaccaggatgcaaaaccctctttcagcatggcgcatttggatggcaatactgagccagggcttacattaggaggctatttctgcccacagtgtcgggcaaagtactgtgagctacctgttgaatgtaaaatctgtggtcttactttggtgtctgctccccacttggcacggtcttaccatcatttgtttcctttggatgcttttcaagaaattcccctagaagaatataatggagaaagattttgttatggatgtcagggggaattgaaagaccaacatgtttatgtttgtgctgtgtgccaaaatgttttctgtgtggactgtgatgtttttgttcatgattctctacactgttgccctggctgtattcataagattccagctccttcaggtgtttgattccagcatgtagtatacattgtatgtgttaaaaagaaatttgcaactgtgaataaaaggacttctttagaagaagcttcatttaaaacatgaaaggataatctgacttaagaaactttttgctaagaaaaggtaatattttattaaattttaaatttgtgttgtcacagaaatacctgaaattcagtagtacttcattcaattaattttgttttctattattttgagttatactgttttcaaagtcattatgcagtatgtataaacttataagaattaaattgatgtgataattttatgtttttataattaaatatagaatctttaaaaaaaaaaaaaaaaaaaaaaaaaaaaaaaaaaaaaaaa

SEQ ID NO: 23 - DNA, 2249 bp, Homo sapiens
cgacgacctgtctcgccgagcgcacgccttgccgccgccccgcagaaatgcttcggttacccacagtctttcgccagatgagaccggtgtccagggtactggctcctcatctcactcgggcttatgccaaagatgtaaaatttggtgcagatgcccgagccttaatgcttcaaggtgtagaccttttagccgatgctgtggccgttacaatggggccaaagggaagaacagtgattattgagcagagttggggaagtcccaaagtaacaaaagatggtgtgactgttgcaaagtcaattgacttaaaagataaatacaaaaacattggagctaaacttgttcaagatgttgccaataacacaaatgaagaagctggggatggcactaccactgctactgtactggcacgctctatagccaaggaaggcttcgagaagattagcaaaggtgctaatccagtggaaatcaggagaggtgtgatgttagctgttgatgctgtaattgctgaacttaaaaagcagtctaaacctgtgaccacccctgaagaaattgcacaggttgctacgatttctgcaaacggagacaaagaaattggcaatatcatctctgatgcaatgaaaaaagttggaagaaagggtgtcatcacagtaaaggatggaaaaacactgaatgatgaattagaaattattgaaggcatgaagtttgatcgaggctatatttctccatactttattaatacatcaaaaggtcagaaatgtgaattccaggatgcctatgttctgttgagtgaaaagaaaatttctagtatccagtccattgtacctgctcttgaaattgccaatgctcaccgtaagcctttggtcataatcgctgaagatgttgatggagaagctctaagtacactcgtcttgaataggctaaaggttggtcttcaggttgtggcagtcaaggctccagggtttggtgacaatagaaagaaccagcttaaagatatggctattgctactggtggtgcagtgtttggagaagagggattgaccctgaatcttgaagacgttcagcctcatgacttaggaaaagttggagaggtcattgtgaccaaagacgatgccatgctcttaaaaggaaaaggtgacaaggctcaaattgaaaaacgtattcaagaaatcattgagcagttagatgtcacaactagtgaatatgaaaaggaaaaactgaatgaacggcttgcaaaactttcagatggagtggctgtgctgaaggttggtgggacaagtgatgttgaagtgaatgaaaagaaagacagagttacagatgcccttaatgctacaagagctgctgttgaagaaggcattgttttgggagggggttgtgccctccttcgatgcattccagccttggactcattgactccagctaatgaagatcaaaaaattggtatagaaattattaaaagaacactcaaaattccagcaatgaccattgctaagaatgcaggtgttgaaggatctttgatagttgagaaaattatgcaaagttcctcagaagttggttatgatgctatggctggagattttgtgaatatggtggaaaaaggaatcattgacccaacaaaggttgtgagaactgctttattggatgctgctggtgtggcctctctgttaactacagcagaagttgtagtcacagaaattcctaaagaagagaaggaccctggaatgggtgcaatgggtggaatgggaggtggtatgggaggtggcatgttctaactcctagactagtgctttacctttattaatgaactgtgacaggaagcccaaggcagtgttcctcaccaataacttcagagaagtcagttggagaaaatgaagaaaaaggctggctgaaaatcactataaccatcagttactggtttcagttgacaaaatatataatggtttactgctgtcattgtccatgcctacagataatttattttgtatttttgaataaaaaacatttgtacattcctgatactgggtacaagagccatgtaccagtgtactgctttcaacttaaatcactgaggcatttttactactattctgttaaaatcaggattttagtgcttgccaccaccagatgagaagttaagcagcctttctgtggagagtgagaataattgtgtacaaagtagagaagtatccaattatgtgacaacctttgtgtaataaaaatttgtttaaagttaaaaaaaaaaaaaaaaaaaa

SEQ ID NO: 24 - DNA, 1911 bp, Homo sapiens
gtgttctagaacagatcagacattttgtaatgatgcctgaaataaacactaaccacctcgacaagcaacaggttcaactcctggcagagatgtgtatccttattgatgaaaatgacaataaaattggagctgagaccaagaagaattgtcacctgaacgagaacattgagaaaggattattgcatcgagcttttagtgtcttcttattcaacaccgaaaataagcttctgctacagcaaagatcagatgctaagattacctttccaggttgttttacgaatacgtgttgtagtcatccattaagcaatccagccgagcttgaggaaagtgacgcccttggagtgaggcgagcagcacagagacggctgaaagctgagctaggaattcccttggaagaggttcctccagaagaaattaattatttaacacgaattcactacaaagctcagtctgatggtatctggggtgaacatgaaattgattacattttgttggtgaggaagaatgtaactttgaatccagatcccaatgagattaaaagctattgttatgtgtcaaaggaagaactaaaagaacttctgaaaaaagcagccagtggtgaaattaagataacgccatggtttaaaattattgcagcgacttttctctttaaatggtgggataacttaaatcatttgaatcagtttgttgaccatgagaaaatatacagaatgtgaatatgtaggtaaatgattacagaaaaatttatctgcttaacaaacttagaatgactttttccttttaaatttagttctatcattaatttatcattaaatttagttctatcatttggtactatcattaatgtattatatacactgatactttaaaacttgtgtggaaaaaactaacttataattttgtatcacacaccctggatatgtgttctgtttctaagcgacatttgtgagagattattgtaaaatgagagcgagcaaataaaacttaatttaatctttgcagatacatacttatgggaaatttgaacaaatgagtgaaactctgtgtttttagtaggctgtgataaacatttccggagcacttgcagaggacttgctatttgccaggtgctttatgtatcattaaatttttctcatagttcagaaaaatgtgcaaaggaaactattgtctcgctccttcaaaacagtcttaattaactttcatattagcagattaaactagcagagcaggttcaagggaaattaaatgatatggaccctaatttgtatcattctgagttgattgtgtggtttattcattctggaaacatgttgatacttacagtcagccactgcttttgataagtgatattgattaggttgaatcttcttgtaaatagtatttaccagttagcaaagtctgtgttttcagaattacagtgagcacagaggtgttcataaaatgggaattgagtcccactcggtaagagttgcttaaacttgacactgttgacatttgggctggataaaacccctgtggtggggtctgtgctgtgcattgcaggatggtgagcagcgtccctctcatgtgacacccacagttatgccggatgttgccagatgcccctaggggacagagtcaacccccaactgaggaccactgtcctacagagtcaggaaatattgtagggagaaaaaaataacaacaacaaaggcctgtgttaatgttaaatagatgagattatggaatgtgtatattaatgttaaaaattgtaccttgatcaatgtactttttataaacttgccatagatatctcagatttgaaacctcaagacagatttattattcttaaatgctgtatgataatgaagaaaaataaaaatttatttcttgcaaagttacaaaaaaaaaaaaaaaaaaaaaaaaaaaaaa

SEQ ID NO: 25 - DNA, 2709 bp, Homo sapiens
gcagaataggagcaagccagcactagtcagctaactaagtgactcaaccaaggccttttttccttgttatctttgcagatacttcattttcttagcgtttctggagattacaacatcctgcggttccgtttctgggaactttactgatttatctcccccctcacacaaataagcattgattcctgcatttctgaagatctcaagatctggactactgttgaaaaaatttccagtgaggctcacttatgtctgtaaagatgggaaaaaaatacaagaacattgttctactaaaaggattagaggtcatcaatgattatcattttagaatggttaagtccttactgagcaacgatttaaaacttaatttaaaaatgagagaagagtatgacaaaattcagattgctgacttgatggaagaaaagttccgaggtgatgctggtttgggcaaactaataaaaattttcgaagatataccaacgcttgaagacctggctgaaactcttaaaaaagaaaagttaaaagtaaaaggaccagccctatcaagaaagaggaagaaggaagtggatgctacttcacctgcaccctccacaagcagcactgtcaaaactgaaggagcagaggcaactcctggagctcagaaaagaaaaaaatcaaccaaagaaaaggctggacccaaagggagtaaggtgtccgaggaacagactcagcctccctctcctgcaggagccggcatgtccacagccatgggccgttccccatctcccaagacctcattgtcagctccacccaacacttcttcaactgagaacccgaaaacagtggccaaatgtcaggtaactcccagaagaaatgttctccaaaaacgcccagtgatagtgaaggtactgagtacaacaaagccatttgaatatgagaccccagaaatggagaaaaaaataatgtttcatgctacagtggctacacagacacagttcttccatgtgaaggttttaaacaccagcttgaaggagaaattcaatggaaagaaaatcatcatcatatcagattatttggaatatgatagtctcctagaggtcaatgaagaatctactgtatctgaagctggtcctaaccaaacgtttgaggttccaaataaaatcatcaacagagcaaaggaaactctgaagattgatattcttcacaaacaagcttcaggaaatattgtatatggggtatttatgctacataagaaaacagtaaatcagaagaccacaatctacgaaattcaggatgatagaggaaaaatggatgtagtggggacaggacaatgtcacaatatcccctgtgaagaaggagataagctccaacttttctgctttcgacttagaaaaaagaaccagatgtcaaaactgatttcagaaatgcatagttttatccagataaagaaaaaaacaaacccgagaaacaatgaccccaagagcatgaagctaccccaggaacagagtcagcttccaaatccttcagaggccagcacaaccttccctgagagccatcttcggactcctcagatgccaccaacaactccatccagcagtttcttcaccaagaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgttgaaaccaagactgaagactgaacctgaagaagtttccatagaagacagtgcccagagtgacctcaaagaagtgatggtgctgaacgcaacagaatcatttgtatatgagcccaaagagcagaagaaaatgtttcatgccacagtggcaactgagaatgaagtcttccgagtgaaggtttttaatattgacctaaaggagaagttcaccccaaagaagatcattgccatagcaaattatgtttgccgcaatgggttcctggaggtatatcctttcacacttgtggctgatgtgaatgctgaccgaaacatggagatcccaaaaggattgattagaagtgccagcgtaactcctaaaatcaatcagctttgctcacaaactaaaggaagttttgtgaatggggtgtttgaggtacataagaaaaatgtaaggggtgaattcacttattatgaaatacaagataatacagggaagatggaagtggtggtgcatggacgactgaccacaatcaactgtgaggaaggagataaactgaaactcacctgctttgaattggcaccgaaaagtgggaataccggggagttgagatctgtaattcatagtcacatcaaggtcatcaagaccaggaaaaacaagaaagacatactcaatcctgattcaagtatggaaacttcaccagactttttcttctaaaatctggatgtcattgacgataatgtttatggagataaggtctaagtgcctaaaaaaatgtacatatacctggttgaaatacaacactatacatacacaccaccatatatactagctgttaatcctatggaatggggtattgggagtgcttttttaatttttcatagtttttttttaataaaatggcatattttgcatctacaacttctataatttgaaaaaataaataaacattatcttttttgtgaaaaaaaaaaaaaaa

SEQ ID NO: 26 - DNA, 1697 bp, Homo sapiens
gggggtgctgcagccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatctttaaattcatcattcctaatgttgtaaaatacagcccgaactgcaagttgcttattgtttcaaatccagtggatatcttgacctacgtggcttggaagataagtggttttcccaaaaaccgtgttattggaagtggttgcaatctggattcagcccgattccgttacctgatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtgaacaaaaaaaaaaaaaaaaaaaaaaaaaaaa

SEQ ID NO: 27 - DNA, 1510 bp, Homo sapiens
cagcctggcccttatctgcactgggccagcatcctccggccgctgcgccgccaggggtgagagggaggaaaccgggccgccgggggcggggagaaggcgggccggcccgggagccgctcactttccctgggggggacctacgcggagacctcggctatcctggccttccgaggcccacgaggaggcgcggcccaacgccggggcctggagcattgaggccggaccctcgcgagacagcagagcctggcctgacgctggaaaccacaccctggcccagactgccagccctgacgggacagagccagggcactcaccaggctgcaagaacagtgctggggtgagtacccccacgtcggggtccatgtgcccgcctcaggcacaggcagaggtgggccccaccatgactgagaaggcagagatggtgtgtgcccccagcccagcgcctgccccaccccctaagcctgcctcgcctgggcccccgcaggtggaggaggtgggccaccgaggaggctcctcgccccccaggctgccacctggtgtaccagtgatcagcctgggccacagcaggcccccaggggtagccatgcccaccacagagctgggcactctgcggcccccgctgctgcaactctccaccctgggaactgccccgcccactttggccctgcactaccaccctcaccccttcctcaacagtgtctacattgggccagcaggaccttttagcatcttccctagcagccggttgaagcggagaccaagccactgtgagctggacctggctgaggggcaccagccccagaaggtggcccggcgcgtgttcaccaacagccgggagcgctggcggcagcagaacgttaacggcgccttcgccgagctgaggaagctgctgccgacgcacccgcccgaccggaagctgagcaagaacgaggtgctccgcctagccatgaagtacatcggcttcctggtgcggctgctgcgcgaccaagccgcagctctggccgcaggccccacccctcccgggcctcgcaaacggccggtgcaccgggtcccagacgacggcgcccgccggggatccggacgcagggccgaggcggcagcgcgctcgcagcccgcgcccccggccgaccccgacggcagccccggtggagcggcccggcccatcaagatggagcaaaccgctttgagcccagaggtgcggtgaccgcacgcggcagcacctctgagccggagggcaccagggactcggcccagggccgtcaaggaaagggcagtggacgtgctgcgcatgttcgggagcgaactcccccgaagaaggaccagtgaagacgtcaggggcaaggtctcgggggtccggaagggtgatcatcgacccccaagggacccgcagacccttaaaaaaatcacccacaaccctctggaagtggccttgcccggtccccttcccaggggcgaggtcggcaaagcaacatggcagagcagtcataggaaaaaaaaaaaaaaaaaaaaa

SEQ ID NO: 28 - DNA, 3297 bp, Homo sapiens
gctccatggtcggcgcccacagcccgcgacggcctgtcttgcgctccacttccttcacatcctcctccgcctcctcgttttcaggcgccgccggcggcgctgtgtggaggcccgcgagctgaaattcgcggtgcgacgggagggagtggagaaggaggtgagggggcccaggatcgcggggcgccctgaggcaaggggacgccggcgggccgaagcgcagcccgccgcccgcaggctcggctccgccactgccgccctcccggtctcctcgcctcggccgccgaggcagggagagaatgagccccgggacccgccgggggacggcccgggccaggcccgggatctagacggccgtagggggaagggagccgccctccccacggcgccttttcggaactgccgtggactcgaggacgctggtcgccggcctcctagggctgtgctgttttgttttgaccctcgcattgtgcagaattaaagtgcagtaaaatgtccactaggaccccattgccaacggtgaatgaacgagacactgaaaaccacacgtcacatggagatgggcgtcaagaagttacctctcgtaccagccgctcaggagctcggtgtagaaactctatagcctcctgtgcagatgaacaacctcacatcggaaactacagactgttgaaaacaatcggcaaggggaattttgcaaaagtaaaattggcaagacatatccttacaggcagagaggttgcaataaaaataattgacaaaactcagttgaatccaacaagtctacaaaagctcttcagagaagtaagaataatgaagattttaaatcatcccaatatagtgaagttattcgaagtcattgaaactgaaaaaacactctacctaatcatggaatatgcaagtggaggtgaagtatttgactatttggttgcacatggcaggatgaaggaaaaagaagcaagatctaaatttagacagattgtgtctgcagttcaatactgccatcagaaacggatcgtacatcgagacctcaaggctgaaaatctattgttagatgccgatatgaacattaaaatagcagatttcggttttagcaatgaatttactgttggcggtaaactcgacacgttttgtggcagtcctccatacgcagcacctgagctcttccagggcaagaaatatgacgggccagaagtggatgtgtggagtctgggggtcattttatacacactagtcagtggctcacttccctttgatgggcaaaacctaaaggaactgagagagagagtattaagagggaaatacagaattcccttctacatgtctacagactgtgaaaaccttctcaaacgtttcctggtgctaaatccaattaaacgcggcactctagagcaaatcatgaaggacaggtggatcaatgcagggcatgaagaagatgaactcaaaccatttgttgaaccagagctagacatctcagaccaaaaaagaatagatattatggtgggaatgggatattcacaagaagaaattcaagaatctcttagtaagatgaaatacgatgaaatcacagctacatatttgttattggggagaaaatcttcagagctggatgctagtgattccagttctagcagcaatctttcacttgctaaggttaggccgagcagtgatctcaacaacagtactggccagtctcctcaccacaaagtgcagagaagtgtttcttcaagccaaaagcaaagacgctacagtgaccatgctggaccagctattccttctgttgtggcgtatccgaaaaggagtcagaccagcactgcagatagtgacctcaaagaagatggaatttcctcccggaaatcaagtggcagtgctgttggaggaaagggaattgctccagccagtcccatgcttgggaatgcaagtaatcctaataaggcggatattcctgaacgcaagaaaagctccactgtccctagtagtaacacagcatctggtggaatgacacgacgaaatacttatgtttgcagtgagagaactacagctgatagacactcagtgattcagaatggcaaagaaaacagcactattcctgatcagagaactccagttgcttcaacacacagtatcagtagtgcagccaccccagatcgaatccgcttcccaagaggcactgccagtcgtagcactttccacggccagccccgggaacggcgaaccgcaacatataatggccctcctgcctctcccagcctgtcccatgaagccacaccattgtcccagactcgaagccgaggctccactaatctctttagtaaattaacttcaaaactcacaaggagtcgcaatgtatctgctgagcaaaaagatgaaaacaaagaagcaaagcctcgatccctacgcttcacctggagcatgaaaaccactagttcaatggatcccggggacatgatgcgggaaatccgcaaagtgttggacgccaataactgcgactatgagcagagggagcgcttcttgctcttctgcgtccacggagatgggcacgcggagaacctcgtgcagtgggaaatggaagtgtgcaagctgccaagactgtctctgaacggggtccggtttaagcggatatcggggacatccatagccttcaaaaatattgcttccaaaattgccaatgagctaaagctgtaacccagtgattatgatgtaaattaagtagcaattaaagtgttttcctgaacactgatggaaatgtatagaataatatttaggcaataacgtctgcatcttctaaatcatgaaattaaagtctgaggacgagagcacgcctgggagcgaaagctggccttttttctacgaatgcactacattaaagatgtgcaacctatgcgccccctgccctacttccgttaccctgagagtcggcgtgtggccccatctccatgtgcctcccgtctgggtgggtgtgagagtggacggtatgtgtgtgaagtggtgtatatggaagcatctccctacactggcagccagtcattactagtacctctgcgggagatcatccggtgctaaaacattacagttgccaaggaggaaaatactgaatgactgctaagaattaaccttaagaccagttcatagttaatacaggtttacagttcatgcctgtggttttgtgtttgttgttttgtgtttttttagtgcaaaaggtttaaatttatagttgtgaacattgcttgtgtgtgtttttctaagtagattcacaagataattaaaaattcactttttctcagtaaaaaaaaaaaaaaaaaa

SEQ ID NO: 29 - DNA, 2129 bp, Homo sapiens
cggagagcgagcgggagtcggtgctcggcctcctgcggggagcagcccgaggaatctgcagggagaggtcgggaggtgacaacgccagcatgccagtgctatcggaggactctggtttgcatgaaaccctggccctgctgacctcccagctcagacctgactccaaccacaaggaggagatgggcttcctgagggatgttttcagtgaaaaaagcctcagttacttaatgaagattcatgagaagcttcgctattatgaaaggcaaagtccaaccccagttctgcacagcgctgtggccctcgctgaggacgtgatggaggagttgcaggccgcctccgtgcacagtgatgagagggagctgctccagctgctgtccaccccgcacctgagggctgtgctcatggtacatgacacggttgcccagaagaattttgaccccgttctcccgcctctgcctgacaatatcgatgaggattttgatgaggaatcggtgaagatcgtccgcttggtgaagaacaaggaacccctgggtgccaccatccggcgggacgagcactcaggggctgttgtggtggccaggatcatgcgaggaggcgcagcagacaggagcggcctggtccacgttggagatgagctccgagaagtgaacgggatcgcagtcctgcacaagcggcccgacgagatcagccagattctggcccagtcccagggatccatcaccctaaaaatcatcccagccacccaggaggaagatcgcttaaaggagagcaaggtgttcatgcgcgccctcttccactacaaccctcgggaggaccgggccatcccttgccaggaggcgggcctgcccttccagcgcaggcaggtcctggaggtggtgagccaggacgaccccacgtggtggcaggccaagcgagtcggggacaccaaccttcgagccggcctcatcccctccaaggggttccaggagagacgactaagctaccggagagccgcgggcaccctgccgagcccccagagcctcaggaagcccccctatgatcagccttgtgacaaagagacctgtgactgtgagggctacctcaaagggcactatgtggctggtcttcggaggagcttccggctgggctgtagggagagactgggtggctcgcaggaaggaaagatgtcctccggagctgagtctccggagctgctgacttacgaagaggtggccaggtaccaacaccagcccggagagcggccccgcctggtggttctgatcgggtctctgggagcccgactgcacgagctgaagcaaaaggtggtggctgagaacccacagcactttggcgtcgctgttccacataccaccaggccccgaaagagccatgagaaggaaggagtggaatatcactttgtgtctaagcaagcatttgaggccgacttacatcacaacaagttcctggaacatggtgaatataaggaaaatctgtatggaaccagcctggaggccattcaggctgttatggccaaaaacaaagtttgtttggtggatgtggagccagaagcactgaaacaactgaggacctcagaatttaaaccctatattatatttgtaaagcctgcaattcaggaaaaaagaaaaacgccacctatgtccccagcttgtgaggacacagcagccccatttgatgagcagcagcaagagatggccgcttctgccgccttcatagaccggcattacgggcacctggtagacgccgtgctggtgaaggaggatctccagggtgcctacagccagctcaaagtggtcttagagaagctgagcaaggacactcactgggtacctgttagttgggtcaggtaactttatcccagaacatccaagctggacgggaccttgaagatcatctagtccagactccctcattttaccatcaaggaatctcaagcgcagagagggagagaattctccacaaattccatcatcgagaagagtataagtgggaagtcttgtttgttgttggtttttgtctgttgtttttcactgcacctctttggatcatgatttgaaaggggcatatcagaaaacaacacatttcatttattaaagtatcacaggcaaaaaaaaaaaaaaaaaaaaaaaaaa

SEQ ID NO: 30 - DNA, 3521 bp, Homo sapiens
ggtcgaggttccggcgcggctgctggccggcggctgagccgggagaaaggagctgcggccaccgttcgccctgcttctgcgcctcggggcgggcgcgccggtaaaaatggcgaaatgggggtaggcggcgctggacctgaagagatggggcgcgcaggtggggcggttgtcagagccccctgacgtgggcgccgggcttttatcggcgatttgatctggcgacctcgggccggcgcctaagaggtcagactgcggagcctgcgggtcgccagcggccccgccgagagccggaggcaatggatgaacagagcgtggagagcattgctgaggttttccgatgtttcatttgtatggagaaattgcgggatgcacgcctgtgtcctcattgctccaaactgtgttgtttcagctgtattaggcgctggctgacagagcagagagctcaatgtcctcattgccgtgctccactccagctacgagaactagtaaattgtcgttgggcagaagaagtaacacaacagcttgatactcttcaactctgcagtctcaccaaacatgaagaaaatgaaaaggacaaatgtgaaaatcaccatgaaaaacttagtgtattttgctgggcttgtaagaagtgtatctgccatcagtgtgcactttggggaggaatgcatggcggacatacctttaaacctttggcagaaatttatgagcaacacgtcactaaagtgaatgaagaggtagccaaacttcgtcggcgtctcatggaactgatcagcttagttcaagaagtggaaaggaatgtagaagctgtaagaaatgcaaaagatgagcgtgttcgggaaattaggaatgcagtggagatgatgattgcacggttagacacacagctgaagaataagcttataacactgatgggtcagaagacatctctaacccaagaaacagagcttttggaatccttacttcaggaggtggagcaccagttgcggtcttgtagtaagagtgagttgatatctaagagctcagagatccttatgatgtttcagcaagttcatcggaagcccatggcatcttttgttaccactcctgttccaccagactttaccagtgaattagtgccatcttacgattcagctacttttgttttagagaatttcagcactttgcgtcagagagcagatcctgtttacagtccacctcttcaagtttcaggactttgctggaggttaaaagtttacccagatggaaatggagttgtgcgaggttactacttatctgtgtttctggagctctcagctggcttgcctgaaacttctaaatatgaatatcgtgtagagatggttcaccagtcctgtaatgatcctacaaaaaatatcattcgagaatttgcatctgactttgaagttggagaatgctggggctataatagatttttccgtttggacttactcgcaaatgaaggatacttgaatccacaaaatgatacagtgattttaaggtttcaggtacgttcaccaactttctttcaaaaatcccgggaccagcattggtacattactcagctggaagctgcacagactagttatatccaacaaataaacaaccttaaagagagacttactattgagctgtctcgaactcagaagtcaagagatttgtcaccaccagataaccatcttagcccccaaaatgatgatgctctggagacacgagctaagaagtctgcatgctctgacatgcttctcgaaggtggtcctactacagcttctgtaagagaggccaaagaggatgaagaagatgaggagaagattcagaatgaagattatcatcacgagctttcagatggagatctggatctggatcttgtttatgaggatgaagtaaatcagctcgatggcagcagttcctctgctagttccacagcaacaagtaatacagaagaaaatgatattgatgaagaaactatgtctggagaaaatgatgtggaatataacaacatggaattagaagagggagaactcatggaagatgcagctgctgcaggacccgcaggtagtagccatggttatgtgggttccagtagtagaatatcaagaagaacacatttatgctccgctgctaccagtagtttactagacattgatccattaattttaatacatttgttggaccttaaggaccggagcagtatagaaaatttgtggggcttacagcctcgcccacctgcttcacttctgcagcccacagcatcatattctcgaaaagataaagaccaaaggaagcaacaggcaatgtggcgagtgccctctgatttaaagatgctaaaaagactcaaaactcaaatggccgaagttcgatgtatgaaaactgatgtaaagaatacactttcagaaataaaaagcagcagtgctgcttctggagacatgcagacaagccttttttctgctgaccaggcagctctggctgcatgtggaactgaaaactctggcagattgcaggatttgggaatggaactcctggcaaagtcatcagttgccaattgttacatacgaaactccacaaataagaagagtaattcgcccaagccagctcgatccagtgtagcaggtagtctatcacttcgaagagcagtggaccctggagaaaatagtcgttcaaagggagactgtcagactctgtctgaaggctccccaggaagctctcagtctgggagcaggcacagttctccccgagccttgatacatggcagtatcggtgatattctgccaaaaactgaagaccggcagtgtaaagctttggattcagatgctgttgtggttgcagttttcagtggcttgcctgcggttgagaaaaggaggaaaatggtcaccttgggggctaatgctaaaggaggtcatctggaaggactgcagatgactgatttggaaaataattctgaaactggagagttacagcctgtactacctgaaggagcttcagctgcccctgaagaaggaatgagtagcgacagtgacattgaatgtgacactgagaatgaggagcaggaagagcataccagtgtgggcgggtttcacgactccttcatggtcatgacacagcccccggatgaagatacacattccagttttcctgatggtgaacaaataggccctgaagatctcagcttcaatacagatgaaaatagtggaagataatttgatttgaaactgacactgcacctgatgggttaacaagatctaggcttcagaaggtgacagatatgagtgaggaccatgtgtggggcaaagcctcagaatgatgaaaaggttccggcactatagttggggccatgttgactccttttcaaccatttgtcacagacgtgagaagaagaaatgacttcaaaatcaagagaaaacaaatactgaaagtctctacttacatccaaattttaaaaaataaaatctgtagattaacaatctaaaaaaaaaaaaaaaaaaaaaaaaaaaaaaaaaaaaaaaaaaaaaaaaaaaaaaaaaaaaaaa

SEQ ID NO: 31 - DNA, 2232 bp, Homo sapiens
tctctctctgcttctttccttttctctctcatggtagggttatgagtcagttgccaaaaggtggggacatttcctgatgcatttgcaacactgagaagttatcttaagggaggctgggccccattctactcatctggcccagaaagtgaacaccttgggggccactaaggcagccctgctaggggagacgctccaacctgtcttctctctgtctcctggcagctctcttggcctcctagtttctacctaatcatgtccctggtggaggccatcagcctctggaatgaaggggtgctggcagcggacaagaaggactggaagggagccctggatgccttcagtgccgtccaggacccccactcccggatttgcttcaacattggctgcatgtacactatcctgaagaacatgactgaagcagagaaggcctttaccagaagcattaaccgagacaagcacttggcagtggcttacttccaacgagggatgctctactaccagacagagaaatatgatttggctatcaaagaccttaaagaagccttgattcagcttcgagggaaccagctgatagactataagatcctggggctccagttcaagctgtttgcctgtgaggtgttatataacattgctttcatgtatgccaagaaggaggaatggaaaaaagctgaagaacagttagcattggccacgagcatgaagtctgagcccagacattccaaaatcgacaaggcgatggagtgtgtctggaagcagaagctatatgagccagtggtgatccctgtgggcaggctgtttcgaccaaatgagagacaagtggctcagctggccaagaaggattacctaggcaaggcaacggtcgtggcatctgtggtggatcaagacagtttctctgggtttgcccctctgcaaccacaggcagctgagcctccacccagaccgaaaaccccagagatcttcagggctctggaaggggaggctcaccgtgtgctatttgggtttgtgcctgagacaaaagaagagctccaggtcatgccagggaacattgtctttgtcttgaagaagggcaatgataactgggccacggtcatgttcaacgggcagaaggggcttgttccctgcaactaccttgaaccagttgagctgcggatccaccctcagcagcagccccaggaggaaagctctccgcagtccgacatcccagctcctcctagttccaaagcccctggaagaccccagctgtcaccaggccagaaacaaaaagaagagcctaaggaagtgaagctcagtgttcccatgccctacacactcaaggtgcactacaagtacacggtagtcatgaagactcagcccgggctcccctacagccaggtccgggacatggtgtctaagaaactggagctccggctggaacaaactaagctgagctatcggcctcgggacagcaatgagctggtgcccctttcagaagacagcatgaaggatgcctggggccaggtgaaaaactactgcctgactctgtggtgtgagaacacagtgggtgaccaaggctttccagatgaacccaaggaaagtgaaaaagctgatgctaataaccagacaacagaacctcagcttaagaaaggcagccaagtggaggcactcttcagttatgaggctacccaaccagaggacctggagtttcaggaaggggatataatcctggtgttatcaaaggtgaatgaagaatggctggaaggggagtgcaaagggaaggtgggcattttccccaaagtttttgttgaagactgcgcaactacagatttggaaagcactcggagagaagtctaggatgtttcacaaactacaaagctgaagaaaatgaagccctattacttgtttgtaagatttagcacccttctgctgtatactgtactgagacattacagtttggaagtgttaactatttattccctgttaaaatttaacctactagacaatgatgtgagtacccaggatgatttcctggggcacagtgggtgaggagatggggacaggtgaatggaggagttaggggagaggaaaagtggatggaagtgtctggaaagggcacgagagagtcttccaggtactgatcctgtttcttgctctgagtgctagctagccagctgtgttcacactgtaaacattcatcaagctgtacatttggtgcacttttctgtgtcataccacaaaaaaaaaaaaaaaaaa

SEQ ID NO: 32 - DNA, 700 bp, Homo sapiens
gcggcgtgagaagccatgagcagcaaagtctctcgcgacaccctgtacgaggcggtgcgggaagtcctgcacgggaaccagcgcaagcgccgcaagttcctggagacggtggagttgcagatcagcttgaagaactatgatccccagaaggacaagcgcttctcgggcaccgtcaggcttaagtccactccccgccctaagttctctgtgtgtgtcctgggggaccagcagcactgtgacgaggctaaggccgtggatatcccccacatggacatcgaggcgctgaaaaaactcaacaagaataaaaaactggtcaagaagctggccaagaagtatgatgcgtttttggcctcagagtctctgatcaagcagattccacgaatcctcggcccaggtttaaataaggcaggaaagttcccttccctgctcacacacaacgaaaacatggtggccaaagtggatgaggtgaagtccacaatcaagttccaaatgaagaaggtgttatgtctggctgtagctgttggtcacgtgaagatgacagacgatgagcttgtgtataacattcacctggctgtcaacttcttggtgtcattgctcaagaaaaactggcagaatgtccgggccttatatatcaagagcaccatgggcaagccccagcgcctatattaaggcacatttgaataaattctattaccagttc

SEQ ID NO: 33 - DNA, 3002 bp, Homo sapiens
gccggcggccccggcccggggcctgcgagcgcctcggggcactcccggccgaggcctgcaggggccgccccgcgcggggatggcgcctcggggagcaggcacctcggggctccgaccctcgcagggtgccagggccagttgggaatgcgtccctgttccccgactcaggcccaggttctcaggccccaggcctggggccacgaatggccctacactaggggacacatgcgcgccctgggcctcggcgatttcctcctggtcgttctggccgcaaacttaaacctgcccttgcactctccactctccggcccttgggacaggaagcctccccggggcccgcgccccccgctccccgctcccagcgagccgggagtgtacctcacagccttctaggatctccagagtggacaggaatctcacttggagggaccatggagcagtatacagcaaacagcaatagttcgacagagcagattgttgtccaggcaggacagattcagcagcaggtccaagggcagccattaatggtgcaggtcagtggaggccagctaatcacatcaactggccaacccatcatggtccaggctgtccctggtgggcaaggtcaaaccatcatgcaagtacctgtttctggaacacagggtttgcagcaaatacagttggtcccacctggacagatccagatccagggtggacaggctgtgcaggtgcagggccagcagggccagacccagcagatcatcatccagcagccccagacggctgtcactgctggccagactcagacacagcagcagattgctgtccagggacagcaagtggcacagactgctgaagggcagaccatcgtctatcaaccagttaatgcagatggcaccattctccagcaagttacagtccctgtttcaggcatgatcactatcccagcagccagtttggcaggagcacagattgttcaaacaggagccaataccaacacaaccagcagtgggcaagggactgtcactgtgacactaccagtggcaggcaatgtggtcaattcaggagggatggtcatgatggttcctggggctggctctgtgcctgctatccaaagaatccctctacctggagcagagatgcttgaagaagagcctctctacgtgaatgccaaacaatacaaccgtattcttaagaggaggcaagcccgagctaaactagaggcagaagggaaaattccaaaggagagaaggaaatacctgcatgagtctcggcaccgtcatgccatggcacggaagcgtggtgaaggtggacgatttttctctccaaaggaaaaggatagtccccatatgcaggatccaaaccaagccgatgaagaagcaatgacacagatcatccgagtgtcctaaccccacgccatgtgatggagctgatcaaggtcatgtttctcactgttccaggaaattgatcaactcttccaatgggacattgatgatcacattctgccctttactacaggacagaaaccacttagtttttaataagtggctcagtattacaattgcagcgatgcctggcaaattgaagttgcaagcctttgtcttactctttcagtcaggacctatttcagactgttgatgacattgacatttcatggattcggatgatgcaaggagacacatgccaccccagcagtacacaaagcacttaccttgactggtgaagtcagccagccatctcagcagggaagaacaaccttctcaaccaaaactgcaatcaggaaagcagcagcccactccttcccatggaatctagacagcatttatccctctggtgggggaatctcacactgactaactgaatggacgctttgtattcagagatggcttccaggcaaggaagttctatcttgtgatacggaaacaataaattcctgagatacattcttccaatctgtggtaataataagaattcctctctgcccagatggtaggttgatatttctggactctagccaagaaaaagctaattctggggatagaatgataaaagactcaagcactaaaaatatacattgccgaatctctttttctagctttgaaaagttgtggcctgcttgtccctctgttgactggtcatctggaccatcgtacttgctgtggctacttctaagacaatgtagagggttattaaaccttgaaactgcctttcctaagtagagaacaagactattcaacaacttctttgctgaagcactgaggagatttgtaatactcctaaaggaagggccaaactagagattttcaatcatagactttgtgacagcatttggggaactaaaagattcatgtgtttcagcctagtgggagagagtgggggagaggaagagagagagagagcatgtatacccgtatgttatcatagagcacgattctccagtggatggatacctggaatggatcattaagatgaagagagtaattcacatttactctagaacctttaacaagcactgaaaggaagaagcctgagatttgatccttgacaatttctggaaagcactggtcagtcacactgtcctggacagagtccaagttacccactaaagcttgaggtggatgttggcttcactcctgcagcacaatgctggccctagaaagcgagcattttcattcttcccatggcagctgattctttggtgccagttttcatgttttctccataggccttgtgcagaaaggttttctcattgcatccatactgtgagaataaactgcagactgaaagaaacatgcctgcagggtcaggaactgggccactgttcccagaacgggttgtgcctcagaaatctatttctcttttagatgttgaaccctgagagaactaagatgatatttcagaagagttggctgccttctctggaaacaaggaaaacccaataattccgaggaacattgggggttaaataccaagaataaaaattggttcttctttgaaaaaaaaaaagaaaaaaaaaaaaaaaa

SEQ ID NO: 34 - DNA, 1862 bp, Homo sapiens
gtgtcgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcatttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactcaaaaaaaaaaaaaaaaaaaaaaaaaaaaa

SEQ ID NO: 35 - DNA, 2146 bp, Homo sapiens
cggagatggatgtctctctttgcccagccaagtgtagtttctggcggattttcttgctgggaagcgtctggctggactatgtgggctccgtgctggcttgccctgcaaattgtgtctgcagcaagactgagatcaattgccggcggccggacgatgggaacctcttccccctcctggaagggcaggattcagggaacagcaatgggaacgccagtatcaacatcacggacatctcaaggaatatcacttccatacacatagagaactggcgcagtcttcacacgctcaacgccgtggacatggagctctacaccggacttcaaaagctgaccatcaagaactcaggacttcggagcattcagcccagagcctttgccaagaacccccatttgcgttatataaacctgtcaagtaaccggctcaccacactctcgtggcagctcttccagacgctgagtcttcgggaattgcagttggagcagaactttttcaactgcagctgtgacatccgctggatgcagctctggcaggagcagggggaggccaagctcaacagccagaacctctactgcatcaacgctgatggctcccagcttcctctcttccgcatgaacatcagtcagtgtgaccttcctgagatcagcgtgagccacgtcaacctgaccgtacgagagggtgacaatgctgttatcacttgcaatggctctggatcaccccttcctgatgtggactggatagtcactgggctgcagtccatcaacactcaccagaccaatctgaactggaccaatgttcatgccatcaacttgacgctggtgaatgtgacgagtgaggacaatggcttcaccctgacgtgcattgcagagaacgtggtgggcatgagcaatgccagtgttgccctcactgtctactatcccccacgtgtggtgagcctggaggagcctgagctgcgcctggagcactgcatcgagtttgtggtgcgtggcaaccccccaccaacgctgcactggctgcacaatgggcagcctctgcgggagtccaagatcatccatgtggaatactaccaagagggagagatttccgagggctgcctgctcttcaacaagcccacccactacaacaatggcaactataccctcattgccaaaaacccactgggcacagccaaccagaccatcaatggccacttcctcaaggagccctttccagagagcacggataactttatcttgtttgacgaagtgagtcccacacctcctatcactgtgacccacaaaccagaagaagacacttttggggtatccatagcagttggacttgctgcttttgcctgtgtcctgttggtggttctcttcgtcatgatcaacaaatatggtcgacggtccaaatttggaatgaagggtcccgtggctgtcatcagtggtgaggaggactcagccagcccactgcaccacatcaaccacggcatcaccacgccctcgtcactggatgcggggcccgacactgtggtcattggcatgactcgcatccctgtcattgagaacccccagtacttccgtcagggacacaactgccacaagccggacacgtgggtcttttcaaacatagacaatcatgggatattaaacttgaaggacaatagagatcatctagtcccatcaactcactatatatatgaggaacctgaggtccagagtggggaagtgtcttacccaaggtcacatggtttcagagaaattatgttgaatccaataagccttcccggacattccaagcctcttaaccatggcatctatgttgaggatgtcaatgtttatttcagcaaaggacgtcatggcttttaaaaactccttttaagcctccttgttttgatgtcaccttggtaggctgggccctctgagaggttggaagctctaggcattgttctctttggatccagggatgctaagtagaaactgcatgagccaccagtgccccggcaccctttaacaccaccagatgggtgttttcccccatccaccactggcagggttgccccttccctccaatcatcactgtgctccttttttcccggcctacgaggcagctcctgccactatctttagagccaataaagagaattaaaaacctgaaaaaaaaaaaaaaa

SEQ ID NO: 36 - DNA, 3010 bp, Homo sapiens
atcctaccattcactgtcttgccggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtcctgcaggaacccgaacagttccccctggtgcagggagtggccataggtggctcctatggacggaaaacagtcttaagaggcaactccgatggtacccttgtcctcttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggttcaccatccaggtgttcacaaaaaatcagagaatctctttcgaggtgctggccgccttcaacgctctgagcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacagtgccagaaaaaaatcaaggatttaccctcgctgtctccgtatgccctggagctgcttacggtgtatgcctgggaacaggggtgcagaaaagacaactttgacattgctgaaggcgtcagaaccgttctggagctgatcaaatgccaggagaagctgtgtatctattggatggtcaactacaactttgaagatgagaccatcaggaacatcctgctgcaccagctccaatcagcgaggccagtaatcttggatccagttgacccaaccaataatgtgagtggagataaaatatgctggcaatggctgaaaaaagaagctcaaacctggttgacttctcccaacctggataatgagttacctgcaccatcttggaatgttctgcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccggggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctctgcatcttctggaaggtcaattacaactttgaagatgagaccgtgaggaagtttctactgagccagttgcagaaaaccaggcctgtgatcttggacccagccgaacccacaggtgacgtgggtggaggggaccgttggtgttggcatcttctggcaaaagaagcaaaggaatggttatcctctccctgcttcaaggatgggactggaaacccaataccaccttggaaagtgccggtaaaagtcatctaaaggaggcgttgtctggaaatagccctgtaacaggcttgaatcgaagaacttctcctactgtagcaacctgaaattaactcagacacaaataaaggaaacccagctcacaggagcttaaacagctggtcagccccctaagcccccactacaagtgatcctcaggcaggtaaccccagattcatgcactgtagggtgctgcgcagcatccctagtctctacccagtagatgccactagccctcctctcccagtgacaaccaaaagtcttcagacattgtcaaacgttcccctgggttcacagatctttctgcctttggcttttggctccaccctctttagctgttaatttgagtacttatggccctgaaagcggccacggtgcctccagatggcaggtttgcaatccaagcaggaagaaggaaaagatacccaaaggtcaagaacacagtgattttattagaagtttcatccgcaaattttcttccatttcattgctcagaaatgtcatgtggctacctgtaacttgaaggtggctacaaagatgactgtggacgtgggttgcactggccacccaaggatgtctgccacacctctccaaagccctccctacctaccaagatatacctgatatattccaccaggatatcctccctccagatatacttggttctctccaccaggttctttctttaaagcaggatttctcaactttgatacttactcacatttggggctagacagttctttgtttggaggctctcttgtgcattgtaggatgttgagcagcatctctggcctgtacccagtagatgccacccagttgtgacaattaaaagtgtcttgagactttaaaaaaaaaaaaaaaaaaaaaaaaaaaaaaaaaaaaaaaaaaaaaaaaaaaaaaaa

SEQ ID NO: 37 - DNA, 4049 bp, Homo sapiens
cagcagcggtagaggcggcggcgaggactggcgacggcgaggagatagtgtctgccttctggagagctgaccaaacactaaggatgctgaagttccgaacagtccatgggggcctgaggctcctgggaatccgccgaacctccaccgcccccgctgcctccccaaatgtccggcgcctggagtataagcccatcaagaaagtcatggtggccaacagaggtgagattgccatccgtgtgttccgggcctgcacggagctgggcatccgcaccgtagccatctactctgagcaggacacgggccagatgcaccggcagaaagcagatgaagcctatctcatcggccgcggcctggcccccgtgcaggcctacctgcacatcccagacatcatcaaggtggccaaggagaacaacgtagatgcagtgcaccctggctacgggttcctctctgagcgagcggacttcgcccaggcctgccaggatgcaggggtccggtttattgggccaagcccagaagtggtccgcaagatgggagacaaggtggaggcccgggccatcgccattgctgcgggtgttcccgttgtccctggcacagatgcccccatcacgtccctgcatgaggcccacgagttctccaacacctacggcttccccatcatcttcaaggcggcctatgggggtggagggcgtggcatgagggtggtgcacagctacgaggagctggaggagaattacacccgggcctactcagaggctctggccgcctttgggaatggggcgctgtttgtggagaagttcatcgagaagccacggcacatcgaggtgcagatcttgggggaccagtatgggaacatcctgcacctgtacgagcgagactgctccatccagcggcggcaccagaaggtggtcgagattgcccccgccgcccacctggacccgcagcttcggactcggctcaccagcgactctgtgaaactcgctaaacaggtgggctacgagaacgcaggcaccgtggagttcctggtggacaggcacggcaagcactacttcatcgaggtcaactcccgcctgcaggtggagcacacggtcacagaggagatcaccgacgtagacctggtccatgctcagatccacgtggctgagggcaggagcctacccgacctgggcctgcggcaggagaacatccgcatcaacgggtgtgccatccagtgccgggtcaccaccgaggaccccgcgcgcagcttccagccggacaccggccgcattgaggtgttccggagcggagagggcatgggcatccgcctggataatgcttccgccttccaaggagccgtcatctcgccccactacgactccctgctggtcaaagtcattgcccacggcaaagaccaccccacggccgccaccaagatgagcagggcccttgcggagttccgcgtccgaggtgtgaagaccaacatcgccttcctgcagaatgtgctcaacaaccagcagttcctggcaggcactgtggacacccagttcatcgacgagaacccagagctgttccagctgcggcctgcacagaaccgggcccaaaagctgttgcactacctcggccatgtcatggtaaacggtccaaccaccccgattcccgtcaaggccagccccagccccacggaccccgttgtccctgcagtgcccataggcccgcccccggctggtttcagagacatcctgctgcgagaggggcctgagggctttgctcgagctgtgcggaaccacccggggctgctgctgatggacacgaccttcagggacgcccaccagtcactgctggccactcgtgtgcgcacccacgatctcaaaaagatcgccccctatgttgcccacaacttcagcaagctcttcagcatggagaactggggaggagccacgtttgacgtcgccatgcgcttcctgtatgagtgcccctggcggcggctgcaggagctccgggagctcatccccaacatccctttccagatgctgctgcggggggccaatgctgtgggctacaccaactacccagacaacgtggtcttcaagttctgtgaagtggccaaagagaatggcatggatgtcttccgtgtgtttgactccctcaactacttgcccaacatgctgctgggcatggaggcggcaggaagtgccggaggcgtggtggaggctgccatctcatacacgggcgacgtggccgaccccagccgcaccaagtactcactgcagtactacatgggcttggccgaagagctggtgcgagctggcacccacatcctgtgcatcaaggacatggccgggctgctgaagcccacggcctgcaccatgctggtcagctccctccgggaccgcttccccgacctcccactgcacatccacacccacgacacgtcaggggcaggcgtggcagccatgctggcctgtgcccaggctggagctgatgtggtggatgtggcagctgattccatgtctgggatgacttcacagcccagcatgggggccctggtggcctgtaccagagggactcccctggacacagaggtgcccatggagcgcgtgtttgactacagtgagtactgggagggggctcggggactgtacgcggccttcgactgcacggccaccatgaagtctggcaactcggacgtgtatgaaaatgagatcccagggggccagtacaccaacctgcacttccaggcccacagcatggggcttggctccaagttcaaggaggtcaagaaggcctatgtggaggccaaccagatgctgggcgatctcatcaaggtgacgccctcctccaagatcgtgggggacctggcccagtttatggtgcagaatggattgagccgggcagaggccgaagctcaggcggaagagctgtcctttccccgctccgtggtggagttcctgcagggctacatcggtgtcccccatggggggttccccgaaccctttcgctctaaggtactgaaggacctgccaagggtggaggggcggcctggagcctccctccctcccctggatctgcaggcactggagaaggagctggtagaccggcatggggaggaggtgacgccggaagatgtgctctcagcagctatgtaccccgatgtgtttgcccacttcaaggacttcactgccacctttggccccctggatagcctgaatactcgcctcttcctgcagggacccaagatcgcagaggagtttgaggtggagctggagcggggcaagacgctgcacatcaaagccctggccgtgagcgacctgaaccgggccggccagaggcaggtcttctttgagctcaatgggcagctgcggtccatcttggtcaaggacacccaggccatgaaggagatgcacttccaccccaaggccctaaaggacgtgaagggccagatcggggcgcccatgcctgggaaggtgatagacatcaaagtggtggcaggggccaaggtggccaagggccagcccctgtgtgtgctcagtgccatgaagatggagactgtggtgacctcacccatggagggtactgtccgcaaggttcatgtgaccaaggacatgacactggaaggtgacgacctcatcctggagatcgagtgatcttgccccagaccggcagcctggccatccccaagccttcaacagaagctgtgctgccacggcaggcccaggccagccagtgcccgaggccaggaaggccgggccgtggaggtcctgtccacagctggacaggagagacaccgcctgcggtggttcattcctttcagccatcgtcctttcctccggcggacagctgcttacatgttcatctcttgccaaataagggtcccctcctcactggagactacaagtggtgggtcaggtggtcctaggacccaggggaggtttaggggtcctatctcctgggggaaggggagatctaagatgtcccaggtcctgggaagtttactcaataaagctggctttcccctgccaaaaaaaaaaaaaaaaaaaaaaaaaaaaaaaaaaaaaaaaaaaaaaaaaaaaaaa

SEQ ID NO: 38 - DNA, 3394 bp, Homo sapiens
agcggatccggtcgccgcgggcgtccgcggagcagcttccccccgcgcctctcttcgcctcgcctcgggacccgcggtccccgctcttgctggatttttcaagccacattcaattgataggatgaaaaaatttaagagaaggctatccctcacactccgaggaagtcagactattgatgaatcattgtctgaattggctgaacaaatgactattgaagaaaacagcagcaaggataatgagcctattgtgaagaatggcaggcctccaacgtctcacagtatgcattccttcctccaccagtacacaggatctttcaagaagcccccattgcggagaccacacagtgttattggagggagccttggctccttcatggcaatgcccagaaatggaagcagattagatattgttcatgaaaatctaaaaatgggatcagatggtgagagtgaccaagcttctgggacatcatctgatgaagtccagtcacctacaggtgtttgtctcagaaatcgtatacatagacggatctcaatggaggatttaaataagcggttatcactgcctgcagacatcagaatacctgatggatatcttgaaaagttgcagataaacagtccaccatttgaccaaccaatgagtcgaaggtctcgtagagcttccttatcagaaattggctttggaaaaatggaaacctacatcaaattggaaaagcttggagagggtacatatgcaacagtatataaaggaagaagtaaattgacagagaatttggtggcattaaaagagatccgattggaacatgaagaaggtgcaccctgcacagctataagagaagtttcactattaaaggatttaaaacatgcaaatatagtaaccttacatgacattgttcacacagataaatccttgactttggtgtttgagtatctggataaagacctgaaacagtacatggatgactgtggaaacatcatgagtatgcacaacgtaaagctgtttctgtaccaaattctacgtggtttggcatattgccatagaagaaaggtattgcatcgagacttgaaaccacagaacctcctcattaatgagaaaggagaattaaagctagcagattttggactagcccgagccaagtcagttcccacaaagacctactcaaatgaagttgtcacactatggtaccggccacctgatgtgcttcttggttcctcggagtactcaacacagattgacatgtggggtgttggttgcattttctttgaaatggcttctggaagacctttatttccaggatcaaccgtggaagatgaactgcacttaattttccgactgctaggaactccatctcaggaaacttggccaggtatttcttcaaatgaggagttcaagaactacaactttccaaaatataaaccacagcctctaattaaccacgcacccaggttagactctgaaggaattgagttgataacaaaatttcttcagtatgaatctaagaaaagggtttcagctgaagaggccatgaaacatgtgtactttcgaagtctgggaccaagaatacatgctttaccagaaagtgtatcaatattcagtttgaaagagattcagttgcaaaaggacccgggttttcgaaattcttcttatccagagacaggacatgggaagaacagaagacagagcatgctcttttaagtctgataacatggtttcaagcccagcccccagcctttcttaccaatcaaggactcagaactgaaggcaattatttcttttggtggacttggaatctccgtttgtctacactgtcctcttcacagtggagtctttttatttcagacctacagattgtttttatatttgttgtcacagtgtgacaattttttgtacagtcggttttaaggtcttctctgccattagagccagtaggttacagctattgatgtaactatagctgcaatttttgtgcaggactgagaaacacagtgcattatttattgcggaatcattgctgctaaataactactgtctgtttatttaacacaacaattgaatgcctgaagaagttgcagtggctttattatatgtgaatgcatctgtttctcctcacgaattgttttactgctgcattttttgtttttaaaattaaactgcagtgtttcctggaatgtaaatttagctttgcttaatagtaaaataagtgagccatcttgaacactttaagtttatactatataatttcttaatgaacattggcaaatagagtagctaagagattgacaagcacattacgtttagataagcatgtgatgcagaagttgattcaagcaagtgaatccctgacattttgaggatctcacattagacaccaacaaattaaagctccaaaattatttattttttatgtgttcctcactttagaaacgtctactgcatattactggatatttgtatactaatacacttacctattttacattgtgttttaaatttagttaaaacttaaccgtagaagtctgttcaaacgcgaaaggtcttgctttgtttgttttaaatacatttattttattagaaaggatgatatttcattggggaatagtttctttcatttgggggctatgtgtttcaataacattcacttggaatcagctgaaagctgtaatatgtctttgaaatgagccatgataaattacaaacaagagaaatgagaacttacaggattgcttgaaattattgtgggtattatctgtttaccagagatactaaatattagttttaattatgcatctctttgaacatcataaacacactttggtgttggtaacagtattttaagcatttttgttcacttttaaggactatttgtttagtgcatcttacaaactataaatcttaattggtatattttgaaatggtcgtgtaaatttttgccttatatatcatgaaaatagtcataacttaatttcttttcctgaaattcactgtaaaacattcaggggtcctaccatttctgttcaggaaatcttgtagtttattgttgttatttaacagtattaagtggatttttgtatatttcattttaactttatttgtgaatagtgattgtggcatgtttggggtgttattttaatatttcacgatactaaaattttaattttaaaaaattctggaagaaacagattcatgtgtaatggtgaatattggaccactactgcttttcacatttgtaaattggttttatgttaaaccttgtagcctaacaaactgctgttctttctaactgacagacatgtattaatattgtactttgaaggttataaatatgtggaaattccttcattttgttttgaaatttataataacaaaatctccatgttgttaaaaaaaaaaaaaaaa

SEQ ID NO: 39 - DNA, 2429 bp, Homo sapiens
ggctgctgctgggagcgcggaggggaagggagcccccagccctgctgggccggcccaggcccctccgcggctcccccttccactacccacctgcccggcaccccctccccagtggttgttaaccccgggactccccaagcccagcctctgtgtgcagcagccccaggcgggctaagtctccaagatgcccttggtggatttcttctgcgagacctgctctaagccttggctggtgggctggtgggaccagttcaaaaggatgttgaaccgtgagctcacacacctgtcagaaatgagcaggtccggaaaccaggtctcagagtacatttccacaacattcctggacaaacagaatgaagtggagatcccatcacccacgatgaaggaacgagaaaaacagcaagcgccgcgaccaagaccctcccagccgcccccgccccctgtaccacacttacagcccatgtcccaaatcacagggttgaaaaagttgatgcatagtaacagcctgaacaactctaacattccccgatttggggtgaagaccgatcaagaagagctcctggcccaagaactggagaacctgaacaagtggggcctgaacatcttttgcgtgtcggattacgctggaggccgctcactcacctgcatcatgtacatgatattccaggagcgggacctgctgaagaaattccgcatccctgtggacacgatggtgacatacatgctgacgctggaggatcactaccacgctgacgtggcctaccataacagcctgcacgcagctgacgtgctgcagtccacccacgtactgctggccacgcctgcactagatgcagtgttcacggacctggagattctcgccgccctcttcgcggctgccatccacgatgtggatcaccctggggtctccaaccagttcctcatcaacaccaattcggagctggcgctcatgtacaacgatgagtcggtgctcgagaatcaccacctggccgtgggcttcaagctgctgcaggaggacaactgcgacatcttccagaacctcagcaagcgccagcggcagagcctacgcaagatggtcatcgacatggtgctggccacggacatgtccaagcacatgaccctcctggctgacctgaagaccatggtggagaccaagaaagtgaccagctcaggggtcctcctgctagataactactccgaccgcatccaggtcctccggaacatggtgcactgtgccgacctcagcaaccccaccaagccgctggagctgtaccgccagtggacagaccgcatcatggccgagttcttccagcagggtgaccgagagcgcgagcgtggcatggaaatcagccccatgtgtgacaagcacactgcctccgtggagaagtctcaggtgggttttattgactacattgtgcacccattgtgggagacctgggcggaccttgtccacccagatgcccaggagatcttggacactttggaggacaaccgggactggtactacagcgccatccggcagagcccatctccgccacccgaggaggagtcaagggggccaggccacccacccctgcctgacaagttccagtttgagctgacgctggaggaggaagaggaggaagaaatatcaatggcccagataccgtgcacagcccaagaggcattgactgcgcagggattgtcaggagtcgaggaagctctggatgcaaccatagcctgggaggcatccccggcccaggagtcgttggaagttatggcacaggaagcatccctggaggccgagctggaggcagtgtatttgacacagcaggcacagtccacaggcagtgcacctgtggctccggatgagttctcgtcccgggaggaattcgtggttgctgtaagccacagcagcccctctgccctggctcttcaaagcccccttctccctgcttggaggaccctgtctgtttcagagcatgccccgggcctcccgggcctcccctccacggcggccgaggtggaggcccaacgagagcaccaggctgccaagagggcttgcagtgcctgcgcagggacatttggggaggacacatccgcactcccagctcctggtggcggggggtcaggtggagaccctacctgatccccagacctctgtccctgttcccctccactcctcccctcactcccctgctcccccgaccacctcctcctctgcctcaaagactcttgtcctcttgtccctcctgagaaaaaaagaaaacgaaaagtggggtttttttctgttttctttttttcccctttccccctgcccccacccacggggcctttttttggaggtgggggctggggaatgaggggctgaggtcccggaagggattttatttttttgaattttaattgtaacatttttagaaaaagaacaaaaaaaaaaaaaaaaaaaa

SEQ ID NO: 40 - DNA, 2264 bp, Homo sapiens
gctgccgctgctttggctgctgcgtcatacgccccagagccgccgggacggaggggctgggcctggggaccccccggcctccgcctgcacgcccccccacgcccggacgtgccctctccgcgcgggggactcgcctaggtctcctacgtctgcccctgcccggctcccggcggccccagctgtcaccggcccccccaggatgcaatggcgcagcccccccggctgagccgctctggtgcctcctcactttgggacccagcttctcctgctcccacctctggccccaggcctcggctttgggagggtcaagatgtgctggccagatggactgatgggctgctatacttgggtaccatcaaaaaggtggacagtgctagggaggtgtgtctggtccagtttgaggatgattcgcagtttctggttctatggaaagacattagccctgctgccctccctggagaggaactcctctgttgtgtctgtcgctctgagactgtggtccctgggaaccggctggtcagctgtgagaagtgtcgccatgcttatcaccaggactgccatgttcccagggctccagcccctggagagggagagggcacatcctgggtatgccgccagtgtgtctttgcgatcgccaccaagaggggaggtgccctgaagaagggcccctatgcccgggccatgctgggtatgaagctttctctgccatatggactgaaggggctggactgggatgctggacatctgagcaaccgacagcagagttactgttactgtggtggccctggggagtggaacctgaaaatgctgcagtgccggagctgcctgcagtggttccatgaggcctgcacccagtgtctgagcaagcccctcctctatggggacaggttctatgaatttgaatgctgtgtgtgtcgcgggggccctgagaaagtccggagactacagcttcgctgggtggatgtggcccatcttgtcctgtatcacctcagtgtttgctgtaagaagaaatactttgattttgatcgtgagatcctccccttcacttctgagaattgggacagtttgctcctgggggagctttcagacacccccaaaggagaacgttcttccaagctcctctctgctcttaacagccacaaggaccgtttcatttcagggagagagattaagaagaggaaatgtttgtttggtctccatgctcggatgcctccccctgtggagccccctactggagatggagcactcaccagcttcccttcagggcagggccctgggggaggggtctcacgtcccctggggaagcgccggaggccggagccagagcccctgaggaggaggcagaaggggaaagtggaggagctggggccaccctcagcagtgcgcaatcagcccgagccccaggagcagagggagcgggctcatctgcagagggcactgcaggcctcagtgtctccaccatcccccagccctaaccagagttaccagggcagcagcggctacaacttccggcccacagatgcccgctgcctgcccagcagccccatccggatgtttgcttccttccacccttctgccagcaccgcagggacctctggggacagtggacccccagacaggtcacccctggaacttcacattggtttccccacagacatccctaaaagtgccccccactcgatgactgcctcatcttcctcagtttcatccccatccccaggtcttcctagacgctcagcacccccttctcccctgtgccgtagtttgtctcctgggactgggggaggagtccgaggtggggttggttacctgtcccgaggggaccctgtccgggtccttgctcggagagtacggcctgatggctctgtgcagtacctggttgagtggggaggagggggcatcttctgaacagcctgcctctgcccagctccccattcacacacaccggcactttcataccctgacctctgacctcacctacagctgggatgtacctggagagatagggggtagttctccctactgcccaggctggaatccaagagtggggagtggggaagaggccctcttctctaccctccttcatgattcctgacccctcccatccttcccatttcctttgatgttattttgttacagctttttaaatattttttaaaattatttaacccctgggggcagagactgaggagggaggatgataagggatcccggactctgtatgattgaaataaagagaaataaacaaaaaaaaaaaaaaaaaa

SEQ ID NO: 41 - DNA, 2332 bp, Homo sapiens
gcacagcagctgcacgcgccgtggctccggatctcttcgtctttgcagcgtagcccgagtcggtcagcgccggaggacctcagcagccatgtcgaagccccatagtgaagccgggactgccttcattcagacccagcagctgcacgcagccatggctgacacattcctggagcacatgtgccgcctggacattgattcaccacccatcacagcccggaacactggcatcatctgtaccattggcccagcttcccgatcagtggagacgttgaaggagatgattaagtctggaatgaatgtggctcgtctgaacttctctcatggaactcatgagtaccatgcggagaccatcaagaatgtgcgcacagccacggaaagctttgcttctgaccccatcctctaccggcccgttgctgtggctctagacactaaaggacctgagatccgaactgggctcatcaagggcagcggcactgcagaggtggagctgaagaagggagccactctcaaaatcacgctggataacgcctacatggaaaagtgtgacgagaacatcctgtggctggactacaagaacatctgcaaggtggtggaagtgggcagcaagatctacgtggatgatgggcttatttctctccaggtgaagcagaaaggtgccgacttcctggtgacggaggtggaaaatggtggctccttgggcagcaagaagggtgtgaaccttcctggggctgctgtggacttgcctgctgtgtcggagaaggacatccaggatctgaagtttggggtcgagcaggatgttgatatggtgtttgcgtcattcatccgcaaggcatctgatgtccatgaagttaggaaggtcctgggagagaagggaaagaacatcaagattatcagcaaaatcgagaatcatgagggggttcggaggtttgatgaaatcctggaggccagtgatgggatcatggtggctcgtggtgatctaggcattgagattcctgcagagaaggtcttccttgctcagaagatgatgattggacggtgcaaccgagctgggaagcctgtcatctgtgctactcagatgctggagagcatgatcaagaagccccgccccactcgggctgaaggcagtgatgtggccaatgcagtcctggatggagccgactgcatcatgctgtctggagaaacagccaaaggggactatcctctggaggctgtgcgcatgcagcacctgattgcccgtgaggcagaggctgccatctaccacttgcaattatttgaggaactccgccgcctggcgcccattaccagcgaccccacagaagccaccgccgtgggtgccgtggaggcctccttcaagtgctgcagtggggccataatcgtcctcaccaagtctggcaggtctgctcaccaggtggccagataccgcccacgtgcccccatcattgctgtgacccggaatccccagacagctcgtcaggcccacctgtaccgtggcatcttccctgtgctgtgcaaggacccagtccaggaggcctgggctgaggacgtggacctccgggtgaactttgccatgaatgttggcaaggcccgaggcttcttcaagaagggagatgtggtcattgtgctgaccggatggcgccctggctccggcttcaccaacaccatgcgtgttgttcctgtgccgtgatggaccccagagcccctcctccagcccctgtcccacccccttcccccagcccatccattaggccagcaacgcttgtagaactcactctgggctgtaacgtggcactggtaggttgggacaccagggaagaagatcaacgcctcactgaaacatggctgtgtttgcagcctgctctagtgggacagcccagagcctggctgcccatcatgtggccccacccaatcaagggaagaaggaggaatgctggactggaggcccctggagccagatggcaagagggtgacagcttcctttcctgtgtgtactctgtccagttcctttagaaaaaatggatgcccagaggactcccaaccctggcttggggtcaagaaacagccagcaagagttaggggccttagggcactgggctgttgttccattgaagccgactctggccctggcccttacttgcttctctagctctctaggcctctccagtttgcacctgtccccaccctccactcagctgtcctgcagcaaacactccaccctccaccttccattttcccccactactgcagcacctccaggcctgttgctatagagcctacctgtatgtcaataaacaacagctgaagcaaaaaaaaaaaaaaaaaaaaaaaaaaaaaa

SEQ ID NO: 42 - DNA, 2038 bp, Homo sapiens
gagaccttcaccatagcgttcgctcaactccagaaccttccgacctccgctagttcctgcgggcctttgcccgcttcccggtgcaccctccccgggagacacctcagacccccgacagcctgggcaggctcggtgcctgcgggtgcgttcctgatcacccctcccctcttccctccccctcatcctccattcccttgttttcaccctctgtcctctgcccgtcactccccttgtcacctcttggagccccctcctaaccagcggccagtgggtttcccataccccaggatgtgagcctctttaacctgtaatgctgtggctagcccttggcccctttcctgccatggagaaccaggtgctggtaattcgcatcaagatcccaaatagtggcgcggtggactggacagtgcactccgggccgcagttactcttcagggatgtgctggatgtgataggccaggttctgcctgaagcaacaactacagcatttgaatatgaagatgaagatggtgatcgaattacagtgagaagtgatgaggaaatgaaggcaatgctgtcatattattattccacagtaatggaacagcaagtaaatggacagttaatagagcctctgcagatatttccaagagcctgcaagcctcctggggaacggaacatacatggcctgaaggtgaatactcgggccggaccctctcaacacagcagcccagcagtctcagattcacttccaagcaatagcttaaagaagtcttctgctgaactgaaaaaaatactagccaatggccagatgaatgaacaagacatacgatatcgggacactcttggtcatggcaacggaggcacagtctacaaagcatatcatgtcccgagtgggaaaatattagctgtaaaggtcatactactagatattacactggaacttcagaagcaaattatgtctgaattggaaattctttataagtgcgattcatcatatatcattggattttatggagcattttttgtagaaaacaggatttcaatatgtacagaattcatggatgggggatctttggatgtatataggaaaatgccagaacatgtccttggaagaattgcagtagcagttgttaaaggccttacttatttgtggagtttaaagattttacatagagacgtgaagccctccaatatgctagtaaacacaagaggacaggttaagctgtgtgattttggagttagcactcagctggtgaattctatagccaagacgtatgttggaacaaatgcttatatggcgcctgaaaggatttcaggggagcagtatggaattcattctgatgtctggagcttaggaatctcttttatggagcttgctcttgggaggtttccatatcctcagattcagaaaaaccagggatctttaatgcctctccagcttctgcagtgcattgttgatgaggattcgcccgtccttccagttggagagttctcggagccatttgtacatttcatcactcagtgtatgcgaaaacagccaaaagaaaggccagcacctgaagaattgatgggccacccgttcatcgtgcagttcaatgatggaaatgccgccgtggtgtccatgtgggtgtgccgggcgctggaggagaggcggagccagcaggggcccccgtgaggctgccgcagggcactgaaagcccaggaccagtaaccaaggagaacaacccacccgtcgcccttctccgtatgctgcctgcgccagaagagctttgctgggccctggcttccctgccctcgccttcacctctgtcagcaggtggccttgcctggggagccccatgtgtggcccaccccaccaggccatccccataccttctggtttgaaggcgctgacactggcagagaggtaaagggtggggcattgagaatggaggctcccagggtccctgcccacttctgttttcctaatgtttttctctataaagggtcaggcccgtcagcatcactgatgggaataaaagtattaatgctttgtgacaaaaaaaaaaaaaaaaaa

SEQ ID NO: 43 - DNA, 2517 bp, Homo sapiens
gcagcagcagcctcccgcgtcgctgtcgctgttgcctccgccacctcctccgccgccgcgcgcccctcggagttccgcgccccaccatgcccaacatcgtgctgttcagcggcagctcgcatcaggacctatcccagcgcgtggccgaccgcctgggcctggagctgggcaaggtggtcacgaagaagttcagcaaccaggagaccagcgtggagattggtgaaagcgtgagaggggaagatgtctacatcatccagagcggctgcggggaaattaacgacaacctgatggaactcctcatcatgatcaatgcctgcaagattgcgtcatcatccagagtaactgccgtgatcccgtgtttcccatacgcccgacaagataaaaaggacaagagtcgtgccccaatttctgcaaaacttgtggccaatatgctgtcggtggctggggcggatcacatcatcaccatggacctgcatgcttctcagatacagggattctttgatattcctgtggataatttgtatgcggagcccgcagtcctgcagtggattcgggaaaacattgccgagtggaagaactgtatcattgtttcacctgacgcagggggagccaaaagggttacatcaattgcagacaggttgaatgtggaatttgctttgatccacaaagagaggaagaaggcgaatgaagtggaccggatggtcctggtgggcgacgtgaaggaccgtgtggccatcctcgtggatgacatggctgacacttgcggcaccatctgccatgctgcggacaagctgctgtcagctggagccaccaaagtgtatgctatccttacccatgggatcttctctggaccagctatttccagaataaataatgccgcctttgaggctgttgtcgtcacaaacacaattccgcaagaggacaaaatgaaacactgcaccaagattcaggtcattgacatttccatgatcttggccgaagcaatccgaaggacacacaatggggaatccgtgtcctacctgttcagccatgtcccgctataaatccagaatgggaagtgtccagcaagcctactctgacttctgacttgtttttgttttctggatttttagctgtaggtattcagcaatgataggttaatcactggcaaaagcatcagatctttgtatatgctaagatttattgtttccccttctaaagctcaagatcatttctttccagtttttggggaaatggtggtggttatttggtctttaagtgaactgtcttaaatgagaaacgtttttgtcattttgacttttaacaggtacaggtgatctcttcctttgttctttcagtactttgaggcgacaactttcaagtatataatttcattgtggaagtcatagtttatatatttcgaggttgccaaaggtgacttcagattaaagccttctgtgtaaatatatactgataatgcctatggacatttgggtaaaaccctgtatagaattaattatccttttactttggagtgaaccttggaaaatttataattataataccatggattttgaattttccttttttttttttttttttggataactcagtttcagataaaccatcttggttactgtgcttaatttggaccaaattttatttagcttaatatggacactgacacattttggggggtatacattagacatatcagagcagtgtatttctggatcattttttaaatgacctcttctaaaacataactgtcacttacctgaaatgctgcatcctaaaattccaaaattatattgagcaatcgccaaggcctaaagccaactgacttaaaggtaatcatttcagctaagattaaatttaaagcctaagaatgtatagagctagttttaaaataatgatctcagatttttaaaaaggatataggaacctgcattgtcattctctgaattaagaactgatggtttctatcattatttagccccacctttgtattttaaaatccttcagaatacatttatgaaccaatgcgactggacttagccacacacaatggaaattcagaccttgactatttggtgtttccagttcacaaaggtgatgaagactgtcttgggagcagcttaatcccaaaatttgtacatttcttgctgctcctggcgtggaaacttaagtgagaccaccaaatacattggtcctgtccaattctactgaatgggggtggacctggcatttatctggccaaaaacaggagccagagaaatatgaatataccaaagttgtttgtttagcctccaacttaaattacattagtcaacttatagatactcatatgatcacttttctttttagatactacatcaactagattcaggagtatatcatttgcagtgcttgtattggtttaaaatgtaagattttaagatcctctaacactgtactaaaacatttcaataaaatcattctgactgcgttaaaaaaaaaaaaaaaaaaaaaaaaaaaaaaaacaaaaaaaaaaaaaaa

SEQ ID NO: 44 - DNA, 1076 bp, Homo sapiens
ccatctttccagcctcagtcggacgggcgcggagacgcttctggaaggaacgccgcgatggctgcgcagggagagccccaggtccagttcaaacttgtattggttggtgatggtggtactggaaaaacgaccttcgtgaaacgtcatttgactggtgaatttgagaagaagtatgtagccaccttgggtgttgaggttcatcccctagtgttccacaccaacagaggacctattaagttcaatgtatgggacacagccggccaggagaaattcggtggactgagagatggctattatatccaagcccagtgtgccatcataatgtttgatgtaacatcgagagttacttacaagaatgtgcctaactggcatagagatctggtacgagtgtgtgaaaacatccccattgtgttgtgtggcaacaaagtggatattaaggacaggaaagtgaaggcgaaatccattgtcttccaccgaaagaagaatcttcagtactacgacatttctgccaaaagtaactacaactttgaaaagcccttcctctggcttgctaggaagctcattggagaccctaacttggaatttgttgccatgcctgctctcgccccaccagaagttgtcatggacccagctttggcagcacagtatgagcacgacttagaggttgctcagacaactgctctcccggatgaggatgatgacctgtgagaatgaagctggagcccagcgtcagaagtctagttttataggcagctgtcctgtgatgtcagcggtgcagcgtgtgtgccacctcattattatctagctaagcggaacatgtgcttcatctgtgggatgctgaaggagatgagtgggcttcggagtgaatgtggcagtttaaaaaataacttcattgtttggacctgcatatttagctgttttggaacgcagttgattccttgagtttcatatataagactgctgcagtcacatcacaatattcagtggtgaaatcttgtttgttactgtcattcccattccttttcgtttagaatcagaataaagttgtatttcaaataaaaaaaaaaaaaaaaaaa

SEQ ID NO: 45 - DNA, 4781 bp, Homo sapiens
cgggggagggcgcgtccggtttttctcaggggacgttgaaattatttttgtaacgggagtcgggagaggacggggcgtgccccgacgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcagcagttgacctagatgagatgtcgttcacttttactgagctacagaaaaacatagaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatgcaagattatttttggatcatgataaaactcttcagactgattctatagacagttttgaaacacagagaacaccacgaaaaagtaaccttgatgaagaggtgaatgtaattcctccacacactccagttaggactgttatgaacactatccaacaattaatgatgattttaaattcagcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgattcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttattttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtcaaaaaaaaaaaaaaaaaaaaaaaaaa

SEQ ID NO: 46 - DNA, 1655 bp, Homo sapiens
ggtggagcagcggcagcagcagcagccgccgccgccgccgccgctgccgccgccgccggaaagggagaggcaggagagcccgagacttggaaaccccaaagtgtccgcgaccctgcacggcaggctcccttccagcttcatgggcaaagtgtggaaacagcagatgtaccctcagtacgccacctactattacccccagtatctgcaagccaagcagtctctggtcccagcccaccccatggcccctcccagtcccagcaccaccagcagtaataacaacagtagcagcagtagcaactcaggatgggatcagctcagcaaaacgaacctctatatccgaggactgcctccccacaccaccgaccaggacctggtgaagctctgtcaaccatatgggaaaatagtctccacaaaggcaattttggataagacaacaaacaaatgcaaaggttatggttttgtcgactttgacagccctgcagcagctcaaaaagctgtgtctgccctgaaggccagtggggttcaagctcaaatggcaaagcaacaggaacaagatcctaccaacctctacatttctaatttgccactctccatggatgagcaagaactagaaaatatgctcaaaccatttggacaagttatttctacaaggatactacgtgattccagtggtacaagtcgtggtgttggctttgctaggatggaatcaacagaaaaatgtgaagctgttattggtcattttaatggaaaatttattaagacaccaccaggagtttctgcccccacagaacctttattgtgtaagtttgctgatggaggacagaaaaagagacagaacccaaacaaatacatccctaatggaagaccatggcatagagaaggagaggctggaatgacacttacttacgacccaactacagctgctatacagaacggattttatccttcaccatacagtattgctacaaaccgaatgatcactcaaacttctattacaccctatattgcatctcctgtatctgcctaccaggtgcaaagtccttcgtggatgcaacctcaaccatatattctacagcaccctggtgccgtgttaactccctcaatggagcacaccatgtcactacagcccgcatcaatgatcagccctctggcccagcagatgagtcatctgtcactaggcagcaccggaacatacatgcctgcaacgtcagctatgcaaggagcctacttgccacagtatgcacatatgcagacgacagcggttcctgttgaggaggcaagtggtcaacagcaggtggctgtcgagacgtctaatgaccattctccatatacctttcaacctaataagtaactgtgagatgtacagaaaggtgttcttacatgaagaagggtgtgaaggctgaacaatcatggatttttctgatcaattgtgctttaggaaattattgacagttttgcacaggttcttgaaaacgttatttataatgaaatcaactaaaactatttttgctataagttctataaggtgcataaaacccttaaattcatctagtagctgttcccccgaacaggtttattttagtaaaaaaaaaaaacaaaaaacaaaaacaaaagatttttatcaaatgttatgatgcaaaaaaaaaaaaaaaaaa

SEQ ID NO: 47 - DNA, 3486 bp, Homo sapiens
ccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccagtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcgtacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtactgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaaaaaaaaaaaaaaaaa

SEQ ID NO: 48 - DNA, 2150 bp, Homo sapiens
caggacggcaccaagggagctgccccatggacagggccccacagagacagcaccgagcctcacgggagctgctggctgcaaagaagacccacacctcacaaattgaagtgatcccttgcaaaatctgtggggacaagtcgtctgggatccactacggggttatcacctgtgaggggtgcaagggcttcttccgccggagccagcgctgtaacgcggcctactcctgcacccgtcagcagaactgccccatcgaccgcaccagccgaaaccgatgccagcactgccgcctgcagaaatgcctggcgctgggcatgtcccgagatgctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagatagagcacctggtgcagagcgtctgcaagtcctacagggagacatgccagctgcggctggaggacctgctgcggcagcgctccaacatcttctcccgggaggaagtgactggctaccagaggaagtccatgtgggagatgtgggaacggtgtgcccaccacctcaccgaggccattcagtacgtggtggagttcgccaagaggctctcaggctttatggagctctgccagaatgaccagattgtgcttctcaaagcaggagcaatggaagtggtgctggttaggatgtgccgggcctacaatgctgacaaccgcacggtcttttttgaaggcaaatacggtggcatggagctgttccgagccttgggctgcagcgagctcatcagctccatctttgacttctcccactccctaagtgccttgcacttttccgaggatgagattgccctctacacagcccttgttctcatcaatgcccatcggccagggctccaagagaaaaggaaagtagaacagctgcagtacaatctggagctggcctttcatcatcatctctgcaagactcatcgccaaagcatcctggcaaagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtggggctgtccaagtgacctggaagagggactccttgcctctccctatggcctgctggcccacctccctggaccccgttccaccctcacccttttcctttcccatgaaccctggagggtggtccccaccagctctttggaagtgagcagatgctgcggctggctttctgtcagcaggccggcctggcagtgggacaatcgccagagggtggggctggcagaacaccatctccagcctcagctttgacctgtctcatttcccatattccttcacacccagcttctggaaggcatggggtggctgggatttaaggacttctgggggaccaagacatcctcaagaaaacaggggcatccagggctccctggatgaatagaatgcaattcattcagaagctcagaagctaagaataagcctttgaaatacctcattgcatttccctttgggcttcggcttggggagatggatcaagctcagagactggcagtgagagcccataaggacctgtataaaatgaatctggagctttaaaaaaaaaaaaaaaaaaaaaaaaaaaaaaaaaaaaaaaaaaaaaaaaaaaaaaa

SEQ ID NO: 49 - DNA, 648 bp, Homo sapiens
cgccatcatgggagtggacatccgccataacaaggaccgaaaggttcggcgcaaggagcccaagagccaggatatctacctgaggctgttggtcaagttatacaggtttctggccagaagaaccaactccacattcaaccaggttgtgttgaagaggttgtttatgagtcgcaccaaccggccgcctctgtccctttcccggatgatccggaagatgaagcttcctggccgggaaaacaagacggccgtggttgtggggaccataactgatgatgtgcgggttcaggaggtacccaaactgaaggtatgtgcactgcgcgtgaccagccgggcccgcagccgcatcctcagggcagggggcaagatcctcactttcgaccagctggccctggactcccctaagggctgtggcactgtcctgctctccggtcctcgcaagggccgagaggtgtaccggcatttcggcaaggccccaggaaccccgcacagccacaccaaaccctacgtccgctccaagggccggaagttcgagcgtgccagaggccgacgggccagccgaggctacaaaaactaaccctggatcctactctcttattaaaaagatttttgctgacaaaaaaaaaaaaaaaaaaaaaaaaaaaaaaaaaa

SEQ ID NO: 50 - DNA, 616 bp, Homo sapiens
cgaacgcggagagcacgccatgaaggcctcgggcacgctacgagagtacaaggtagtgggtcgctgcctgcccacccccaaatgccacacgccgcccctctaccgcatgcgaatctttgcgcctaatcatgtcgtcgccaagtcccgcttctggtactttgtatctcagttaaagaagatgaagaagtcttcaggggagattgtctactgtgggcaggtgtttgagaagtcccccctgcgggtgaagaacttcgggatctggctgcgctatgactcccggagcggcacccacaacatgtaccgggaataccgggacctgaccaccgcaggcgctgtcacccagtgctaccgagacatgggtgcccggcaccgcgcccgagcccactccattcagatcatgaaggtggaggagatcgcggccagcaagtgccgccggccggctgtcaagcagttccacgactccaagatcaagttcccgctgccccaccgggtcctgcgccgtcagcacaagccacgcttcaccaccaagaggcccaacaccttcttctaggtgcagggccctcgtccgggtgtgccccaaataaactcaggaacgccaaaaaaaaaaaaaaaaaaa

SEQ ID NO: 51 - DNA, 4222 bp, Homo sapiens
gctgcggagggagccgccgccatgtctgcgcatctgcaatggatggtcgtgcggaactgctccagtttcctgatcaagaggaataagcagacctacagcactgagcccaataacttgaaggcccgcaattccttccgctacaacggactgattcaccgcaagactgtgggcgtggagccggcagccgacggcaaaggtgtcgtggtggtcattaagcggagatccggccagcggaagcctgccacctcctatgtgcggaccaccatcaacaagaatgctcgcgccacgctcagcagcatcagacacatgatccgcaagaacaagtaccgccccgacctgcgcatggcagccatccgcagggccagcgccatcctgcgcagccagaagcctgtgatggtgaagaggaagcggacccgccccaccaagagctcctgagccccctgcccccagagcaataaagtcagctggctttctcacctgcctcgactgggcctccctttttgaaacgctctggggagctctggccctgtgtgttgtcattcaggccatgtcatcaaaactctgcatgtcaccttgtccatctggaggtgatgtcaatggctggccatgcaggaggggtggggtagctgccttgtccctggtgagggcaagggtcactgtcttcacagaaaaagtttgctgacttgtgattgagacctactgtcccattgtgaggtggcctgaagaatcccagctggggcagtggcttccattcagaagaagaaaggccttttctagcccagaagggtgcaggctgagggctgggccctgggccctggtgctgtagcacggtttggggacttggggtgttcccaagacctgggggacgacagacatcacgggaggaagatgagatgacttttgcatccagggagtgggtgcagccacatttggaggggatgggctttacttgatgcaacctcatctctgagatgggcaacttggtgggtggtggcttataactgtaagggagatggcagccccagggtacagccagcaggcattgagcagccttagcattgtccccctactcccgtcctccaggtgtccccatccctcccctgtctctttgagctggctcttgtcacttaggtctcatctcagtggccgctcctgggccaccctgtcacccaagctttcctgattgcccagccctcttgtttcctttggcctgtttgctccctagtgtttattacagcttgtgaggccaggagtttgagaccatcctgggcaacatgatgagacaccgtctctaaaataaaattagctgggtgtggtggtgcaccgcctgtggtcccagctcctcagaggttgagtagaggctgaggtgagcggagcacttgagccaagagtatgaggctgcagtgagcccatgagccccaccactacactccagcctggaagacaccatgacacacaggcctggatggggaaagagtcctgctgttgatcctcacatgtttcctgggcacctaactctgtcagccactgccagggaccaaggatccagcatccatggcacccctggttcctgccatcctggggtacccgattcaaagagggactctgctccctgtctgagaccacccccggctctgactgagagtaaggggactgtcagggcctcgacttgccattggttggggtcgtacggggctgggagccctgcgttttgaggcagaccactgcccttccgacctcagtcctgtctgctccagtcttgcccagctcgaaggagagcagatctgaccacttgccagcccctgtctgctgtgaattaccatttccttcgtccttcccttagttgggtctattagctcagattgagaggtgttgccttaaaactgagttgggtgacttggtacctgctcaggaccccccgcactgtcccaatcccactcaggcccacctccagctggcctcactccgctggtgacttcgtacctgctcaggagcccccactgtcccagtcccactcaggcccatctctggctggcctcactgcgctgggactccgccttcataaggagagctcactgctcacgttagtagatggccccttctcgtgaggcctctcccctggcacctgcttcagttgtcctccacagcactgatttgcagcccacaagctggcaggtttatctgtctcatgtttgtcttgtgctggtgggcaaggggtttgtctagcacaccagcatataatgagatgcttgatgaatggtgcatattgaatgtataaagcccaccggtcctgagagtttgctcactggagactttctggagatggagtctcgctctgttgcccaggctggcgagtgcagtggcgcgatcttggctcactgcaacctccacctcctgggttcaggcgattctcctgcctcagcctcccgagtagctgggattacaggtgggtgtcaccacacccagctcagtattgtatttttagcagagatggggtttcaccattttgcccaggctggtttggaactcctgacttcaaattacccacctgcctcagcctcccaaagtgctggcattacaggcgctcgaggctttctgatgtggctgctgctgctcagaaggccttgtccttaaccacctccttgcctgccctggaggcttgtgcctctaggccccaccccctgtggagtcctgctggctttctccatccctatctgaatcctccctgctgtgtggcctcccctggtctcatccgtaacacagcccagcttagtgggcctctgttcctgcgggtggccagcctgtctgtgtggctgggctggggaggccacgtctggtatctgaatgctatcggtgggttggggtggaggaaccaggagagggctggagggagggagatggtctcagccccacagagtttggagtcctcagtgtgctgagcaaacgtggagacaccatttccctcctctagacctcatcttggagagagagatgttggatggggccatctattccagctttattcacacaaatcatgtctgttggcctggaaattggaaaaccagttaaaccaaaaacatgatattaagaaaacaggcaggctcaccatagtaaaaatgctgaaagccaaagacaaaattgggagaacaaaagaaaagcgtcttgtcacatacagaaggtccctgataaagttagtagctgccctcatcagaaaccaggcccaggcagtggggacacatccagagtgctgaaagaacctcccccaggtcatcctatccccaagagtgatgcccggcagcattcccagctcagggctaatggttcacggaagccaggaatcaaactgcctgggttccagtcccagctctgccagttatgcccagctgtggggacttgggcagctcgtttagtagcaccgtgcctcagtttcccatatgtaaaaggccattttgagtgcctttcacagccctgcataaggcaggtgtctcagtgttcactgctgtctctccagctcttagtccagtagctgcatggtgagtgagcgtagggcgcaccctggaaggctgccaagcccaaagttgtgcagagcgctggggactccagactccccacagcagcagagactcgggactgaggcatcctctgttcacaggacatgctggcatctactgggtcagggctctgctgctcggtggctgtgcaaccttgggcaagttcctcaacctctctgtgtcttcgtaccctcatctgtaacatgcgtgtcgatagaccctactactcagggttgatgagaagattaaatgtgcaaaacctgcttgactgtgcccacaaatcctgattgtaggaataaattaatgactttttataaatattttgatcagatggactcagatgatcacagatgtcttcacatgcctatgactaatttgtacacaaactaatgctcgtgtttcccaagcacctggaagacatgccagatccatgtgcagtaatgcctggtggctccaggtctgccccgccgtcctgtggggctgtgagctttcccagcctcctgcccgtgtttgtgaatatcattctgtcctcagctgcatttccagcccaggctgtttggcgctgcccaggaatggtatcaattcccctgtttctcttgtagccagttactagaataaaatcatctactttaaaaaaaaaaaaaaaaaaaaaaaaaaaaaaaaaaaaaaaa

SEQ ID NO: 52 - DNA, 439 bp, Homo sapiens
gaatggctcccgcaaagaagggtggcgagaagaaaaagggccgttctgccatcaacgaagtggtaacccgagaatacaccatcaacattcacaagcgcatccatggagtgggcttcaagaagcgtgcacctcgggcactcaaagagattcggaaatttgccatgaaggagatgggaactccagatgtgcgcattgacaccaggctcaacaaagctgtctgggccaaaggaataaggaatgtgccataccgaatccgtgtgcggctgtccagaaaacgtaatgaggatgaagattcaccaaataagctatatactttggttacctatgtacctgttaccactttcaaaaatctacagacagtcaatgtggatgagaactaatcgctgatcgtcagatcaaataaagttataaaattgccttcaaaaaaaaaaaaaaaaaa

SEQ ID NO: 53 - DNA, 489 bp, Homo sapiens
gccatctccttctcggcatcatggccgccctcagaccccttgtgaagcccaagatcgtcaaaaagagaaccaagaagttcatccggcaccagtcagaccgatatgtcaaaattaagcgtaactggcggaaacccagaggcattgacaacagggttcgtagaagattcaagggccagatcttgatgcccaacattggttatggaagcaacaaaaaaacaaagcacatgctgcccagtggcttccggaagttcctggtccacaacgtcaaggagctggaagtgctgctgatgtgcaacaaatcttactgtgccgagatcgctcacaatgtttcctccaagaaccgcaaagccatcgtggaaagagctgcccaactggccatcagagtcaccaaccccaatgccaggctgcgcagtgaagaaaatgagtaggcagctcatgtgcacgttttctgtttaaataaatgtaaaaactgccaaaaaaaaaaaaaaa

SEQ ID NO: 54 - DNA, 506 bp, Homo sapiens
agctggcctccggggcaccgaccgctataaggccagtcggactgcgacacagcccatcccctcgaccgctcgcgtcgcatttggccgcctccctaccgctccaagcccagccctcagccatggcatgccccctggatcaggccattggcctcctcgtggccatcttccacaagtactccggcagggagggtgacaagcacaccctgagcaagaaggagctgaaggagctgatccagaaggagctcaccattggctcgaagctgcaggatgctgaaattgcaaggctgatggaagacttggaccggaacaaggaccaggaggtgaacttccaggagtatgtcaccttcctgggggccttggctttgatctacaatgaagccctcaagggctgaaaataaatagggaagatggagacaccctctgggggtcctctctgagtcaaatccagtggtgggtaattgtacaataaattttttttggtcaaatttaaaaaaaaaaaaaaaaaa

SEQ ID NO: 55 - DNA, 2060 bp, Homo sapiens
gggagctctggtgcagtctccgcctgtcagtgccggcagtcgtccgcggcgcccgccccggtcccgcactggtgcagccatgtcctcttccccgtgggagcctgcgaccctgcgccgggtgttcgtggtgggggttggcatgaccaagtttgtgaagcctggagctgagaattcaagagactaccctgacttggcagaagaagcaggcaagaaggctttagctgatgcacagatcccttattcagcagtggaccaggcatgtgttggctatgtttttggtgtggcagaatgtgtcttggctcttgggtttgagaagatgagtaagggaagccttggaataaaattttcagatagaaccattcccactgataagcatgttgacctcctgatcaataagtatggattgtctgctcacccagttgctcctcagatgtttgggtatgctggaaaagaacatatggaaaaatatggaacaaaaattgaacactttgcaaaaattggatggaaaaatcataaacattcagttaataacccgtattcccagttccaagatgaatacagtttagatgaagtgatggcatctaaagaagtttttgattttttgactatcttacaatgttgtcccacttcagatggtgctgcagcagcaattttggccagtgaagcatttgtacagaagtatggcctgcaatccaaagctgtggaaattttggcacaagaaatgatgactgatttgccaagctcgtttgaagaaaaaagcattattaaaatggttggctttgatatgagtaaagaagctgcaagaaaatgctatgagaaatctggcctgacaccaaatgatattgacgtaatagaacttcacgattgcttttctaccaacgaactccttacttatgaagcactgggactctgtccagaaggacaaggtgcaacgctggttgatagaggagataatacatatggaggaaagtgggtcataaatcctagtggtggactgatttcaaagggacacccactaggcgctacaggaggacattcctgctcttgaaatttcctgataacccggacaagttcatggaaagcttgatctacattcatcctaatctttgctgatgcctccatgtatgttaccttaagttgccgtgctaactgccgtccttcttcctgtgttacctgtctttgatgatccagatctgctttattaccaattaaaatcattgggaactcatcacgatcctttactctgagaatctgtctttgaaacttatagatttcttcaaaactgcctctatctgtgactgaaaagaccaacaggaagccctcgccagtcctcatatactgttctctcatggctccaagctcttcttgtcctgcttatccaaaatatctagccgggctgctctgtcatctatcacacactgctttgtgtaagaatcttcattggttggatcataatccgttacaaaataggactcccaacttaggaaacaaaacatttgtaatatgattgaagactcctgcatgcctcctcaataatattcccctctctcctcccccaacaccaagaggtaatcaatatcctgaatttggtatctgtcattctcatgcatttatacaacattagtaatcatatgatagtattcctaagctatgttttggagaggacaccactgcaaatcttcagccctgtactagcttttatcggtagccatcaattgcagttttaatagaaatacaaatacatcaagtgtctgttctttaatttgtaaagaaaatttgttttctagcgtgacaccctcaaggtaccgtatctgtatgtaaatgttgtgcccaatgctacacagacattattttaaaactattttctgcctttggtaaatagactaaactgttttctttattcctcaaaagaattagatcatacttcagtgttgattttgttgctaatgggacatggagatgtctggatgataataaatattcctgtggtatggtcactgaaaaaaaaaaaaaaaaaaaaaaaaaaaaaaaaaaaaaaaaaaaaaaaaaaaaaaaaaaaa

SEQ ID NO: 56 - DNA, 2454 bp, Homo sapiens
ccagcgcgtcgccccctgcatccgtggcctccactggagctgggcaggaccctacccagtgaatctggagaaaacaaacttgggagacagacgaaagcttagggcacattggaggacagcgcagctgtggctcccatttttggagatgcagtcgaatttgagctcacagggaggtgtggttgcctcctggggatggaaaggcttcctttctccacctctgtaactggtgcttctgagaagtaaatggtatttggatcctgacctcagacgtgaatttgggtcttctgtgcttaggagcagaaagagcccaggaggggcctgttcctttacttcttgggggaaacgcaatgcgtggcctgacttctcatgacgggaaaggctactccaccttctctgtactcctggaggggagtcttgttcacatgtttaccagcggccaggacaaggaagagaaaagaaatgagccgtcagactgctacagcattacctaccggtacctcgaagtgtccaccatcccagagggtgcctgccctgactggcacaactgcatccaacaatgacttggcgagtctttttgagtgtccagtctgctttgactatgtgttaccgcccattcttcaatgtcagagtggccatcttgtttgtagcaactgtcgcccaaagctcacatgttgtccaacttgccggggccctttgggatccattcgcaacttggctatggagaaagtggctaattcagtacttttcccctgtaaatatgcgtcttctggatgtgaaataactctgccacacacagaaaaagcagaccatgaagagctctgtgagtttaggccttattcctgtccgtgccctggtgcttcctgtaaatggcaaggctctctggatgctgtaatgccccatctgatgcatcagcataagtccattacaaccctacagggagaggatatagtttttcttgctacagacattaatcttcctggtgctgttgactgggtgatgatgcagtcctgttttggctttcacttcatgttagtcttagagaaacaggaaaaatacgatggtcaccagcagttcttcgcaatcgtacagctgataggaacacgcaagcaagctgaaaattttgcttaccgacttgagctaaatggtcataggcgacgattgacttgggaagcgactcctcgatctattcatgaaggaattgcaacagccattatgaatagcgactgtctagtctttgacaccagcattgcacagctttttgcagaaaatggcaatttaggcatcaatgtaactatttccatgtgttgaaatggcaatcaaacattttctggccagtgtttaaaacttcagtttcacagaaaataaggcacccatctgtctgccaacctaaaactctttcggtaggtggaagctagacacatgaaggtaaataaaaagaaaggctgttaaatacaggaaacagttgcatgtagtaacactaatatatttaaaaataagtcaacagtaaaccactgaaaaaatatatgtatatacacccaagatgggcatcttttgtattaagaaaggaagcattgtaaaataattctgagttttgtgtttgttgtagattgattgtattgttgaaaaagtttgtttttgcgtgggagtgtgtgcctgcgtgggtgtgtgcgtgtttgggtttttttcctttaactgacaagccatcttgagtggtcatgggccactgcttttccctttgtgagtcaatacatagtgctgctgtgtgctttttttgtgtgtatttgctaatttttattaattttagtttttcattaaataaatttgacttttctgtaattcaggtttttcctttttttgtaccattttaaagttagtatcttttgatatgcatatttgtttatggtaaaaaatttataacgtgttcaatattttcttttcccccattaatcagttcattagaaatattttaaaatcagctattttgtgaagccatgagttccagaaagtaaaggtgacatcggaaaaataatcaaaagctatttaaagcatctataaggtgctctctttctgtcttctacagatgagtcacacctttgagcttaatctttgaaaggttagagaataaattgatttttataaatactgcaaatcaggcttttgtttcctttttcagatatcttggacaaatcacatattttaaaatttgttcttgtatttattggttttgcagaagaaggcatcgtcatgcacagtatttgtaattaaaagcaaatcatttgtttaaaaaggcagtttgcaaaaaatgtttttggtcttttataattctcattaaaagaatatctgtcaaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 57 - DNA, 3326 bp, Homo sapiens
ctgagcctcccgtgccggccggccggccgggggaacaggcgggcgctcggggggcgctcggggggcggggggagttccggttccggttctttgtgcggctgcatcggcggctccgggaagatggcggcccgggcgggtttccagtctgtggctccaagcggcggcgccggagcctcaggaggggcgggcgcggctgctgccttgggcccgggcggaactccggggcctcctgtgcgaatgggcccggctccgggtcaagggctgtaccgctccccgatgcccggagcggcctatccgagaccaggtatgttgccaggcagccgaatgacacctcagggaccttccatgggaccccctggctatggggggaacccttcagtccgacctggcctggcccagtcagggatggatcagtcccgcaagagacctgcccctcagcagatccagcaggtccagcagcaggcggtccaaaatcgaaaccacaatgcaaagaaaaagaagatggctgacaaaattctacctcaaaggattcgtgaactggtaccagaatcccaggcctatatggatctcttggcttttgaaaggaaactggaccagactatcatgaggaaacggctagatatccaagaggccttgaaacgtcccatcaagcaaaaacggaagctgcgaattttcatttctaacactttcaatccggctaagtcagatgccgaggatggggaagggacggtggcttcctgggagcttcgggtagaaggacggctcctggaggattcagccttgtccaaatatgatgccactaaacaaaagaggaagttctcttccttttttaagtccttggtgattgaactggacaaagacctgtatgggccagacaaccatctggtagaatggcacaggaccgccactacccaggagaccgatggctttcaggtgaagcggccgggagacgtgaatgtacggtgtactgtcctactgatgctggattaccagcctccccagtttaaattagacccccgcctagctcgactcctgggcatccatacccagactcgtccagtgatcatccaagcactgtggcaatatattaagacacataagctccaggaccctcacgagcgggagtttgtcatctgtgacaagtacctgcagcagatctttgagtctcaacgtatgaagttttcagagatccctcagcggctccatgccttgcttatgccaccagaacctatcatcattaatcatgtcatcagtgttgacccgaatgatcagaaaaagacagcttgttatgacattgatgttgaagtggatgacaccttgaagacccagatgaattcttttctgctgtccactgccagccaacaggagattgctactctagacaacaagatccatgagacaatagaaaccatcaaccagctgaagactcagcgggagttcatgctgagctttgccagagaccctcagggtttcatcaatgactggcttcagtcccagtgcagggacctcaagacaatgactgatgtggtgggtaacccagaggaggagcgccgagctgagttctacttccagccctgggctcaggaggctgtgtgccgatacttctactccaaggtgcagcagagacgacaagaattagagcaagccctgggaatccggaatacatagggcctctcccacagccctgattcgactgcaccaattcttgatttgggccctgtgctgcctgcctcatagtatctgccttggtcttgcttggggcgttccaggggatgctgttggttcaaggacaacaccagaatgaagagggtctcacaagacacctgttatcctcttctttcaccctatctcttcccacccccagcttccctttgccccacaaagttcccatgtgcctgtaccctcccctggtctacataggacctctagatagtgttagagagagaacatgtagtggtaatgagtgcttggaatggattgggcctcaggccaggtggtcttcaaggggaccagctaactgatcctgcccttcagagacccaggagttgggagctttcgctccttctccaagactcaggcctgtgggcactctataagctagttgatcttggctctcctgataacagaatccaatttccttccttccctccacaggtttggaacaaactctcccttcacttgttgccctgtagcactacagaaaccctggttcttgggctccactgagccccaggtcagtccccagccctctgggttggcctgctgtcagtgcttctctcactccttagttggggtccacatcagtattggagttttgttctttattgctccctcccagacactccctgtggctgccctttgtgattccctcagatctgccctaatcccgggcatttgggtgggggaatcttgcctttccctttcagagccccagggatctcatctggggaactgtcattgccagcagaggctgttccttcctgctgtttggagatgtgactcattcattcactcactccaccctgcctctgcatcccttaatggagaaacgggcctaaaaccaaacgggtaaaaagccctgggccatccctgtcttcctgtcccttgtctgcccagttgacacctactggtgacttctagggcactgaggagtgaaagcgcctagggctggagaatagcgctgagttgggtttgtgactcttccctctccctgcctcacaggattgtgactccccagcccctgccctcaaagcttcagacccctcaggtagcagcaggaccttgtgatcttggccccttggatctgagatggtttttgcatctttccaggagagcctcacattcttcttccaggttgtatcacccccgagttagcatatcccaggctcgcagactcaacacagcaagggtgggagacagctgggcacaaagggggaattccgttcagcatgggctctaaacccacagaactgacaaagcccctgcttccccaccccctcctcaggctcctgcgagcacacccccacccccaaatccctccctgttctacactggggacagcagaattttctccccgtcttccccttcctgccattttccctcccttgaaaggttgacactggacaaccttggggcagctgagccctggccgcctcctggctggaaccatgagaaggaagctcagtacttcccacagtgtccctgttgataactgtttttattaactgaattgtttttttcatggaccaaacttttttttgtactgtccccttattgatgttacccagttttaataaaagaatcttctgaaaaaaaaaaaaaaaaaa

SEQ ID NO: 58 - DNA, 1454 bp, Homo sapiens
ccggacgcgaggggcggggcgagcgcgggacaaagggaagcgaagccggagctgcgggcgctttttctgcccgcggtgtctcagattcattcttaaggaactgagaacttaatcttccaaaatgtcaaaaagaccatcttatgccccacctcccaccccagctcctgcaacacaaatgcccagcacaccagggtttgtgggatacaatccatacagtcatctcgcctacaacaactacaggctgggagggaacccgagcaccaacagccgggtcacggcatcctctggtatcacgattccaaaacccccaaagccaccagataagccgctgatgccctacatgaggtacagcagaaaggtctgggaccaagtaaaggcttccaaccctgacctaaagttgtgggagattggcaagattattggtggcatgtggcgagatctcactgatgaagaaaaacaagaatatttaaacgaatacgaagcagaaaagatagagtacaatgaatctatgaaggcctatcataattcccccgcgtaccttgcttacataaatgcaaaaagtcgtgcagaagctgctttagaggaagaaagtcgacagagacaatctcgcatggagaaaggagaaccgtacatgagcattcagcctgctgaagatccagatgattatgatgatggcttttcaatgaagcatacagccaccgcccgtttccagagaaaccaccgcctcatcagtgaaattcttagtgagagtgtggtgccagacgttcggtcagttgtcacaacagctagaatgcaggtcctcaaacggcaggtccagtccttaatggttcatcagcgaaaactagaagctgaacttcttcaaatagaggaacgacaccaggagaagaagaggaaattcctggaaagcacagattcatttaacaatgaacttaaaaggttgtgcggtctgaaagtagaagtggatatggagaaaattgcagctgagattgcacaggcagaggaacaggcccgcaaaaggcaggaggaaagggagaaggaggccgcagagcaagctgagcgcagtcagagcagcatcgttcctgaggaagaacaagcagctaacaaaggcgaggagaagaaagacgacgagaacattccgatggagacagaggagacacaccttgaagaaacaacagagagccaacagaatggtgaagaaggcacgtctactcctgaggacaaggagagtgggcaggagggggtcgacagtatggcagaggaaggaaccagtgatagtaacactggctcggagagcaacagtgcaacagtggaggagccaccaacagatcccataccagaagatgagaaaaaagaataagtgttgccttgttttgtgtgttctaaatactttttttaatgaaaaaatgttttttggttttaatggggaaaaaaaaaaaaaaaaaaaaaaaaaaaaa

SEQ ID NO: 59 - DNA, 940 bp, Homo sapiens
cttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgatcataccctaatgatcccagcaagataacgtcctgtcttctaagatgtgcatcaagcctggtacatactgaaaaccctataaggtcctggataatttttgtttgattattcattgaagaaacatttattttccaattgtgtgaagtttttgactgttaataaaagaatctgtcaaccatcaaaaaaaaaaaaaaa

SEQ ID NO: 60 - DNA, 1172 bp, Homo sapiens
cccgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaaaaaaaaaaaaaaaaaaaaaaaaaaaaa

SEQ ID NO: 61 - DNA, 4388 bp, Homo sapiens
agcgggagcgggctcggttttgggccgcggcgggagcgggagtcgccgccactcgagtgcgcaggcgcctggcgattaccggtctcaccatggagcggaaagtgcttgcgctccaggcccgaaagaaaaggaccaaggccaagaaggacaaagcccaaaggaaatctgaaactcagcaccgaggctctgctccccactctgagagtgatctaccagagcaggaagaggagattctgggatctgatgatgatgagcaagaagatcctaatgattattgtaaaggaggttatcatcttgtgaaaattggagatctattcaatgggagataccatgtgatccgaaagttaggctggggacacttttcaacagtatggttatcatgggatattcaggggaagaaatttgtggcaatgaaagtagttaaaagtgctgaacattacactgaaacagcactagatgaaatccggttgctgaagtcagttcgcaattcagaccctaatgatccaaatagagaaatggttgttcaactactagatgactttaaaatatcaggagttaatggaacacatatctgcatggtatttgaagttttggggcatcatctgctcaagtggatcatcaaatccaattatcaggggcttccactgccttgtgtcaaaaaaattattcagcaagtgttacagggtcttgattatttacataccaagtgccgtatcacccacactgacattaaaccagagaacatcttattgtcagtgaatgagcagtacattcggaggctggctgcagaagcaacagaatggcagcgatctggagctcctccgccttccggatctgcagtcagtactgctccccagcctaaaccagctgacaaaatgtcaaagaataagaagaagaaattgaagaagaagcagaagcgccaggcagaattactagagaagcgaatgcaggaaattgaggaaatggagaaagagtcgggccctgggcaaaaaagaccaaacaagcaagaagaatcagagagtcctgttgaaagacccttgaaagagaacccacctaataaaatgacccaagaaaaacttgaagagtcaagtaccattggccaggatcaaacgcttatggaacgtgatacagagggtggtgcagcagaaattaattgcaatggagtgattgaagtcattaattatactcagaacagtaataatgaaacattgagacataaagaggatctacataatgctaatgactgtgatgtccaaaatttgaatcaggaatctagtttcctaagctcccaaaatggagacagcagcacatctcaagaaacagactcttgtacacctataacatctgaggtgtcagacaccatggtgtgccagtcttcctcaactgtaggtcagtcattcagtgaacaacacattagccaacttcaagaaagcattcgggcagagataccctgtgaagatgaacaagagcaagaacataacggaccactggacaacaaaggaaaatccacggctggaaattttcttgttaatccccttgagccaaaaaatgcagaaaagctcaaggtgaagattgctgaccttggaaatgcttgttgggtgcacaaacatttcactgaagatattcaaacaaggcaatatcgttccttggaagttctaatcggatctggctataatacccctgctgacatttggagcacggcatgcatggcctttgaactggccacaggtgactatttgtttgaacctcattcaggggaagagtacactcgagatgaagatcacattgcattgatcatagaacttctggggaaggtgcctcgcaagctcattgtggcaggaaaatattccaaggaatttttcaccaaaaaaggtgacctgaaacatatcacgaagctgaaaccttggggcctttttgaggttctagtggagaagtatgagtggtcgcaggaagaggcagctggcttcacagatttcttactgcccatgttggagctgatccctgagaagagagccactgccgccgagtgtctccggcacccttggcttaactcctaagcccctgcccagcaccacagcagagatcacacactgaccctccgcccttccccttcaagcattttcctcttcccttttcagggtgaagctcttccttcaagagtttctagatcttgtttttttttttaatccaacatgttcatttgggtttgcttacttgaccctgtggagatccccacagccattgggcatcctaggtgaatttggccttggttgggctctgccaaagactaatggactaaaatgtgaaacagcctcttgccctgtacctttccttcccattaggacatcctttaaattataagcatcctttttgaaaagagctatgaaggtgtatgagcccatccttttattcattgactctaagagtcaaattttctagtgcatatcctattgccagcataaggatgaggagggggaaagggtcttaattctatgtacagcagagacattaaacttgctgtgtccgggctgcatcatcttcctggactgtttctgttgttctctgtgttcacattttttcctgcaacttttaagctactgtcttttttaaatagctatatgaacaccaaatttgggtaccattttatcactgttcaaagcactgtcaaattcctttcatcctttaatagttaagatctttgaatcttcagtctgatttttaatgtaagcaaaaacagaaccattgaatagtaatttcttgagaacctcaggtgttctataaacagtcctttcctgtatgtcttctattaccctaagaccagagttattttggttggttgttttgttttattttttgtttttgtatccatggctggcactttactcattgcacttgagtttattgccccataactaaaggatcaggatgatggtagaacggagatctgggtttcagagctttcccatttaagaaaaatagatcttgagattctaattcttttccaaacagtcccctgctttcatgtacagctttttctttaccttacccaaaattctggccttgaagcagttttcctctatggctttgcctttctgattttctcagaggctcgagtctttaatataaccccaaatgaaagaaccaaggggaggggtgggatggcacttttttttgttggtcttgttttgttttgttttttggttggttggttcgttattttttaagattagccattctctgctgctatttccctacataatgtcaatttttaaccataattttgacatgattgagatgtacttgaggcttttttgttttaattgagaaaagactttgcaattttttttttaggatgagcctctcctagacttgacctagaatattacatattcctccagtaagtaatactgaagagcaaaagagaggcaggattggggtcacagccgcttcttcagcatggaccaagtgggccttggggattgcagcgttctcgaagtggctgtaggactcgaatttacagaaagccacagaggtgcaacttgaggctctgctagcaagccaccagtgaggctattgggtaaccacctttctatacaggagattggaatctactttgtcatttatccaccacagtgacaaaggaaaagtggtgccgttatgcaatccatttaactcataaacatattactctgagtaactggccagccattcatcggatccttcattgggtactcctgaaatcagacatgttcctgtagaaagaattttaagttaggctttctatgcacctatcaagaatcaagagaatagattgtatcaaacaacggcagggaaatccttcagcaattctaatccactttgggttttcagctgtttttacatctaaagcaatagactagaactgaattatcttctacatagtaaaatcacaattgtggaattacaggaattctggtgatattaaggtgaaataacaaaacacaaaaggccctattttaacagttgatgtgacagtaagttttaatagaacctgtaacttcattttggaaatgcttctccaccaaataagggctttttcccctatttaaggagccagatggattgaaagatgtggaaataggcagctgtagatcttgatcttccaggtaccccatgtacctttattgagcttaattataatactgtcaaattgccacgatctcactaaaggatttctatttgctgtcagttaaaaataaagccctaaatacatttttattctttctactgagggcattgtctgttttctttgtaaatgccgtacaataaacaaattatttaataacctaaaaaaaaaaaaaaaaaaaaaaaaaaaaaaaaaaaaaaaaaaatcaaaaaaaaaaaaaaaa

SEQ ID NO: 62 - DNA, 1256 bp, Homo sapiens
gaggaatgcggtcgcccccttgggaaagtacatatctgggagaagcaggcggctccgcgctcgcactcccgctcctccgcccgaccgcgcgctcgccccgccgctcctgctgcagccccagggcccctcgccgccgccaccatggacgccatcaagaagaagatgcagatgctgaagctcgacaaggagaacgccttggatcgagctgagcaggcggaggccgacaagaaggcggcggaagacaggagcaagcagctggaagatgagctggtgtcactgcaaaagaaactcaagggcaccgaagatgaactggacaaatattctgaggctctcaaagatgcccaggagaagctggagctggcagagaaaaaggccaccgatgctgaagccgacgtagcttctctgaacagacgcatccagctggttgaggaagagttggatcgtgcccaggagcgtctggcaacagctttgcagaagctggaggaagctgagaaggcagcagatgagagtgagagaggcatgaaagtcattgagagtcgagcccaaaaagatgaagaaaaaatggaaattcaggagatccaactgaaagaggcaaagcacattgctgaagatgccgaccgcaaatatgaagaggtggcccgtaagctggtcatcattgagagcgacctggaacgtgcagaggagcgggctgagctctcagaaggccaagtccgacagctggaagaacaattaagaataatggatcagaccttgaaagcattaatggctgcagaggataagtactcgcagaaggaagacagatatgaggaagagatcaaggtcctttccgacaagctgaaggaggctgagactcgggctgagtttgcggagaggtcagtaactaaattggagaaaagcattgatgacttagaagacgagctgtacgctcagaaactgaagtacaaagccatcagcgaggagctggaccacgctctcaacgatatgacttccatgtaaacgttcatccactctgcctgcttacaccctgccctcatgctaatataagtttctttgcttcacttctcccaagactccctcgtcgagctggatgtcccacctctctgagctctgcatttgtctattctccagctgaccctggttctctctcttagcatcctgccttagagccaggcacacactgtgctttctattgtacagaagctcttcgtttcagtgtcaaataaacactgtgtaagctaaaaaaaaaaaaaaaaaaa

SEQ ID NO: 63 - DNA, 1855 bp, Homo sapiens
cgagccgagaaagaggtcacgaactctgaccccccagaaatacccaaacacagaaagctctctccgccgtgaatctcgatcccacatcccgtcggctttcttcaacctctcttcccggagcgccccccaatccacgagtggcagccgcgggactgtcgcgtcggcgcccgacgccggagtcagcagggcgcaaaagcgccggtagatcatggcaaccatagaagaaattgcacatcaaattattgaacaacagatgggagagattgttacagagcagcaaactgggcagaaaatccagattgtgacagcacttgatcataatacccaaggcaagcagttcattctgacaaatcacgacggctctactccaagcaaagtcattctggccaggcaagattccactccgggaaaagttttccttacaactccagatgcagcaggtgtcaaccagttattttttaccactcctgatctgtctgcacaacacctgcagctcctaacagataattctccagaccaaggaccaaataaggtttttgatctttgcgtagtatgtggagacaaagcatcaggacgtcattatggagcagtaacttgtgaaggctgcaaaggattttttaaaagaagcatccgaaaaaatttagtatattcatgtcgaggatcaaaggattgtattattaataagcaccaccgaaaccgctgtcaatactgcaggttacagagatgtattgcgtttggaatgaagcaagactctgtccaatgtgaaagaaaacccattgaagtatcacgagaaaaatcttccaactgtgccgcttcaacagaaaaaatctatatccgaaaggaccttcgtagcccattaactgcaactccaacttttgtaacagatagtgaaagtacaaggtcaacaggactgttagattcaggaatgttcatgaatattcatccatctggagtaaaaactgagtcagctgtgctgatgacatcagataaggctgaatcatgtcagggagatttaagtacattggccaatgtggttacatcattagcgaatcttggaaaaactaaagatctttctcaaaatagtaatgaaatgtctatgattgaaagcttaagcaatgatgatacctctttgtgtgaatttcaagaaatgcagaccaacggtgatgtttcaagggcatttgacactcttgcaaaagcattgaatcctggagagagcacagcctgccagagctcagtagcgggcatggaaggaagtgtacacctaatcactggagattcaagcataaattacaccgaaaaagaggggccacttctcagcgattcacatgtagctttcaggctcaccatgccttctcctatgcctgagtacctgaatgtgcactacattggggagtctgcctccagactgctgttcttatcaatgcactgggcactttcgattccttctttccaggctctagggcaagaaaacagcatatcactggtgaaagcttactggaatgaactttttactcttggtcttgcccagtgctggcaagtgatgaatgtagcaactatattagcaacatttgtcaattgtcttcacaatagtcttcaacaagcagaggggtaatcaccttaaaatgtcatcaaaaatagatctactagaaggcagcatcacattcccatcttacttatggactcctacccctggttcatgtcttatatgcctgtaatggttataaagcctaccttcaggaaagctatggttgactaattactaatggatgggttttaaacatgtccctctacaataaattaaaatctttattgtaaaactttaaaaaaaaaaaaaaaaaaaaaaaaaaaaaaaaaa

SEQ ID NO: 64 - DNA, 1673 bp, Homo sapiens
cggaagagggggtgaaggccagaggctcggggcttcaagaccgctgtctggagtccccctttccaggccatgtcggggcccacctggctgcccccgaagcagccggagcccgccagagcccctcaggggagggcgatcccccgcggcaccccggggccaccaccggcccacggagcagcactccagccccaccccagggtcaatttttgcccccttccatctgagcagtgttaccaggccccagggggaccggaggatcgggggccggcgtgggtggggtcccatggagtactccagcacacgcaggggctccctgcagacagggggggccttcgccctggaagcctggacgccgagatagacttgctgagcagcacgctggccgagctgaatgggggtcggggtcatgcgtcacggcgaccagaccgacaggcatatgagcccccgccacctcctgcctaccgcacgggctccctgaagccaaatccagcctcgccgctcccagcgtctccctatgggggccccactccagcctcttacactaccgccagcaccccggctggcccagccttccccgtgcaagtgaaggtggcacagccagtgaggggctgcggcccacccaggcggggagcctctcaggcctctgggcccctcccgggcccccactttcctctcccaggccgaggtgaagtctgggggcctggctataggagccagagagagccagggccaggggccaaagaggaagctgctggggtctctggccctgcaggaagaggaagaggaggcgagcacgggccccaggtgcccctgagccagcctccagaggatgagctggataggctgacgaagaagctggttcacgacatgaaccacccgcccagcggggagtactttggccagtgtggtggctgcggagaagatgtggttggggatggggctggggttgtggcccttgatcgcgtctttcacgtgggctgctttgtatgttctacatgccgggcccagcttcgcggccagcatttctacgccgtggagaggagggcatattgcgagggctgctacgtggccaccctggagaaatgtgccacgtgctcccagcccatcctggaccggatcctgcgggctatggggaaggcctaccaccctggctgcttcacctgcgtggtgtgtcaccgcggcctcgacggcatccccttcacagtggatgctacgagccagatccactgcattgaggactttcacaggaagtttgccccaagatgctcagtgtgcggtggggccataatgcctgagccaggtcaggaggagactgtgagaattgttgctctggatcgaagttttcacattggctgttacaagtgcgaggagtgtgggctgctgctctcctctgagggcgagtgtcagggctgctacccgctggatgggcacatcttgtgcaaggcctgcagcgcctggcgcatccaggagctctcagccaccgtcaccactgactgctgagtcttcctagaagtacctgctgggttctcagttccagttcccatcctttgattgatcactctccctgacatccacctgtatgactttgtcaccaaatgctgtcttctctttctccaatcaagaaataataatccctcgagtttacaaaacaaaaaaaaaaaaaaaaaaaaaaa

SEQ ID NO: 65 - DNA, 3483 bp, Homo sapiens
cttgtacgacagaggtggtttgctcttccgttgccccgtggcttcagctcatctttggcaggaaggcgaggcttccgcccggcacaggggatgtccagctcgccgctgtccaagaaacgtcgcgtgtccgggcctgatccaaagccgggttctaactgctcccctgcccagtccgtgttgtccgaagtgccctcggtgccaaccaacggaatggccaagaacggcagtgaagcagacatagacgagggcctttactcccggcagctgtatgtgttgggccatgaggcaatgaagcggctccagacatccagtgtcctggtatcaggcctgcggggcctgggcgtggagatcgctaagaacatcatccttggtggggtcaaggctgttaccctacatgaccagggcactgcccagtgggctgatctttcctcccagttctacctgcgggaggaggacatcggtaaaaaccgggccgaggtatcacagccccgcctcgctgagctcaacagctatgtgcctgtcactgcctacactggacccctcgttgaggacttccttagtggtttccaggtggtggtgctcaccaacacccccctggaggaccagctgcgagtgggtgagttctgtcacaaccgtggcatcaagctggtggtggcagacacgcggggcctgtttgggcagctcttctgtgactttggagaggaaatgatcctcacagattccaatggggagcagccactcagtgctatggtttctatggttaccaaggacaaccccggtgtggttacctgcctggatgaggcccgacacgggtttgagagcggggactttgtctccttttcagaagtacagggcatggttgaactcaacggaaatcagcccatggagatcaaagtcctgggtccttatacctttagcatctgtgacacctccaacttctccgactacatccgtggaggcatcgtcagtcaggtcaaagtacctaagaagattagctttaaatccttggtggcctcactggcagaacctgactttgtggtgacggacttcgccaagttttctcgccctgcccagctgcacattggcttccaggccctgcaccagttctgtgctcagcatggccggccacctcggccccgcaatgaggaggatgcagcagaactggtagccttagcacaggctgtgaatgctcgagccctgccagcagtgcagcaaaataacctggacgaggacctcatccggaagctggcatatgtggctgctggggatctggcacccataaacgccttcattgggggcctggctgcccaggaagtcatgaaggcctgctccgggaagttcatgcccatcatgcagtggctatactttgatgcccttgagtgtctccctgaggacaaagaggtcctcacagaggacaagtgcctccagcgccagaaccgttatgacgggcaagtggctgtgtttggctcagacctgcaagagaagctgggcaagcagaagtatttcctggtgggtgcgggggccattggctgtgagctgctcaagaactttgccatgattgggctgggctgcggggagggtggagaaatcatcgttacagacatggacaccattgagaagtcaaatctgaatcgacagtttcttttccggccctgggatgtcacgaagttaaagtctgacacggctgctgcagctgtgcgccaaatgaatccacatatccgggtgacaagccaccagaaccgtgtgggtcctgacacggagcgcatctatgatgacgattttttccaaaacctagatggcgtggccaatgccctggacaacgtggatgcccgcatgtacatggaccgccgctgtgtctactaccggaagccactgctggagtcaggcacactgggcaccaaaggcaatgtgcaggtggtgatccccttcctgacagagtcgtacagttccagccaggacccacctgagaagtccatccccatctgtaccctgaagaacttccctaatgccatcgagcacaccctgcagtgggctcgggatgagtttgaaggcctcttcaagcagccagcagaaaatgtcaaccagtacctcacagaccccaagtttgtggagcgaacactgcggctggcaggcactcagcccttggaggtgctggaggctgtgcagcgcagcctggtgctgcagcgaccacagacctgggctgactgcgtgacctgggcctgccaccactggcacacccagtactcgaacaacatccggcagctgctgcacaacttccctcctgaccagctcacaagctcaggagcgccgttctggtctgggcccaaacgctgtccacacccgctcacctttgatgtcaacaatcccctgcatctggactatgtgatggctgctgccaacctgtttgcccagacctacgggctgacaggctctcaggaccgagctgctgtggccacattcctgcagtctgtgcaggtccccgaattcacccccaagtctggcgtcaagatccatgtttctgaccaggagctgcagagcgccaatgcctctgttgatgacagtcgtctagaggagctcaaagccactctgcccagcccagacaagctccctggattcaagatgtaccccattgactttgagaaggatgatgacagcaactttcatatggatttcatcgtggctgcatccaacctccgggcagaaaactatgacattccttctgcagaccggcacaagagcaagctgattgcagggaagatcatcccagccattgccacgaccacagcagccgtggttggccttgtgtgtctggagctgtacaaggttgtgcaggggcaccgacagcttgactcctacaagaatggtttcctcaacttggccctgcctttctttggtttctctgaaccccttgccgcaccacgtcaccagtactataaccaagagtggacattgtgggatcgctttgaggtacaagggctgcagcctaatggtgaggagatgaccctcaaacagttcctcgactattttaagacagagcacaaattagagatcaccatgctgtcccagggcgtgtccatgctctattccttcttcatgccagctgccaagctcaaggaacggttggatcagccgatgacagagattgtgagccgtgtgtcgaagcgaaagctgggccgccacgtgcgggcgctggtgcttgagctgtgctgtaacgacgagagcggcgaggatgtcgaggttccctatgtccgatacaccatccgctgaccccgtctgctcctctaggctggccccttgtccacccctctccacaccccttccagcccagggttcccatttggcttctggcagtggcccaactagccaagtctggtgttccctcatcatccccctacctgaacccctcttgccactgccttctaccttgtttgaaacctgaatcctaataaagaattaataactcccaaaaaaaaaaaaaaaaaa

SEQ ID NO: 66 - DNA, 5138 bp, Homo sapiens
agtctgtctcttcgccggttcccggccccgtggatcctacttctctgtcgcccgcggttcgccgccccgctcgccgccgcgatgccagtgtttcatacgcgcacgatcgagagcatcctggagccggtggcacagcagatctcccacctggtgataatgcacgaggagggcgaggtggacggcaaagccattcctgacctcaccgcgcccgtggccgccgtgcaggcggccgtcagcaacctcgtccgggttggaaaagagactgttcaaaccactgaggatcagattttgaagagagatatgccaccagcatttattaaggttgagaatgcttgcaccaagcttgtccaggcagctcagatgcttcagtcagacccttactcagtgcctgctcgagattatctaattgatgggtcaaggggcatcctctctggaacatcagacctgctccttaccttcgatgaggctgaggtccgtaaaattattagagtttgcaaaggaattttggaatatcttacagtggcagaggtggtggagactatggaagatttggtcacttacacaaagaatcttgggccaggaatgactaagatggccaagatgattgacgagagacagcaggagctcactcaccaggagcaccgagtgatgttggtgaactcgatgaacaccgtgaaagagttgctgccagttctcatttcagctatgaagatttttgtaacaactaaaaactcaaaaaaccaaggcatagaggaagctttaaaaaatcgcaattttactctagaaaaaatgagtgctgaaattaatgagataattcgtgtgttacaactcacctcttgggatgaagatgcctgggccagcaaggacactgaagccatgaagagagcattggcctccatagactccaaactgaaccaggccaaaggttggctccgtgaccctagtgcctccccaggggatgctggtgagcaggccatcagacagatcttagatgaagctggaaaagttggtgaactctgtgcaggcaaagaacgcagggagattctgggaacttgcaaaatgctagggcagatgactgatcaagtggctgacctccgtgccagaggacaaggatcctcaccggtggccatgcagaaagctcagcaggtatctcagggtctggatgtgctcacagcaaaagtggaaaatgcagctcgcaagctggaagccatgaccaactcaaagcagagcattgcaaagaagatcgatgctgctcagaactggcttgcagatccaaatggtggaccggaaggagaagagcagattcgaggtgctttggctgaagctcggaaaatagcagaattatgtgatgatcctaaagaaagagatgacattctacgttcccttggggaaatatctgctctgacttctaaattagcagatctacgaagacaggggaaaggagattctccagaggctcgagccttggccaaacaggtggccacggccctgcagaacctgcagaccaaaaccaaccgggctgtggccaacagcagaccggccaaagcagctgtacaccttgagggcaagattgagcaagcacagcggtggattgataatcccacagtggatgaccgtggagtcggtcaggctgccatccgggggcttgtggccgaagggcatcgtctggctaatgttatgatggggccttatcggcaagatcttctcgccaagtgtgaccgagtggaccagctgacagcccagctggctgacctggctgccagaggggaaggggagagtcctcaggcacgagcacttgcatctcagctccaagactccttaaaggatctaaaagctcggatgcaggaggccatgactcaggaagtgtcagatgttttcagcgataccacaactcccatcaagctgttggcagtggcagccacggcgcctcctgatgcgcctaacagggaagaggtatttgatgagagggcagctaactttgaaaaccattcaggaaagcttggtgctacggccgagaaggcggctgcggttggtactgctaataaatcaacagtggaaggcattcaggcctcagtgaagacggcccgagaactcacaccccaggtggtctcggctgctcgtatcttacttaggaaccctggaaatcaagctgcttatgaacattttgagaccatgaagaaccagtggatcgataatgttgaaaaaatgacagggctggtggacgaagccattgataccaaatctctgttggatgcttcagaagaagcaattaaaaaagacctggacaagtgcaaggtagctatggccaacattcagcctcagatgctggttgctggggcaaccagtattgctcgtcgggccaaccggatcctgctggtggctaagagggaggtggagaattccgaggatcccaagttccgtgaggctgtgaaagctgcctctgatgaattgagcaaaaccatctccccgatggtgatggatgcaaaagctgtggctggaaacatttccgaccctggactgcaaaagagcttcctggactcaggatatcggatcctgggagctgtggccaaggtcagagaagccttccaacctcaggagcctgacttcccgccgcctccaccagaccttgaacaactccgactaacagatgagcttgctcctcccaaaccacctctgcctgaaggtgaggtccctccacctaggcctccaccaccagaggaaaaggatgaagagttccctgagcagaaggccggggaggtgattaaccagccaatgatgatggctgccagacagctccatgatgaagctcgcaaatggtccagcaagggcaatgacatcattgcagcagccaagcgcatggctctgctgatggctgagatgtctcggctggtaagagggggcagtggtaccaagcgggcactcattcagtgtgccaaggacatcgccaaggcctcagatgaggtgactcggttggccaaggaggttgccaagcagtgcacagataaacggattagaaccaacctcttacaggtatgtgagcgaatcccaaccataagcacccagctcaaaatcctgtccacagtgaaggccaccatgctgggccggaccaacatcagtgatgaggagtctgagcaggccacagagatgctggttcacaatgcccagaacctcatgcagtctgtgaaggagactgtgcgggaagctgaagctgcttcaatcaaaattcgaacagatgctggatttacactgcgctgggttagaaagactccctggtaccagtaggcacctggctgagcctggctggcacagaaacctctactaaaaagaaggaaaatgatctgagtcccaggagctgcccagagttgctgggagctgaaaaatcacatcctggcctggcacatcagaaaggaatgggggcctcttcaaattagaagacatttatactcttttttcatggacactttgaaatgtgtttctgtataaagcctgtattctcaaacacagttacacttgtgcaccctctatcccaataggcagactgggtttctagcccatggacttcacataagctcagaatccaagtgaacactagccagacactctgctctgcccttgttccctaggggacacttccctgtttctctttccttggctcccattcactcttccagaatcccaagacccagggcccaggcaaatcagttactaagaagaaaattgctgtgcctcccaaaattgttttgagctttccatgttgctgccaaccataccttccttccctgggctgtgctacctgggtccttttcagaagtgagctttgctgctacaggggaaggtggcctctgtggagccccagcatatgggggcctggattcatttcctgcccttcctcagtttaatccttctagtttcccacaatataaaactgtacttcactgtcaggaagaaatcacagaatcacatgattctgcttttaccatgcccctgagcaatgtctgtgctagggaaacttcccgtcccatatcctgcctcagcccgccaaggtagccatcccatgaacacactgtgtcctggtgctctctgccactggaagggcagagtagccagggtgtggccctgccatcttcccagcagggccactcccggcactccatgcttagtcactgcctgcagaggtctgtgctgaggccttatcattcattcttagctcttaattgttcattttgagctgaaatgctgcattttaattttaaccaaaacatgtctcctatcctggtttttgtagccttcctccacatcctttctaaacaagattttaaagacatgtaggtgtttgttcatctgtaactctaaaagatcctttttaaattcagtcctaagaaagaggagtgcttgtcccctaagagtgtttaatggcaaggcagccctgtctgaaggacacttcctgcctaagggagagtggtatttgcagactagaattctagtgctgctgaagatgaatcaatgggaaatactactcctgtaattcctacctccctgcaaccaactacaaccaagctctctgcatctactcccaagtatggggttcaagagagtaatgggtttcatatttcttatcaccacagtaagttcctactaggcaaaatgagagggcagtgtttcctttttggtacttattactgctaagtatttcccagcacatgaaaccttattttttcccaaagccagaaccagatgagtaaaggagtaagaaccttgcctgaacatccttccttcccacccatcgctgtgtgttagttcccaacatcgaatgtgtacaacttaagttggtcctttacactcaggctttcactattcctttataatgaggatgattattttcaaggccctcagcatatttgtatagttgcttgcctgatataaatgcaatattaatgcctttaaagtatgaatctatgccaaagatcacttgttgttttactaaagaaagattacttagaggaaataagaaaaatcatgtttgctctcccggttcttccagtggtttgagacactggtttacactttatgccggatgtgcttttctccaatatcagtgctcgagacacagtgaagcaaattaaaaaaaaaaaaaaaaa

SEQ ID NO: 67 - DNA, 3427 bp, Homo sapiens
cggaggctccccggtagggacggcctgaggccgagggcaccctgagggcggaagaggcaaagcgcctcgcccgccggggcctcggaggtgactaactcctagaggctgcggcggaggggagaagcctggtggtacattccatggcccagtgcctcggccactggaaaatcccacaggggtatatcgagtaggggaccgcgggctccgcggcagaaatgaccgcccttccaagatggcggctcccgtagagacactcccaaggagacagccatgttccttgcctgagagtcactgaaggaaattggaaagcccgcctctccttcgacgcacacgcgcacgcgcacgcgcgcgcacgcacgcaccggcgcggtcatccagcacagtgagcggcccctgggcaccaccagaaaacatttgtgagtgaacgtggagtgatgaccatgtggacactgtctttccagggtttcttggagcctctgcagagtctgggggccaggcttcttagtggaaactgcaggatcttccttctgacccctgcttgcctcgccccagcagctccggttgagtccacagatgccagctcagcttccagcttggggaagagtggggttgtcagcaggagagcctgagggctgaggcccagcgtgaacatggcagctaatggggactctcccccatggtccccggccctggctgcagagggacgtggcagctcatgtgaggcttcagtgtcatttgaggacgtgactgtggacttcagcaaggaggagtggcagcacttggaccctgcccagagacgcctgtactgggatgtgacactagagaactacagccacctgctctcagtggggtaccaaattcccaagtcagaggctgccttcaagttggagcaaggagaggggccatggatgctggagggggaagccccacatcagagctgttcaggtgaggctattgggaaaatgcagcaacagggaattcctggaggaattttcttccactgtgagagatttgatcaacccataggagaagattcattatgttctattttagaagaactgtggcaagataatgaccagctagagcaacgtcaggaaaaccagaataaccttttaagtcatgtgaaagtattgattaaggagaggggctatgaacataaaaacattgaaaaaataattcatgtgactaccaagcttgttccttcaattaaaagactccataactgtgacacaattttgaagcatactttaaactcacataatcataatagaaacagtgcaacaaagaaccttggcaagatttttggaaatggtaacaatttcccccatagcccttcctctactaagaatgagaatgctaaaacaggagcaaattcctgtgaacatgaccactatgaaaaacatctcagccacaaacaagctcccacccaccatcagaaaattcatcctgaggagaagctttatgtgtgtactgaatgtgtaatgggcttcactcagaagtcacatctgtttgagcatcagagaattcatgctggagaaaagtcccgtgaatgtgacaaaagcaacaaagtcttcccccagaaaccccaggttgatgtacatccaagtgtttatacaggagaaaaaccctatctgtgtactcaatgtgggaaagtctttaccctcaaatcaaacctcattacacatcaaaaaattcataccgggcagaaaccctacaaatgcagtgaatgtggaaaagcctttttccagagatcagacctctttagacatctgagaattcatacaggagaaaaaccttatgaatgcagtgaatgtggaaaaggcttctcccagaactcagacctcagtatacatcagaaaactcataccggagagaaacactatgaatgcaatgaatgtgggaaggctttcacaagaaaatcagcactcaggatgcatcagagaatccacacgggagagaaaccttatgtatgcgctgactgtgggaaggccttcatccagaaatcacatttcaacacacatcagagaattcatactggagaaaagccgtatgaatgcagtgactgtgggaaatccttcactaagaagtcacaactccatgtgcatcaaagaattcacaccggagagaaaccctatatatgtacagaatgtggaaaggtcttcactcacaggacaaacctcaccacacatcagaaaactcatactggggaaaaaccctatatgtgtgctgaatgtggaaaggcttttactgaccagtcaaatctcattaaacaccagaaaactcacactggagagaaaccctataagtgcaatggctgtggaaaagccttcatatggaagtcgcgcctcaaaatacatcagaaatctcatattggagagagacactatgaatgcaaggactgcgggaaagccttcatccagaaatcaacactaagcgtgcatcagagaatccatacaggagagaaaccgtacgtttgtcctgaatgcgggaaggcctttatccagaaatcgcacttcattgcgcatcatagaatccatactggagagaagccttatgaatgcagcgactgtgggaaatgcttcactaagaagtcacaactccgtgtgcatcagaaaatccacacaggtgagaagcccaatatatgtgctgaatgtggaaaggccttcactgaccgatcaaatctcataacacatcagaaaatccacactagggagaaaccctatgaatgtggtgactgcgggaaaaccttcacctggaagtcacgcctcaatatacatcagaagtctcatactggagaaagacactatgaatgtagtaaatgtgggaaagctttcatccagaaagccacactaagtatgcatcagataattcatacaggaaagaaaccttatgcttgtacagaatgtcagaaggcctttactgacagatcgaatctcattaaacaccagaaaatgcatagtggagaaaaacgctataaagccagtgactgagaaagtcttcacctggaaatcacaactgggtatgcatcaggtatctaatagcagggaggaggaaggcctgttgctgcaatcattgtacagggggaaatgggtatgagagactgaggcttgagtgaactgaaggcaaacagagccaagtatccttcagtccactggaaagacaagttcctgcatcaccacagtgtatatgcaattctatgcataggaagagcctttagagaaagcatttgagcgaaagtcatgtttggtgacatgattcatacagacctcttgagctcctggaaatagtagaattaagacctgaggagatgactcgtcatctcttatgcttcactttttggataaagaactttttaaattcagtactgattggttcagcatatcctgggacataaaaatactcactactgggaaaaaaaaaaaaaaaa

SEQ ID NO: 68 - DNA, 2698 bp, Homo sapiens
cttcagaaggaggagagacaccgggcccagggcaccctcgcgggcggacccaagcagtgagggcctgcagccggccggccagggcagcggcaggcgcggcccggacctacgggaggaagccccgagccctcggcgggctgcgagcgactccccggcgatgcctcacaactccatcagatctggccatggagggctgaaccagctgggaggggcctttgtgaatggcagacctctgccggaagtggtccgccagcgcatcgtagacctggcccaccagggtgtaaggccctgcgacatctctcgccagctccgcgtcagccatggctgcgtcagcaagatccttggcaggtactacgagactggcagcatccggcctggagtgatagggggctccaagcccaaggtggccacccccaaggtggtggagaagattggggactacaaacgccagaaccctaccatgtttgcctgggagatccgagaccggctcctggctgagggcgtctgtgacaatgacactgtgcccagtgtcagctccattaatagaatcatccggaccaaagtgcagcaaccattcaacctccctatggacagctgcgtggccaccaagtccctgagtcccggacacacgctgatccccagctcagctgtaactcccccggagtcaccccagtcggattccctgggctccacctactccatcaatgggctcctgggcatcgctcagcctggcagcgacaagaggaaaatggatgacagtgatcaggatagctgccgactaagcattgactcacagagcagcagcagcggaccccgaaagcaccttcgcacggatgccttcagccagcaccacctcgagccgctcgagtgcccatttgagcggcagcactacccagaggcctatgcctcccccagccacaccaaaggcgagcagggcctctacccgctgcccttgctcaacagcaccctggacgacgggaaggccaccctgaccccttccaacacgccactggggcgcaacctctcgactcaccagacctaccccgtggtggcagatcctcactcacccttcgccataaagcaggaaacccccgaggtgtccagttctagctccaccccttcctctttatctagctccgcctttttggatctgcagcaagtcggctccggggtcccgcccttcaatgcctttccccatgctgcctccgtgtacgggcagttcacgggccaggccctcctctcagggcgagagatggtggggcccacgctgcccggatacccaccccacatccccaccagcggacagggcagctatgcctcctctgccatcgcaggcatggtggcaggaagtgaatactctggcaatgcctatggccacaccccctactcctcctacagcgaggcctggcgcttccccaactccagcttgctgagttccccatattattacagttccacatcaaggccgagtgcaccgcccaccactgccacggcctttgaccatctgtagttgccatggggacagtgggagcgactgagcaacaggaggactcagcctgggacaggccccagagagtcacacaaaggaatctttatttattacatgaaaaataaccacaagtccagcattgcggcacactccctgtgtggttaatttaatgaaccatgaaagacaggatgaccttggacaaggccaaactgtcctccaagactccttaatgaggggcaggagtcccagggaaagagaaccatgccatgctgaaaaagacaaaattgaagaagaaatgtagcccccagccggtacccaccaaaggagagaagaagcaatagccgaggaacttggggggatggcgaatggttcctgcccgggcccaaggggtgcacagggcacctccatggctccattattaacacaactctagcaattatggaccataagcacttccctccagcccacaagtcacagcctggtgccgaggctctcctcaccagccacccagggagtcacctccctcagcctcccgcctgccccacacggaggctctggctgtcctctttctccactccatttgcttggctctttctacacctccctcttgggcatgggctgagggctggagcgagtccctcagaaattccaccaggctgtcagctgacctcttttgcctgctgctgtgaaggtatagcaccaccccaggtcctcctgcagtgcggcatccccttggcagctgccgtcagccaggccagccccagggagcttaaaacagacattccacagggcctgggcccctgggaggtgaggtgtggtgtgcggcttcacccagggcagaacaaggcagaatcgcaggaaacccgcttccccttcctgacagctcctgccaagccaaatgtgcttcctgcagctcacgcccaccagctactgaagggacccaaggcaccccctgaagccagcgatagagggtccctctctgctccccagcagctcctgcccccaaggcctgactgtatatactgtcaatgaaactttgtttgggtcaagcttccttctttctaccccccagactttggcctctgagtgaaatgtctctctttgccctgtggggcttctctccttgatgcttctttctttttttaaagacaacctgccattaccacatgactcaataaaccattgctcttcaaaaaaaaaaaaaaaaaa

SEQ ID NO: 69 - DNA, 4310 bp, Homo sapiens
ggaaaaggaatatattcactaaggattctatctgcttactgctacagacctatgtgttaaggaattcttctcctcctccttgcgtagaagttgatcagcactgtggtcagactgcatttatcttgtcattgccagaagaaatcttggacagaatgtaacagtacgtctctctctgattgcgatggaaggtgataaactgatactcctttattaaagttacatcgcactcaccacagaaaaccattctttaaagtgaatagaaaccaagcccttgtgaacacttctattgaacatgactcatggagaagagcttggctctgatgtgcaccaggattctattgttttaacttacctagaaggattactaatgcatcaggcagcagggggatcaggtactgccgttgacaaaaagtctgctgggcataatgaagaggatcagaactttaacatttctggcagtgcatttcccacctgtcaaagtaatggtccagttctcaatacacatacatatcaggggtctggcatgctgcacctcaaaaaagccagactgttgcagtcttctgaggactggaatgcagcaaagcggaagaggctgtctgattctatcatgaatttaaacgtaaagaaggaagctttgctagctggcatggttgacagtgtgcctaaaggcaaacaggatagcacattactggcctctttgcttcagtcattcagctctaggctgcagactgttgctctgtcacaacaaatcaggcagagcctcaaggagcaaggatatgccctcagtcatgattctttaaaagtggagaaggatttaaggtgctatggtgttgcatcaagtcacttaaaaactttgttgaagaaaagtaaagttaaagatcaaaagcctgatacgaatcttcctgatgtgactaaaaacctcatcagagataggtttgcagagtctcctcatcatgttggacaaagtggaacaaaggtcatgagtgaaccgttgtcatgtgctgcaagattacaggctgttgcaagcatggtggaaaaaagggctagtcctgccacctcacctaaacctagtgttgcttgtagccagttagcattacttctgtcaagcgaagcccatttgcagcagtattctcgagaacacgctttaaaaacgcaaaatgcaaatcaagcagcaagtgaaagacttgctgctatggccagattgcaagaaaatggccagaaggatgttggcagttaccagctcccaaaaggaatgtcaagccatcttaatggtcaggcaagaacatcatcaagcaaactgatggctagcaaaagtagtgctacagtgtttcaaaatccaatgggtatcattccttcttcccctaaaaatgcaggttataagaactcactggaaagaaacaatataaaacaagctgctaacaatagtttgcttttacatcttcttaaaagccagactatacctaagccaatgaatggacacagtcacagtgagagaggaagcatttttgaggaaagtagtacacctacaactattgatgaatattcagataacaatcctagttttacagatgacagcagtggtgatgaaagttcttattccaactgtgttcccatagacttgtcttgcaaacaccgaactgaaaaatcagaatctgaccaacctgtttccctggataacttcactcaatccttgctaaacacttgggatccaaaagtcccagatgtagatatcaaagaagatcaagatacctcaaagaattctaagctaaactcacaccagaaagtaacacttcttcaattgctacttggccataagaatgaagaaaatgtagaaaaaaacaccagccctcagggagtacacaatgatgtgagcaagttcaatacacaaaattatgcaaggacttctgtgatagaaagccccagtacaaatcggactactccagtgagcactccacctttacttacatcaagcaaagcagggtctcccatcaatctctctcaacactctctggtcatcaaatggaattccccaccatatgtctgcagtactcagtctgaaaagctaacaaatactgcatctaaccactcaatggaccttacaaaaagcaaagacccaccaggagagaaaccagcccaaaatgaaggtgcacagaactctgcaacgtttagtgccagtaagctgttacaaaatttagcacaatgtggaatgcagtcatccatgtcagtggaagagcagagacccagcaaacagctgttaactggaaacacagataaaccgataggtatgattgatagattaaatagccctttgctctcaaataaaacaaatgcagttgaagaaaataaagcatttagtagtcaaccaacaggtcctgaaccagggctttctggttctgaaatagaaaatctgcttgaaagacgtactgtcctccagttgctcctggggaaccccaacaaagggaagagtgaaaaaaaagagaaaactcccttaagagatgaaagtactcaggaacactcagagagagctttaagtgaacaaatactgatggtgaaaataaaatctgagccttgtgatgacttacaaattcctaacacaaatgtgcacttgagccatgatgctaagagtgccccattcttgggtatggctcctgctgtgcagagaagcgcacctgccttaccagtgtccgaagactttaaatcggagcctgtttcacctcaggatttttctttctccaagaatggtctgctaagtcgattgctaagacaaaatcaagatagttacctggcagatgattcagacaggagtcacagaaataatgaaatggcacttctagaatcaaagaatctttgcatggtccctaagaaaaggaagctttatactgagccattagaaaatccatttaaaaagatgaaaaacaacattgttgatgctgcaaacaatcacagtgccccagaagtactgtatgggtccttgcttaaccaggaagagctgaaatttagcagaaatgatcttgaatttaaatatcctgctggtcatggctcagccagcgaaagtgaacacaggagttgggccagagagagcaaaagctttaatgttctgaaacagctgcttctctcagaaaactgtgtgcgagatttgtccccgcacagaattaactctgtggctgacagtaaaaagaaaggacacaaaaataatgtgaccaacagcaaacctgaatttagcatttcttctttaaatggactgatgtacagttccactcagcccagcagttgcatggataacaggacattttcatacccaggtgtagtaaaaactcctgtgagtcctactttccctgagcacttgggctgtgcagggtctagaccagaatctgggcttttgaatgggtgttccatgcccagtgagaaaggacccattaagtgggttatcactgatgcggagaagaatgagtatgaaaaagactctccaagattgaccaaaaccaacccaatactatattacatgcttcaaaaaggaggcaattctgttaccagtcgagaaacacaagacaaggacatttggagggaggcttcatctgctgaaagtgtctcacaggtcacagccaaagaagagttacttcctactgcagaaacgaaagcttctttctttaatttaagaagcccttacaatagccatatgggaaataatgcttctcgcccacacagcgcaaatggagaagtttatggacttctgggaagcgtgctaacgataaagaaagaatcagaataaaatgtacctgccatccagttttggatctttttaaaactaatgagtatgaacttgagatctgtataaataagagcatgatttgaaaaaaagcatggtataattgaaacttttttcattttgaaaagtattggttactggtgatgttgaaatatgcatactaatttttgcttaacattagatgtcatgaggaaactactgaactagcaattggttgtttaacacttctgtatgcgtcagataacaactgtgagtagcctatgaatgaaattcttttataaatattaggcataaattaaaatgtaaaactccattcatagtggattaatgcattttgctgcctttattagggtactttattttgcttttcagaagtcagcctacataacacatttttaaagtctaaactgttaaacaactctttaaaggataattatccaataaaaaaaaacctagtgctgattcacagcttattatccaattcaaaaataaattagaaaaatatatgcttacatttttcacttttgctaaaaaaaaaaaaaaa

SEQ ID NO: 70 - DNA, 1103 bp, Homo sapiens
gcgggggtggggatcggtccgggggaggcctgaggccgctggcttgtgcgctgtctccgccgcccccctctttcgccgccgccgccgccgccccgggcatgtcgtccaactgcaccagcaccacggcggtggcggtggcgccgctcagcgccagcaagaccaagaccaagaagaagcatttcgtgtgccagaaagtgaagctattccgggccagcgagccgatcctcagcgtcctgatgtggggggtgaaccacacgatcaatgagctgagcaatgttcctgttcctgtcatgctaatgccagatgacttcaaagcctacagcaagatcaaggtggacaatcatctcttcaataaggagaacctgcccagccgctttaagtttaaggagtattgccccatggtgttccgaaaccttcgggagaggtttggaattgatgatcaggattaccagaattcagtgacgcgcagcgcccccatcaacagtgacagccagggtcggtgtggcacgcgtttcctcaccacctacgaccggcgctttgtcatcaagactgtgtccagcgaggacgtggcggagatgcacaacatcttaaagaaataccaccagtttatagtggagtgtcatggcaacacgcttttgccacagttcctgggcatgtaccgcctgaccgtggatggtgtggaaacctacatggtggttaccaggaacgtgttcagccatcggctcactgtgcatcgcaagtatgacctcaagggttctacggttgccagagaagcgagcgacaaggagaaggccaaggacttgccaacattcaaagacaatgacttcctcaatgaagggcagaagctgcatgtgggagaggagagtaaaaagaacttcctggagaaactgaagcgggacgttgaggagattctcgttctgtccccaggcaggagaatcgcttgaacccaggaggcggaggttgcggtgaccctccagcttgggcaacatgttatgaatgaaactccatctcaaaaaataaaaaaaaaaaaaggaagcaaaaaaaaaaaaaaaaaaaaaaaaaaaaaaaaaaaaaaaaaaaaaaaaaaaaaaaaaaaaaaaaaa

SEQ ID NO: 71 - DNA, 569 bp, Homo sapiens
ggagcccatcatggcgacgccccctaagcggcgggcggtggaggccacgggggagaaagtgctgcgctacgagaccttcatcagtgacgtgctgcagcgggacttgcgaaaggtgctggaccatcgagacaaggtatatgagcagctggccaaataccttcaactgagaaatgtcattgagcgactccaggaagctaagcactcggagttatatatgcaggtggatttgggctgtaacttcttcgttgacacagtggtcccagatacttcacgcatctatgtggccctgggatatggttttttcctggagttgacactggcagaagctctcaagttcattgatcgtaagagctctctcctcacagagctcagcaacagcctcaccaaggactccatgaatatcaaagcccatatccacatgttgctagaggggcttagagaactacaaggcctgcagaatttcccagagaagcctcaccattgacttcttccccccatcctcagacattaaagagcctgaatgcctttgaaaaaaaaaaaaaaaaaaaaaaaaaaaaaaaaaaaaaaaa

SEQ ID NO: 72 - DNA, 3694 bp, Homo sapiens
agtgcgggtagtgggtttggagaagttccgaggcggcggtggcgccggtcaggacaaggatagcggaaccgggccctgggcttgtcgctcaccatgccgacagtagaggagctttaccgcaattatggcatcctggccgatgccacggagcaagtgggccagcataaagatgcctatcaagtgatattggatggtgtgaaaggtggtactaaggaaaagcgattagcagctcaatttattccgaaattctttaagcattttccagaattggctgattctgctatcaatgcacagttagacctctgtgaggatgaagatgtatctattcgacgtcaagcaattaaagaactgcctcaatttgccactggagaaaatcttcctcgagtggcagatatactaacgcaacttttgcagacagatgactctgcagaatttaacctagtgaacaatgccctattaagtatatttaaaatggatgcaaaagggactttaggtgggttgttcagccaaatacttcaaggagaggacattgttagagaacgagcaattaaattcctttctacaaaacttaagactttaccagatgaagtcttaacaaaggaagtggaagagcttatactaactgaatccaaaaaggtcctagaagatgtgactggtgaagaatttgttctatttatgaagatactgtctgggttaaaaagcttacagacagtgagtggaagacagcaacttgtagagttggtggctgaacaggccgacctagaacagaccttcaatccctcggatcctgactgtgtggacaggctcttacagtgcactcggcaggcagtacccctcttctctaaaaatgtccattccacaaggtttgtgacatatttctgtgagcaggttctccctaacctcggtaccttgactaccccagtggaaggtcttgatatacagttggaggtattgaaattgttggcggagatgagttcattttgtggtgacatggaaaaactagaaacaaatttaaggaaactatttgataagttattggaatacatgcccctccctccagaagaggcagaaaatggagagaatgctggtaatgaagaacccaagctacagttcagttatgtggaatgtttgttgtacagttttcaccagttgggccgaaaacttccagatttcttaacagccaaactgaatgcagaaaagctcaaagatttcaaaatcaggctgcagtactttgcacggggcctgcaagtttatatcagacaacttcgcttagctctccagggtaaaacgggtgaggccttaaaaacagaagagaacaagattaaagtcgttgcattgaaaataacaaacaatatcaatgttttaatcaaggatctcttccacattcctccttcttataagagcacagtaacactatcctggaaacctgtacaaaaggttgagattgggcaaaagagagccagtgaagatacaacttcaggttcaccacccaagaaatcttcagcaggaccaaaaagagatgccaggcagatttataaccctcccagtgggaaatatagcagcaatttgggcaactttaattatgagaggagccttcaggggaagtagaggtggccgaggttggggcacacgaggaaatcgtagtcggggaagactctactgaataagacatcagcattcttcagcattgtcatgagcttaatatacttaaattctactactcattggattgccggggatgtccctttaaacagactgctgccttcagctaaaaacttaatgttctttatacctttgtatgtatgacctacttttgtaacagaccatggttgtgtccaaggtaaaaccacagtgatatttttggatgctttgtctgcaatcttgacttgtttttgcagtatcattattcagacttcaaattgtgaatcttttaaacatcttgataatttgttgttgagagctgttcattctaaaatgtaatgaaattcagtctagttctgctgataaagatcatcagttttgaaaggttactgattttcctcttccctcttagttttttacccaatatatggagaagagtaatggtcaatcttaacattttgttttaattgtttaataaagctgctgggcagtggtgcagcattcctacctagtgtcataaaagcaaaatacttacatagctttcttaaaatataggaatgacattacatttttaggagaaagtaagttgctttgcaccgcctacttaattcttttccatatattgtgatacaaacttttgaatatggaatcttactatttgaatagaaatgtgtatgtataatatacatacatacataagcatatatgtgtgtgtgtgtgtgtatatatatatatatgcatgctgtgaaacttgactacacaacataaatcactttttaaattccaggaacgggtagtctgacacggtgattatccttttgaggctgaatccgttattaacttgttatttaggtttttactcccagtagcaagggattctaagttagttgcacttacatgattattgttatttaaaactaagaataaaggctgcattttcaaagataaattggaattgctgttggtgaaataacaaccaaaatactgaatctgatgtacatacaggtttctacaggaagagatggtataatttacaatttggagatttaataaccagggctacccagaaaaagtgacttgataacatggtaccaataagtaagggatgctctctcggtttgcttttgccactttcaagattttaacttctcaggttattaatcaaaattattgtataagttagccaatagaatttttaggttaaaacaacagatggggggtttgtggagtgtttaatgtcatgggcatttttagtagcatagaccctttgttctgcatttgaatgtttcgtatatttttgtttcacagttaatcttccctccccaagtttgctattcaaatcaactgcctgaatgacatttctagtagtctgatgtatttttctgaggaatagtttgtgattccaatgcaggtgtcttcattaccattacctctacactgcagaagaagcaaaactcctttattagaattactgcacatgtgtatggggaaaatagttctgaaaggctagaatgatacaagtgagcaaaagttggtcagcttggctatggagtggtggcaataatctctaaacattccaaaagaccatgagctgaacctaaactcccttggaatctgaacaaaggaatataaaattgccatttgaaaactgaccagctaatctggacctcagagatagatcagccagtggcccaaagccatttcaagtacagaaattatagagactacagctaaataaatttgaacattaaatataattttaccactttttgtctttataagcatatttgtaaactcagaactgagcagaagtgactttactttctcaagtttgatactgagttgactgttcccttatccctcacccttccccttccctttcctaaggcaatagtgcacaacttaggttatttttgcttccgaatttgaatgaaaaacttaatgccatggatttttttcttttgcaagacacctgtttatcatcttgtttaaatgtaaatgtccccttatgcttttgaaataaatttccttttgtaaaaaaaaaaaaaaaaaa

SEQ ID NO: 73 - DNA, 2736 bp, Homo sapiens
gcgaccgctccccggcgggagccagcgaaggtttccatgtcagaggccgatggagaactgaagattgccacctacgcacaaaggccattgagacacttcgtgtagctggaagacaccaacttcctgacaggagctttatttcatttgggatttcaagtttacagatggtatcttctcaaaagttggaaaaacctatagagatgggcagtagcgaaccccttcccatcgcagatggtgacaggaggaggaagaagaagcggaggggccgggccactgactccttgccaggaaagtttgaagatatgtacaagctgacctctgaattgcttggagagggagcctatgccaaagttcaaggtgccgtgagcctacagaatggcaaagagtatgccgtcaaaatcatcgagaaacaagcagggcacagtcggagtagggtgtttcgagaggtggagacgctgtatcagtgtcagggaaacaagaacattttggagctgattgagttctttgaagatgacacaaggttttacttggtctttgagaaattgcaaggaggttccatcttagcccacatccagaagcaaaagcacttcaatgagcgagaagccagccgagtggtgcgggacgttgctgctgcccttgacttcctgcataccaaagacaaagtctctctctgtcacctaggctggagtgctatggcgccatcagggctcactgcagccccaacctccctgggctccagtgatcctcccacctcagcctcccaagtagctgggactacaggcattgctcatcgtgatctgaaaccagaaaatatattgtgtgaatctccagaaaaggtgtctccagtgaaaatctgtgactttgacttgggcagtgggatgaaactgaacaactcctgtacccccataaccacaccagagctgaccaccccatgtggctctgcagaatacatggcccctgaggtagtggaggtcttcacggaccaggccacattctacgacaagcgctgtgacctgtggagcctgggcgtggtcctctacatcatgctgagtggctacccacccttcgtgggtcactgcggggccgactgtggctgggaccggggcgaggtctgcagggtgtgccagaacaagctgtttgaaagcatccaggaaggcaagtatgagtttcctgacaaggactgggcacacatctccagtgaagccaaagacctcatctccaagctcctggtgcgagatgcaaagcagagacttagcgccgcccaagttctgcagcacccatgggtgcaggggcaagctccagaaaagggactccccacgccgcaagtcctccagaggaacagcagcacaatggacctgacgctcttcgcagctgaggccatcgcccttaaccgccagctatctcagcacgaagagaacgaactagcagaggagccagaggcactagctgatggcctctgctccatgaagctttcccctccctgcaagtcacgcctggcccggagacgggccctggcccaggcaggccgtggtgaagacaggagcccgcccacagcactctgaaatgctccagtcacaccttataggccctaggcctggccaggcattgtcccctggaaacctgtgtggctaaagtctgctgagcaggcagcagcctctgctctgtggctccattcaggctttttcatctacgaaggccctgaggttcccatcaacccccatttccctagggtcctggaggaaaaagctttttccaaaggggttgtctttgaaaaggaaagcaatcacttctcactttgcataattgcctgcagcaggaacatctcttcactgggctccacctgctcacccgcctgcagatctgggatccagcctgctctcaccgctgtagctgtggcggctggggctgcagcctgcagggagaagcaagaagcatcagttgacagaggctgccgacacgtgcctcttccctctcttctctgtcaccctcctctggcggtccttccaccttcctctgtcctccggatgtcctctttgcccgtcttctcccttggctgagcaaagccatcccctcaattcagggaagggcaaggagccttcctcattcaggaaatcaaatcagtcttccggtctgcagcacggaaaagcacataatctttctttgctgtgactgaaatgtatccctcgtttatcatcccctttgtttgtgattgctgctaaagtcagtagtatcgtttttttaaaaaaaaagtttggtgtttttaaccatgctgttccagcaaagatgataccttaaactcccactgcaagcccatgaacttcccagagagtggaacggcttgctcttctttctagaatgtccatgcacttgggttttaatcagcagttccctattattctgattttaagctgttcctgtgatgaacttagagacagcatcggtgtctgctgctgtgtccccaggtcttgtgtgggtggcacagatctgggcagttagatagtgctctgtgcctaaggtgaagccacactagggtgaagcctcacttccctgtttgagcaatgcagtgcctgctgcccgtgtgcatgaaggtacagccattcagataagtggaactattgagttacataaagaaaatagatttgcatttgtcaggcagacgtttatacaacaccacggtgcttttatacattgtgcttattttaataaaactgaaattctaaaaaaaaaaaaaaaaaa

SEQ ID NO: 74 - DNA, 2130 bp, Homo sapiens
gactcagaaatggcggcctccatgttctacggcaggctagtggccgtggccacccttcggaaccaccggcctcggacggcccagcgggctgctgctcaggttctgggaagttctggattgtttaataaccatggactccaagtacagcagcaacagcaaaggaatctctcactacatgaatacatgagtatggaattattgcaagaagctggtgtctccgttcccaaaggatatgtggcaaagtcaccagatgaagcttatgcaattgccaaaaaattaggttcaaaagatgtcgtgataaaggcacaggttttagctggtggtagaggaaaaggaacatttgaaagtggcctcaaaggaggagtgaagatagttttctctccagaagaagcaaaagctgtttcttcacaaatgattgggaaaaaattgtttaccaagcaaacgggagaaaagggcagaatatgcaatcaagtattggtctgtgagcgaaaatatcccaggagagaatactactttgcaataacaatggaaaggtcatttcaaggtcctgtattaataggaagttcacatggtggtgtcaacattgaagatgttgctgctgagtctcctgaagcaataattaaagaacctattgatattgaagaaggcatcaaaaaggaacaagctctccagcttgcacagaagatgggatttccacctaatattgtggaatcagcagcagaaaacatggtcaagctttacagcctttttctgaaatacgatgcaaccatgatagaaataaatccaatggtggaagattcagatggagctgtattgtgtatggatgcaaagatcaattttgactctaattcagcctatcgccaaaagaaaatctttgatctacaggactggacccaggaagatgaaagggacaaagatgctgctaaggcaaatctcaactacattggcctcgatggaaatataggctgcctagtaaatggtgctggtttggctatggccacaatggatataataaaacttcatggagggactccagccaacttccttgatgttggtggtggtgctacagtccatcaagtaacagaagcatttaagcttatcacttcagataaaaaggtactggctattctggtcaacatttttggaggaatcatgcgctgtgatgttattgcacagggtatagtcatggcagtaaaagacttggaaattaaaatacctgttgtggtacggttacaaggtacacgagtcgatgatgctaaggcactgatagcggacagtggacttaaaatacttgcttgtgatgacttggatgaagctgctagaatggttgtaaagctctctgaaatagtgaccttagcgaagcaagcacatgtggatgtgaaatttcagttgccaatatgatctgaaaacccagtggatggctgaaggtgttaaatgtgctataatcattaagaatactgtgttctgtgttattgttctttttctttttagtgtgtggagattgtaattgccatctaggcacacaaacatttaaaaggatttggactgcatttaattgtaccattcagaatggactgtttgtacgaagcatgtataatgcagttatcttctttctttcgtcgcagccagtcttttttgcttctcctacaaaacgtaacttgcaatttgccagtttattattgttggatacaaagttcttcattgataagagtcctataaataagataaatacgaagataaagctttattctttagtgttaaaatacagtatatctaataactagcctcattagtagagcagtatattaaaacaatgttttatgtaaaaagtgtttatcttcagcaccaaatacatgataaatgtatcaatcactatttataaacagagctttcaaacactcctcagaatattcttctaagtattttgatgaagtaactttgtaattatttgaacattgttttaatcattaggaaacactgattaactgcaagtcttcatgattctgtcatattaagaaacacctgtaggtttgcttcaaataaaggcatatataccaaggacttacagacaaaattaagaatgtcaatttaagttaataaaaatctcccaatatgaaaaaaaaaaaaaaa

SEQ ID NO: 75 - DNA, 2292 bp, Homo sapiens
ggagaaaggcaggcagccggcgggcgctgcgtgtgtgtttgagtgtgcgagtgtgagtgagtgtgagtgccgggtcctgcctctcctggtgcccccagcagaggggcccggtggcctggggagggcgggcgggctgctcggaggcagaggcagcaagaggcggcggcggggagagccgagaaggaggaggaggagagaggaaagagcggagcagccattgttgagggaaaccttgcaaacaaagaaggctccggactccccgcggccccccgccccccaggccggccccctggcccccggcccccggcccagtggccattgtaactttcccccgggacagcggccgccgcggggcgggccgaggctgtgcgtgcgagtctttgtgcggccggcggactggagctcccagtcgggcctgcccgtcccactcgggacatgagctgctgaagttgtccccccgtcagccctcacccctgccaggactcctctgggcttgcagtcgctcccccgtacctctcctggccccctcagttctcccaggttgttcctcaaagtcattcaagctgtactcgccgaaggagcccccgaacggcaacgcctttccccccttccatcccggcaccatgctggatagggatgtgggcccaactcccatgtatccgcctacatacctggagccagggattgggaggcacacaccatatggcaaccaaactgactacagaatatttgagcttaacaaacggcttcagaactggacagaggagtgtgacaatctctggtgggatgcattcacgactgagttctttgaggatgatgccatgttgaccatcactttctgcctggaggatggaccaaagagatataccattggccggaccctgatcccacgctacttccgcagcatctttgaggggggtgctacggagctgtactatgttcttaagcaccccaaggaggcattccacagcaactttgtgtccctcgactgtgaccagggcagcatggtgacccagcatggcaagcccatgttcacccaggtgtgtgtggagggccggttgtacctggagttcatgtttgacgacatgatgcggataaagacgtggcacttcagcatccggcagcaccgagagctcatcccccgcagcatccttgccatgcatgcccaagacccccagatgttggatcagctctccaaaaacatcactcggtgtgggctgtccaattccactctcaactacctccgactctgtgtgatactcgagcccatgcaagagctcatgtcacgccacaagacctacagcctcagcccccgcgactgcctcaagacctgccttttccagaagtggcagcgcatggtagcaccccctgcggagcccacacgtcagcagcccagcaaacggcggaaacggaagatgtcagggggcagcaccatgagctctggtggtggcaacaccaacaacagcaacagcaagaagaagagcccagctagcaccttcgccctctccagccaggtacctgatgtgatggtggtgggggagcccaccctgatgggcggggagttcggggacgaggacgagaggctcatcacccggctggagaacacccagtttgacgcagccaacggcattgacgacgaggacagctttaacaactcccctgcactgggcgccaacagcccctggaacagcaagcctccgtccagccaagaaagcaaatcggagaaccccacgtcacaggcctcccagtaaaaggccctgctgtggccacccagtgctctgcctacctgccccacccctcacagccccagggagcagaagacagaatgaagagactgagcatctaaaatgggcagcctccatccacccgcttcagggaggaactggactctctgggggcaggtgccaagatttgcccccccagtggccctgggctgggcctggcctctacagagccttttagggggagggggtactcatgtggatgcctacttggaacctgggcctctgagacatgtcctgaccaccccccagggcatttgcctccatcttcaccccagattgtgggtttcccatccccctgactttacccctttcaagcctggggttgtctatacccacagcccttagggacttaaagttacggggcttcgttgaatgctccttcccctaggcagctggtaatagaggggtaaagctctgggcccctcctccctgctgccacttatcccagctccaagtttttttaaaaataatattattaaaaatgtaagttaaaaaaaaaaaaaaaaaa

SEQ ID NO: 76 - DNA, 1760 bp, Homo sapiens
gcgcggccatggcgcagctgggaaagctgctcaaggagcagaagtacgaccggcagctgaggttgtggggtgatcatgggcaagaggctttagaatctgctcatgtttgcctaataaatgcaacagccacaggaactgaaattcttaaaaacttggtactaccaggtattggttcgtttacaattattgatggaaatcaggtcagcggagaagatgctggaaacaatttcttccttcaaagaagcagtatcggcaagaaccgagctgaagctgccatggaattcttacaagaattaaatagcgatgtctctggaagttttgtggaagagagtccagaaaaccttctagacaatgatccctcatttttctgtaggtttactgttgtagttgcaactcagcttcctgaaagcacttcactacgcttagcagatgtcctctggaattcccagattcctcttttgatctgtaggacatatggactagttggttatatgaggatcattataaaagaacatccagtaatagaatctcatccagataatgcattagaggatctacgactagataagccatttcctgaactgagagaacattttcagtcctatgatttggatcatatggaaaaaaaggaccacagtcatactccatggattgtgatcatagctaaatatttagcacagtggtatagtgaaacaaatggacgaatacctaaaacgtataaagaaaaagaggacttcagagatttgattagacaaggaattctaaaaaatgaaaatggggctccagaagatgaagagaattttgaagaagctattaaaaatgtgaacacagcactaaatacaactcagatcccaagcagtattgaagatatatttaatgatgatcgctgcataaatatcaccaaacagactccatcattttggattttagctcgtgccttaaaggaatttgtggccaaagagggtcaaggaaatttacctgttcgaggcacaattcctgatatgattgcagattcaggcaaatatataaaactgcaaaacgtttaccgtgaaaaagcaaagaaagatgctgccgctgtgggtaatcatgttgccaaattgctgcagtccattggccaggcaccagagtccatttcagagaaagaattaaaattactctgcagcaattctgcatttcttcgagtggtaagatgtcgatccttagctgaagaatatggtttggatacaattaacaaggatgaaattatttctagcatggacaatccagataatgaaatagtgttgtacttaatgttacgggctgttgatagatttcataaacaacagggtagatatccaggagtatctaactatcaagttgaagaagatataggaaagttgaagtcttgtctcactggcttccttcaggaatatggtttatctgtaatggtgaaagatgattatgtccacgaattttgccgatatggagctgctgagccacataccattgctgcattcttggggggagctgctgctcaagaggtcatcaaaataatcaccaaacaatttgtaatttttaataatacttacatttacagtggcatgtcacaaacttcagcaactttccagttgtagagtaagcaagcaccttaagtagtgtgttaatgattgaaactgtaattgccttcgggttgtgctttagtctgtaaaattctaaaggagagctgctaaattgttttcttaataaacatttttctcatttgtaaaaaaaaaaaaaaaaaa

SEQ ID NO: 77 - DNA, 2415 bp, Homo sapiens
gtccggcagccgctgctgctgctgctgctgctgctgccgccgccgccgccgccgtccctgcgtccttcggtctctgctcccgggacccgggctccgccgcagccagccagcatgtcggggatcaagaagcaaaagacggagaaccagcagaaatccaccaatgtagtctatcaggcccaccatgtgagcaggaataagagagggcaagtggttggaacaaggggtgggttccgaggatgtaccgtgtggctaacaggtctctctggtgctggaaaaacaacgataagttttgccctggaggagtaccttgtctcccatgccatcccttgttactccctggatggggacaatgtccgtcatggccttaacagaaatctcggattctctcctggggacagagaggaaaatatccgccggattgctgaggtggctaagctgtttgctgatgctggtctggtctgcattaccagctttatttctccattcgcaaaggatcgtgagaatgcccgcaaaatacatgaatcagcagggctgccattctttgaaatatttgtagatgcacctctaaatatttgtgaaagcagagacgtaaaaggcctctataaaagggccagagctggggagattaaaggatttacaggtattgattctgattatgagaaacctgaaactcctgagcgtgtgcttaaaaccaatttgtccacagtgagtgactgtgtccaccaggtagtggaacttctgcaagagcagaacattgtaccctatactataatcaaagatatccacgaactctttgtgccggaaaacaaacttgaccacgtccgagctgaggctgaaactctcccttcattatcaattactaagctggatctccagtgggtccaggttttgagcgaaggctgggccactcccctcaaaggtttcatgcgggagaaggagtacttacaggttatgcactttgacaccctgctagatgatggcgtgatcaacatgagcatccccattgtactgcccgtctctgcagaggataagacacggctggaagggtgcagcaagtttgtcctggcacatggtggacggagggtagctatcttacgagacgctgaattctatgaacacagaaaagaggaacgctgttcccgtgtttgggggacaacatgtacaaaacacccccatatcaaaatggtgatggaaagtggggactggctggttggtggagaccttcaggtgctggagaaaataagatggaatgatgggctggaccaataccgtctgacacctctggagctcaaacagaaatgtaaagaaatgaatgctgatgcggtgtttgcattccagttgcgcaatcctgtccacaatggccatgccctgttgatgcaggacactcgccgcaggctcctagagaggggctacaagcacccggtcctcctactacaccctctgggcggctggaccaaggatgacgatgtgcctctagactggcggatgaagcagcacgcggctgtgctcgaggaaggggtcctggatcccaagtcaaccattgttgccatctttccgtctcccatgttatatgctggccccacagaggtccagtggcactgcaggtcccggatgattgcgggtgccaatttctacattgtggggagggaccctgcaggaatgccccatcctgaaaccaagaaggatctgtatgaacccactcatgggggcaaggtcttgagcatggcccctggcctcacctctgtggaaatcattccattccgagtggctgcctacaacaaagccaaaaaagccatggacttctatgatccagcaaggcacaatgagtttgacttcatctcaggaactcgaatgaggaagctcgcccgggaaggagagaatcccccagatggcttcatggcccccaaagcatggaaggtcctgacagattattacaggtccctggagaagaactaagcctttggctccagagtttctttctgaagtgctctttgattaccttttctatttttatgattagatgctttgtattaaattgcttctcaatgatgcattttaatcttttataatgaagtaaaagttgtgtctataattaaaaaaaaatatatatatatacacacacacatatacatacaaagtcaaactgaagaccaaatcttagcaggtaaaagcaatattcttatacatttcataataaaattagctctatgtattttctactgcacctgagcaggcaggtcccagatttcttaaggctttgtttgaccatgtgtctagttacttgctgaaaagtgaatatattttccagcatgtcttgacaacctgtactcttccaatgtcatttatcagttgtaaaatatatcagattgtgtcctcttctgtacaattgacaaaaaaaaaaaaaaaaaaaaaaaa

SEQ ID NO: 78 - DNA, 3009 bp, Homo sapiens
gcgcaggcgcggcggccgatgcgagtgtgtatgtgcgggcgagaagatggcggcggcgggggaagcagcgtgagcagccggaggatcgcggagtcccaatgaaacgggcagccatggccctccacagcccgcagtatatttttggagattttagccctgatgaattcaatcaattctttgtgactcctcgatcttcagttgagcttcctccatacagtggaacagttctgtgtggcacacaggctgtggataaactacctgatggacaagaatatcagagaattgagtttggtgtcgatgaagtcattgaacccagtgacactttgccgagaacccccagctacagtatttcaagcacactgaaccctcaggcccctgaatttattctcggttgtacagcttccaaaataacccctgatggtatcactaaagaagcaagctatggctccatcgactgccagtacccaggctctgccctcgctttggatggaagttctaatgtggaggcggaagttttggaaaatgatggtgtctcaggtggtcttggacaaagggagcgtaaaaagaagaaaaagcggccacctggatattacagctatttgaaagatggtggcgatgatagtatctccacagaagccctggtcaatggccatgccaattcagcagtcccgaacagtgtcagtgcagaggatgcagaatttatgggtgacatgcctccgccacttacgcccaggacttgtaacagcccccagaactccacagactctgtcagtgacattgtgcctgacagtcctttccccggagcactcggcagtgacaccaggactgcagggcagccagaggggggccccggggctgattttggtcagtcctgcttccctgcagaggctggcagagacaccctgtcaaggacagctggggctcagccctgcgttggtaccgatactactgaaaaccttggagttgctaatggacaaatacttgaatcctcgggtgagggcacagctaccaacggggtggagttgcacaccacggaaagcatagacttggacccaaccaaacccgagagtgcatcacctcctgctgacggcacgggctctgcatcaggcacccttcctgtcagccagcccaagtcctgggccagcctctttcatgattctaagccctcttcctcctcgccggtggcctatgtggaaactaagtattcccctcccgccatatctcccctggtttctgaaaagcaggttgaagtcaaagaagggcttgttccggtttcagaggatcctgtagccataaagattgcagagttgctggagaatgtaaccctaatccataaaccagtgtcgttgcaaccccgtgggctgatcaataaagggaactggtgctacattaatgctacactgcaggcattggttgcttgcccgccgatgtaccacctgatgaagttcattcctctgtattccaaagtgcaaaggccttgtacgtcaacacccatgatagacagctttgttcggctaatgaatgagttcactaatatgccagtacctccaaaaccccgacaagctcttggagataaaatcgtgagggatattcgccctggagctgcctttgagcccacatatatttacagactcctgacagttaacaagtcaagcctgtctgaaaagggtcgacaagaagatgctgaggaatacttaggcttcattctaaatggacttcatgaggaaatgttgaacctaaagaagcttctctcaccaagtaatgaaaaacttacgatttccaacggccccaaaaaccactcggtcaatgaagaagagcaggaagaacaaggtgaaggaagcgaggatgaatgggaacaagtgggcccccggaacaagacttccgtcacccgccaggcggattttgttcagactccaatcaccggcatttttggtggacacatcaggtctgtggtttaccagcagagttcaaaagaatctgccactttgcagccatttttcacgttgcagttggatatccagtcagacaagatacgcacagtccaggatgcactggagagcttggtggcaagagaatctgtccaaggttataccacaaaaaccaaacaagaggttgagataagtcgaagagtgactctggaaaaactccctcctgtcctcgtgctgcacctgaaacgattcgtttatgagaagactggtgggtgccagaagcttatcaaaaatattgaatatcctgtggacttggaaattagtaaagaactgctttctccaggggttaaaaataagaattttaaatgccaccgaacctatcggctctttgcagtggtctaccatcacggcaacagtgcgacgggcggccattacactacagacgtcttccagatcggtctgaatggctggctgcgcatcgatgaccagacagtcaaggtgatcaaccagtaccaggtggtgaaaccaactgctgaacgcacagcctacctcctgtattaccgccgagtggacctgctgtaaaccctgtgtgcgctgtgtgtgcgcccagtgcccgcttcgtaggacaccacctcacactcacttcccgcctctctttagtggctctttagagagaaactctttctccctttgcaaaaatgggctagaatgaaaaggagatgccttggggttcgtgcacaacacagcttctgttgactctaacttccaaatcaaaatcatttggttgaaacagactgttgcttgattttagaaaatacacaaaaacccatatttctgaaataatgctgattcctgagataagaaagtggatttgatccccagtctcattgcttagtagaataaatcctgcaccagcaacaacacttgtaaatttgtgaaaatgaattttatctttccttaaaaaagaaattttttaatccatcacacttttcttccctaccctttagtttttgataaatgataaaaatgagccagttatcaaagaagaactagttcttacttcaaaaaaaaaaaaaaaaaa

SEQ ID NO: 79 - DNA, 2756 bp, Homo sapiens
gaaagggagtgttcgggtttcgctggggcctcgcggctccagagcccagcatggcttcctcgcgagcctcttccacgcaggcaaccaaaactaaagcacccgacgacttagttgctccggtcgtgaagaaaccacacatctattatggaagtttggaagagaaggagagggagcgtctggccaaaggagagtctgggattttggggaaagacggacttaaagcagggatcgaagctggaaatattaatataacctctggagaagtgtttgaaattgaagagcatatcagcgagcgacaggcagaagtattggctgagtttgagagaaggaagcgagcccggcagatcaatgtttccacagatgactcagaggtcaaagcttgccttagagccttgggggaacccatcacactttttggagagggtcctgctgaaagaagagaaaggttaagaaatatcctctcagttgtcggtactgatgccttgaaaaagaccaaaaaggatgatgagaagtctaaaaagtccaaagaagagtatcagcaaacctggtatcatgaaggaccaaatagcttgaaggtggcaagactatggattgctaattattcgttgcccagggcaatgaaacgcttggaagaggcccgactccataaggagattcctgagacaacaaggacctcccagatgcaagagctgcacaagtctctccggtctttgaataatttttgcagtcagattggggatgatcggcctatctcctactgtcactttagtcccaattccaagatgctggccacagcttgttggagtgggctttgcaagctctggtctgttcctgattgcaacctccttcacactcttcgagggcataacacaaatgtaggagcaattgtattccatcccaaatccactgtctccttggacccaaaagatgtcaacctggcctcttgtgcggctgatggctctgtgaagctttggagtctcgacagtgatgaaccagtggcagatattgaaggccatacagtgcgtgtggcgcgggtaatgtggcatccttcaggacgtttcctgggcaccacctgctatgaccgttcatggcgcttatgggatttggaggctcaagaggagatcctgcatcaggaaggccatagcatgggtgtgtatgacattgccttccatcaagatggctctttggctggcactgggggactggatgcatttggtcgagtttgggacctacgcacaggacgttgtatcatgttcttagaaggccacctgaaagaaatctatggaataaatttctcccccaatggctatcacattgcaaccggcagtggtgacaacacctgcaaagtgtgggacctccgacagcggcgttgcgtctacaccatccctgctcatcagaacttagtgactggtgtcaagtttgagcctatccatgggaacttcttgcttactggtgcctatgataacacagccaagatctggacgcacccaggctggtccccgctgaagactctggctggccacgaaggcaaagtgatgggcctagatatttcttccgatgggcagctcatagccacttgctcatatgacaggaccttcaagctgtggatggctgaatagatgacaatgggaaaaggacttgaacctcaagctctctctaaggagctgttttcctcaaacgagaagaattgaagtgtttagttctatcatgttttctgccaattaccatgcatagaccctcagtagaattggatttccatgtcagcccccactccaggaaggcagcccaatccctaggtgatggggaacccctctcacggttgaaaatttattacctttttacgccctgccacgaactgtgtagacattgtttttattaatcttttgtttggccgggcgtggtggctcacgcctgtaatcctagcactttgggaggccgaggtgggtagatcgcttgagctcaggagttcaagacgagcctgggcaacatggcaaatgccgtctctgcaaaaaaatactaaaattagctggtcgcggtggcttctgcctgtgattccggctacttgggaggctgaggtgggagggattgcttaagcctgggaggcagaggttgcagtgagccgagattgcgccattgcactctagcctgtgtgacagagcaagaccctgtctcaaaaaaaaaaaaaatttgttcgaatgccttatagccttcctcacagcacccaggattgtgactgactctgcatttttaattcttgaaacttggctttccataacatggtacatgcttcaggactacatatgacccagagagcaaggtggctgaactatagtctggaagccctcaggtaaagaggcacatctcaccactcattggttaaacaatgcatcatagcgagcacttttcctttccctggagaatgggatgtgaagcagtagaccgcagccacgccgatggttatacagtgaagaagacttcacctcttcctattgagtttgcttggaatgctgacagcatcaggcaactctgaactgaacatttgctttgtcagaaaatatctttttttttactttgaagtttggcaaccttcatgttaccccaaagcaaaaccattgtgtcaggagtcaaacaaatgtttagaaagcaaacatgacgtctctattgtacaacctcctttctcttggctgtttaaaggatgtacttcgtgtattaaagggtactttatgttgaagtaaaaaaaaaaaaaaaaaaaaaaa

SEQ ID NO: 80 - DNA, 1518 bp, Homo sapiens
ggggagccaattagagctccagttgtcactcctacccacactgggcctgggggtgaagggaagtgtttattaggggtacatgtgaagccgtccagaagtgtcagagtctttgtagctttgaaagtcacctaggttatttgggcatgctctcctgagtcctctgctagttaagctctctgaaaagaaggtggcagacccggtttgctgatcgccccagggatcaggaggctgatcccaaagttgtcagatggagagtaaatacaaggagatactcttgctaacaggcctggataacatcactgatgaggaactggataggtttaagttctttctttcagacgagtttaatattgccacaggcaaactacatactgcaaacagaatacaagtagctaccttgatgattcaaaatgctggggcggtgtctgcagtgatgaagaccattcgtatttttcagaagttgaattatatgcttttggcaaaacgtcttcaggaggagaaggagaaagttgataagcaatacaaatcggtaacaaaaccaaagccactaagtcaagctgaaatgagtcctgctgcatctgcagccatcagaaatgatgtcgcaaagcaacgtgctgcaccaaaagtctctcctcatgttaagcctgaacagaaacagatggtggcccagcaggaatctatcagagaagggtttcagaagcgctgtttgccagttatggtactgaaagcaaagaagcccttcacgtttgagacccaagaaggcaagcaggagatgtttcatgctacagtggctacagaaaaggaattcttctttgtaaaagtttttaatacactgctgaaagataaattcattccaaagagaataattataatagcaagatattatcggcacagtggtttcttagaggtaaatagcgcctcacgtgtgttagatgctgaatctgaccaaaaggttaatgtcccgctgaacattatcagaaaagctggtgaaaccccgaagatcaacacgcttcaaactcagccccttggaacaattgtgaatggtttgtttgtagtccagaaggtaacagaaaagaagaaaaacatattatttgacctaagtgacaacactgggaaaatggaagtactgggggttagaaacgaggacacaatgaaatgtaaggaaggagataaggttcgacttacattcttcacactgtcaaaaaatggagaaaaactacagctgacatctggagttcatagcaccataaaggttattaaggccaaaaaaaaaaaacatagagaagtaaaaaggaccaattcaagccaactggtctaagcagcatttaattgaagaatatgtgatacagtctcttcaatcagattgtaagttacctgaaagctgcagttcacaggctcctctctccaccaaattaggatagaataattgctggataaacaaattcagaatatcaacagatgatcacaataaacatctgtttctcattcaaaaaaaaaaaaaaaaaaaaaaaaaaaaaa

SEQ ID NO: 81 - DNA, 1311 bp, Homo sapiens
atttattgtcgcggggaagctgcggccgcctcgcacccggaacaacaaagcaaggaagacggagtccgagcctcgggggctcctagcaacgggccggggcgggagttccatggagactggggagcgcgcccgtctcatcctcatccttgtcctccagcttctccttcgcatccgacgcaaccggcagcagcgctgccgccgcgtcctcagccaccgctccctcttcccacggatgtgatcttcgtggtggaaagctaaattttaaaaccaccccaatggatgcagacagtgatgttgcattggacattctaattacaaatgtagtctgtgtttttagaacaagatgtcatttaaacttaaggaagattgctttggaaggagcaaatgtaatttataaacgtgatgctggaaaagtattaatgaagcttagaaaacctagaattacagctacaatttggtcctcaggaaaaattatttgcactggagcaacaagtgaagaagaagctaaatttggtgccagacgcttagcccgtagtctgcagaaactaggttttcaggtaatatttacagattttaaggttgttaacgttctggcagtgtgtaacatgccatttgaaatccgtttgccagaattcacaaagaacaatagacctcatgccagttacgaacctgaacttcatcctgctgtgtgctatcggataaaatctctaagagctacattacagattttttcaacaggaagtatcacagtaacagggcccaatgtaaaggctgttgctactgctgtggaacagatttacccatttgtgtttgaaagcaggaaagaaattttataattcaccacttaattggttagaatctctaactgagcaccttttaaacctgctgcacattggactcaaaaggaaaactggaccaacaataattgaggaaatagactcttttattcattcacggctacagtgtaagctccagtccctttggattttattccaaaccttgctgtaatataaaaggaagtttacaagacatgatattgctgcttttacaaaaggacattctatttattttcgcagtaattctcatgtccccataagcagagctgtcacagtgtgcactaccttagattgttttattgtcgtcattgttatttttttccattttgagctaatgtgttttatttgtgaatagtcttttacatttttgtatgctgaatatgggcaccaaagaacctgtaaaagttatctttttcaattgaatgtgcacaaataaaagtttggaaaagaaaaaaaaaaaaaaaaaaaaaaaaaa

SEQ ID NO: 82 - DNA, 2041 bp, Homo sapiens
gcccggagccgggagcgccgctccagcgaggcgcgggctgtggggccgccgcgtgcctggccccgctcgcccgtgccggccgctcgcccgccatgcctggcttcgactacaagttcctggagaagcccaagcgacggctgctgtgcccactgtgcgggaagcccatgcgcgagcctgtgcaggtttccacctgcggccaccgtttctgcgatacctgcctgcaggagttcctcagtgaaggagtcttcaagtgccctgaggaccagcttcctctggactatgccaagatctacccagacccggagctggaagtacaagtattgggcctgcctatccgctgcatccacagtgaggagggctgccgctggagtgggccactacgtcatctacagggccacctgaatacctgcagcttcaatgtcattccctgccctaatcgctgccccatgaagctgagccgccgtgatctacctgcacacttgcagcatgactgccccaagcggcgcctcaagtgcgagttttgtggctgtgacttcagtggggaggcctatgagagccatgagggtatgtgcccccaggagagtgtctactgtgagaataagtgtggtgcccgcatgatgcggcggctgctggcccagcatgccacctctgagtgccccaagcgcactcagccctgcacctactgcactaaggagttcgtctttgacaccatccagagccaccagtaccagtgcccaaggctgcctgttgcctgccccaaccaatgtggtgtgggcactgtggctcgggaggacctgccaggccatctgaaggacagctgtaacaccgccctggtgctctgcccattcaaagactccggctgcaagcacaggtgccctaagctggcaatggcacggcatgtggaggagagtgtgaagccacatctggccatgatgtgtgccctggtgagccggcaacggcaggagctgcaggagcttcggcgagagctggaggagctatcagtgggcagtgatggcgtgctcatctggaagattggcagctatggacggcggctacaggaggccaaggccaagcccaaccttgagtgcttcagcccagccttctacacacataagtatggttacaagctgcaggtgtctgcattcctcaatggcaatggcagtggtgagggcacacacctctcactgtacattcgtgtgctgcctggtgcctttgacaatctccttgagtggccctttgcccgccgtgtcaccttctccctgctggatcagagcgaccctgggctggctaaaccacagcacgtcactgagaccttccaccccgacccaaactggaagaatttccagaagccaggcacgtggcggggctccctggatgagagttctctgggctttggttatcccaagttcatctcccaccaggacattcgaaagcgaaactatgtgcgggatgatgcagtcttcatccgtgctgctgttgaactgccccggaagatcctcagctgagtgcaggtggggttcgaggggaaaggacgatggggcatgacctcagtcaggcactggctgaacttggagagggggccggacccccgtcagctgcttctgctgcctaggttctgttaccccatcctccctcccccagccaccaccctcaggtgcctccaattggtgcttcagccctggcccctgtggggaacaggtcttggggtcatgaagggctggaaacaagtgaccccagggcctgtctcccttcttgggtagggcagacatgccttggtgccggtcacactctacacggactgaggtgcctgctcaggtgctatgtcccaagagccataagggggtgggaattggggagggagaaagggtagttcaaagagtctgtcttgagatctgattttttccccctttacctagctgtgccccctctggttatttatttccttagtgccaggagggcacagcaggggagccctgatttttaataaatccggaattgtatttattaaaaaaaaaaaaaaaaaaaaaaaaaaaaaaaaaaa

SEQ ID NO: 83 - DNA, 3364 bp, Homo sapiens
agcggggggtttggggggaggggggaggggaaacgggtgaagaaggggaggcggcggcctggcgggggcatccggcggagctggggtccccgggctccgtccggaggaagcgacgctgcgctcgctgggcagtcggaggggacgggacgcaccagagggcaggcggactcgccctgtcggtgactgcgccgtccgggcccgtcctgcctggccgcaggtgccctggatgaggccgccccgcgcgccccaaacgattttataatcaatggataaagtgggaaaaatgtggaataacttcaaatacaggtgtcagaatctcttcggtcatgagggaggaagccgtagtgaaaatgtggacatgaactccaacagatgtttgtctgtcaaagagaaaaacatcagcataggagactcaactcctcagcaacaaagcagtcccttaagagaaaatattgccttacaactgggattaagcccttcgaagaattcttcaaggagaaatcaaaattgtgccacagaaatccctcaaattgttgaaataagcatcgaaaaggataatgattcttgtgttacgccaggaacaagacttgcacgaagagattcctactctcgacatgctccatggggtgggaagaaaaaacattcctgttctacaaagacccagagttcattggatgctgataaaaagtttggtagaactcgaagtggacttcaaaggagagagaggcgctacggcgtaagttctgtacacgacatggacagtgtttccagcagaactgtaggaagtcgctctctaagacagaggttgcaggatactgtgggcttgtgttttcccatgagaacttacagcaagcagtcaaagcctctcttttccaataaaagaaaaatccatctctctgaattaatgcttgagaaatgcccttttcctgctggctcagatttagcccaaaaatggcatttgattaaacagcatacagctcctgtgagcccacattcaacattttttgatacatttgatccatctttggtttctacagaagatgaagaagataggcttagagagagaaggcggcttagtattgaagaaggggttgatccccctcccaatgcacaaatacatacatttgaagctactgcacaggttaatccattatataaactgggaccaaaattagctcctggaatgactgaaataagtggggacagttctgcaattccacaagctaattgtgactcggaagaggatacaaccaccctgtgtttgcagtcacggaggcagaagcagcgtcagatatctggagacagccatacccatgttagcagacagggagcttggaaagtccacacacagattgattacatacactgcctcgtgcctgatttgcttcaaattacagggaatccctgttactggggagtgatggaccgttatgaagcagaagcccttctcgaagggaaacctgaaggcacgtttttgctcagggactctgcgcaagaggactacctcttctctgtgagcttccgccgctacaacagatccctgcatgcccgaattgagcagtggaatcacaactttagtttcgacgcccatgacccgtgtgtatttcactcctccactgtaacgggacttttagaacattataaagatcccagttcgtgcatgttttttgaaccattgcttactatatcactaaatatgactttcccttttagcctgcagtatatctgtcgcgcggtaatctgcaggtgcactacgtatgatggaattgatgggctccctctaccctcaatgttacaggattttttaaaagagtatcattataaacaaaaagttagagttcgctggttggaacgagaaccagtcaaggcaaagtaaactctccggtccccaaaggttgttaactaggtccgctttcatgtgcatcagacagtacacctatagcaagcacacgtagcagtgttaggctttttcatacagtatgtaagcttagtgttagtatctgtcagatgctacctgctgttacttattcagataaacatggtgcctattggaacaatagcggatagagctacaggtgttcagtaagactacaaaaacattttgcctatttcgctaacagtttggtttttaatggctgtggtatttgagtgaggcaactctgggtcatttgttatgaagaattctatttcttactgaagaacaaattattaatattggatgagtatttcaacagtgtgactaatgtttgaaattattttttctaagagtttttctataaccttccaaaagtcgtgatgtttgtagttactataaatcaagctttggaagtccaaaaagaataaaagactgccttccttttagaaaaaaatgcaattttctggccacaagggcatagtgcagttcacttacgtgttgatgtagtttataatcagacgccttttctcttctgcaaaaggtactgttaagtaaaccagattttctaaataggcattcttaaaatttcagacttacaaatctagtagtagaattttattgaaaggcctaggtattaattttttaaatgagtgctttaacttaaaacaggcgtttggaatagctgctgcaatgtagtcttgtgtgtgatttttttttaagttgatgtgcagtctaattgttgtttcataaaagttggatctgttcctatgcccaggatgattttgtgaaccgtgaagtacgtgagactagaagacgcccaaacaagtcagataatagtaactacaatggttgctgatgttgagattattgttgaactataattaataatttggatggcagaatttatctcttttttgtaaactctcataactgaattgcttaagtataatttatagaatttcagtgcagttcattcttaatggaaaatctgaaacctaaattgcagatttaaaaggtactgtacaaccattatatctgtaaataacttagcacctttttgtcactttgaataatatgtactactacttgagtgagcgcttttggaagttatatcaagttctagtgtttgcttcttagtaactgaactgaatttacagttctgtcctagacattttgcactaaagtagccgaatccactctcatgtcttttcgttaatgtgctctgtaccactggtgagtgctccatagtttccttacctgctgctacagaatgttattttacatccctatggctattgccaaggctacaaaaaaggaaagctatatttgtatgcaacactaaccttttgactgctaatgtatgtttctgcttgctgtgccttgttatggctgctttttttgtgctaataaagtatgtttggtgttctccttgcaaaaaaaaaaaaaaaa

SEQ ID NO: 84 - DNA, 3931 bp, Homo sapiens
gggtcccaaaagggttcgaggcctcttgccttcgctccgtaggagggagttctcttctggccctgggggccgcaggactgtccaggaatacagtttccaggtgagatcttacttgaaacaccggtagttctagcacagctaaaatggcatctacttgggctatccaggcccacatggaccaggatgaacctttggaagtaaagatagaggaagagaaatataccaccagacaggattgggacctgcgtaaaaacaacacccatagcagagaggtcttccgtcagtacttcagacagttctgctaccaggagacatctggtccccgtgaggctttgagccgactccgagaactttgccatcagtggctgaggccagagacccacaccaaagaacagattctggagctgctggtgctggagcagttcctgaccatcctacctgaggagctccaggcctgggtgcaggagcagcatccagagagtggggaggaggtggtgactgtgctggaggatttagagagagaactggatgaaccaggagagcaggtctcagtccacactggggaacaggaaatgttcttgcaggagacggtacgtctacgaaaagaaggagaacccagtatgtccctccagtccatgaaagcccagccaaagtatgaatctccagaacttgaatcccaacaggagcaagttttagatgttgagactggaaatgagtatgggaatttaaagcaagaagtttctgaagaaatggaaccacatgggaagacatccagtaaatttgaaaatgatatgtccaagtctgctaggtgtggagaaactcgtgaacctgaagaaataacagaagagccctctgcttgctccagagaagataaacaacctacctgtgatgaaaatggagtaagcctgactgagaactctgaccatactgaacatcagagaatctgtccaggagaagaatcttacggatgtgatgactgtggaaaagcttttagtcagcactcacacctcatagaacatcagaggatccatactggagatagaccctacaaatgcgaagaatgtggaaaagctttccgtgggagaactgtgcttattcgacacaaaataatacacactggagagaaaccgtataagtgtaatgaatgtggcaaagcctttggccggtggtcagctcttaaccaacatcagagacttcacacaggagaaaaacactatcactgtaatgactgtggcaaagcctttagtcagaaagcaggcctctttcaccatatcaagatccacacaagagacaaaccttatcagtgcactcagtgtaataaaagttttagtcggcgttccatacttactcagcatcaaggagttcataccggcgccaagccgtatgagtgcaacgagtgtggaaaagcctttgtttataactcatcccttgtttcccatcaggaaatccaccacaaagaaaaatgctatcagtgtaaggaatgtgggaaatccttcagtcagagtggccttattcagcatcagagaattcacactggggaaaaaccctacaaatgtgacgtatgtgaaaaagcctttattcaaaggacaagtcttacagaacatcagcgaattcacactggagaaagaccctataaatgtgataagtgtgggaaggcgtttactcaaagatcagtcctcacggaacatcagagaatccacactggagagaggccctacaagtgtgatgagtgtgggaatgccttccgaggaatcaccagcctcattcagcatcagagaatccacactggggagaagccctaccaatgtgatgagtgtggaaaagccttcagacagaggaagaagaccagctacaaggaaatacttctgaaaaatcactctgaaccccaagctggagtgaatcttctcctgagctctctcattccagaatggcaatcttgtttcaggagagatctctaatagtgactgatggtaaaggaagaaggctgtgcctagaacaaagaatcaggctggcaaagaaatcacaggtctttcctcaaactttaaattgctactctctggagatagctacctggactttacagagctgtcagaagacttgggaaggtttgccagcattggaaaaagccacttggacttcacagagctgtcagaagacttaggaagctttgccagtagttcctgagaccagaaatacatattatgaagcaggtgctggaacaggccctggcccctacttgtgaagctgcagatatagatggtgttactgagaaggaaactaaggacttggctccaaaacaaaacaaaacaaaatctggagatgctgaattttgttggggggaatgtctgtagaactgcaacatcaatgcagtaagtgtagtaaaagctttagtttatggtgagtcattactcagcatcatggagtccacgatgaagagaatctcttttcaatctgtgcaatcaacaagtttttgaaaggcttttgtttatagtggaaccctcttttccccctagaagaaccaccacaaggaaaaatgctgtcaccataaggaatgtgggaaagttttcagccacatggccttatttgacttcaaagaatccacatgagagagagactgcataaatgtaataactgaggaggcctttgttcagcaaagaaaccttatacaaactcgtataaagaatgtaggaagaccttcacccagaagccaggactgactgtgcaccagagaatctgcacaataaacctgatgaatgtggagatgcttgcttcagcattggagaattcacagtaaagggaaaccctgccagtgtgatgagtacggaaaagtcttcagaggttggaacttgggaaaaatcagtgagtgaattcatattagagaggctcttataaatgagaggattgtggaaacttgtatagacagagatcagacctcaacaagggagcctccacagtattgaaaagttaatataattaatgtaagtgggcaagttgtatggggaatgggtagtctaaattgctcattttagccagtaaaaacattaaattagtttcttagatcaagaaaaggaaatgttagaagttatttttcccttccagaggggtctatatgtgtaagcaaccaggacttttgaaataagttgcatctctggatttgtgatctgtctcctggatgattatctctgttttgggagacaggtattttttcttcatgcctgctctagtctcatctcatatctttaattcattcagcaagtatacacaatagtattatcgtctactgtttgttgtaaatatatatatattattttctttttggtctgagtaggcttgtattggtaccctaggacataaaaatcacatatatcatgtttcagtaaatgcagtgaaaggaactatattattaaattgctgggaatttctgtaaatttgagactacctttatattctccaacacaagaccgtgtaggtgtcccttcagagagtatatttttctagcttggcttctttattttctttatttttttgagatggagtcttgctcctgtcgcccaggctggagtgcaatggcacgatctcggctcactgcaacctccgcctcccaggttcaagtgattctcctgtctcagtctcccgagtagctgggattacaggcacttgccaccattcccagctaatttttgtatttttagtagagacggggtttcaccatgttggccaggctggtctcgaactcctgaccttgtgatctgcccaccttggcctcccaaagttcagggattacaggcatgagccaccatgcccggctgttttttttaagatagaaaaatatacttcatgtatagcctattgacagataatgactgatattttttcttatagataacacataataaacattgtgaagactaaaaaaaaaaaaaaaaaaaaa

SEQ ID NO: 85 - DNA, 3791 bp, Homo sapiens
ccacgcgtccggacgctggagtggggagaagcatcatggttcaacgttcatctgcagaagccagcggacgcctctgttcaacttgtgggttacctggctcatgagaccttgccggcgaggctcggcgcttgaacgtctgtgacccagccctcaccgtcccggtacttgtatgtgttggtgggagtttggagctcgttggagctatcgtttccgtggaaattttgagccatttcgaatcacttaaaggagtggacattgctagcaatgagctcccaaagccatccagatggactttctggccgagaccagccagtggagctgctgaatcctgcccgcgtgaaccacatgcccagcacgaggcgcagctccacgagcacatcaagcaacaacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacgccgagagactcacccttcccgccctccagcagaggctctcccttttccccggcacccacctcactccctacctgagcacctcgcccttggagcgggacggaggggcagcgcacagccctcttctgcagcacatggtcttactggagcagccgccggcacaagcacccctcgtcacagactggtatctttcaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccccagcgtcctgtacatgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctgtagcactccctcgaagctgctgttctcttgtctgtctgtctctgtcttgaagctcagccaagaaactttcccgtgtcacgcctgcgtcccaccgtggggctctcttggagcacccagggacacccagcgtgcaacagccacgggaagcctttctgccgcccaggcccacaggtctcgagacgcacatgcacgcctgggcgtggcagcctcacagggaacacgggacagacgccggcgacgcgcagacacacggacacgcggaagccaagcacactctggcgggtcccgcaagggacgccgtggaagaaaggagcctgtggcaacaggcggccgagctgccgaattcagttgacacgaggcacagaaaacaaatatcaaagatctaataatacaaaacaaacttgattaaaactggtgcttaaagtttattacccacaaaaaaaaaaaaaaaaaaaaaaaaaaaaaaaaaaaaaaaaaaaa

SEQ ID NO: 86 - DNA, 1435 bp, Homo sapiens
gaaacaccctcggctgggaagtcagttcgttctctcctctcctctcttcttgtttgaacatggtgcggactaaagcagacagtgttccaggcacttacagaaaagtggtggctgctcgagcccccagaaaggtgcttggttcttccacctctgccactaattcgacatcagtttcatcgaggaaagctgaaaataaatatgcaggagggaaccccgtttgcgtgcgcccaactcccaagtggcaaaaaggaattggagaattctttaggttgtcccctaaagattctgaaaaagagaatcagattcctgaagaggcaggaagcagtggcttaggaaaagcaaagagaaaagcatgtcctttgcaacctgatcacacaaatgatgaaaaagaatagaactttctcattcatctttgaataacgtctccttgtttaccctggtattctagaatgtaaatttacataaatgtgtttgttccaattagctttgttgaacaggcatttaattaaaaaatttaggtttaaatttagatgttcaaaagtagttgtgaaatttgagaatttgtaagactaattatggtaacttagcttagtattcaatataatgcattgtttggtttcttttaccaaattaagtgtctagttcttgctaaaatcaagtcattgcattgtgttctaattacaagtatgttgtatttgagatttgcttagattgttgtactgctgccatttttattggtgtttgattattggaatggtgccatattgtcactccttctacttgctttaaaaagcagagttagatttttgcacattaaaaaattcagtattaattaaacattacttattctaccctcttttttggcaaggaggacaaatacgcaatgttggaaaaccttggatggatatcttctctttaaaaaaatgtaaagataatttggtcttgagggtttaaacggttgataatgcctctacaacaacaagaaaaaagataaaatactaggatagaatcatggtgggcacagtggcttctcaggaggctgaggagggaggtttgcttgagtccaggagttggagaccagcccaggcaacatagcgtaaaccctatctctaaaacaatttttagccaggtgcggtggctcacgcctgtaatcccagcactctgggaggccgaggcgggtggatcatgaggtcaggagatcgagaccatcctgcctaacaaggtgaaaccccgtctctactaaaaatacaaaaaattagccgggcgcggtggcgggcgcctgtagtcccagctactcgggaggctgaggcaggagaatggcgtgaacccgggaagtggagcttgcagtgagccgagattgcgccactgcagtcggcagtccggcttgggcgacagagcgagactccgtctcaaaaaaaaaaaaaaaaaaaaaaaa

SEQ ID NO: 87 - DNA, 1723 bp, Homo sapiens
agctgaatagaatctgaccattggctttcacctggccaggaccttctatgtagctctccttttgtggcccatgtgctgcatcctctgccctcagtgtgcaactggcccccaacgcaatgtgtgtttgtcaaaccatggaagtggggcagtatggcaagaatgcaagtcgggctggagaccggggagtcctcctggagcccttcatccaccaagtaggcggacacagcagcatgatgcgttacgacgatcacactgtgtgcaagcccctcatctcccgggaacagcgcttttacgagtccctccctcccgaaatgaaggagttcacccctgaatacaaaggcgtggtatctgtctgttttgagggggacagtgatggttacatcaacttagtggcctatccttatgtggaaagtgagactgtggaacaggatgacacaacagaacgggagcaacctcggcgcaaacactcccgccggagcctgcaccggtcaggcagtggcagtgaccacaaggaggagaaagccagcctgtcccttgagacctctgagagctcacaggaggcaaagagtccgaaggtggagctgcacagccactcagaggtccctttccagatgctagatggcaacagtggcttgagttctgagaagatcagccacaacccctggagcctgcgttgtcacaagcagcagctgagccgcatgcgctccgagtccaaggaccgaaagctctacaagttcctcctgcttgagaacgtggtgcaccacttcaagtacccctgcgtgttggacctgaagatgggcacgcggcagcatggcgatgacgcgtcagctgagaaggcagcccggcagatgcggaaatgcgagcagagcacatcagccacgctgggcgtcagggtctgcggcatgcaggtgtaccagctggacacagggcattacctctgcaggaacaagtactatggccgtgggctctccattgaaggcttccgcaatgccctctatcaatatctgcacaatggcctggacctgcgacgtgacctgtttgagcctatcctgagcaaactgcggggcctgaaagctgtgctggagcggcaggcctcttaccgcttctactccagttccctgcttgtcatctatgatggcaaggagtgccgggctgagtcctgcctggaccgccggtctgagatgcgtctcaagcacctggacatggtgctccctgaggtggcgtcatcctgtggccccagcaccagccccagcaacaccagccccgaggcgggtccctcctctcagcccaaggtggatgtccgcatgattgactttgcacacagcacattcaagggcttccgggatgaccccaccgtgcatgatgggccagacagaggctacgtgtttggcctggagaacctcatcagcatcatggaacagatgcgggacgagaaccagtaggccctgttctgggcccccagaaccccttcctctccactgcaggcagggaccattgttctgaacttgccgtgaggacacacagacttgcttttaaagggttatatttctctttggtgtaaactaaaagaaatgtttttagctgtagcctggaatccatatatataaagtgaaggagggcagaccacacgccctctcagccaggctcctcaaaaaaaaaaaaaaaaaaaaaaaaaaaaaaaaaaaaaaaaaaaaaaaaaaaaaaaaaaaaaaaaaaaaaaaaa

SEQ ID NO: 88 - DNA, 4249 bp, Homo sapiens
gctgaccctcctgctggtgacggaagtaccgcctcctcccgtctgacgcccctcaggggaccctgcatcgctccagccgccgcggccatgtctgggccaggcaacaaacgcgccgccggcgacgggggctcagggcccccggaaaagaagctgagtcgtgaggagaagaccaccacgactcttatcgagcccattcgtcttggaggcatctcttccacggaggagatggacctgaaggtactacagttcaagaacaagaaactggcagagcggctggaacaacggcaggcttgtgaagatgaactccgagaacgaattgagaagttggagaagcggcaggccacagatgatgccacactcctcatcgtcaatcgctactgggcccagctggatgaaactgtggaagcccttctccgatgccatgagagccagggggagctgtcttcagcgcctgaggcacctgggacccaggaggggccaacatgtgatgggactcctctcccagagccggggacatcagagctgagagaccccttgctgatgcagctgcggccccctctcagtgagccggccttggcttttgtggtggcactgggtgccagcagcagtgaggaggtggagctggagctgcaaggccgaatggagttctccaaggcagctgtgtctcgtgtggtagaggcctcagaccgcctacagcgccgggtggaggaactctgtcagcgagtgtacagccgaggggacagtgagcccctcagtgaggcggctcaggcacacacccgagagctgggccgtgagaaccggcgactgcaggacttggccactcagctgcaggagaaacaccaccgcatctcattggagtactccgagctccaggataaagtgacatcggcagagaccaaggtgctggagatggagacaacagtggaggacttgcagtgggacatcgagaagctgcggaagcgagagcaaaagctcaataagcacctggcagaggccttagagcagcttaactctggctactatgtatctgggagctcctcaggcttccaggggggccagatcacactcagcatgcagaagtttgagatgctgaatgcagagttagaggaaaaccaggaactggccaacagccgtatggcagagctggagaaactgcaggccgaacttcagggggctgtgcggaccaatgagcgcctcaaggtggccctgcggagccttcctgaggaggtagtgcgggagacgggggagtaccgcatgctgcaggcccaattctcactgctctacaacgagtctctgcaagtgaagacccagctagacgaggctcggggcctgctgctggccacaaagaactcccacctgcgacacatcgagcacatggagagcgacgagctggggctgcagaagaagctacgcacagaggtcattcagctggaggacacgctggcccaggtacgcaaggagtatgagatgctgcgcatcgagtttgagcagaatctggcggccaacgagcaggcggggcccatcaaccgtgagatgcgccacctgattagtagtcttcaaaaccacaaccaccagctaaaaggggacgcccagcgatacaagcggaagcttcgagaagtacaagctgagattggcaagctccgggcccaggccagtggctctgcccactccacccccaacctgggccacccagaggattctggcgtcagtgccccagccccagggaaagaggagggtgggccaggccctgtcagtacccccgacaacagaaaggagatggctccagtgcctggcaccaccactactaccacttcagtgaagaaggaggagctggtcccctctgaagaggacttccagggtataacccctggggcccagggcccttcctcccggggccgagaacctgaggccaggcccaagcgggagcttcgggaacgggaaggtcccagcctaggacctccacctgtagcctccgctctctcaagggctgatcgggagaaggccaaggtggaagaaaccaagcggaaggaatcagaactcctcaagggtctccgagcagagctcaagaaggcccaggagagccagaaggagatgaaactgctgctggatatgtacaagtcagcgcccaaggagcagcgggataaggtgcagctcatggcagcggaacgcaaggctaaggccgaggttgatgagctgcggagccgcatccgggaattggaggagagggatcgaagggagagcaagaagatcgcggatgaggatgccctgcggcgcattcggcaggcagaggagcagatagaacacctgcagcgcaagctgggtgccaccaagcaggaggaggaggctctgctctcagagatggatgtgacaggtcaggcttttgaggacatgcaggaacagaacgggcggctgctacagcagttgcgggaaaaggatgatgccaactttaagctaatgtcagagcggatcaaggccaaccagattcacaagctgctgcgggaggagaaggatgagttgggcgagcaggtccttggcctcaagtcccaggtggatgcccagctgctgactgtgcagaagctggaggagaaggagcgagccttgcagggcagcctcgggggtgtggagaaggagctgacgctgcgcagccaagccctggagctcaacaagcggaaggctgtagaagccgcccagctggccgaggacctgaaggtgcagctggagcacgtgcagactcggctgcgggagatccagccctgcctggcagagagccgggctgctcgtgagaaagagagcttcaacctcaagagggctcaggaggacatctcacggctgcggcgcaagctggaaaagcagaggaaggtggaggtctacgcagatgccgacgaaatcctccaggaggagatcaaggagtacaaggcgcggttgacctgcccctgctgtaacacccgcaagaaggatgcagtccttaccaagtgcttccacgttttctgcttcgagtgcgtgcggggccgctatgaggcccgccagaggaagtgccccaagtgcaacgcggcctttggtgcccacgacttccatcgtatctacatcagctgaacctgaaactcaggggactctggaacaccatggaccctgggggctgtgcccccatctcctccccaccccaggtctagtggccccaccctccattccggaccccatgggcccagcccctgcccatctagttggtttggggaccctggtgcatgctagtgggcatgggatcagccaagcttcgttccatcttttcctaaaggtcagagctgcagcctagggggcactgccctacagaaaaggtctgcctgagaggcctgaggagcccagagcacttgactgagcttcccggaaactggccctaacctgtctgtctccgtggatgcatcctaaccctaaggaaaattccccaggctgtgatctaccctagagaaggctcgctccctgcctattggctcacaaatgaggaccagtgagccatgtccttgttccttgtttgagactgggctgcaggccccaggaagactttccttcacccaccatccccctaacctcggcagggcttctgtcctgtggagttccctggacaccttggtctggctcttgtgccaagggctgaaggaggtaccctcttggcagatgggggcatcacttgcttcctttgggaagctctaaggttgctgcagtcaccttcctcatcttgcaggtgctgaaccaacatcatcagtttctattctaatcaggccccttcccaatctccatttctctgccaagcccatttacccccacctcatgcatcccaaggctctactgggtccctggacctaaccctgctttcatcctggtggccttaactacagtggaggtggaacttcccaggaggggaagggacagaccagccccagccgctgggccaacttccaatcattccagctagaagagcttccccctgacaccctgtgactgagcctgtgtcctgtctgcctgcccagccatgctccatcggctgtgagggcagtgcccggagaggccagagggttggagctgcagggacccgtttggacccacagcctctgttctagagatgcttgtataggctgttaattgtgatgaataaacgttcaaccctcggcctgcaaaaaaaaaaaaaaaaaaaaaaaaaaaaaaaaaaaaaaaaaaaaaaaaaaaaaaaaaaaaaaaaaaaaaaaaaaacctcggg

SEQ ID NO: 89 - DNA, 3199 bp, Homo sapiens
gtgctgggaaacatggcgaccgagggaatgatccttactaaccacgaccatcaaatccgtgtcggagtccttacagtgagtgatagttgcttcaggaatcttgcagaagaccgcagtgggataaatctcaaagatctcgtacaagatccttctttgttgggtgggactatatcagcatacaagatagtaccagatgaaatagaagaaatcaaggaaaccctgatagattggtgtgatgaaaaggaacttaatttgatattaacaactggaggaacaggatttgcaccacgagatgtcactccagaggccacaaaagaagtaatagaacgggaagcaccagggatggccctggcaatgctgatgggatcacttaatgttacacctctgggcatgctctctaggcctgtatgtggaatcagagggaaaacgcttataattaacctgccaggtagcaagaaaggatctcaggaatgctttcaattcatactgccagctctacctcatgccattgaccttttacgtgatgccattgtaaaagtaaaggaggtgcatgatgaacttgaagatttgccttccccacctccccctctttcccctcctcctactaccagcccccataaacagacagaagacaaaggagttcaatgtgaggaagaggaagaagagaagaaagacagtggtgttgcttcaacagaagatagttcctcatcacatataactgcagcagccattgctgccaagaagcatccattctacaccagtcctgctgttgtcatggcacacggtgaacagcccatccctggtctcatcaattattcccatcattcaacagatgaacggattccagactccatcatttctcgtggtgttcaggtgctcccacgagacacagcctccctcagcactactccttcagaatcgcctcgtgctcaggctacatctcgcctctctacagcttcctgcccaacaccaaaagtccagtccaggtgcagcagcaaggagaacattctcagagccagtcacagtgctgtcgatatcaccaaggtggctagaagacatcgcatgtctccttttcctctgacatctatggacaaagcctttatcacagtcctggagatgactccggtgcttgggacagaaatcatcaattaccgagatggaatggggcgagtccttgctcaagatgtatatgcaaaagacaatttaccccccttcccagcatcagtaaaagatggctatgctgtccgagctgctgatggcccaggagatcgtttcatcattggggaatcccaagctggtgaacagccaactcagacagtaatgccaggacaagtcatgcgggttacaacaggtgctccaataccctgcggtgctgatgcagtagtacaagtggaagataccgaacttatcagggaatcagatgatggcactgaagaacttgaagtgcgaattctggtgcaagctcggccaggccaagatatcaggcccatcggccatgacattaaaagaggggaatgtgttttggccaaaggaacccacatgggcccctcagagattggtcttctggcaactgtaggtgtcacagaggttgaagttaataagtttccagtggttgcagtcatgtcaacagggaatgagctgctaaatcctgaagatgacctcttaccagggaagattcgagacagcaatcgttcaactcttctagcaacaattcaggaacatggttaccccacgatcaacttgggtattgtaggagacaacccagatgacttactcaatgccttgaatgagggtatcagtcgtgctgatgtcatcatcacatcagggggtgtatccatgggggaaaaggactatctcaagcaggtgctggacattgatcttcatgctcagatccattttggcagggtttttatgaaaccaggcttgccaacaacatttgcaactttggatattgatggtgtaagaaaaataatctttgcactacctgggaatcctgtatcggctgtggtcacctgcaatctctttgttgtgcctgcactgaggaaaatgcagggcatcttggatcctcggccaaccatcatcaaagcaaggttatcatgtgatgtaaaacttgatcctcgtccagaataccatcggtgtatactaacttggcatcaccaagaaccactaccttgggcacagagtacaggtaatcaaatgagcagccgtctgatgagcatgcgcagtgccaatggattgttgatgctacctccaaagacagaacagtacgtggagctccacaaaggcgaggtggtggatgtcatggtcattggacggctatgatggtcaccagcaggagaaagctttgatgcatgtccacatatcattgactgtatcctgtaatatgcaacggcacagctagttttcccgatttggataaaagttgatctgtatagtcaacatcttgaactatatttcaaatgaatttaaatatcttttaaagaaaaaaacacctaaaaataaatcttaacagaaaattctgttctgattatatcaaggcaaatttttcctttcttgcaaattgctttgtgtgttcaatgctaggtctgatagcgatagcttttagtagacagcggtaggtgcctgcagaacttgtgtttttctcatctttaaaatacaactacttatgctcttaaatcaaggctgtctgcttatttatactagcgtaggcaacacttggatttcccttcttagtatgcttcataactgctttacagagagcttttgcttgttctttctcatgtatctcgtgtttatgtgcacagtgccaaaagaagactgactgggtggaggctctgccttgcctcaagaaccatcccctgcagagcatccagggaggtttctcgccccaatagcctcacggcacagtactcttgggcagtaactggacaccttttatttgaagaaacaaactgaagaaaaaatgcttccttaagtgctgacagcctttttaaccaatacatttaaaattgtacagaacaaaaaaataaaatcaaagactgatcttgtacagatattagtgttaccagcattcatgtggaagtcaagagcaaagacaaaataatgttaaacaattctgtaccataacattttctgtaatgatactgaaacttaatgaataaaaaaattccttgatcattatttaaaaaaaaaaaaaaaa

SEQ ID NO: 90 - DNA, 988 bp, Homo sapiens
gccagcagggctggtgctggggcttcttctcctgaaggggctgcaagagggaaggcttagccatgtcgtccttgatcagaagggtgatcagcaccgcgaaagccccaggggccattggaccctacagtcaagctgtattagtcgacaggaccatttacatttcaggacagataggcatggacccttcaagtggacagcttgtgtcaggaggggtagcagaagaagctaaacaagctcttaaaaacatgggtgaaattctgaaagctgcaggctgtgacttcactaacgtggtgaaaacaactgttcttctggctgacataaatgacttcaatactgtcaatgaaatctacaaacagtatttcaagagtaattttcctgctagagctgcttaccaagttgctgctttacccaaaggcagccgaattgaaattgaagcagtagctatccaaggaccactgacaacggcatcactataagtgggcccagtgctgtgtagtctggaattgttaacattttaatttttacaattgatgtaacatcttaattaaccttttaattttcacaattgatgacagggtgagtttgatgaaaatatctgaagctattatggaaataccatgtaatagggagagttgaacatgaatattagagaaggaatccagttacttttttaaattacacctgtgtgcacctgtattactgaatataggaaagagatacccattacatagttactcagtaaacaaaagagaaataccaggtaggaaagaagagttactattcctgagaaataatcaagaacatatttaatttaaactaatgatgtgaactatttagttttgatgtccgttatgtgattctgcttttacttgagtaaaattaaagtgtttaaatttgagatcaaggagaagatagtggaacaaaatgttatatagataatatttttctaatggaaataaaataggcagatttcaaaaaaaaaaaaaaa

SEQ ID NO: 91 - DNA, 1298 bp, Homo sapiens
gagcggatcgcgggctcgggctgcggggctccggctgcgggcgctgggccgcgaggcgcggagcttgggagcggagcccaggccgtgccgcgcggcgccatgaagggcaaggaggagaaggagggcggcgcacggctgggcgctggcggcggaagccccgagaagagcccgagcgcgcaggagctcaaggagcagggcaatcgtctgttcgtgggccgaaagtacccggaggcggcggcctgctacggccgcgcgatcacccggaacccgctggtggccgtgtattacaccaaccgggccttgtgctacctgaagatgcagcagcacgagcaggccctggccgactgccggcgcgccctggagctggacgggcagtctgtgaaggcgcacttcttcctggggcagtgccagctggagatggagagctatgatgaggccatcgccaatctgcagcgagcttacagcctggccaaggagcagcggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggactactgaggttccctgccctacctggcgtcctggtccaggggagccctgggcagaagcccccggcccctatacatagtttatgttcctggccaccccgaccgcttcccccaagttctgctgttggactctggactgtttcccctctcagcatcgcttttgctgggccgtgatcgtccccctttgtgggctggaaaagcaggtgagggtgggctgggctgaggccattgccgccactatctgtgtaataaaatccgtgagcacgaaaaaaaaaaaaaaaaaaaaaaaaaaaaaaa

SEQ ID NO: 92 - DNA, 2002 bp, Homo sapiens
gaagcaagcggaggcgcgggggcgcgtctacgaagccggacctgtagcagtttctttggctgcctgggccccttgagtccagccatcatgcctatccgtgctctgtgcactatctgctccgacttcttcgatcactcccgcgacgtggccgccatccactgcggccacaccttccacttgcagtgcctaattcagtggtttgagacagcaccaagtcggacctgcccacagtgccgaatccaggttggcaaaagaaccattatcaataagctcttctttgatcttgcccaggaggaggagaatgtcttggatgcagaattcttaaagaatgaactggacaatgtcagagcccagctttcccagaaagacaaggagaaacgagacagccaggtcatcatcgacactctgcgggatacgctggaagaacgcaatgctactgtggtatctctgcagcaggccttgggcaaggccgagatgctgtgctccacactgaaaaagcagatgaagtacttagagcagcagcaggatgagaccaaacaagcacaagaggaggcccgccggctcaggagcaagatgaagaccatggagcagattgagcttctactccagagccagcgccctgaggtggaggagatgatccgagacatgggtgtgggacagtcagcggtggaacagctggctgtgtactgtgtgtctctcaagaaagagtacgagaatctaaaagaggcacggaaggcctcaggggaggtggctgacaagctgaggaaggatttgttttcctccagaagcaagttgcagacagtctactctgaattggatcaggccaagttagaactgaagtcagcccagaaggacttacagagtgctgacaaggaaatcatgagcctgaaaaagaagctaacgatgctgcaggaaaccttgaacctgccaccagtggccagtgagactgtcgaccgcctggttttagagagcccagcccctgtggaggtgaatctgaagctccgccggccatccttccgtgatgatattgatctcaatgctacctttgatgtggatactcccccagcccggccctccagctcccagcatggttactacgaaaaactttgcctagagaagtcacactccccaattcaggatgtccccaagaagatatgcaaaggccccaggaaggagtcccagctctcactgggtggccagagctgtgcaggagagccagatgaggaactggttggtgccttccctatttttgtccggaatgccatcctaggccagaaacagcccaagaggcccaggtcagagtcctcttgcagcaaagatgtggtaaggacaggcttcgatgggctcggtggccggacaaaattcatccagcctactgacacagtcatgatccgcccattgcctgttaagcccaagaccaaggttaagcagagggtgagggtgaagacagtgccttctctcttccaggccaagctggacaccttcctgtggtcgtgagaacagtgagtctgaccaatggccagacacatgcctgcaacttgtaggtcaaggactgtccaggcaggggttttgtggacagagccccactttcgggaccagcctgaggtgtaagggcagacaaacaggtgaggatgagtgtggcacccagagactgctcttcctgccctcaccctgccccactcctacgactgggagctgacatgaccagcccactgatcctgtcagcaggtcctgctcctgttgccaggctcctgtttatagccatgatcagatgtggtcagactctttctgggcctggagaccacggtcacttgttgactgtctctgtggaccagagtgcttgaggcatctcaggcagcctcagcccaagcttctacctgcctttgacttgcttctaggcatagcctgggccaagcagggtgggtaatggaggatagcatgggatgtatggagaggatggaagattttcatgtaaaataaaattaaaaaaaaaaaaaaaaaa

SEQ ID NO: 93 - DNA, 3225 bp, Homo sapiens
accaccagcctagctcggacggcaagcggcgggagattttcaaaatgggagcccagaggcaccgcccaggcctcggaaggtgtcagggagaactttccgtggtttcagcgtcgtcgcctggagcggcggtttagagagccgagcctgatgggcgccaaggccggctggctgcttggagcgctgcctcgaagggactgcgtgaaggaagctaatccggagaacccaggccagagcctggaaatatggcgacctgcatcggggagaagatcgaggattttaaagttggaaatctgcttggtaaaggatcatttgctggtgtctacagagctgagtccattcacactggtttggaagttgcaatcaaaatgatagataagaaagccatgtacaaagcaggaatggtacagagagtccaaaatgaggtgaaaatacattgccaattgaaacatccttctatcttggagctttataactattttgaagatagcaattatgtgtatctggtattagaaatgtgccataatggagaaatgaacaggtatctaaagaatagagtgaaacccttctcagaaaatgaagctcgacacttcatgcaccagatcatcacagggatgttgtatcttcattctcatggtatactacaccgggacctcacactttctaacctcctactgactcgtaatatgaacatcaagattgctgattttgggctggcaactcaactgaaaatgccacatgaaaagcactatacattatgtggaactcctaactacatttcaccagaaattgccactcgaagtgcacatggccttgaatctgatgtttggtccctgggctgtatgttttatacattacttatcgggagaccacccttcgacactgacacagtcaagaacacattaaataaagtagtattggcagattatgaaatgccatcttttttgtcaatagaggccaaggaccttattcaccagttacttcgtagaaatccagcagatcgtttaagtctgtcttcagtattggaccatccttttatgtcccgaaattcttcaacaaaaagtaaagatttaggaactgtggaagactcaattgatagtgggcatgccacaatttctactgcaattacagcttcttccagtaccagtataagtggtagtttatttgacaaaagaagacttttgattggtcagccactcccaaataaaatgactgtatttccaaagaataaaagttcaactgatttttcttcttcaggagatggaaacagtttttatactcagtggggaaatcaagaaaccagtaatagtggaaggggaagagtaattcaagatgcagaagaaaggccacattctcgataccttcgtagagcttattcctctgatagatctggcacttctaatagtcagtctcaagcaaaaacatatacaatggaacgatgtcactcagcagaaatgctttcagtgtccaaaagatcaggaggaggtgaaaatgaagagaggtactcacccacagacaacaatgccaacatttttaacttctttaaagaaaagacatccagtagttctggatcttttgaaagacctgataacaatcaagcactctccaatcatctttgtccaggaaaaactccttttccatttgcagacccgacacctcagactgaaaccgtacaacagtggtttgggaatctgcaaataaatgctcatttaagaaaaactactgaatatgacagcatcagcccaaaccgggacttccagggccatccagatttgcagaaggacacatcaaaaaatgcctggactgatacaaaagtcaaaaagaactctgatgcttctgataatgcacattctgtaaaacagcaaaataccatgaaatatatgactgcacttcacagtaaacctgagataatccaacaagaatgtgtttttggctcagatcctctttctgaacagagcaagactaggggtatggagccaccatggggttatcagaatcgtacattaagaagcattacatctccgttggttgctcacaggttaaaaccaatcagacagaaaaccaaaaaggctgtggtgagcatacttgattcagaggaggtgtgtgtggagcttgtaaaggagtatgcatctcaagaatatgtgaaagaagttcttcagatatctagtgatggaaatacgatcactatttattatccaaatggtggtagaggttttcctcttgctgatagaccaccctcacctactgacaacatcagtaggtacagctttgacaatttaccagaaaaatactggcgaaaatatcaatatgcttccaggtttgtacagcttgtaagatctaaatctcccaaaatcacttattttacaagatatgctaaatgcattttgatggagaattctcctggtgctgattttgaggtttggttttatgatggggtaaaaatacacaaaacagaagatttcattcaggtgattgaaaagacagggaagtcttacactttaaaaagtgaaagtgaagttaatagcttgaaagaggagataaaaatgtatatggaccatgctaatgagggtcatcgtatttgtttagcactggaatccataatttcagaagaggaaaggaaaactaggagtgctccctttttcccaataatcataggaagaaaacctggtagtactagttcacctaaggccttatcacctcctccttctgtggattcaaattacccaacgagagagagagcatctttcaacagaatggtcatgcatagtgctgcttctccaacacaggcaccaatccttaatccctctatggttacaaatgaaggacttggtcttacaactacagcttctggaacagacatctcttctaatagtctaaaagattgtcttcctaaatcagcacaacttttgaaatctgtttttgtgaaaaatgttggttgggctacacagttaactagtggagctgtgtgggttcagtttaatgatgggtcccagttggttgtgcaggcaggagtgtcttctatcagttatacctcaccaaatggtcaaacaactaggtatggagaaaatgaaaaattaccagactacatcaaacagaaattacagtgtctgtcttccatccttttgatgttttctaatccgactcctaattttcattgattaaaactcctttcagacatataagtttaataaataacttttttgttgactttcaaaaaaaaaaaaaaaa

SEQ ID NO: 94 - DNA, 1817 bp, Homo sapiens
ccgcgagcggacgcggcagcgcctctgtctcgctttttcttatttttcccccctttcccctttctttttttttttttcttttcttttctcccctccccccctttcaccatttcccctcggaggcgctttccccgggcaggggcagagccggtctcaccccccgcctctccccggcccccgccgccctatggcgagagggagccccctcccaacccgggctcgagcggcggcggcctcaggccgggggtcatcatggaactaattcgctgaccgacccagcggccgcagccgtgcgtcccgctcgagcgccagcgcccgcgcccgcgccccccgatccgcttcccctttctccctcctcagttggccgagtcgtcccgcgcgcaccgcctccgcgcgcctatgagaatgaggtggtaacgggcccccggatgaccccgcgtcaccactgtgaggcctacagctctgccggggaggaggaggaggaggaagaggaggagaaggtagctacagcaagctgggtagcaggcagatccaaaggatatcatgaagtttccagggcctttggaaaaccagagattgtctttcctgttggaaaaggcaatcactagggaagcacagatgtggaaagtgaatgtgcggaaaatgccttcaaatcagaatgtttctccatcccagagagatgaagtaattcaatggctggccaaactcaagtaccaattcaacctttacccagaaacatttgctctggctagcagtcttttggataggtttttagctaccgtaaaggctcatccaaaatacttgagttgtattgcaatcagctgttttttcctagctgccaagactgttgaggaagatgagagaattccagtactaaaggtattggcaagagacagtttctgtggatgttcctcatctgaaattttgagaatggagagaattattctggataagttgaattgggatcttcacacagccacaccattggattttcttcatattttccatgccattgcagtgtcaactaggcctcagttacttttcagtttgcccaaattgagcccatctcaacatttggcagtccttaccaagcaactacttcactgtatggcctgcaaccaacttctgcaattcagaggatccatgcttgctctggccatggttagtctggaaatggagaaactcattcctgattggctttctcttacaattgaactgcttcagaaagcacagatggatagctcccagttgatccattgtcgggagcttgtggcacatcacctttctactctgcagtcttccctgcctctgaattccgtttatgtctaccgtcccctcaagcacaccctggtgacctgtgacaaaggagtgttcagattacatccctcctctgtcccaggcccagacttctccaaggacaacagcaagccagaagtgccagtcagaggtacagcagccttttaccatcatctcccagctgccagtgggtgcaagcagacctctactaaacgcaaagtagaggaaatggaagtggatgacttctatgatggaatcaaacggctctataatgaagataatgtctcagaaaatgtgggttctgtgtgtggcactgatttatcaagacaagagggacatgcttccccttgtccacctttgcagcctgtttctgtcatgtagtttcaacaagtgctacctttgagtgtaaactaaggtagactactttgggaatgagaacatgcaaaatcaggaaaggctgtagaaggaaatataccttaacaggctgatttggagtgagccagaaaaaaaaaaaaaaaaaa

SEQ ID NO: 95 - DNA, 1309 bp, Homo sapiens
atgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagggacaagacatgactgtgatgaggagctgctttcgccaatttaacaccaagaagaattgaggctgcttgggaggaaggccaggaggaacacgagactgagagatgaattttcaacagaggctgcaaagcctgtggactttagccagcagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtcaaaaaaaaaaaaaaaa

SEQ ID NO: 96 - DNA, 1879 bp, Homo sapiens
ggagaagtggagtttggaagttcaggggcacaggggcacaggcccacgactgcagcgggatggaccagtactgcatcctgggccgcatcggggagggcgcccacggcatcgtcttcaaggccaagcacgtggagactggcgagatagttgccctcaagaaggtggccctaaggcggttggaagacggcttccctaaccaggccctgcgggagattaaggctctgcaggagatggaggacaatcagtatgtggtacaactgaaggctgtgttcccacacggtggaggctttgtgctggcctttgagttcatgctgtcggatctggccgaggtggtgcgccatgcccagaggccactagcccaggcacaggtcaagagctacctgcagatgctgctcaagggtgtcgccttctgccatgccaacaacattgtacatcgggacctgaaacctgccaacctgctcatcagcgcctcaggccagctcaagatagcggactttggcctggctcgagtcttttccccagacggcagccgcctctacacacaccaggtggccaccaggtctgtgggctgcatcatgggggagctgttgaatgggtccccccttttcccgggcaagaacgatattgaacagctttgctatgtgcttcgcatcttgggcaccccaaaccctcaagtctggccggagctcactgagctgccggactacaacaagatctcctttaaggagcaggtgcccatgcccctggaggaggtgctgcctgacgtctctccccaggcattggatctgctgggtcaattccttctctaccctcctcaccagcgcatcgcagcttccaagctcccctgcctgcccatccatctgagctgccgattcctcagcgtctagggggacctgcccccaaggcccatccagggcccccccacatccatgacttccacgtggaccggcctcttgaggagtcgctgttgaacccagagctgattcggcccttcatcctggaggggtgagaagttggccctggtcccgtctgcctgctcctcaggaccactcagtccacctgttcctctgccacctgcctggcttcaccctccaaggcctccccatggccacagtgggcccacaccacaccctgccccttagcccttgcgagggttggtctcgaggcagaggtcatgttcccagccaagagtatgagaacatccagtcgagcagaggagattcatggcctgtgctcggtgagccttaccttctgtgtgctactgacgtacccatcaggacagtgagctctgctgccagtcaaggcctgcatatgcagaatgacgatgcctgccttggtgctgcttccccgagtgctgcctcctggtcaaggagaagtgcagagagtaaggtgtccttatgttggaaactcaagtggaaggaagatttggtttggttttattctcagagccattaaacactagttcagtatgtgagatatagattctaaaaacctcaggtggctctgccttatgtctgttcctccttcatttctctcaagggaaatggctaaggtggcattgtctcatggctctcgtttttggggtcatggggagggtagcaccaggcatagccacttttgccctgagggactcctgtgtgcttcacatcactgagcactcatttagaagtgagggagacagaagtctaggcccagggatggctccagttggggatccagcaggagaccctctgcacatgaggctggtttaccaacatctactccctcaggatgagcgtgagccagaagcagctgtgtatttaaggaaacaagcgttcctggaattaatttataaatttaataaatcccaatataatcccaaaaaaaaaaaaaaaaaaaa

SEQ ID NO: 97 - DNA, 2777 bp, Homo sapiens
gagagtgcggagtgtgtgctccgggctcggaacacacatttattattaaaaaatccaaaaaaaatctaaaaaaatcttttaaaaaaccccaaaaaaaaatttacaaaaaatccgcgtctcccccgccggagacttttattttttttcttcctcttttataaaataacccggtgaagcagccgagaccgaccctcgctccccgccggcagccggcagccagcggcagtggatcgaccccgttctgcggccgttgagtagttttcaattccggttgatttttgtccctctgcgcttgctccccgctcccctccccccggctccggcccccagccccggcactcgctctcctcctctcacggaaaggtcgcggcctgtggccctgcgggcagccgtgccgagatgaaccccagtgcccccagctaccccatggcctcgctctacgtgggggacctccaccccgacgtgaccgaggcgatgctctacgagaagttcagcccggccgggcccatcctctccatccgggtctgcagggacatgatcacccgccgctccttgggctacgcgtatgtgaacttccagcagccggcggacgcggagcgtgctttggacaccatgaattttgatgttataaagggcaagccagtacgcatcatgtggtctcagcgtgatccatcacttcgcaaaagtggagtaggcaacatattcattaaaaatctggacaaatccattgataataaagcactgtatgatacattttctgcttttggtaacatcctttcatgtaaggtggtttgtgatgaaaatggttccaagggctatggatttgtacactttgagacgcaggaagcagctgaaagagctattgaaaaaatgaatggaatgctcctaaatgatcgcaaagtatttgttggacgatttaagtctcgtaaagaacgagaagctgaacttggagctagggcaaaagaattcaccaatgtttacatcaagaattttggagaagacatggatgatgagcgccttaaggatctctttggcaagtttgggcctgccttaagtgtgaaagtaatgactgatgaaagtggaaaatccaaaggatttggatttgtaagctttgaaaggcatgaagatgcacagaaagctgtggatgagatgaacggaaaggagctcaatggaaaacaaatttatgttggtcgagctcagaaaaaggtggaacggcagacggaacttaagcgcaaatttgaacagatgaaacaagataggatcaccagataccagggtgttaatctttatgtgaaaaatcttgatgatggtattgatgatgaacgtctccggaaagagttttctccatttggtacaatcactagtgcaaaggttatgatggagggtggtcgcagcaaagggtttggttttgtatgtttctcctccccagaagaagccactaaagcagttacagaaatgaacggtagaattgtggccacaaagccattgtatgtagctttagctcagcgcaaagaagagcgccaggctcacctcactaaccagtatatgcagagaatggcaagtgtacgagctgttcccaaccctgtaatcaacccctaccagccagcacctccttcaggttacttcatggcagctatcccacagactcagaaccgtgctgcatactatcctcctagccaaattgctcaactaagaccaagtcctcgctggactgctcagggtgccagacctcatccattccaaaatatgcccggtgctatccgcccagctgctcctagaccaccatttagtactatgagaccagcttcttcacaggttccacgagtcatgtcaacacagcgtgttgctaacacatcaacacagacaatgggtccacgtcctgcagctgcagccgctgcagctactcctgctgtccgcaccgttccacagtataaatatgctgcaggagttcgcaatcctcagcaacatcttaatgcacagccacaagttacaatgcaacagcctgctgttcatgtacaaggtcaggaacctttgactgcttccatgttggcatctgcccctcctcaagagcaaaagcaaatgttgggtgaacggctgtttcctcttattcaagccatgcaccctactcttgctggtaaaatcactggcatgttgttggagattgataattcagaacttcttcatatgctcgagtctccagagtcactccgttctaaggttgatgaagctgtagctgtactacaagcccaccaagctaaagaggctgcccagaaagcagttaacagtgccaccggtgttccaactgtttaaaattgatcagggaccatgaaaagaaacttgtgcttcaccgaagaaaaatatctaaacatcgaaaaacttaaatattatggaaaaaaaacattgcaaaatataaaataaataaaaaaaggaaaggaaactttgaaccttatgtaccgagcaaatgccaggtctagcaaacataatgctagtcctagattacttattgatttaaaaacaaaaaaacacaaaaaaatagtaaaatataaaaacaaattaatgttttatagaccctgggaaaaagaattttcagcaaagtacaaaaatttaaagcattcctttctttaattttgtaattctttactgtggaatagctcagaatgtcagttctgttttaagtaacagaattgataactgagcaaggaaacgtaatttggattataaaattcttgctttaataaaaattccttaaacagtgcaaaaaaaaaaaaaaaaaaaaa

SEQ ID NO: 98 - DNA, 1436 bp, Homo sapiens
gctctgcgcgtgcgccggtggcgggactctggggaaaatggctgcgtcttcgagtggtgagaaggagaaggagcggctgggaggcggtttgggagtggcgggtggtaacagcacacgagagcggctgctgtctgcgcttgaggacttggaggtcctgtctagggaacttatagaaatgctggcaatttcaagaaaccagaagttgttacaggctggagaggaaaaccaggtcctggagttgttaattcaccgagatggggaatttcaagaactaatgaaattggcacttaatcagggaaaaattcatcatgaaatgcaagttttagaaaaagaagtagagaagagagacggtgatattcagcagctacaaaaacagctaaaggaagcagaacaaatactggcaacagctgtttaccaagcgaaggagaaactcaagtcaatagaaaaagcaagaaaaggtgctatctcctctgaagaaataattaagtatgcacataggatcagtgcaagtaatgctgtatgtgctccactgacctgggttccaggggacccccggagaccctacccaactgatttagagatgagaagtgggttactgggtcagatgaacaatccttccactaatggcgtgaatggccatttaccaggagatgcacttgcagcaggaagattgccagatgtccttgctccacagtatccatggcagtcaaatgacatgtcgatgaatatgttaccaccaaatcatagtagtgactttttgttggaacctcctgggcataataaagaagatgaagatgatgtagagattatgtcaacggactcctcaagcagtagtagtgagtctgattgaaaaaccttaaaagacaatatacagaattgaatactgtagaattctgtttctttaacagtagcagggaaatgtaaactacaggtgacaaaaaatacccaggtaaacactggctttggtagaattgtgcagtcattaaaagtcaaaattttttgactttctttttaaagccaaagaccatagttttagttttaagccactaggtagatatttaggggaatagtcaaaatttactgttgaaaaagcagttgctatgtgctttcttaccctgttctgttccagttttgctggatttgtacatagccattctagaaatagagttgagggaaattatccatatacatatcactaataggcttcttggaattatttagaaaagcatttttaaactggcagtggatgactgaataggcatcatatttctttttgtgtgtcatttaaaagtaacaaaaactgccatttgacagtaaaggctcttggcttctgttggaggcatgggaaattgtctcaatttgtacagtttgtaattgtaatttttgtaaataaatttgtttgtacataaaaaaaaaaaaaaaaaaaaaaaaaaaaa

SEQ ID NO: 99 - DNA, 1493 bp, Homo sapiens
gcctgagtaaccgtatgatggtggtggtggtggtgtcttcctgtctcaacgatacctattttctagtgctgagatcctgagacaatgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttctctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtggtgtggaaagtaaagaagtcacaggttgggacatttagacaagagtgaatcacacacgaggaatgtgttcattcttttattgtccgttgttttaacctgactgaatacaagatcaacaagagcactgtactcctggcaattattacatatgttagaacatggattttgcactgtagacaacatttaacaccagtctatggggtactgcattgctttttataaagttcaaaataaagatttattttcaaaaaaaaaaaaaaaaaaaaaaaaaaaaaa

SEQ ID NO: 100 - DNA, 1378 bp, Homo sapiens
acccgcagctgcagcacagtcatggcccaggcgtcgccgccccggcccgagagggtgctcggcgccagcagcccggaggcccggcccgcgcaggaggcgctcctccttcccaccggtgtctttcaggttgcagaaaagatggaaaaaaggacatgtgcactctgccccaaagatgtcgaatataatgtcctgtactttgcacaatcagagaatatagctgctcatgagaattgtttgctgtattcttcaggacttgtggaatgtgaggatcaggatccacttaatcctgatagaagttttgatgtggaatcagtaaagaaagaaatccagagaggaaggaagttgaaatgcaaattttgtcataaaagaggagccaccgtgggatgtgatttaaaaaactgtaacaagaattaccactttttctgtgccaagaaggacgacgcagttccacagtctgatggagttcgaggaatttataaactgctttgccagcaacatgctcaattcccgatcatcgctcaaagtgctaaattttcaggagtgaaaagaaaaagaggaaggaagaaacccctctcaggcaatcatgtacagccacccgaaacaatgaaatgtaatacattcataagacaagtgaaagaagagcatggcagacacacagatgcaactgtgaaagttccttttcttaagaaatgcaaggaagcaggacttcttaattacttacttgaagaaatattagacaaagttcattcaattccagaaaaactcatggatgagactacttcagaatcagactatgaagaaatcgggagtgcactttttgactgtagattgttcgaagacacatttgtaaattttcaagcagcaatagagaaaaaaattcatgcatctcaacaaaggtggcagcagttgaaggaagagattgagctacttcaggacttaaaacaaaccttgtgctcttttcaagaaaatagagatcttatgtcaagttctacatcaatatcatccctgtcttattagggattaccgtttcctaagccaagagtcatgtcaaattgcaatcaggctcaaaaccagagaccaggctgtgaaatccacacatctttagaactagtcgtctcctcttggcctcagcagctcttccctgttcttactggttgacattttgatcactctttgcacactcttgtgttttttgctcactgtcacattcccagcacctagtatgctcagtaaatgtttgtggaataagtgcataaaatgttcttaacctttgattctacttacagcccatgatagcctcttcttagatataataaatttggattatactaaaaaaaaaaaaaaaaaaaaaaaaaaaaaaaaaaaaaaaaaaaaaaaa

SEQ ID NO: 101 - DNA, 1629 bp, Homo sapiens
gggggcttcctagttcggctggttcttctgtcgccggcttcagcagcccgcgcccgggcaggaatagaagatgaacaaacccataacaccatcaacatatgtgcgctgcctcaatgttggactaattaggaagctgtcagattttattgatcctcaagaaggatggaagaagttagctgtagctattaaaaaaccatctggtgatgatagatacaatcagtttcacataaggagatttgaagcattacttcaaactggaaaaagtcccacttctgaattactgtttgactggggcaccacaaattgcacagttggtgatcttgtggatcttttgatccaaaatgaattttttgctcctgcaagtcttttgctcccagatgctgttcccaaaactgctaatacactaccttctaaagaagctataacagttcagcaaaaacagatgcctttctgtgacaaagacaggacattgatgacacctgtgcagaatcttgaacaaagctatatgccacctgactcctcaagtccagaaaataaaagtttagaagttagtgatacacgttttcacagtttttcattttatgaattgaagaatgtcacaaataactttgatgaacgacccatttctgttggtggtaataaaatgggagagggaggatttggagttgtatataaaggctacgtaaataacacaactgtggcagtgaagaagcttgcagcaatggttgacattactactgaagaactgaaacagcagtttgatcaagaaataaaagtaatggcaaagtgtcaacatgaaaacttagtagaactacttggtttctcaagtgatggagatgacctctgcttagtatatgtttacatgcctaatggttcattgctagacagactctcttgcttggatggtactccaccactttcttggcacatgagatgcaagattgctcagggtgcagctaatggcatcaattttctacatgaaaatcatcatattcatagagatattaaaagtgcaaatatcttactggatgaagcttttactgctaaaatatctgactttggccttgcacgggcttctgagaagtttgcccagacagtcatgactagcagaattgtgggaacaacagcttatatggcaccagaagctttgcgtggagaaataacacccaaatctgatatttacagctttggtgtggttttactagaaataataactggacttccagctgtggatgaacaccgtgaacctcagttattgctagatattaaagaagaaattgaagatgaagaaaagacaattgaagattatattgataaaaagatgaatgatgctgattccacttcagttgaagctatgtactctgttgctagtcaatgtctgcatgaaaagaaaaataagagaccagacattaagaaggttcaacagctgctgcaagagatgacagcttcttaaaactttattggaaaagactcttgactttttatatacacctatctcaaccatttttttaactgatttttttcctaaatattcttctttacctttaacaaggcataggctgttgcaggacagtggttattaaagcatgggttgaacttccaaaaaaaaaaaaaaaaaaaaaaaaaaaa

SEQ ID NO: 102 - DNA, 2350 bp, Homo sapiens
ggagtttggggatgaggcagctgaagaacagagtcttgtgatggtggaaccaggacttcgttgttccttcattgtgtgggcagaagaggtggaggattttaggcgccacctggtcttgtccccactgcccagacaggcagggaatccctcttcttccttttgctgaatcagctggagaagaaagcccagcaggaggcctgctagcctcagcctcgggccctaccagggtcctaggtactggaatggaacttcgccgaggtcaccgctcaaacggccacaacgagggagccgatttagatcctctgggcctgttccttccttttctttaaacgtcccagtctagcttagaggaggacctgttttgttagataaatggcaagcaaaaaacgagaagtccagttacagacagtcatcaataatcaaagcctgtgggatgagatgttgcagaacaaaggcttaacagtgattgatgtttaccaagcctggtgtggaccttgcagagcaatgcaacctttattcagaaaattgaaaaatgaactgaacgaagacgaaattctgcattttgctgtcgcagaagctgacaacattgtgactttgcagccatttagagataaatgtgaacctgtttttctctttagtgttaatggcaaaattatcgaaaagattcagggtgcaaatgcaccgcttgttaataaaaaagttattaatttgatcgatgaggagagaaaaattgcagcaggtgaaatggctcgacctcagtatcctgaaattccattagtagactcagattcagaagttagtgaagaatcaccatgtgaaagtgttcaggaattatacagtattgctattatcaaaccggatgctgtgattagtaaaaaagttctagaaattaaaagaaaaattaccaaagctggatttattatagaagcagagcataagacagtgctcactgaagaacaagttgtcaacttctatagtcgaatagcagaccagcgtgacttcgaagagtttgtctcttttatgacaagtggcttaagctatattctagttgtatctcaaggaagtaaacacaatcctccctctgaagaaaccgaaccacagactgacaccgaacctaacgaacgatctgaggatcaacctgaggtcgaagcccaggttacacctggaatgatgaagaacaaacaagacagtttacaagaatatctggaaagacaacatttagctcagctctgtgacattgaagaggatgcagctaatgttgctaagttcatggatgctttcttccccgattttaaaaaaatgaaaagcatgaaattagaaaagacattggcattacttcgaccaaatctctttcatgaaaggaaagatgatgttttgcgtattattaaagatgaagacttcaaaatactggagcaaagacaagtagtattatcggaaaaagaagcacaagcactgtgcaaggaatatgaaaatgaagactattttaataaacttatagaaaacatgaccagtggtccatctctagcccttgttttattgagagacaatggcttgcaatactggaaacaattactgggaccaagaactgttgaagaagccattgaatattttccagagagtttatgtgcacagtttgcgatggacagtttgccggtcaaccagttgtatggcagcgattcattagaaaccgctgaaagggaaatacagcatttctttcctcttcaaagcactttaggcttgattaaacctcatgcaacaagtgaacaaagagagcagatcctgaagatagttaaggaggctggatttgatctgacacaggtgaagaaaatgttcctaactcctgagcaaactgagaaaatttatccaaaagtaacaggaaaagacttttataaagatttattggaaatgttatctgtgggtccatctatggtcatgattctgaccaagtggaatgctgttgcagaatggagacgattgatgggcccaacagacccagaagaagcaaaattactttcccctgactccatccgagcccagtttggaataagtaaattgaaaaacattgtccatggagcatctaacgcctatgaagcaaaagaggttgttaatagactctttgaggatcctgaggaaaactaaagtatatactgtgaaaactttgagaagataatacatatgttcacgtcaatatacaaccatttggcacagcttcctgggaggaataataagaaaaacatgctttggaggaaaactcaagatacaaaaatgaatggctatgcataataacaataaaaatgtattccccaaccaaaaaaaaaaaaaaaaaaaaaaaaaaaaaaaaaaaaaaaaa

SEQ ID NO: 103 - DNA, 3139 bp, Homo sapiens
gtggcgccgggcggcggggtcctgcgtggagagtgggacgcaacgccgagaccgcgagcagaggctgcgcacagccggatccggcactcagcgaccggacccaaggatccgccggggaacaagccacaggagagcgactcaggaacaagtgtgggagaggaagcggcggcggcggcgccgggcccgggggtggtgacagcaggtctgaggttgcatcataaatacaaaggactgaagttataaaagagaaaagagaagtttgctgctaaaatgaatctgagcaatatggaatattttgtgccacacacaaaaaggtactgaagatttaccccccaaaaaaaattgtcaatgagaaataaagctaactgatatcaaaaagcagagcctgctctactggccatcatgcgtaaaggggtgctgaaggacccagagattgccgatctattctacaaagatgatcctgaggaactttttattggtttgcatgaaattggacatggaagttttggagcagtttattttgctacaaatgctcacaccagtgaggtggtggcaattaagaagatgtcctatagtgggaagcagacccatgagaaatggcaagatattcttaaggaagttaaatttttacgacaattgaagcatcctaatactattgagtacaaaggctgttacttgaaagaacacactgcttggttggtgatggaatattgcttaggctcagcctctgatttattagaagttcataaaaaaccacttcaggaagtggagatcgctgccattactcatggagccttgcatggactagcctacctacattctcatgcattgattcatagggatattaaagcaggaaatattcttctaacagagccaggtcaggtaaaactagctgattttggatctgcttcaatggcttctcctgccaactccttcgtgggcacaccttactggatggctccagaggtgatcttagctatggatgaaggacagtatgatgggaaagttgatatttggtcacttggcatcacttgtattgaattggcggaacggaagccgccccttttcaacatgaatgcaatgagtgccttatatcacattgcccagaatgactccccaacgttacagtctaatgaatggacagactcctttaggagatttgttgattactgcttgcagaaaatacctcaggaaaggccaacatcagcagaactattaaggcatgactttgttcgacgagaccggccactacgtgtcctcattgacctcatacagaggacaaaagatgcagttcgtgagctagataacctacagtaccgaaaaatgaaaaaaatacttttccaagagacacggaatggacccttgaatgagtcacaggaggatgaggaagacagtgaacatggaaccagcctgaacagggaaatggacagcctgggcagcaaccattccattccaagcatgtccgtgagcacaggcagccagagcagcagtgtgaacagcatgcaggaagtcatggacgagagcagttccgaacttgtcatgatgcacgatgacgaaagcacaatcaattccagctcctccgtcgtgcataagaaagatcatgtattcataagggatgaggcgggccacggcgatcccaggcctgagccgcggcctacccagtcagttcagagccaggccctccactaccggaacagagagcgctttgccacgatcaaatcagcatctttggttacacgacagatccatgagcatgagcaggagaacgagttgcgggaacagatgtcaggttataagcggatgcggcgccagcaccagaagcagctgatcgccctggagaacaagctgaaggctgagatggacgagcaccgcctcaagctacagaaggaggtggagacgcatgccaacaactcgtccatcgagctggagaagctggccaagaagcaagtggctatcatagaaaaggaggcaaaggtagctgcagcagatgagaagaagttccagcaacagatcttggcccagcagaagaaagatttgacaactttcttagaaagtcagaagaagcagtataagatttgtaaggaaaaaataaaagaggaaatgaatgaggaccatagcacacccaagaaagagaagcaagagcggatctccaaacataaagagaacttgcagcacacacaggctgaagaggaagcccaccttctcactcaacagagactgtactacgacaaaaattgtcgtttcttcaagcggaaaataatgatcaagcggcacgaggtggagcagcagaacattcgggaggaactaaataaaaagaggacccagaaggagatggagcatgccatgctaatccggcacgacgagtccacccgagagctagagtacaggcagctgcacacgttacagaagctacgcatggatctgatccgtttacagcaccagacggaactggaaaaccagctggagtacaataagaggcgagaaagagaactgcacagaaagcatgtcatggaacttcggcaacagccaaaaaacttaaaggccatggaaatgcaaattaaaaaacagtttcaggacacttgcaaagtacagaccaaacagtataaagcactcaagaatcaccagttggaagttactccaaagaatgagcacaaaacaatcttaaagacactgaaagatgagcagacaagaaaacttgccattttggcagagcagtatgaacagagtataaatgaaatgatggcctctcaagcgttacggctagatgaggctcaagaagcagaatgccaggccttgaggctacagctccagcaggaaatggagctgctcaacgcctaccagagcaaaatcaagatgcaaacagaggcacaacatgaacgtgagctccagaagctagagcagagagtgtctctgcgcagagcacaccttgagcagaagattgaagaggagctggctgcccttcagaaggaacgcagcgagagaataaagaacctattggaaaggcaagagcgagagattgaaacttttgacatggagagcctcagaatgggatttgggaatttggttacattagattttcctaaggaggactacagatgagattaaattttttgccatttacaaaaaaaaaaaaaaaaaa

SEQ ID NO: 104 - DNA, 1389 bp, Homo sapiens
ctggcaaccccggtgtttggctgggttctagcgtagccgtctgtgtggccggtgggggacctgcggtcggagtgggagggccagtctgcacccaagaggtggaagaggacgggctttaggctggaagcgccttagaggagccatttttccaggtggggccccaggcagaggctccgacagggagcctggccatagtcgcgcagccggggaggtggagcgcgtcccagacccgagcccccgacctcagccaaacccattccttctgtccttggaggccagaggggactctgagcatcggaaagcaggatgcctggtttgcttttatgtgaaccgacagagctttacaacatcctgaatcaggccacaaaactctccagattaacagaccccaactatctctgtttattggatgtccgttccaaatgggagtatgacgaaagccatgtgatcactgcccttcgagtgaagaagaaaaataatgaatatcttctcccggagtctgtggacctggagtgtgtgaagtactgcgtggtgtatgataacaacagcagcaccctggagatactcttaaaagatgatgatgatgattcagactctgatggtgatggcaaagatcttgtgcctcaagcagccattgagtatggcaggatcctgacccgcctcacccaccaccccgtctacatcctgaaagggggctatgagcgcttctcaggcacgtaccactttctccggacccagaagatcatctggatgcctcaggaactggatgcatttcagccataccccattgaaatcgtgccagggaaggtcttcgttggcaatttcagtcaagcctgtgaccccaagattcagaaggacttgaaaatcaaagcccatgtcaatgtctccatggatacagggcccttttttgcaggcgatgctgacaagcttctgcacatccggatagaagattccccggaagcccagattcttcccttcttacgccacatgtgtcacttcattgaaattcaccatcaccttggctctgtcattctgatcttttccacccagggtatcagccgcagttgtgccgccatcatagcctacctcatgcatagtaacgagcagaccttgcagaggtcctgggcctatgtcaagaagtgcaaaaacaacatgtgtccaaatcggggattggtgagccagctgctggaatgggagaagactatccttggagattccatcacaaacatcatggatccgctctactgatcttctccgaggcccaccgaagggtactgaagagcctcacctgggggcattttgtgggtggagggccagagtgtgtatacccaggcttgtctggaaggagaaggcctttgctgcctgaaagtcaaaaaaaaaaaaaaaaaa

SEQ ID NO: 105 - DNA, 1331 bp, Homo sapiens
cggcgccggaagtggtcgcgggtcgttctgcttcctgcccgaggggcgtgcgcgggtcaggggcggccgcggaggcggaagcatccatggcggagggcggcagcccagacgggcgggcagggccgggctccgcaggtcgtaatctgaaggagtggctgagggagcaattttgtgatcatccgctggagcactgtgaggacacgaggctccatgatgcagcttacgtcggggacctccagaccctcaggagcctattgcaagaggagagctaccggagccgcatcaacgagaagtctgtctggtgctgtggctggctcccctgcacaccgttgcgaatcgcggccactgcaggccatgggagctgtgtggacttcctcatccggaagggggccgaggtggatctggtggacgtaaaaggacagacggccctgtatgtggctgtggtgaacgggcacctagagagtacccagatccttctcgaagctggcgcggaccccaacggaagccggcaccatcgcagcacccctgtctaccacgcctctcgcgtgggccgggcagacatcctgaaggccctcatcaggtacggggctgatgttgacgtcaaccaccacctgactcctgatgtccagcctcgattctcccggcggctcacctccttggtggtctgccccttgtacatcagcgcagcctaccacaacctccagtgcttccggctgctcctcctggctggcgcgaaccctgacttcaactgcaatggtcctgtcaacacacagggattctacaggggctcccctgggtgcgtcatggatgctgttctgcgccacggctgtgaggcagccttcgtgagcctgctggtagaatttggagccaacctgaatctagtgaagtgggaatcgctgggcccagagtcgagaggaagaagaaaagtggaccctgaggccttgcaggtctttaaagaggccagaagtgttcccagaaccttgctgtgtctgtgccgtgtggctgtgagaagagctcttggcaaacaccggcttcatctgattccttcgctgcctctgccagaccccataaagaagtttctactccatgagtagactccaagtgctgcggttgattccagtgagggagaaagtgatctgcagggaggtggacaccgagccctgagtgctgtgctgctgctggtctcctgatggctgttgctgcagaagatgtcctcgtagactgtcattgctcctcaggtgcctgggccgctgaacagtccttgggtcattgtcagctgagaggcttatactaaagttattattgtttttcccaaaaaaaaaaaaaaaaaa

SEQ ID NO: 106 - DNA, 3352 bp, Homo sapiens
accgcctcccaggccaggcgagcaggcgggtggctggggcgcctccacctcctcttcctaaagcggcgaggcgcagaggagcggcatcactcgagcccaggtcccagccaccaccactcacagcgctcggcgttcaggaagaggagcagcagcggaggcggctgcttcagcggcgggcgggcgccagaaaggccccgatcgaaaagcctgggagggccgccgaactacccccggagggaggagccagtccgaacccaaggcgccaccgccgcagaagcggagcgaggcagcattcgcctccatggcccactcgccggtggctgtccaagtgcctgggatgcagaataacatagctgatccagaagaactgttcacaaaattagagcgcattgggaaaggctcatttggggaagttttcaaaggaattgataaccgtacccagcaagtcgttgctattaaaatcatagaccttgaggaagccgaagatgaaatagaagacattcagcaagaaataactgtcttgagtcaatgtgacagctcatatgtaacaaaatactatgggtcatatttaaaggggtctaaattatggataataatggaatacctgggcggtggttcagcactggatcttcttcgagctggtccatttgatgagttccagattgctaccatgctaaaggaaattttaaaaggtctggactatctgcattcagaaaagaaaattcaccgagacataaaagctgccaatgtcttgctctcagaacaaggagatgttaaacttgctgattttggagttgctggtcagctgacagatacacagattaaaagaaatacctttgtgggaactccattttggatggctcctgaagttattcaacagtcagcttatgactcaaaagctgacatttggtcattgggaattactgctattgaactagccaagggagagccacctaactccgatatgcatccaatgagagttctgtttcttattcccaaaaacaatcctccaactcttgttggagactttactaagtcttttaaggagtttattgatgcttgcctgaacaaagatccatcatttcgtcctacagcaaaagaacttctgaaacacaaattcattgtaaaaaattcaaagaagacttcttatctgactgaactgatagatcgttttaagagatggaaggcagaaggacacagtgatgatgaatctgattccgagggctctgattcggaatctaccagcagggaaaacaatactcatcctgaatggagctttaccaccgtacgaaagaagcctgatccaaagaaagtacagaatggggcagagcaagatcttgtgcaaaccctgagttgtttgtctatgataatcacacctgcatttgctgaacttaaacagcaggacgagaataacgctagcaggaatcaggcgattgaagaactcgagaaaagtattgctgtggctgaagccgcctgtcccggcatcacagataaaatggtgaagaaactaattgaaaaatttcaaaagtgttcagcagacgaatccccctaagaaacttattattggcttctgtttcatatggacccagagagccccaccaaacctacgtcaagattaacaatgcttaacccatgagctccatgtgccttttggatctttgcaacactgaagatttggaagaagctattaaactattttgtgatggcgtttatcattttatattttgaaaggattattttgtaaggaataacttttaatactatagtttcacctgtattctagtaaatgttgagacaccgttttgcttttaagtatccctatttcttaagttacgaggatgaatacctttcacattttgatctttagttgactctacagtcatgaaacatacaggtctttcaaagtcattctcaatattcagcttttgtaaattatcaagcttcaaaaagcttttttttttaaaaaaaaacatgcatattctaaaaatgactattggtggggaggtgtaaataagtcataccttcttaaaacagaaaatttaagtaaagtcttttaaatgaaacctgtaaaagtattgactcttctaccaagttggtatgatattccaggcagctcaatgattatcacatttgagaccctgtgtttgaagcatttacaggcaatgtacagcaacagaggtacctcttggtgtatagtatttacattctcttttaggtagaagaggcaattttacccttatttcacatggttagaaatttaaagcaagatcatttacccaaggataggtgtttggtaatgttgaaggagttagtctggcttcatgttttacatcttcaactaaaatcccatactatctgcttggatttggagagccaaaaaataaagctgattgtcatgtgattaaatatctgatcaacaggtatgaatataacttaaatcagcatatttttgccatggtaataaattgtcctataaactatttatatatttttgttcttcataattatcactaataagcatcagtttgttgtttttaaaaggatatttaagtgagcattttctagttcatatgaaaataaccatagtacaggatgatttctgtccacacaaaggttaaattagattgcacagttaattttcacttatatttatggtactattatgtgggtgatgcctttttcttttaagcccagtacatatattatgcctgcctaagttctgaactggggctgtatttcagtagttgtagaattattgatatttagttttgatagctaatgtttaattgtttggatctgcacagtttggtttttgcacaaaagtcatttaaaaaaatctgagtaattgtcaaatattaaaagaaagatattcttcctgtaaggaatacagtttttagtcaaagtggccattacatcctctttttaatttacataatacagatacttgagaaagttgttgtggtgttgtatgccaagaaaattctttttattggtgcctatattgtaacaattatttttaatgcattgtattttgaagtaacggttcagttaaatttttcacctgctgtgtaactgaaacacaattacagtttataatcatctgtagaagtctggagataattttgcaactcatgttatgggttaaatgaatatttttgtaaaagtaaaagcaacaaatttataaattgattatttgaaactttacaacacaattgcatcccaaatacaaattgtattgcttattcattatagctattcgtcctgtaatctgtttctaggtgaagcatactccagtgttttaggggttttgaaaataaatatttaaatttcacagtcaaaaaaaaaaaaaaaa

SEQ ID NO: 107 - DNA, 1654 bp, Homo sapiens
gccagcgggaactgtgtaggggtagattttcgctgcagtgttccccgagcctgttagacgcagcgcgccgggagactgagagaggaaaggatagaggaagtgctgccctaggctgcatgagtcgaagcaagcgtgtttccttcccgccaggcaagtgcccttagaaaccgggccccgcccccttcctggcctgcattcccatcccctctcccggggcggaggtgaggacctccttggttcctttggttctgtcagtgagccccttccttggccatgaagctcgtgaggaagaacatcgagaaggacaatgcgggccaggtgaccctggtccccgaggagcctgaggacatgtggcacacttacaacctcgtgcaggtgggcgacagcctgcgcgcctccaccatccgcaaggtacagacagagtcctccacgggcagcgtgggcagcaaccgggtccgcactaccctcactctctgcgtggaggccatcgacttcgactctcaagcctgccagctgcgggttaaggggaccaacatccaagagaatgagtatgtcaagatgggggcttaccacaccatcgagctggagcccaaccgccagttcaccctggccaagaagcagtgggatagtgtggtactggagcgcatcgagcaggcctgtgacccagcctggagcgctgatgtggcggctgtggtcatgcaggaaggcctcgcccatatctgcttagtcactcccagcatgaccctcactcgggccaaggtggaggtgaacatccctaggaaaaggaaaggcaattgctctcagcatgaccgggccttggagcggttctatgaacaggtggtccaggctatccagcgccacatacactttgatgttgtaaagtgcatcctggtggccagcccaggatttgtgagggagcagttctgcgactacatgtttcaacaagcagtgaagaccgacaacaaactgctcctggaaaaccggtccaaatttcttcaggtacatgcctcctccggacacaagtactccctgaaagaggccctttgtgaccctactgtggctagccgcctttcagacactaaagctgctggggaagtcaaagccttggatgacttctataaaatgttacagcatgaaccggatcgagctttctatggactcaagcaggtggagaaggccaatgaagccatggcaattgacacattgctcatcagcgatgagctcttcaggcatcaggatgtagccacacggagccggtatgtgaggctggtggacagtgtgaaagagaatgcaggcaccgttaggatattctctagtcttcacgtttctggggaacagctcagccagttgactggggtagctgccattctccgcttccctgttcccgaactttctgaccaagagggtgattccagttctgaagaggattaatgattgaaacttaaaattgagacaatcttgtgtttcctaaactgttacagtacatttctcagcatccttgtgacagaaagctgcaagaagggcactttttgattcatacagggatttcttatgtctttggctacactagatattttgtgattggcaagacatgtatttaaacaataaactaaaaggaaataatctccacgtactaaaaaaaaaaaaaaaaaaa

SEQ ID NO: 108 - DNA, 2472 bp, Homo sapiens
cggcgggccgcgagccggcgatcgaagcgggcagctcgcgcctgagtcatggacttcccctggcccctcctctaccactcccactccctcgccggacccccccgccggggctagcgtctgccgcggctccgagggggtggggctgctgggaatggctgtgcccccttcggcccctcagccgcgcgcgtcctttcacctgaggaggcacacgccttgcccgcagtgctcatggggcatggaggagaaggcggcggccagcgccagctgccgggagccgccgggccccccgagggccgccgccgtcgcgtacttcggcatttccgtggacccggacgacatccttcccggggccctgcgcctcatccaggagctgcggccgcattggaaacccgagcaagttcggaccaagcgcttcacggatggcatcaccaacaagctggtggcctgctatgtggaggaggacatgcaggactgcgtgctggtccgggtgtatggggagcggacggagctgctggtggaccgggagaatgaggtcagaaacttccagctgctgcgagcacacagctgtgcccccaaactctactgcaccttccagaatgggctgtgctatgagtacatgcagggtgtggccctggagcctgagcacatccgtgagccccggcttttcaggttaatcgccttagaaatggcaaagattcatactatccacgccaacggcagcctgcccaagcccatcctctggcacaagatgcacaattatttcacgcttgtgaagaacgagatcaaccccagcctttctgcagatgtccctaaggtagaggtgttggaacgggagctggcctggctgaaggagcatctgtcccagctggagtcccctgtggtgttttgtcacaatgacctgctctgcaagaatatcatctatgacagcatcaaaggtcacgtgcggttcattgactatgaatatgctggctacaactaccaagcttttgacattggcaaccatttcaatgagtttgcaggcgtgaatgaggtggattactgcctgtacccggcgcgggagacccagctgcagtggctgcactactacctgcaggcacaaaaggggatggccgtgacccccagggaggtgcaaaggctctacgtgcaagtcaacaagtttgccctggcgtctcacttcttctgggctctctgggccctcatccagaaccagtactccaccatcgactttgatttcctcaggtacgcagtgatccgattcaaccagtacttcaaggtgaagcctcaagcgtcagccttggagatgccaaagtgaccagccaccccatccctcccctacccatctgtctggccagacctgttctccagagctcaattctgcactctgggatccacacccttggacagggtgggagaggggacacatgggtgtccagggagaaggctctgtccctgccgccagaccccagtggttgccactgaagacctcattctcctgtctggaggggctgataggacccccttccgggggtccccttcaccccaccaggcttgggaggaagtgcctgcagccaggtcctgaaccataaccacccctgggaaacacatcattcccagcctcaggccctgctggaattggggctgccttatatgtgtgtttaccccttcctggcctggggaaggaggcggggagggctcctttctacctccagtgccctgagcctccagtccgtctccccctgcatgccccatgtgggaggtgctgagctccaaaccagcatcacaccaactctgacacatggatgtacctatcttggtgatgggtgggggccaagaattgagcatgacatcttccccagcagccacctcctctgagatccctcaccttctccaaaccagatccaatcaaacctcagcccgaggaaacatgctccccaacgtgctctcctgtgcttctgttttgtccccctgctggggggacaggagagggagtggtgaggccctgggcctccagagcctggctctgctttgtgctgtggcttagccggaggggacgtggccaagggtgaggtggccaaaaccagaaccagcagtctcctgccttgttccctccctggccctcaggccctccttccagggatgtctctccagctctactttatgtcctgaagctgacccgaggtcttcctatctggaatgactagagggagccaagaggatggggtgggggccagggccccccagggcctatcgtgggagagcctgggcaggatcccatcagaaaggtgctgactaaactggttgcccggacactcaacagcctccacctccctttctaccctcacagctcctggggccttcctggctctggcccagaaagtgattcatttgtaaattatcatggttttctttctgcattaaaatgctcatttccggaaaaaaaaaaaaaaaaaaaaaaaaaaaaaaaaaaaaaaa

SEQ ID NO: 109 - DNA, 2617 bp, Homo sapiens
acttcggctgtctccgctgccctccagcggagacgcagctcctcaggcgcccggcggtatttgttgggtcggcggcgtcagggattcgcagtggcctgtggtcggcgtcgtccggccactggtgcgcccccgcgacaggcagagctcacgctcctgtcccccggctggtccggggtctgggcgccgcgtcgacggcggctccgcaggacgcgcagaccgggccgcagcccatgccccgagcggactgcattatgaggcacctgccttacttctgccggggtcaagtggtgcggggcttcggccgcggctccaagcagctgggcatccccacagctaattttcctgagcaagtggtagataatcttccagctgatatatccactggtatttactatggttgggccagtgttggaagtggagatgtccataagatggtggtgagcataggatggaacccatattacaagaatacgaagaagtctatggaaacacatatcatgcataccttcaaagaggacttctatggggaaatcctcaatgtcgccattgttggctacctgagaccagaaaagaactttgattctttagagtcacttatttcagcaattcaaggtgatattgaagaagctaagaaacgactagagttaccagaacatttgaaaatcaaagaagacaatttcttccaggtttctaaaagcaaaataatgaatggccactgatgaaaaattgtattatttattcattcactgttttctagtgtttctgtgtttattactgttcagcatcatcttggttatatactgaaaatcaaacactttactacagttgtgaattagtttaaaccgtacaatgttgttatcatatcatgcttcaactattaaattaagcccatcatatcatagcactaaaaagattgagttaaaaataagatttaaaaaatatatattgattaaaatgtaccactagaaaggtgttacatgtgtataagtatgggtaaaaaggcaagtcactagtttgagatgaaaactaaaattttcatggtaaaatgccagcatgtatgtacttgtgtagtgtgtacttctaaacagagaaggcttataaaagtaaatgcattaaggccaagtgcagttgctcacaccggtaatcccagcactttgggaggccgaggtgggaggattgcttgagctcatgagttcaagaccagtctgggcaacatagcgagactccatctctataaataaaaaaaaaggaaatgcattaaatagaaggttgatagtttatcataaacctgcggggaagaaactcagactttctatttttgatattttgtgcattaatacattacaaacatgaagatgtttgtaaagttttgttttagtcattttctttgcagtcacatcattgtaccaccttcattattgtatagagctgcttcaattctgagtcctctggacttcccctgcctatctgtttgttgttgttgttgaagacagtaaaataagtgttttcaaatctatatgtctcctttttgttggataatgtttcttctttttaatggtaaatattcttgttcttcaacatttttctttggttcttttttccttttttaggaaaaacaaaacaacagacttcatccttaggttttctcaagatttaagtgaacacatttacacatatcaatttcttaaagaacacaaaatgtttcctccctagcaaaactatttaagagccaattaaactatgaaatagttataaaattatttttaatatttaaggccttgaaattatattccaaggcaaatctaaatgggtgtagttgtatatgcacgatgtaatttaatgctaaagagtacttacagcttgagggaaaagcataaagttttagttaaatagccttcatactgtggatttttcctcctactaactaatatgtgggaaattaagattacagatgctttgggccccctcaaacagtacattgctacagttcactgtagcaacagatttggtggtgatggggggatagaggtagaagcagatagaaggtaattttaaagttaatttattatgaatcattttcccctaatctgcataaatgaattagaactgattttaataaaaatattctgtgtaaaaagtactcagaaaaacatctaaagctgttggagaagagggactcatataaatagttggattgcgttttgctttttatttctttataaaaatatgtcaaaaacagtttttaattttagaaatcttatacttgaaagaaacctcaattatcagtggctttatgaagtaaagttaaaaacaaacataggaaatgtgaaaatatttgtgtttgttttgtctgactaacttgctgctaaatgctatctttaggtgtgtgaaagttctttgatatgttattgtccttttacatcttgttaaatagccattgcttttgagaatcagatagtgtgttgtttaggggttaattgtgtctatatctttcaaaaacttttatattatgtattataagaagttacagtagtaccattttgattagtctgaatgttttcaggccttgaaataaattttgactaagtaaaaaaaaaaaaaaaaaaaaaaaaaaaaa

SEQ ID NO: 110 - DNA, 2852 bp, Homo sapiens
ctggtggggtcggaccaggccccgggccgggacaagaacatccccgccgggctgcagtccatgaaccaggcgttgcagaggcgcttcgccaagggggtgcagtacaacatgaagatagtgatccggggagacaggaacacgggcaagacagcgctgtggcaccgcctgcagggccggccgttcgtggaggagtacatccccacacaggagatccaggtcaccagcatccactggagctacaagaccacggatgacatcgtgaaggttgaagtctgggatgtagtagacaaaggaaaatgcaaaaagcgaggcgacggcttaaagatggagaacgacccccaggaggcggagtctgaaatggccctggatgctgagttcctggacgtgtacaagaactgcaacggggtggtcatgatgttcgacattaccaagcagtggaccttcaattacattctccgggagcttccaaaagtgcccacccacgtgccagtgtgcgtgctggggaactaccgggacatgggcgagcaccgagtcatcctgccggacgacgtgcgtgacttcatcgacaacctggacagacctccaggttcctcctacttccgctatgctgagtcttccatgaagaacagcttcggcctaaagtaccttcataagttcttcaatatcccatttttgcagcttcagagggagacgctgttgcggcagctggagacgaaccagctggacatggacgccacgctggaggagctgtcggtgcagcaggagacggaggaccagaactacggcatcttcctggagatgatggaggctcgcagccgtggccatgcgtccccactggcggccaacgggcagagcccatccccgggctcccagtcaccagtggtgcctgcaggcgctgtgtccacggggagctccagccccggcacaccccagcccgccccacagctgcccctcaatgccgccccaccatcctctgtgccccctgtaccaccctcagaggccctgcccccacctgcgtgcccctcagcccccgccccacggcgcagcatcatctctaggctgtttgggacgtcacctgccaccgaggcagcccctccacctccagagccagtcccggccgcagagggcccagcaacggtccagagtgtggaggactttgttcctgacgaccgcctggaccgcagcttcctggaagacacaacccccgccagggacgagaagaaggtgggggccaaggctgcccagcaggacagcgacagtgatggggaggccctgggcggcaacccgatggtggcagggttccaggacgatgtggacctcgaagaccagccacgtgggagtcccccgctgcctgcaggccccgtccccagtcaagacatcactctttcgagtgaggaggaagcagaagtggcagctcccacaaaaggccctgccccagctccccagcagtgctcagagccagagaccaagtggtcctccataccagcttcgaagccacggagggggacagctcccacgaggaccgcagcacccccctggccaggcggtgtctctgttcgcacaggtccggagaagcgcagcagcaccaggccccctgctgagatggagccggggaagggtgagcaggcctcctcgtcggagagtgaccccgagggacccattgctgcacaaatgctgtccttcgtcatggatgaccccgactttgagagcgagggatcagacacacagcgcagggcggatgactttcccgtgcgagatgacccctccgatgtgactgacgaggatgagggccctgccgagccgcccccaccccccaagctccctctccccgccttcagactgaagaatgactcggacctcttcgggctggggctggaggaggccggacccaaggagagcagtgaggaaggtaaggagggcaaaaccccctctaaggagaagaagaagaagaagaaaaaaggcaaagaggaagaagaaaaagctgccaagaagaagagcaaacacaagaagagcaaggacaaggaggagggcaaggaggagcggcgacggcggcagcagcggcccccgcgcagcagggagaggacggctgccgatgagctggaggctttcctggggggcggggccccgggcggccgccaccctgggggtggcgactacgaggagctctaggccggcgtgggcagtggccgccctggggcggggggcgtgcctgtcactgcctggggaggcatttgcctctgtaccatcgcctttgccgctgccccgtggctgccgtgtgcgcttctgagctggaagaggccgggcattggtggtccccaggctgggccctgcaggtgctgggccttcaggcccagtgtgagcctgctctgcaagaagggaggggacagctggcttcagccaggctcggtggacaccctggccctctcggggcagagccgccagtgtttctcagggatgtgactgaggcccaggagggacctgtgagggtctgtttacagaggctgggcaggggccgcttggctgtggggtgtgcgctgccccggcacctgcttgccctccgcgctcatctggggccgcagcatgcctatggttccgcttccggccgggagccctgaacacgggtgtgcagactcaccctaaagggcggcccaggccccacgctagaaggctggcgagaccgaaggcagcatgtgaggcctctcctgggagtgggggttgtgtttcccacagtggcctcagctgcgcccccgctcaggtgagcccgaaggcaggagccgggaggcactcctcccaaacactccactcagaccataaagcactcctgtttcactctgaaaaaaaaaaaaaaaaaa

SEQ ID NO: 111 - DNA, 2161 bp, Homo sapiens
ggcgtaggagcgcgccatggagcatgtgacagagggctcctgggagtcgctgcctgtgccgctgcacccgcaggtgctgggcgcgctgcgggagctgggcttcccgtacatgacgccggtgcagtccgcaaccatccctctgttcatgcgaaacaaagatgtcgctgcagaagcggtcacaggtagtggcaaaacactcgcttttgtcatccccatcctggaaattcttctgagaagagaagagaagttaaaaaagagtcaggttggagccataatcatcacccccactcgagagctggccattcaaatagacgaggtcctgtcgcatttcacgaagcacttccccgagttcagccagattctttggatcggaggcaggaatcctggagaagatgttgagaggtttaagcaacaaggtgggaacatcattgtggccactccaggccgcttggaggacttgttccggaggaaggccgaaggcttggatctggccagctgtgtgcgatccctggatgtcctggtgttggatgaggcagacagacttctggacatggggtttgaggcaagcatcaacaccattctggagtttttgccaaagcagaggagaacaggccttttctctgccactcagacgcaggaagtggagaacctggtgagagcgggcctccggaaccctgtccgggtctcagtgaaggagaagggcgtggcagccagcagtgcccagaagaccccctcccgcctggaaaactactacatggtatgcaaggcagatgagaaatttaatcagctggtccattttcttcgcaatcataagcaggagaaacacctggtcttcttcagcacctgtgcctgtgtggaatactatgggaagactctggaagtgctggtgaagggcgtgaagattatgtgcattcacggaaagatgaaatataaacgcaataagatcttcatggagttccgcaaattgcaaagtgggattttagtgtgcactgatgtgatggcccggggaattgatattcctgaagtcaactgggttttgcagtatgaccctcccagcaatgcaagtgccttcgtgcatcgctgcggtcgcacagctcgcattggccacgggggcagcgctctggtgttcctcctgcccatggaagagtcatacatcaatttccttgcaattaaccaaaaatgccccctgcaggagatgaagccccagagaaacacagcggaccttctgccaaaactcaagtccatggccctggctgacagagctgtgtttgaaaagggcatgaaagcttttgtgtcatatgtccaagcttatgcaaagcatgaatgcaacctgattttcagattaaaggatcttgattttgccagccttgctcgaggttttgccctgctgaggatgcccaagatgccagaattgagagggaagcagtttccagattttgtgcccgtggacgttaataccgacacgattccatttaaagataaaatcagagaaaagcagaggcagaaactcctggagcaacaaagaagagagaaaacagaaaatgaagggagaagaaaattcataaaaaataaagcttggtcaaagcagaaggccaaaaaagaaaagaagaaaaaaatgaatgagaaaaggaaaagggaagagggttctgatattgaagatgaggacatggaagaacttcttaatgacacaagactcttgaaaaaacttaagaaaggcaaaataactgaagaagaatttgagaagggcttgttgacaactggcaaaagaacaatcaagacagtggatttagggatctcagatttggaagatggctgctaattccagtgccacagatgaacccacaaggacatagctgttccctaacttggtggatggctccagtttgcttttaacgaaaatcacaacttcaggagacatctgaaaagaatgatgtctctgaaagctgtcctttcagatgagggagaaatgaaggatttcacacttcagaatattttactaaaaacattccagtcttggccgggtgcggtggctcctgcctataatcccagcactttgggaggctgaggcaggaggatcacttgagcccaggagttcaagaccagcctgggaacacagcgagaccctctcattaaaaacaacaaaacaaaaaaaaaaaaaaaa

SEQ ID NO: 112 - DNA, 1466 bp, Homo sapiens
gctggttgaagatgaaatccactgaggagggaagtccagcaccctgtgtgccagtccagaactggcccatctgtagaccccctgaaaatcatatgggcttggatttggatattctcaacagaaagggttaaaggctgatggtacctaaagcctggtacttgaattttgatcaagataagctgccttaagttctcttcattacacaaatgatcctagataattgatagatcctgtggttcaactggatttctagatagaagctggattcatgtgatgccagaggagtaaaatttcaagagactgaaaccagatctgagtttcgctgttccagtctggacctctttggtgctgtaaatcctggatatactgtagatgagtactgcgtttttcttttatggcctctcttcagcttctggagacctcactatcctattatgtctttgtgtgaagacatgctgctttgtaattatcgaaagtgtcgcatcaaactctctggctatgcatgggtcactgcctgctctcacatcttctgtgatcagcatggcagtggtgagtttagtcgctcaccagctatctgtcctgcctgcaacagtaccctttctggaaagctagatattgtccgcacagaactcagtccatcagaggaatataaagctatggtattggcaggactgcgaccagagatcgtgttggacattagctcccgagcgctggccttctggacatatcaggtacatcaggaacgtctctatcaagaatacaatttcagcaaggctgagggccatctgaaacagatggagaagatatatactcagcaaatacaaagcaaggatgtagaattgacctctatgaaaggggaggttacctccatgaagaaagtactagaagaatacaagaaaaagttcagtgacatctctgagaaacttatggagcgcaatcgtcagtatcaaaagctccaaggcctctatgatagccttaggctacgaaacatcactattgctaaccatgaaggcacccttgaaccatccatgattgcacagtctggtgttcttggcttcccattaggtaacaactccaagtttcctttggataatacacctgttcgaaatcggggcgatggagatggagattttcagttcagaccattttttgcgggttctcccacagcacctgaacccagcaacagcttttttagttttgtctctccaagtcgtgaattagagcagcagcaagtttctagcagggccttcaaagtaaaaagaatttgagccacgcatagtgtcacgcacctgtgatcccagctacttaggaggttgaggctgggaggatcacttgagcccaggagtctgaggctttagtgatctaagatcatgccactgcactccagcctgggcaacagagtgagaccctgtttctaaaaaaaaataaagataatttagctaactttaaaaaaaaaaaaaaaaaaa

SEQ ID NO: 113 - DNA, 2058 bp, Homo sapiens
ggctccagagcctccagagcctccgggtctgggcggcgcttcggctcctcccgagccgcctgctagccccgcgccgcactccatccccacaggctggggacgggcccggtgcggctgtgtgggttcgggagcggagttgcagaatccaaggacccattttgttctttctccgcactgctttatgggaggcattatggcccccaaagacataatgacaaatactcatgctaaatccatcctcaattcaatgaactccctcaggaagagcaataccctctgtgatgtgacattgagagtagagcagaaagacttccctgcccatcggattgtgctggctgcctgtagtgattacttctgtgccatgttcactagtgagctctcagagaaggggaaaccttatgttgacatccaaggtttgactgcctctaccatggaaattttattggactttgtgtacacagaaacagtacatgtgacagtggagaatgtacaagaactgcttcctgcagcctgtctgcttcagttgaaaggtgtgaaacaagcctgctgtgagttcttagaaagtcagttggacccttctaattgcctgggtattagggattttgctgaaacccacaattgtgttgacctgatgcaagcagctgaggtttttagccagaagcattttcctgaagtggtacagcatgaagagttcattcttctgagtcaaggagaggtggaaaagctaatcaagtgcgacgaaattcaggtggattctgaagagccagtctttgaggctgtcatcaactgggtgaagcatgccaagaaagagcgggaagaatccttgcctaacctgctacagtatgtgcggatgcccctactaacccccaggtatatcacagatgtaatagatgctgagcctttcatccgctgtagtttacaatgcagggatctggttgatgaagcaaagaagtttcatctgaggcctgaacttcggagtcagatgcagggacccaggacaagggctcgcctaggagccaatgaagtgcttttggtggttgggggctttggaagccagcagtctcccattgatgtggtagagaaatatgaccccaagactcaggagtggagctttttgccaagcatcactcgtaagagacgttatgtggcctcagtgtcccttcatgaccggatctacgtcattggtggctatgatggccgttcccgccttagttcagtggaatgtctagactacacagcagatgaggatggggtctggtattctgtggcccctatgaatgtccgacgaggtcttgctggagccaccaccctgggagatatgatctatgtctctggaggctttgatggaagcaggcgtcacaccagtatggagcgctatgatccaaacattgaccagtggagcatgctgggagatatgcagacagcccgggaaggtgccggactcgtagtggccagtggagtgatctactgtctaggaggatatgacggcttgaatatcttaaattcagttgagaaatacgaccctcatacaggacattggactaatgttacaccaatggccaccaagcgttctggtgcaggagtagccctgctgaatgaccatatttatgtggtggggggatttgatggtacagcccacctttcttccgttgaagcatacaacattcgcactgattcctggacaactgtcaccagtatgaccactccacgatgctatgtaggggccacagtgcttcgggggagactctatgcaattgcaggatatgatggtaattccctgctaagtagcattgaatgttatgaccctatcatcgacagctgggaagtcgtgacatccatgggaacccagcgctgtgatgctggtgtttgtgttctccgcgagaagtgaccattgttggagcaccatccagagctagtgaccagtccagtggacagttagtgggagaatcaaaaatcctttccagaatgtctgtttctcactacgtgcaccgggtgattacaggcaccagtgcagtgatgattgtacttatttgacacataaaaaaaaaaaaaaaaaaa

SEQ ID NO: 114 - DNA, 1839 bp, Homo sapiens
ctggttggggttattcggcggcccgcggcggaccatggccctggcccggcgtcgctgggctttcctcacggcgtccccgagcagcgtcgcagagcgggccgacttccgggaaggaactgaccagcgactgagcggcggccggcgcgcttagcgccctgaacatgcggcagtccctgcgggcgaccccgggctccggacaggcggcggcggaggcggcggctcgggagggaaggaggcggcggcgccggcggaggtggcggcggagacggccggcgcccggcgcggagccctagggaggcagttcagcgcggcctcgggcctcgtcgagaaggatgctgtcccgaaagaaaaccaaaaacgaagtgtccaagccggccgaggtgcaggggaagtacgtgaagaaggagacgtcgcctctgcttcggaatcttatgccttcattcatccggcatggtccaacaattccaagacgaactgatatctgtcttccagattcaagccctaatgccttttcaacttctggagatgtagtttcaagaaaccagagtttccttagaactccaattcaaagaacacctcatgaaataatgagaagagaaagcaacagattatctgcaccttcttatcttgccagaagtctagcagatgtccctagagagtatggttcttctcagtcatttgtaacggaagttagttttgctgttgaaaatggagactctggttcccgatattattattcagacaatttttttgatggtcagagaaagcggccacttggagatcgtgcacatgaagactacagatattatgaatacaaccatgatctcttccaaagaatgccacagaatcaggggaggcatgcttcaggtattgggagagttgctgctacatctttaggaaatttgactaaccatggttctgaagatttaccccttcctcctggctggtctgtggactggacaatgagagggagaaaatattatatagatcataacacaaatacaactcactggagccatcctcttgagcgagaaggacttcctcctggatgggaacgagttgagtcatccgaatttggaacctattatgtagatcacacaaataagaaggcccaatacaggcatccctgtgctcctagtgtacctcggtatgatcaaccacctcctgtcacataccagccacagcaaactgaaagaaatcagtcccttctggtacctgcaaatccatatcatactgcagaaattcctgactggcttcaggtttacgcacgagcccctgtgaaatatgaccacattctgaagtgggaactcttccagctggctgacctggatacataccagggaatgctaaagttgctcttcatgaaagaattggagcagattgttaaaatgtatgaagcatacagacaagcccttcttacagagttggaaaaccgaaagcagagacagcagtggtatgcccaacaacatggaaaaaatttttgagctgattttttaaaaatttaagttttgtaagagctttaaaatattttcacagataaaaaattgcaaacaagtactctggttaataaatgctgcttcctttgtggaaattataaaattctaactttacatgtattttgttattagaaattttcttttattgaatgagaaaaattagtctatcattttaagagccaatatggcaaacactttcaaatactgtatattaggaaactgttttggtattcttgatggaaaaaaatgcagcggaaatgtcattatgaacagatgttaaataggaaattattacttgttaacttcttacagcagtagtaccttctttaaaaaaaaaaaaaaa

SEQ ID NO: 115 - DNA, 3003 bp, Homo sapiens
ctagccgaagcggctgcatctggcgccgcgtctgccccgcgtgctcggagcggattctgcccgccgtccccggagccctcggcgccccgctgagcccgcgatcacttcctccctgtgaccaaccggcgctgcaggttagagcctggcaatgccgtttgggtgtgtgactctgggcgacaagaagaactataaccagccatcggaggtgactgacagatatgatttgggacaggtcatcaagactgaggagttttgtgaaatcttccgggccaaggacaagacgacaggcaagctgcacacctgcaagaagttccagaagcgggacggccgcaaggtgcggaaagctgccaagaacgagataggcatcctcaagatggtgaagcatcccaacatcctacagctggtggatgtgtttgtgacccgcaaggagtactttatcttcctggagctggccacggggagggaggtgtttgactggatcctggaccagggctactactcggagcgagacacaagcaacgtggtacggcaagtcctggaggccgtggcctatttgcactcactcaagatcgtgcacaggaatctcaagctggagaacctggtttactacaaccggctgaagaactcgaagattgtcatcagtgacttccatctggctaagctagaaaatggcctcatcaaggagccctgtgggacccccgagtatctggccccagaggtggtaggccggcagcggtatggacgccctgtggactgctgggccattggagtcatcatgtacatcctgctttcaggcaatccacctttctatgaggaggtggaagaagatgattatgagaaccatgataagaatctcttccgcaagatcctggctggtgactatgagtttgactctccatattgggatgatatttcgcaggcagccaaagacctggtcacaaggctgatggaggtggagcaagaccagcggatcactgcagaagaggccatctcccatgagtggatttctggcaatgctgcttctgataagaacatcaaggatggtgtctgtgcccagattgaaaagaactttgccagggccaagtggaagaaggctgtccgagtgaccaccctcatgaaacggctccgggcaccagagcagtccagcacggctgcagcccagtcggcctcagccacagacactgccacccccggggctgcaggtggggccacagctgcagctgcgagtggagctacctcagcccctgagggtgatgctgctcgtgctgcaaagagtgataatgtggcccccgcagaccgtagtgccaccccagccacagatggaagtgccaccccagccactgatggcagtgtcaccccagccaccgatggaagcatcactccagccactgatgggagtgtcaccccagccactgacaggagcgctactccagccactgatgggagagccacaccagccacagaagagagcactgtgcccaccacccaaagcagtgccatgctggccaccaaggcagctgccacccctgagccggctatggcccagccggacagcacagccccagagggcgccacaggccaggctccaccctctagtaaaggggaagaggctgctggttgtgcccaggagtctcaaagggaggaggccagctgagtaggcagcctggtgagggggggcaggggatgggcaggagggtgggagagtggatgaggggcttctcactgtacatagagtcactggcatgatgccctcgctcccccatgcccccacatcccagtggggcataactaggggtcacgggagagcagtctcgtctcctgtgtgtatgtgtgtgagtggtgggcaggccagtggcagggccggccccagcccctgcatggattccttgtggcttttctgtcttttgctagcttcaccagtttctgttccttgtgggatgctgctctagggatactcagggggctcctgctctccttccccttcccttcttgcctcaccattcccctaggcaggccctgcaggtcccacactctcccaggccctaaacttgggcggccttgccctgagagctggtcctccagcgaggccctgtcagcggtcttaggctcctgcacatgaaggtgtgtgcctgtggtgtgtgggctgctctaggagcagatacaggctggtatagaggatgcagaaaggtagggcagtatgtttaagtccagacttggcacatggctagggatactgctcactagctgtggaggtcctcaggagtggagagaatgagtaggagggcagaagcttccatttttgtccttcctaagaccctgttatttgtgttatttcctgcctttccgagtcctgcagtgggctgccctgtaccctgaacctcatgagcctctaagggaaaggaggaacaattaggacgtggcaatgagacctggcagggcagagtacaagcccagcacccagtgtcccagccttactgggtccttaccctgggccaaacagggagggctgatacctccttgctcttcctagatgcccacctcctacaatctcagcccacaagtcctctccaccctagggggcttgctgcatggcaataactcataatctgatttggaggtttgccctttacaggggcagattttctgctcagttcaacaatgaaatgaagaggaactccctctttctacagctcacttctatcagaggcccaggtgcctcagagccacattgagttgctttttctgggatgaggaagtagggttaaactccccagtttcctgagggaggctcctgacaggtgccctttgtcagaccctaccacagcctggataggcagccacattggtcctcgcccttgctcggcactccgtggtggtcctgcccttctccctgcatgcctgtgggtctgctctggtgtgtgaaggtcggtgggttaactgtgtgcctactgaacctggcaaataaacatcaccctgcaaagccaaaaaaaaaaaaaaaaaaaa

SEQ ID NO: 116 - DNA, 1549 bp, Homo sapiens
gaaaactcatttgctcttggagcagtcaggcagtgactgccttcggctttttttctgctgactaagatctcctatagagagctacaacaatgcccaaaagaaaggctgcaggtcaaggtgatatgaggcaggagccaaagagaagatctgccaggttgtctgctatgcttgtgccagttacaccagaggtgaagcctaaaagaacatcaagttcaaggaaaatgaagacaaaaagtgatatgatggaagaaaacatagatacaagtgcccaagcagttgctgaaaccaagcaagaagcagttgttgaagaagactacaatgaaaatgctaaaaatggagaagccaaaattacagaggcaccagcttctgaaaaagaaattgtggaagtaaaagaagaaaatattgaagatgccacagaaaagggaggagaaaagaaagaagcagtggcagcagaagtaaaaaatgaagaagaagatcagaaagaagatgaagaagatcaaaacgaagagaaaggggaagctggaaaagaagacaaagatgaaaaaggggaagaagatggaaaagaggataaaaatggaaatgagaaaggagaagatgcaaaagagaaagaagatggaaaaaaaggtgaagacggaaaaggaaatggagaagatggaaaagagaaaggagaagatgaaaaagaggaagaagacagaaaagaaacaggagatggaaaagagaatgaagatggaaaagagaagggagataaaaaagaggggaaagatgtaaaagtcaaagaagatgaaaaagagagagaagatggaaaagaagatgaaggtggaaatgaggaagaagctggaaaagagaaagaagatttaaaagaagaggaagaaggaaaagaggaagatgagatcaaagaagatgatggaaaaaaagaggagccacagagtattgtttaaaactgccctatgtagtttcataatttggtaacatgtaccttcatgttgtaaagttaatagagataaatatttttatcaaaaattttataaacacagcctttctttagcattgatttaatttcagaacatcttcatattgattattagccataaagtttctaacatgaaacatttatctataaattttgtgattatagtagtggaatacatagaaaaaaatatgctttcaactttgtgagtgaatttcgtgttgtgtaagttatatgtcaaatctttgaattttaattttactcttttatacatgtgataatttcataaagtgagggatcccaaaaaaagagtttcatccaacattcttgttctgcaggttgcttttataaagaaggtgaactattttcatgtaatgttaagagttaaacttatctttcccaaatataactttattattagcttgggaaaaatgaaattgtattcccatttttaaaataaatacaaatgtttatttcagaagggcagttttgattatatgtgaatacacaaattttactggatttatcttaataaaaagactctgacgaccaaaaaaaaaaaaaaaaaaaaaaaaaaaaaaa

SEQ ID NO: 117 - DNA, 4315 bp, Homo sapiens
ataccagaattaactctgctcagagttagatttgctggtcttaaagtacttttcctctttaagataaaagaagttcttctaaatcaggaatggattgaaatctaatgaaccgaaactttgggtaattgatcaccctagacccagggacacccaattcatcgtaatcatcatgaataatcaaaaagtggtagctgtgctactgcaagagtgcaagcaagtgctggatcagctcttgttggaagcgccagatgtgtcggaagaggacaagagcgaggaccagcgctgcagagctttactccccagcgagttaaggaccctgatccaggaggcaaaggaaatgaagtggcccttcgtgcctgaaaagtggcagtacaaacaagccgtgggcccagaggacaaaacaaacctgaaggatgtgattggcgccgggttgcagcagttactggcgtccctgagggcctccatcctcgctcgggactgtgcggctgcggcggctattgtgttcttggtggaccggttcctgtatgggctcgacgtctctggaaaacttctgcaggtcgccaaaggtctccacaagttgcagccagccacgccaattgccccgcaggtggttattcgccaagcccgaatctccgtgaactcaggaaaacttttaaaagcagagtatattctgagcagtctaataagcaacaatggagcaacgggtacctggctgtacagaaatgaaagtgacaaggtcctggtgcagtcggtctgtatacagatcagagggcagattctgcaaaagctggggatgtggtacgaagcagcagagttaatatgggcctccattgtaggatatttggcacttcctcagccggataaaaagggcctctccacgtcgctaggtatactggcagacatctttgtttccatgagcaagaacgattatgaaaagtttaaaaacaatccacaaattaatttgagcctgctgaaggagtttgaccaccatttgctgtccgctgcagaagcctgcaagctggcagctgccttcagtgcctatacgccgctcttcgtgctcacagctgtgaatatccgtggcacgtgtttattgtcctacagtagttcaaatgactgtcctccagaattgaaaaacttacatctgtgtgaagccaaagaggcctttgagattggcctcctcaccaagagagatgatgagcctgttactggaaaacaggagcttcacagctttgtcaaagctgctttcggtctcaccacagtgcacagaaggctccatggggagacagggacggtccatgcagcaagtcagctctgtaaggaagcaatggggaagctgtacaatttcagcacttcctccagaagtcaggacagagaagctctgtctcaagaagttatgtctgtgattgcccaggtgaaggaacatttacaagttcaaagcttctcaaatgtagatgacagatcttatgttcccgagagtttcgagtgcaggttggataaacttatcttgcatgggcaaggggatttccaaaaaatccttgacacctattcacagcaccatacttcggtgtgtgaagtatttgaaagtgattgtggaaacaacaaaaatgaacagaaagatgcaaaaacaggagtctgcatcactgctctaaaaacagaaataaaaaacatagatactgtgagtactactcaagaaaagccacattgtcaaagagacacaggaatatcttcctccctaatgggtaagaatgttcagagggaactcagaaggggaggaaggagaaactggacccattctgatgcatttcgagtctccttggatcaagatgtggagactgagactgagccatcggactacagcaatggtgagggagctgttttcaacaagtctctgagtggcagccagacttccagtgcttggagcaacttatcagggtttagttcctctgcaagctgggaggaagtgaattatcacgttgacgacaggtcagccagaaaagagcctggcaaagaacatctggtggacactcagtgttccactgccttgtctgaggagctagagaatgacagggaaggcagagctatgcattcattgcattcacagcttcatgatctctctcttcaggaacccaacaatgacaatttggagccttctcaaaatcagccacagcaacagatgcccttgacacccttctcgcctcataataccccaggcattttcttggcccctggtgcagggcttctagaaggagctccagaaggtatccaggaagtcagaaatatgggacccagaaatacttctgctcactccagaccctcatatcgttctgcttcttggtcttctgattctggtaggcccaagaatatgggcacacatccttcagtccaaaaagaagaagcctttgaaataattgttgagtttccagaaaccaactgcgatgtcaaagacaggcaggggaaagagcagggagaagaaattagtgaaagaggcgcaggccctacatttaaagctagtccctcctgggttgacccagaaggagaaacagcagaaagcactgaagatgcacccttagactttcacagggtcctgcacaattctctgggaaacatttccatgctgccatgtagctccttcacccctaattggcctgttcaaaatcctgactccagaaaaagtggtggcccagtcgcagagcagggcatcgaccctgatgcctccacagtggatgaggaggggcaactgctcgacagcatggatgttccctgcacaaatgggcacggctctcatagactgtgcattctgagacagccgcctggtcagagggcggagacccccaattcctctgtaagcggtaacatcctcttccctgtcctcagcgaggactgcactaccacagaggaaggaaatcagcctggaaacatgctaaactgcagccagaactccagctcatcctcagtgtggtggctgaaatcacctgcattttccagtggttcttctgagggggacagcccttggtcctatctgaattccagtgggagttcttgggtttcattgccgggaaagatgaggaaagagatccttgaggctcgcaccttgcaacctgatgactttgaaaagctgttggcaggagtgaggcatgattggctgtttcagagactagagaatacgggggtttttaagcccagtcaactccaccgagcacatagtgctcttttgttaaaatattcaaaaaaatctgaactgtggacggcccaggaaactattgtctatttgggggactacttgactgtgaagaaaaaggcagacaaagaaatgctttttgggttcatcatcttcatcaagaagaaattctggggaggtatgttgggaaagactataaggagcagaaggggctctggcaccacttcactgatgtggagcgacagatgaccgcacagcactatgtgacagaatttaacaagagactctatgaacaaaacattcccacccagatattctacatcccatccacaatactactgattttagaggacaagacaataaagggatgtatcagtgtggagccttacatactgggagaatttgtaaaattgtcaaataacacgaaagtggtgaaaacagaatacaaagccacagaatatggcttggcctatggccatttttcttatgagttttctaatcatagagatgttgtggtcgatttacaaggttgggtaaccggtaatggaaaaggactcatctacctcacagatccccagattcactccgttgatcagaaagttttcactaccaattttggaaagagaggaattttttacttctttaataaccagcatgtggaatgtaatgaaatctgccatcgtctttctttgactagaccttcaatggagaaaccatgcacatagaatacggcacagtctggtcctttggggcttgggcagggccgtgacacaggttctggccaatgatttgcaagaggaattgatcagtatcactttaagtcctgcatttaattggcagcacaagatcctgcagagcctctttccctctgccacagttatcaagaatgggtcaggagaccgctgcttctgggcataagtcctgcaaggaaagcaacatggaaaacagccccaactcacccatgagggatgaaaagcactcttgagaaaggcatgtgttgtttaagccattgagattttagagctttttgtcactatctgtcaagactgatactactggggcttttcctattgatttgggagttctttacatattaaaaaaatgtgagcctttgtgatacgaaaaaaaaaaaaaaaa

SEQ ID NO: 118 - DNA, 2356 bp, Homo sapiens
ccgaggcggcgggacccccaagtttggaaagctcttttaacggctatgcgaaagttctgtccaaaggctgctcacagcccctgtctgactggttgtcatgatgaattgagctctctgttacctgcgcatcacaaccgatagtaactgtccctactgaaggccccaggagtgctccagccagagctgaggcttcgaagacctcagaggacttctctcagcactcacagaaacctcctacaccctcggatggcacaaagggactgttttcttactcttagtctgagtgactgccaaggaaggcaaaggtagagcaactggatctctggctctccacatagcttctgatctcagaccttactaaaatgctttctgggcccaaggacaaagctcacatgaacaaatgattttgagtcatgaatgaaaaatcttgctctttccatagtaaagaagaattaagagatggacagggtgaaagattgtctgctggatattctccatcatatgacaaggacaagagtgttctggctttcagaggaatccctatctcagagttgaagaaccatggcattctccaggctctgaccacagaagcttatgaatgggagccacgtgttgtgagtacagaggtggtcagagcccaagaagaatgggaagctgtggacaccatccagccagagacagggagccaagctagctcagagcagcctgggcagctaatctccttcagtgaggccctgcagcacttccagactgtggacctttcccccttcaagaaaagaatccagccaactattcgaaggactgggctcgccgccctccgacactacctcttcgggcctccaaagctccaccagcgccttcgggaagaaagggacttggtcctgaccattgctcagtgtggcctggatagccaagacccagtgcatggccgagtcctccagaccatctataagaagctgaccggctccaagtttgactgtgcccttcatggaaaccactgggaggacctgggctttcagggagcgaatccagccacagacctgagaggcgcaggcttccttgccctcctgcatctgctctacctggtgatggactcaaagaccttgccgatggcgcaggagattttccgcctgtctcgtcaccacatccagcaattccctttctgtttgatgtccgtgaacatcacccacattgccatccaggccttgagagaggagtgtctctccagagagtgtaatcggcagcagaaggtcatccccgtggtgaacagcttctatgccgccacattcctccatctcgcacatgtctggaggacacagcggaagaccatctcagactcgggctttgtcctcaaagagttggaagtattggccaagaagagcccactgcggctgctcaagaccctggagctgtacttggccagggtgtcaaagggacaggcctccttgttgggagcacagaagtgctatgggccagaagcccctcccttcaaggatctcaccttcacaggtgagagtgacctgcagtctcactcatccgaaggcgtatggctgatctgacctccgagatgaatggaggcttaaaggctgagctgcaggggctttcagggggtcatgtcaggagcctggccaggccacaccccttgctgtctcagcagatgggatataggaagctcctgggcttagctgtgggaagccaagtaccctcaccggcatgggacatgaggggcagctagacttcacccccttcccgcagacctgcctccagagcaaggagaattctgccttaatctgttgggctccagtctccgggttgaattccagtgtatcccactgggagtgaatggatcatgaggtgggatggccccatctggatgttctgcagatccccacatgggaggagattcccaagtagaggcagccagaattctgactccctggcagcagcagggctttcaggcaccagtgtctgtgttgttagaactcagaggaagaggcaggggcagcagccgacagggggcagcatgacccaagcaaggggcctgaggcctcagtgggggtagggcaaggaggtacctcacaggtgggtgtgaggccccctctggagtttctggccattcacttacccactctcttctccccctgacccccgctccattgtttatgatggaaaaacggacatttggctaggtgtctctcacggctgctccatcctagcccccacaagctggggcttcctcctgtagatgctgtgcatgccccatgatgagtttctggcctaattgagggaaggaggaaattcataccagcagttttcaaataaaagaattgttctaaaaaaaaaaaaaaaaaaaaaa

SEQ ID NO: 119 - DNA, 1369 bp, Homo sapiens
ggccgacagtgcctgatttgagatggggtcccaggtctcggtggaatcgggagctctgcacgtggtgattgtgggtgggggctttggcgggatcgcagcagccagccagctgcaggccctgaacgtccccttcatgctggtggacatgaaggactccttccaccacaatgtggctgctctccgagcctccgtggagacagggttcgccaaaaagacattcatttcttactcggtgactttcaaggacaacttccggcaggggctagtagtggggatagacctgaagaaccagatggtgctgctgcagggtggcgaggccctgcccttctctcatcttatcctggccacgggcagcactgggcccttcccgggcaagtttaatgaggtttccagccagcaggccgctatccaggcctatgaggacatggtgaggcaggtccagcgctcacggttcatcgtggtggtgggaggaggctcggctggagtggagatggcagcagagattaaaacagaatatcctgagaaagaggtcactctcattcactcccaagtggccctggctgacaaggagctcctgccctccgtccggcaggaagtgaaggagatcctcctccggaagggcgtgcagctgctgctgagtgagcgggtgagcaatctggaggagctgcctctcaatgagtatcgagagtacatcaaagtgcagacggacaaaggcacagaggtggccaccaacctggtgattctctgcaccggcatcaagatcaacagctccgcctaccgcaaagcatttgagagcagactagccagcagtggtgctctgagagtgaacgagcacctccaggtggagggccacagcaacgtctacgccattggtgactgtgccgacgtgaggacgcccaagatggcctatcttgccggcctccacgccaacatcgccgtggccaacatcgtcaactctgtgaagcagcggcctctccaggcctacaagccgggtgcactgacgttcctcctgtccatggggagaaatgacggtgtgggccaaatcagtggcttctatgtgggccggctcatggttcggctgaccaagagccgggacctgttcgtctctacgagctggaaaaccatgaggcagtctccaccttgatggagaggccaggcgggagaactaccgcagcaggtgggcgtacggactgcttggcgcatggcacccgcctggcaagtgctagaactaatgctattcttctggaataagatgccaatgatgtggtggctagaaatgcaacttgtataaaacaaaaatgggagagagagaggtattaaacaaataccccccttagaggataaaaaaaaaaaaaaaaaaaaaaaaaaa

SEQ ID NO: 120 - DNA, 2509 bp, Homo sapiens
gagcgaaaggcttgaggaccaggtcggggccgggttccgggtcggggagcggctccgggcggcagctatgaggcgggacgtgcgcatcctgttactgggcgaggcccaggtggggaagacgtcgctgatcctgtccctggtgggcgaggagttccccgaggaggtccctccccgcgcggaggagatcacgatccccgcggacgtcaccccggagaaggtgcccacccacatcgtggactactcagaagccgagcagacggacgaggagctgcgggaggagatccacaaggcaaacgtggtgtgtgtggtgtatgacgtctctgaggaggccaccattgagaagattcgaactaagtggatcccactggtgaatggggggaccacgcaggggcccagggtgcccatcatcctagtgggcaacaagtcagacctgcggtcggggagctccatggaggccgtgctccccatcatgagccagtttcccgagattgagacctgcgtggagtgttcggccaagaacctgaggaacatctcagagctgttctactacgcccagaaggccgtcctgcatcccacagcccccctctatgaccctgaggccaagcagttgaggcccgcgtgcgcccaggcgctgacgcgcatcttcaggctctcagatcaggacctggaccaggcgctcagtgacgaagagctcaacgctttccagaaatcctgctttgggcaccccctggccccgcaggccctggaggacgtgaagacggtggtgtgcaggaacgtggcgggcggcgtgcgggaggaccggctgaccctggatggtttcctcttcctgaacacgctcttcatccagcgcggccggcacgagaccacctggaccatcctgcggcgcttcggctacagcgatgccctggagctgactgcggactatctctcccctctgatccacgtgccccccggctgcagcacggagctcaaccaccttggctaccagtttgtgcagagagtgtttgagaagcacgaccaggaccgcgacggcgccctctcgcccgtggagctgcaaagccttttcagtgtgttcccagcagcgccctggggccccgagctcccacgcacagtccgcacagaggccggccggttgcccctgcacggatacctctgccagtggaccctggtgacctacctggacgtccggagctgccttggacacctaggctacctgggctaccccaccctctgtgagcaggaccaggcccatgccatcacagtcactcgtgagaagaggctggaccaggagaagggacagacgcagcggagcgtcctcctgtgcaaggtggtaggggcccgtggagtgggcaagtctgccttcctgcaggcttttctcggccgcggcctggggcaccaggacacgagggagcagcctcccggctacgccatcgacacggtgcaggtcaatggacaggagaagtacttgatcctctgtgaggtgggcacagatggtctgctggccacatcgctggacgccacctgtgacgttgcctgcttgatgtttgatggcagtgacccaaagtcctttgcacattgtgccagcgtctacaagcaccattacatggacgggcagaccccctgcctctttgtctcctccaaggccgacctgcccgaaggtgtcgcggtgtctggcccatcaccggccgagttttgccgcaagcaccggctacccgctcccgtgccgttctcctgtgctggcccagccgagcccagcaccaccatcttcacccagctcgccaccatggccgccttcccacatttggtccacgcagagctgcatccctcttccttctggctccgggggctgctgggggttgtcggggccgccgtggccgcagtcctcagcttctcactctacagggtcctggtgaagagccagtgaggcccctggtacccaagccccctcccctgacctgggtgtgcctcgctgctggggctctgcaggggcagcacagctggggtgcaggccaggctgccactccgggaacgcctttgcgccgggactttttgtttctgaaggcagtcgatctgcagcggggccttatgctgccatgcactgccctggctcctgccggacccccagggtgggccgtggcaggtggctgagcaggagctcccaagtgccggccaccgctgtcagggattgcccacccctgggcatcatgtgtgtggggccggggagcacaggtgtgggagctggtgaccccagacccagaattctcagggctctacccccctttcctggtcctaggtggccagtgggtatgaggagggctggaaggcagagctttgggccaaaagcaggcgttggggggtcccccctcaagtttggagccgtttccgtggttgtagcagaggaccggaggttgggttcctgattaaacttcactgtgtgttttctatctcggatcccagtctctgaagacaacttgctttgattcaacctaaaaaaaaaaaaaaaaaaaaa

SEQ ID NO: 121 - DNA, 3190 bp, Homo sapiens
gtctggagagagcgcgcagtttgcgcggcggctcggcgcttccctgtcgtcgcactttgtggttgctgcagctcgggggcctgggctgcccctgacaccccttctgggcgatggtgcagcccaagggcgcctccatcccccgccgctgccgctaacccgggtcccccactccatggccgcctcggcgccgcccccaccggacaagctggagggaggtggcggccccgcaccgccccctgcgccgcccagcaccgggaggaagcagggcaaggccggtctgcaaatgaagagtccagaaaagaagcgaaggaagtcaaatactcagggccctgcatactcacatctgacggagtttgcaccacccccaactcccatggtggatcacctggttgcatccaacccttttgaagatgacttcggagcccccaaagtgggggttgcagcccctccattccttggcagtcctgtgcccttcggaggcttccgtgtgcaggggggcatggcgggccaggtacccccaggctacagcactggaggtggagggggcccccagccactccgtcgacagccaccccccttccctcccaatcctatgggccctgctttcaacatgcccccccagggtcctggctacccacccccaggcaacatgaactttcccagccaacccttcaaccagcctctgggtcaaaactttagtcctcccagtgggcagatgatgccgggcccagtggggggatttggtcccatgatctcacccaccatgggacagcctcccagagcagagctgggcccaccttctctgtcccaacgatttgctcagccaggggctccttttggcccttctcctctccagagacctggtcaggggctccccagcctgccgcctaacacaagtccctttcctggtccggaccctggctttcctggccctggtggtgaggatggggggaagcccttgaatccacctgcttctactgcttttccccaggagccccactcaggctccccggctgctgctgttaatgggaaccagcccagtttccccccgaacagcagtgggcggggtgggggcactccagatgccaacagcttggcaccccctggcaaggcaggtgggggctccgggccccagcctcccccaggcttggtgtacccatgtggtgcctgtcggagtgaggtgaacgatgaccaggatgccattctgtgtgaggcctcctgccagaaatggttccaccgtgagtgcacaggcatgactgagagcgcctatgggctgctgaccactgaagcttctgccgtctgggcctgcgatctctgcctcaagaccaaggagatccagtctgtctacatccgtgagggcatggggcagctggtggctgctaacgatgggtgacgctggtgaagtggcccagggaagtgcacatgtctctccctgctcttccagggtgatttttttgatgtttggctcttggtccttgtttccactggctttccatccccatggggcagaaacagtggctcctgggagcagaaaaggaattgaggtgggcaggcagaagagcctggattgctcactgttttgggaaacttacatgttgagatctacagagatccaggaaaccaaagccctgctgagcagagccattttgtggctatttctggaggcccaggagtgtggctgcaagagaaaaggggctggaggaagatccggagggcaggggtgttccctctgctgatgatggatgcccctaacacctgtgcctaacacccctactgaaccccacagctccagccttagtttttggagtcaagtgttaaaggtttctggccagaggaattggggtcttgccatccctgcaatagcccttttatgggctctgggagacagctttagggaataaatggggattttcccctttttctacccactcctttgcttcctccaagacttacccaactccttccccctcagagaaccaaatagcctgaggaagcaggagagttcctggttatggcagtttcttggtgatttggggcttcaagacagtaggtgagagatgctgtcaggacgtatcttcttcataccaaagtcactggtcctttctcagcctctctcgtgcttttctcctaatgaccatatttttgccaaaaattgggatatgttatctgacagaccagaatatttgaagtttgggctgtcctgaaagtctggactttggtggtaccctcctcccccagcccatctgttgcacattatactccgtgtgttcttcaactttcggcgcccttattcccctgccttcctggcttgattgaaggaaagcttgaaaaggcgcagagccctatacctcatttcctccatgataaaaggatccaagtgaggccctgtcacagcctgtgggtaggggatgcggcgggatcctcattgccatggtactcaaaggtagaagagcctggagtttgttgcttctctttgctattctttcatatcctcttgggcctggtgattaattagcaattctcattcctctcagccaaaggcctgcactgggctttatttgtctttttttattttttaagcactgcctgccagagatgggcctggggcctgatgaggaccttagcgctgctcgttctccttttctgttcatgcacacattcctccatggggtggggaaggcaggcatggggtgtggccctcggagaagttaggagtcccccagctcaagatacagtggcaaagacctagtggtcccctacccccacttctctcagttcctggcatgaggagagaagaccctgctctggtggagctgacaacctttgaggctgggaggagagcagcctctgggcatcgttcccagtgtccctcacactaaaacggcgtagatggcaaccccccacccccaccccgctgctcaactcttgtgtttgttgttctgtttgccccatttatctgttgctgtttttgtgttgtcttcccctgctccgcattttgtaaaatggcccctgggggagtgtttttgctggatctgctccctctcgctctctcactccactactttttggacaaagtgatggcagaatgcggtggtggtgggggtcttttgtactgttggattaataaaatgattttaaaatcccaaaaaaaaaaaaaaaaaaaaaaaaaaaaaaaaaaaaaaaaaaaaaa

SEQ ID NO: 122 - RNA, 22 nt, Epstein-Barr virus
uccugugguguuuggugugguu

SEQ ID NO: 123 - RNA, 22 nt, Epstein-Barr virus
uaaaugcugcaguaguagggau

SEQ ID NO: 124 - RNA, 24 nt, Epstein-Barr virus
uuagauagagugggugugugcucu

SEQ ID NO: 125 - RNA, 21 nt, Epstein-Barr virus
uagcaggcaugucuucauucc

SEQ ID NO: 126 - RNA, 22 nt, Epstein-Barr virus
uauuuucugcauucgcccuugc

SEQ ID NO: 127 - RNA, 22 nt, Homo sapiens
ugagguaguagguugugugguu

SEQ ID NO: 128 - RNA, 22 nt, Homo sapiens
ugagguaguagauuguauaguu

SEQ ID NO: 129 - RNA, 22 nt, Homo sapiens
ugagguaguagauuguauaguu

SEQ ID NO: 130 - RNA, 22 nt, Homo sapiens
ugagguaguaguuuguacaguu

SEQ ID NO: 131 - RNA, 23 nt, Homo sapiens
agcagcauuguacagggcuauga

SEQ ID NO: 132 - RNA, 23 nt, Homo sapiens
uacccuguagaaccgaauuugug

SEQ ID NO: 133 - RNA, 27 nt, Homo sapiens
cacuguaggugauggugagagugggca

SEQ ID NO: 134 - RNA, 21 nt, Homo sapiens
gugccagcugcagugggggag

SEQ ID NO: 135 - RNA, 21 nt, Homo sapiens
uggcagggaggcugggagggg

SEQ ID NO: 136 - RNA, 22 nt, Homo sapiens
uggagugugacaaugguguuug

SEQ ID NO: 137 - RNA, 19 nt, Homo sapiens
gugaggacucgggaggugg

SEQ ID NO: 138 - RNA, 22 nt, Homo sapiens
guggguacggcccagugggggg

SEQ ID NO: 139 - RNA, 22 nt, Homo sapiens
guggguacggcccagugggggg

SEQ ID NO: 140 - RNA, 22 nt, Homo sapiens
ucaccagcccuguguucccuag

SEQ ID NO: 141 - RNA, 20 nt, Homo sapiens
ucacaccugccucgcccccc

SEQ ID NO: 142 - RNA, 22 nt, Homo sapiens
ucggccugaccacccaccccac

SEQ ID NO: 143 - RNA, 21 nt, Homo sapiens
uccuucugcuccgucccccag

SEQ ID NO: 144 - RNA, 20 nt, Homo sapiens
cuuccucgucugucugcccc

SEQ ID NO: 145 - RNA, 24 nt, Homo sapiens
ucccugagacccuuuaaccuguga

SEQ ID NO: 146 - RNA, 18 nt, Homo sapiens
aucccaccucugccacca

SEQ ID NO: 147 - RNA, 19 nt, Homo sapiens
aucccaccacugccaccau

SEQ ID NO: 148 - RNA, 17 nt, Homo sapiens
ucccaccgcugccaccc

SEQ ID NO: 149 - RNA, 17 nt, Homo sapiens
ucgccuccuccucuccc

SEQ ID NO: 150 - RNA, 19 nt, Homo sapiens
uggauuuuuggaucaggga

SEQ ID NO: 151 - RNA, 24 nt, Homo sapiens
uggcccugacugaagaccagcagu

SEQ ID NO: 152 - RNA, 21 nt, Homo sapiens
cuuuuugcggucugggcuugc

SEQ ID NO: 153 - RNA, 24 nt, Homo sapiens
uugcagcugccugggagugacuuc

SEQ ID NO: 154 - RNA, 23 nt, Homo sapiens
uauggcuuuuuauuccuauguga

SEQ ID NO: 155 - RNA, 23 nt, Homo sapiens
uauggcuuuuuauuccuauguga

SEQ ID NO: 156 - RNA, 22 nt, Homo sapiens
uaacacugucugguaaagaugg

SEQ ID NO: 157 - RNA, 21 nt, Homo sapiens
cauaaaguagaaagcacuacu

SEQ ID NO: 158 - RNA, 23 nt, Homo sapiens
ucuggcuccgugucuucacuccc

SEQ ID NO: 159 - RNA, 22 nt, Homo sapiens
ucucccaacccuuguaccagug

SEQ ID NO: 160 - RNA, 22 nt, Homo sapiens
ucucccaacccuuguaccagug

SEQ ID NO: 161 - RNA, 21 nt, Homo sapiens
uccugcgcgucccagaugccc

SEQ ID NO: 162 - RNA, 23 nt, Homo sapiens
aacauucaacgcugucggugagu

SEQ ID NO: 163 - RNA, 18 nt, Homo sapiens
uccagugcccuccucucc

SEQ ID NO: 164 - RNA, 22 nt, Homo sapiens
caaagaauucuccuuuugggcu

SEQ ID NO: 165 - RNA, 23 nt, Homo sapiens
uaaggugcaucuagugcagauag

SEQ ID NO: 166 - RNA, 23 nt, Homo sapiens
uaaggugcaucuagugcaguuag

SEQ ID NO: 167 - RNA, 23 nt, Homo sapiens
caacggaaucccaaaagcagcug

SEQ ID NO: 168 - RNA, 22 nt, Homo sapiens
uucaccaccuucuccacccagc

SEQ ID NO: 169 - RNA, 22 nt, Homo sapiens
gguccagaggggagauagguuc

SEQ ID NO: 170 - RNA, 22 nt, Homo sapiens
uccuucauuccaccggagucug

SEQ ID NO: 171 - RNA, 21 nt, Homo sapiens
gguucuuagcauaggaggucu

SEQ ID NO: 172 - RNA, 21 nt, Homo sapiens
augaccuaugaauugacagac

SEQ ID NO: 173 - RNA, 22 nt, Homo sapiens
aagcugccaguugaagaacugu

SEQ ID NO: 174 - RNA, 22 nt, Homo sapiens
ugucaguuugucaaauacccca

SEQ ID NO: 175 - RNA, 22 nt, Homo sapiens
ucugcaagugucagaggcgagg

SEQ ID NO: 176 - RNA, 22 nt, Homo sapiens
aucacauugccagugauuaccc

SEQ ID NO: 177 - RNA, 22 nt, Homo sapiens
uucaaguaauccaggauaggcu

SEQ ID NO: 178 - RNA, 22 nt, Homo sapiens
aaggagcucacagucuauugag

SEQ ID NO: 179 - RNA, 22 nt, Homo sapiens
aaggagcucacagucuauugag

SEQ ID NO: 180 - RNA, 23 nt, Homo sapiens
uagcaccauuugaaaucaguguu

SEQ ID NO: 181 - RNA, 22 nt, Homo sapiens
uguaaacauccucgacuggaag

SEQ ID NO: 182 - RNA, 22 nt, Homo sapiens
uguaaacauccuacacucagcu

SEQ ID NO: 183 - RNA, 23 nt, Homo sapiens
ucgaggacugguggaagggccuu

SEQ ID NO: 184 - RNA, 24 nt, Homo sapiens
uguggacagugagguagagggagu

SEQ ID NO: 185 - RNA, 23 nt, Homo sapiens
uuuguauggauauguguguguau

SEQ ID NO: 186 - RNA, 22 nt, Homo sapiens
aaagaucuggaagugggagaca

SEQ ID NO: 187 - RNA, 19 nt, Homo sapiens
uggggcggagcuuccggag

SEQ ID NO: 188 - RNA, 21 nt, Homo sapiens
ggccagccaccaggagggcug

SEQ ID NO: 189 - RNA, 17 nt, Homo sapiens
cgcgccgggcccggguu

SEQ ID NO: 190 - RNA, 18 nt, Homo sapiens
cggggcggcaggggccuc

SEQ ID NO: 191 - RNA, 22 nt, Homo sapiens
uauugcacauuacuaaguugca

SEQ ID NO: 192 - RNA, 20 nt, Homo sapiens
aaaagcuggguugagagggu

SEQ ID NO: 193 - RNA, 23 nt, Homo sapiens
cgcauccccuagggcauuggugu

SEQ ID NO: 194 - RNA, 22 nt, Homo sapiens
cuggcccucucugcccuuccgu

SEQ ID NO: 195 - RNA, 22 nt, Homo sapiens
cuagguauggucccagggaucc

SEQ ID NO: 196 - RNA, 20 nt, Homo sapiens
gugcauugcuguugcauugc

SEQ ID NO: 197 - RNA, 21 nt, Homo sapiens
aggggugcuaucugugauuga

SEQ ID NO: 198 - RNA, 22 nt, Homo sapiens
uggcagugucuuagcugguugu

SEQ ID NO: 199 - RNA, 20 nt, Homo sapiens
gaaucggaaaggaggcgccg

SEQ ID NO: 200 - RNA, 21 nt, Homo sapiens
agccgcggggaucgccgaggg

SEQ ID NO: 201 - RNA, 18 nt, Homo sapiens
cggcuggaggugugagga

SEQ ID NO: 202 - RNA, 21 nt, Homo sapiens
gcuggucugcguggugcucgg

SEQ ID NO: 203 - RNA, 22 nt, Homo sapiens
aaagacccauugaggagaaggu

SEQ ID NO: 204 - RNA, 22 nt, Homo sapiens
gaaguuguucgugguggauucg

SEQ ID NO: 205 - RNA, 22 nt, Homo sapiens
uguguggauccuggaggaggca

SEQ ID NO: 206 - RNA, 23 nt, Homo sapiens
acaggcggcuguagcaauggggg

SEQ ID NO: 207 - RNA, 18 nt, Homo sapiens
ccagagguggggacugag

SEQ ID NO: 208 - RNA, 23 nt, Homo sapiens
aaugacacgaucacucccguuga

SEQ ID NO: 209 - RNA, 20 nt, Homo sapiens
ucagggagucaggggagggc

SEQ ID NO: 210 - RNA, 18 nt, Homo sapiens
cagcagucccucccccug

SEQ ID NO: 211 - RNA, 18 nt, Homo sapiens
gggucccggggagggggg

SEQ ID NO: 212 - RNA, 18 nt, Homo sapiens
gggcucacaucaccccau

SEQ ID NO: 213 - RNA, 17 nt, Homo sapiens
accccacuccugguacc

SEQ ID NO: 214 - RNA, 22 nt, Homo sapiens
uaauacugucugguaaaaccgu

SEQ ID NO: 215 - RNA, 19 nt, Homo sapiens
ugcccuccuuucuucccuc

SEQ ID NO: 216 - RNA, 20 nt, Homo sapiens
agcccccuggccccaaaccc

SEQ ID NO: 217 - RNA, 18 nt, Homo sapiens
cagccccacagccucaga

SEQ ID NO: 218 - RNA, 23 nt, Homo sapiens
auacacauacacgcaacacacau

SEQ ID NO: 219 - RNA, 22 nt, Homo sapiens
aagacgggaggaaagaagggag

SEQ ID NO: 220 - RNA, 22 nt, Homo sapiens
ucaggcucaguccccucccgau

SEQ ID NO: 221 - RNA, 22 nt, Homo sapiens
agaggcuggccgugaugaauuc

SEQ ID NO: 222 - RNA, 22 nt, Homo sapiens
uccuguacugagcugccccgag

SEQ ID NO: 223 - RNA, 21 nt, Homo sapiens
uugaaaggcuauuucuugguc

SEQ ID NO: 224 - RNA, 21 nt, Homo sapiens
cagcagcacacugugguuugu

SEQ ID NO: 225 - RNA, 21 nt, Homo sapiens
gugucuuuugcucugcaguca

SEQ ID NO: 226 - RNA, 21 nt, Homo sapiens
gugucuuuugcucugcaguca

SEQ ID NO: 227 - RNA, 22 nt, Homo sapiens
cucuagagggaagcacuuucug

SEQ ID NO: 228 - RNA, 23 nt, Homo sapiens
agugccugagggaguaagagccc

SEQ ID NO: 229 - RNA, 22 nt, Homo sapiens
gaugagcucauuguaauaugag

SEQ ID NO: 230 - RNA, 23 nt, Homo sapiens
guuugcacgggugggccuugucu

SEQ ID NO: 231 - RNA, 22 nt, Homo sapiens
caaaguuuaagauccuugaagu

SEQ ID NO: 232 - RNA, 20 nt, Homo sapiens
guccgcucggcgguggccca

SEQ ID NO: 233 - RNA, 23 nt, Homo sapiens
ugagugugugugugugagugugu

SEQ ID NO: 234 - RNA, 23 nt, Homo sapiens
ugagugugugugugugagugugu

SEQ ID NO: 235 - RNA, 21 nt, Homo sapiens
gaagugugccguggugugucu

SEQ ID NO: 236 - RNA, 22 nt, Homo sapiens
aguauucuguaccagggaaggu

SEQ ID NO: 237 - RNA, 22 nt, Homo sapiens
agacacauuuggagagggaccc

SEQ ID NO: 238 - RNA, 19 nt, Homo sapiens
aagcagcugccucugaggc

SEQ ID NO: 239 - RNA, 22 nt, Homo sapiens
cuugguucagggagggucccca

SEQ ID NO: 240 - RNA, 22 nt, Homo sapiens
uacccauugcauaucggaguug

SEQ ID NO: 241 - RNA, 22 nt, Homo sapiens
aggcggggcgccgcgggaccgc

SEQ ID NO: 242 - RNA, 23 nt, Homo sapiens
uauucauuuauccccagccuaca

SEQ ID NO: 243 - RNA, 17 nt, Homo sapiens
ucucgcuggggccucca

SEQ ID NO: 244 - RNA, 22 nt, Homo sapiens
ggggcuggggccggggccgagc

SEQ ID NO: 245 - RNA, 22 nt, Homo sapiens
acuccagccccacagccucagc

SEQ ID NO: 246 - RNA, 20 nt, Homo sapiens
guagaggagauggcgcaggg

SEQ ID NO: 247 - RNA, 21 nt, Homo sapiens
uacucaaaaagcugucaguca

SEQ ID NO: 248 - RNA, 22 nt, Homo sapiens
ugugcgcagggagaccucuccc

SEQ ID NO: 249 - RNA, 21 nt, Homo sapiens
aaggcagggcccccgcucccc

SEQ ID NO: 250 - RNA, 22 nt, Homo sapiens
uucaacggguauuuauugagca

SEQ ID NO: 251 - RNA, 23 nt, Herpes simplex virus I
uacaccccccugccuuccacccu

SEQ ID NO: 252 - RNA, 24 nt, Herpes simplex virus I
ccugagccagggacgagugcgacu

SEQ ID NO: 253 - RNA, 21 nt, Herpes simplex virus I
cacuucccguccuuccauccc

SEQ ID NO: 254 - RNA, 21 nt, Herpes simplex virus I
uuuggaucccgaccccucuuc

SEQ ID NO: 255 - RNA, 20 nt, Herpes simplex virus I
uauauagggucaggggguuc

SEQ ID NO: 256 - RNA, 22 nt, Herpes simplex virus II
aggggucuggacgugggugggc

SEQ ID NO: 257 - RNA, 21 nt, Herpes simplex virus II
cugcgcggcggagaccgggac

SEQ ID NO: 258 - RNA, 20 nt, Herpes simplex virus II
aauggaaggcgaggggaugc

SEQ ID NO: 259 - RNA, 23 nt, Herpes simplex virus II
cccaucuucugcccuuccauccu

SEQ ID NO: 260 - RNA, 22 nt, Kaposi′s sarcoma-associated herpesvirus
uaguguuguccccccgaguggc

SEQ ID NO: 261 - RNA, 22 nt, Kaposi′s sarcoma-associated herpesvirus
ugggggagggugcccugguuga

SEQ ID NO: 262 - RNA, 22 nt, Kaposi′s sarcoma-associated herpesvirus
acucccucacuaacgccccgcu

SEQ ID NO: 263 - RNA, 19 nt, Homo sapiens
aggcacggugucagcaggc

SEQ ID NO: 264 - RNA, 22 nt, Homo sapiens
agagguaguagguugcauaguu

SEQ ID NO: 265 - RNA, 22 nt, Homo sapiens
cuauacgaccugcugccuuucu

SEQ ID NO: 266 - RNA, 22 nt, Homo sapiens
uagcaccaucugaaaucgguua

SEQ ID NO: 267 - RNA, 22 nt, Homo sapiens
agaguugagucuggacgucccg

SEQ ID NO: 268 - RNA, 22 nt, Homo sapiens
gaggguuggguggaggcucucc

SEQ ID NO: 269 - RNA, 21 nt, Homo sapiens
gaacggcuucauacaggaguu

SEQ ID NO: 270 - RNA, 21 nt, Homo sapiens
aauaauacaugguugaucuuu

SEQ ID NO: 271 - RNA, 22 nt, Homo sapiens
uuuugcaauauguuccugaaua

SEQ ID NO: 272 - RNA, 22 nt, Homo sapiens
aagacgggaggaaagaagggag

SEQ ID NO: 273 - RNA, 21 nt, Homo sapiens
uuuugcaccuuuuggagugaa

SEQ ID NO: 274 - RNA, 22 nt, Homo sapiens
aucgugcauccuuuuagagugu

SEQ ID NO: 275 - RNA, 22 nt, Homo sapiens
aaagugcauccuuuuagagugu

SEQ ID NO: 276 - RNA, 22 nt, Homo sapiens
caaagugccucccuuuagagug

SEQ ID NO: 277 - RNA, 24 nt, Homo sapiens
acaaagugcuucccuuuagagugu

SEQ ID NO: 278 - RNA, 22 nt, Homo sapiens
auucuaauuucuccacgucuuu

SEQ ID NO: 279 - RNA, 21 nt, Homo sapiens
uagauaaaauauugguaccug

SEQ ID NO: 280 - RNA, 19 nt, Homo sapiens
aggcugcggaauucaggac

SEQ ID NO: 281 - RNA, 21 nt, Homo sapiens
ugagcuaaaugugugcuggga

SEQ ID NO: 282 - RNA, 24 nt, Homo sapiens
gaccuggacauguuugugcccagu

SEQ ID NO: 283 - RNA, 22 nt, Homo sapiens
aggcauugacuucucacuagcu

SEQ ID NO: 284 - RNA, 22 nt, Homo sapiens
uaguacugugcauaucaucuau

SEQ ID NO: 285 - RNA, 17 nt, Homo sapiens
uucaaguaauucaggug

SEQ ID NO: 286 - RNA, 22 nt, Human cytomegalovirus
gauugugcccggaccgugggcg

SEQ ID NO: 287 - RNA, 21 nt, Herpes simplex virus I
gauggaaggacgggaagugga

SEQ ID NO: 288 - RNA, 20 nt, Homo sapiens
guagaggagauggcgcaggg

SEQ ID NO: 289 - RNA, 22 nt, Homo sapiens
ugagguaggagguuguauaguu

SEQ ID NO: 290 - RNA, 22 nt, Homo sapiens
cuauacggccuccuagcuuucc

SEQ ID NO: 291 - RNA, 23 nt, Homo sapiens
ucuuugguuaucuagcuguauga

SEQ ID NO: 292 - RNA, 22 nt, Homo sapiens
uauugcacucgucccggccucc

SEQ ID NO: 293 - RNA, 23 nt, Homo sapiens
uuuggcacuagcacauuuuugcu

SEQ ID NO: 294 - RNA, 22 nt, Homo sapiens
aaucaugugcagugccaauaug

SEQ ID NO: 295 - RNA, 23 nt, Homo sapiens
aaaagugcuuacagugcagguag

SEQ ID NO: 296 - RNA, 22 nt, Homo sapiens
cugcaauguaagcacuucuuac

SEQ ID NO: 297 - RNA, 23 nt, Homo sapiens
caaagugcuuacagugcagguag

SEQ ID NO: 298 - RNA, 22 nt, Homo sapiens
acugcagugaaggcacuuguag

SEQ ID NO: 299 - RNA, 21 nt, Homo sapiens
uaaagugcugacagugcagau

SEQ ID NO: 300 - RNA, 22 nt, Homo sapiens
ccgcacuguggguacuugcugc

SEQ ID NO: 301 - RNA, 22 nt, Homo sapiens
ucggauccgucugagcuuggcu

SEQ ID NO: 302 - RNA, 21 nt, Homo sapiens
ucacagugaaccggucucuuu

SEQ ID NO: 303 - RNA, 22 nt, Homo sapiens
uagguuauccguguugccuucg

SEQ ID NO: 304 - RNA, 23 nt, Homo sapiens
uuaaugcuaaucgugauaggggu

SEQ ID NO: 305 - RNA, 22 nt, Homo sapiens
uauggcacugguagaauucacu

SEQ ID NO: 306 - RNA, 22 nt, Homo sapiens
gugaauuaccgaagggccauaa

SEQ ID NO: 307 - RNA, 22 nt, Homo sapiens
uguaacagcaacuccaugugga

SEQ ID NO: 308 - RNA, 22 nt, Homo sapiens
uagguaguuuccuguuguuggg

SEQ ID NO: 309 - RNA, 22 nt, Homo sapiens
ucgacagcacgacacugccuuc

SEQ ID NO: 310 - RNA, 22 nt, Homo sapiens
gugaaauguuuaggaccacuag

SEQ ID NO: 311 - RNA, 22 nt, Homo sapiens
uucccuuugucauccuaugccu

SEQ ID NO: 312 - RNA, 22 nt, Homo sapiens
accuggcauacaauguagauuu

SEQ ID NO: 313 - RNA, 23 nt, Homo sapiens
agcuacauugucugcuggguuuc

SEQ ID NO: 314 - RNA, 21 nt, Homo sapiens
aucauagaggaaaauccacgu

SEQ ID NO: 315 - RNA, 21 nt, Homo sapiens
aacauagaggaaauuccacgu

SEQ ID NO: 316 - RNA, 22 nt, Homo sapiens
aucacacaaaggcaacuuuugu

SEQ ID NO: 317 - RNA, 21 nt, Homo sapiens
ugguagacuauggaacguagg

SEQ ID NO: 318 - RNA, 23 nt, Homo sapiens
agcucggucugaggccccucagu

SEQ ID NO: 319 - RNA, 22 nt, Homo sapiens
cagcagcaauucauguuuugaa

SEQ ID NO: 320 - RNA, 21 nt, Homo sapiens
caaaacgugaggcgcugcuau

SEQ ID NO: 321 - RNA, 23 nt, Homo sapiens
aaugacacgaucacucccguuga

SEQ ID NO: 322 - RNA, 22 nt, Homo sapiens
aucgggaaugucguguccgccc

SEQ ID NO: 323 - RNA, 22 nt, Homo sapiens
acccuaucaauauugucucugc

SEQ ID NO: 324 - RNA, 23 nt, Homo sapiens
uagugcaauauugcuuauagggu

SEQ ID NO: 325 - RNA, 21 nt, Homo sapiens
cggggcagcucaguacaggau

SEQ ID NO: 326 - RNA, 22 nt, Homo sapiens
ugauugguacgucuguggguag

SEQ ID NO: 327 - RNA, 21 nt, Homo sapiens
auugacacuucugugaguaga

SEQ ID NO: 328 - RNA, 22 nt, Homo sapiens
ugugacagauugauaacugaaa

SEQ ID NO: 329 - RNA, 22 nt, Homo sapiens
ucaguaaauguuuauuagauga

SEQ ID NO: 330 - RNA, 22 nt, Homo sapiens
ucagcaaacauuuauugugugc

SEQ ID NO: 331 - RNA, 19 nt, Homo sapiens
agcugucugaaaaugucuu

SEQ ID NO: 332 - RNA, 22 nt, Homo sapiens
gaugguugaccagagagcacac

SEQ ID NO: 333 - RNA, 22 nt, Homo sapiens
uuugugaccugguccacuaacc

SEQ ID NO: 334 - RNA, 22 nt, Homo sapiens
uggugguuuacaaaguaauuca

SEQ ID NO: 335 - RNA, 21 nt, Homo sapiens
agaggauacccuuuguauguu

SEQ ID NO: 336 - RNA, 23 nt, Homo sapiens
ccucagggcuguagaacagggcu

SEQ ID NO: 337 - RNA, 22 nt, Homo sapiens
cccuguucuaugcccugaggga

SEQ ID NO: 338 - RNA, 22 nt, Homo sapiens
acaguagucugcacauugguua

SEQ ID NO: 339 - RNA, 22 nt, Homo sapiens
acaguagucugcacauugguua

SEQ ID NO: 340 - RNA, 21 nt, Homo sapiens
agggggaaaguucuauagucc

SEQ ID NO: 341 - RNA, 22 nt, Homo sapiens
gacuauagaacuuucccccuca

SEQ ID NO: 342 - RNA, 21 nt, Homo sapiens
agaccuggcccagaccucagc

SEQ ID NO: 343 - RNA, 23 nt, Homo sapiens
acuugggcacugaaacaaugucc

SEQ ID NO: 344 - DNA, 950 bp, Homo sapiens
ggcagccgaggagaccccgcgcagtgctgccaacgccccggtggagaagctgaggtcatcatcagatttgaaatatttaaagtggatacaaaactatttcagcaatgcagacaattaagtgtgttgttgtgggcgatggtgctgttggtaaaacatgtctcctgatatcctacacaacaaacaaatttccatcggaatatgtaccgactgtttttgacaactatgcagtcacagttatgattggtggagaaccatatactcttggactttttgatactgcagggcaagaggattatgacagattacgaccgctgagttatccacaaacagatgtatttctagtctgtttttcagtggtctctccatcttcatttgaaaacgtgaaagaaaagtgggtgcctgagataactcaccactgtccaaagactcctttcttgcttgttgggactcaaattgatctcagagatgacccctctactattgagaaacttgccaagaacaaacagaagcctatcactccagagactgctgaaaagctggcccgtgacctgaaggctgtcaagtatgtggagtgttctgcacttacacagaaaggcctaaagaatgtatttgacgaagcaatattggctgccctggagcctccagaaccgaagaagagccgcaggtgtgtgctgctatgaacatctctccagagccctttctgcacagctggtgtcggcatcatactaaaagcaatgtttaaatcaaactaaagattaaaaattaaaattcgtttttgcaataatgacaaatgccctgcacctacccacatgcactcgtgtgagacaaggcccataggtatggccccccccttccccctcccagtactagttaattttgagtaattgtattgtcagaaaagtgattagtactattttttttttgttgtttcaaaaaaaaaaaaaaaaaaa

SEQ ID NO: 345 - DNA, 3859 bp, Homo sapiens
gtccgggcagcccccggcgcagcgcggccgcagcagcctcctccccccgcacggtgtgagcgcccgccgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacagggacaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctggtgagtggcttgtctggaaacagtcctgctcctcaacctcctcgacccactcagcagcagccagtctccagtgtccaagccaggtgctccctccagcatctccagagggggaaacagtggcagatttgcagacacagtgaagggcgtaaggagcagataaacacatgaccgagcctgcacaagctctttgttgtgtctggttgtttgctgtacctctgttgtaagaatgaatctgcaaaatttctagcttatgaagcaaatcacggacatacacatctgtatgtgtgagtgttcatgatgtgtgtacatctgtgtatgtgtgtgtgtgtatgtgtgtgtttgtgacagatttgatccctgttctctctgctggctctatcttgacctgtgaaacgtatatttaactaattaaatattagttaatattaataaattttaagctttatccagaaaaaaaaaaaaaaaa

SEQ ID NO: 346 - DNA, 5179 bp, Homo sapiens
ggctttgccgcgctcgctgcacttgggcgagagctggaacgtggaccagagctcggatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccatagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacatgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatttagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaactaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttggatattcttgaaagtttatatttttataattttttcttacatcagatgtttctttgcagtggcttaatgtttgaaattattttgtggctttttttgtaaatattgaaatgtagcaataatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattctaataaaatgtacatatataaatcaaaaaaaaaaaaaaaa

SEQ ID NO: 347 - DNA, 1767 bp, Homo sapiens
ccagaagcctgcatttctgcattctgcttaattccctttccttagatttgaaagaagccaacactaaaccacaaatatacaacaaggccattttctcaaacgagagtcagcctttaacgaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaaaaaaaaaaaaaaa

SEQ ID NO: 348 - DNA, 2473 bp, Homo sapiens
gctttcaccactgcccctcccggggggacctgaaggagagggtttgaggccggtctttgcccgccgaggtctgcgtgtccggtctgggaggaggcctaggagggctcgcgggccacggcatccttgggcccgagttctggggtgcggacggacgtctcgagagtgggtgccgcgactcgggacccacggccctcgccgggcacggacagttgcggagcagggctctgaggattgtgcagtgccctgggtccctgcctactcctgggctcaggaatggagaagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggagttgctgctgggagctccagctcagtgaaatggacagaagggcagagcaaccacagcacagggtacgagagcgataaccacacaacgcccatcctctgcggagcccaatacagaatgcacacgcacggtgtcttcagaggcattcaggatgtgcggcgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttctcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttgaggggtctccctcggggaccgttcagtgtcccaggcagcacagtgtgtgaactgctttcaagtctgactctccactcctcctcactaaaaaggaaacttcagttgatcttcttcatccaacttccaagacaagataccggtgcttctggaaactaccaggtgtgcctggaagagttggtctctgccctgcctacttttagttgactcacaggccctggagaagcagctaacaatgtctggttagttaaaagcccattgccatttggtctggattttctactgtaagaagagccatagctgatcatgtccccctgacccttcccttctttttttatgctcgttttcgctggggatggaattattgtaccattttctatcatggaatatttataggccagggcatgtgtatgtgtctgctaatgtaaactttgtcatggtttccatttactaacagcaacagcaagaaataaatcagagagcaaggcatcgggggtgaatcttgtctaacattcccgaggtcagccaggctgctaacctggaaagcaggatgtagttctgccaggcaacttttaaagctcatgcatttcaagcagctgaagaaagaatcagaactaaccagtacctctgtatagaaatctaaaagaattttaccattcagttaattcaatgtgaacactggcacactgctcttaagaaactatgaagatctgagatttttttgtgtatgtttttgactcttttgagtggtaatcatatgtgtctttatagatgtacatacctccttgcacaaatggaggggaattcattttcatcactgggagtgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaaatgagacttactgggtgaggaaatccattgtttaaagatggtcgtgtgtgtgtgtgtgtgtgtgtgtgtgttgtgttgtgttttattttttaagggagggaatttattatttaccgttgcttgaaattactgtgtaaatatatgtctgttaatgatttgctctttgacaactaaaattaggactgtataagtactagatgcatcactgggtgttgatcttacaagatattgatgataacacttaaaattgtaacctgcatttttcactttgctctcaattaaagtctattcaaaaggaaaaaaaaaaaaaaaa